(19) United States
(12) Patent Application Publication
(10) Pub. No.: US 2025/0296915 A1
(43) Pub. Date: Sep. 25, 2025

(54) NOVEL AMINO ACID DERIVATIVES

(71) Applicant: PeptiDream Inc., Kawasaki-shi (JP)

(72) Inventors: Haruaki KURASAKI, Kawasaki-shi (JP); Naoki OKADA, Kawasaki-shi (JP); Katsuma MATSUI, Kawasaki-shi (JP); Kyosuke UEDA, Kawasaki-shi (JP); Masatoshi MATSUMOTO, Kawasaki-shi (JP); Atsushi YOSHIZAWA, Kawasaki-shi (JP); Akihiro KUROO, Kawasaki-shi (JP); Ayumu MATSUDA, Kawasaki-shi (JP); Motoki MURAI, Kawasaki-shi (JP); Yutaka KOBAYASHI, Kawasaki-shi (JP); Keiichi MASUYA, Kawasaki-shi (JP); Yoshihide MIZUKOSHI, Kawasaki-shi (JP); Douglas Robert CARY, Kawasaki-shi (JP); Makoto JITSUOKA, Kawasaki-shi (JP); Yoshihiro SHIBATA, Kawasaki-shi (JP); Kotaro TOKUMOTO, Kawasaki-shi (JP); Motoi KUWABARA, Kawasaki-shi (JP); Yusaku TAKAHASHI, Kawasaki-shi (JP); Tomokazu TAMAOKI, Kawasaki-shi (JP)

(21) Appl. No.: 19/084,270

(22) Filed: Mar. 19, 2025

Related U.S. Application Data
(60) Provisional application No. 63/567,948, filed on Mar. 21, 2024.

(51) Int. Cl. C07D 213/81; C07C 229/14; C07C 229/16; C07C 229/22; C07C 229/26; C07C 229/28; C07C 229/36; C07C 229/38; C07C 237/12; C07C 237/30; C07C 311/06; C07C 311/37; C07D 205/04; C07D 209/20; C07D 211/14; C07D 211/34; C07D 211/52; C07D 213/30; C07D 213/55; C07D 213/64; C07D 213/643; C07D 213/65; C07D 213/75; C07D 213/79; C07D 215/14; C07D 215/227; C07D 215/48; C07D 231/12; C07D 231/56; C07D 239/30; C07D 239/42; C07D 239/84; C07D 265/36; C07D 277/30; C07D 295/15; C07D 295/215; C07D 309/06; C07D 311/88; C07D 317/30; C07D 319/18; C07D 401/06; C07D 401/12; C07D 403/04; C07D 471/04; C07D 513/14

(52) U.S. Cl. CPC C07D 213/81; C07C 229/14; C07C 229/16; C07C 229/22; C07C 229/26; C07C 229/28; C07C 229/36; C07C 229/38; C07C 237/12; C07C 237/30; C07C 311/06; C07C 311/37; C07D 205/04; C07D 209/20; C07D 211/14; C07D 211/34; C07D 211/52; C07D 213/30; C07D 213/55; C07D 213/64; C07D 213/643; C07D 213/65; C07D 213/75; C07D 213/79; C07D 215/14; C07D 215/227; C07D 215/48; C07D 231/12; C07D 231/56; C07D 239/30; C07D 239/42; C07D 239/84; C07D 265/36; C07D 277/30; C07D 295/15; C07D 295/215; C07D 309/06; C07D 311/88; C07D 317/30; C07D 319/18; C07D 401/06; C07D 401/12; C07D 403/04; C07D 471/04; C07D 513/14; C07C 2601/02; C07C 2601/08; C07C 2601/14

(57) ABSTRACT

A novel amino acid derivative is provided, wherein the amino acid derivative is expected to improve binding affinity of polypeptides comprising the derivative therein.

### CROSS REFERENCE TO RELATED APPLICATION

This application claims the benefit of U.S. Provisional Patent Application Ser. No. 63/567,948, filed Mar. 21, 2024. The entire content of the above-referenced patent application is incorporated by reference in its entirety herein.

### FIELD OF THE DISCLOSURE

This disclosure relates to novel amino acid derivatives.

### BACKGROUND OF THE DISCLOSURE

Recently peptides are paid attention to as pharmaceutical substances. Such peptides interact with the pharmaceutical target molecule to act and exhibit any pharmacological effect. One of the important features needed for these peptides is binding affinity. The strength of affinity varies on target and the purpose of such pharmaceuticals.

The binding affinity of a polypeptide is primarily dependent on the physicochemical properties of its amino acids. To improve affinity, there is a demand for novel amino acid having various type of functional groups on their side chains to fit the structure of target molecules.

### SUMMARY OF THE DISCLOSURE

Generally, however, demand for polypeptides with improved binding affinity remains high.

In one aspect, a novel amino acid derivative which is expected to improve the binding affinity of polypeptides is provided.

In another aspect, a novel amino acid derivative wherein one or more functional groups within the amino acid are protected by protecting groups is provided.

In a further aspect, an enantiomer of the novel amino acid derivative is pro vided.

### DETAILED DESCRIPTION OF THE DISCLOSURE

### Abbreviation

AcOH acetic acid (CAS: 64-19-7)
aq. aqueous
Boc tert-butyloxycarbonyl
Boc₂O di-tert-butyl decarbonate (CAS: 24424-99-5)
br broad
BSA N,O-Bis(trimethylsilyl)acetamide (CAS: 10416-59-8)
conc. concentrated
CPME cyclopentyl methyl ether (CAS: 5614-37-9)
CSA 10-Camphorsulfonic acid (CAS: 5872-08-2)
d doublet
DCE 1,2-dichloroethane (CAS: 107-06-2)
DCM dichloromethane (CAS: 75-09-2)
dd double doublet
DIPEA, DIEA N,N-diisopropylethylamine (CAS: 7087-68-5)
DMA N,N-dimethylacetamide (CAS: 127-19-5)
DMAP 4-dimethylaminopyridine (CAS: 1122-58-3)
DME 1,2-dimethoxyethane (CAS: 110-71-4)
DMF N,N-dimethylformamide (CAS: 68-12-2)
EDCI·HCl ({[3-(dimethylamino)propyl]imino}methylidene)(ethyl)amine hydrochloride (CAS: 25952-53-8)
EtOAc ethyl acetate (CAS: 141-78-6)
Et ethyl
Et₂O diethyl ether (CAS: 60-29-7)
Et₃N triethylamine (CAS: 121-44-8)
Fmoc 9-fluorenylmethoxycarbonyl
Fmoc-OSu 2,5-dioxopyrrolidin-1-yl 9H-fluoren-9-ylmethyl carbonate (CAS: 82911-69-1)
h hour
HATU 1-[bis(dimethylamino)methylene]-1H-1,2,3-triazolo[4,5-b]pyridinium 3-oxide hexafluorophosphate (CAS: 148893-10-1)
IPE, iPr₂O diisopropyl ether (CAS: 108-20-3)
iPrOAc isopropyl acetate (CAS: 108-21-4)
iPrOH 2-propanol, isopropanol (CAS: 67-63-0)
iPr isopropyl
M molar
m multiplet
MeCN acetonitrile (CAS: 75-05-8)
MeOH methanol (CAS: 67-56-1)
min minute
MOM methoxymethyl
MSA N-trimethylsilyl-N-methylacetamide (CAS: 7449-74-3)
MTBE methyl tert-butyl ether (CAS: 1634-04-4)
MTHP 4-Methyltetrahydropyran (CAS: 4717-96-8)
n Normal
n-BuLi normal-butyllithium (CAS: 109-72-8)
Pd₂(dba)₃ Tris(dibenzylideneacetone)dipalladium(0) (CAS: 51364-51-3)
Pd(dppf)Cl₂ Bis(diphenylphosphino)ferrocene]dichloropalladium(II) (CAS: 72287-26-4)
Pd(PPh₃)₄ Tetrakis(triphenylphosphine)palladium(0) (CAS: 14221-01-3)
Ph phenyl
PPTS pyridinium p-toluenesulfonate (CAS: 24057-28-1)
quant quantitative
RP-HPLC reverse-phase HPLC
rt room temperatures
s singlet
satd. saturated
SPhos dicyclohexyl(2′,6′-dimethoxy[1,1′-biphenyl]-2-yl)phosphane (CAS: 657408-07-6)
t triplet
t-BuOH tert-Butyl Alcohol, 2-Methyl-2-propanol (CAS: 75-65-0)
tert tertiary
TFA trifluoroacetic acid (CAS: 76-05-1)
THF tetrahydrofuran (CAS: 109-99-9)
T₃P 1,3,5-tripropyl-1lambda5,3lambda5,5lambda5-triphosphinane-1,3,5-trione (CAS: 68957-94-8 as 50% solution in EtOAc)
Xphos Dicyclohexyl(2′,4′,6′-triisopropyl-[1,1′-biphenyl]-2-yl)phosphine (CAS: 564483-18-7)

### [A Novel Amino Acid Derivative]

The novel amino acid derivative of this disclosure is at least one selected from the group consisting of:

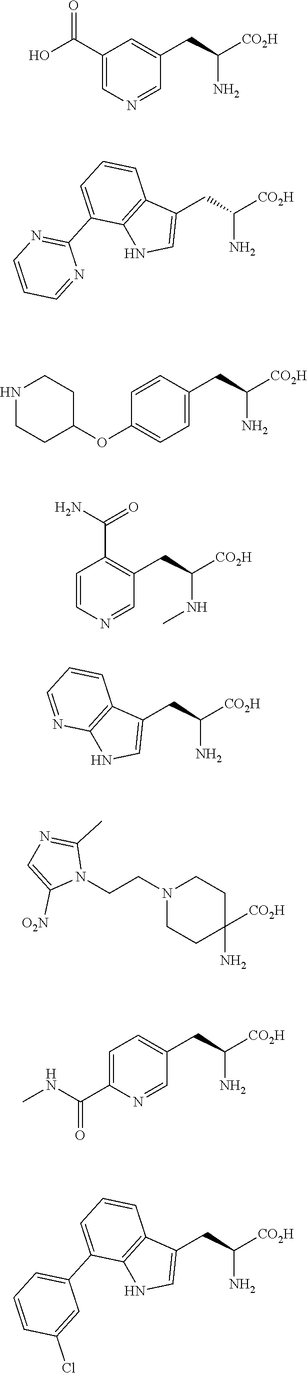

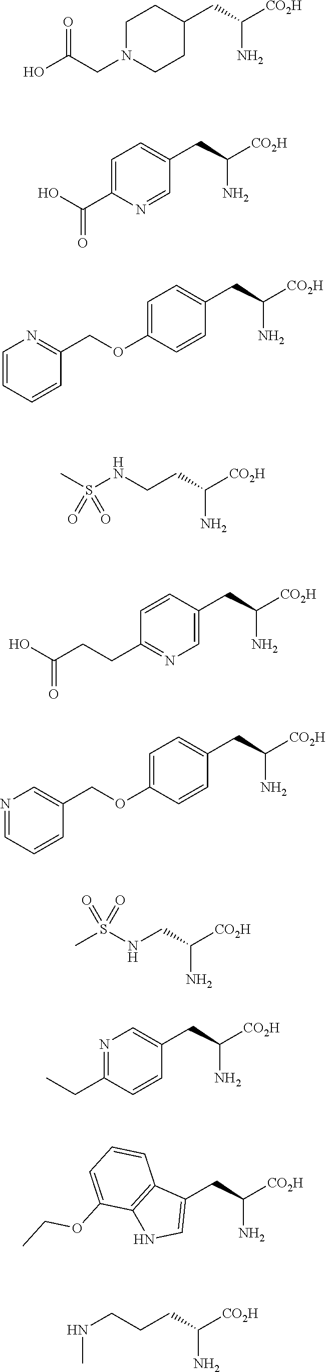

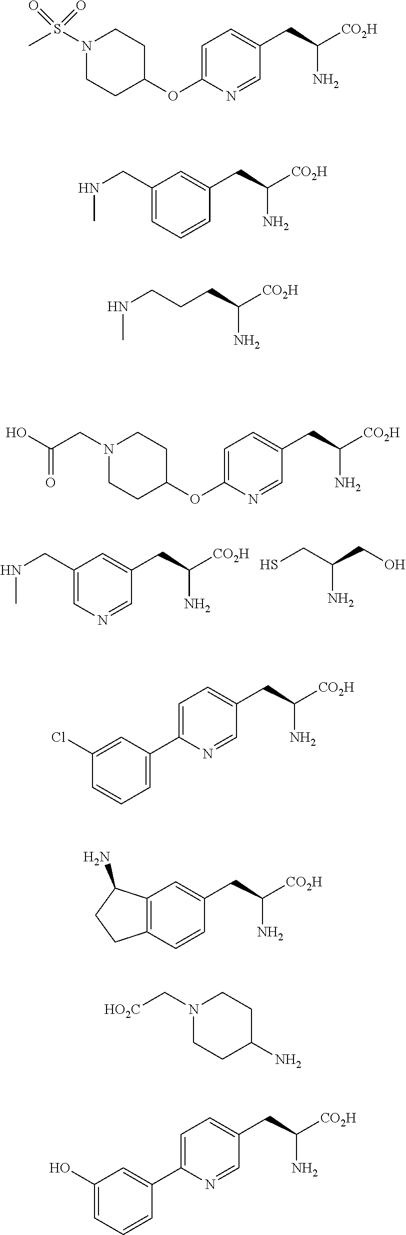

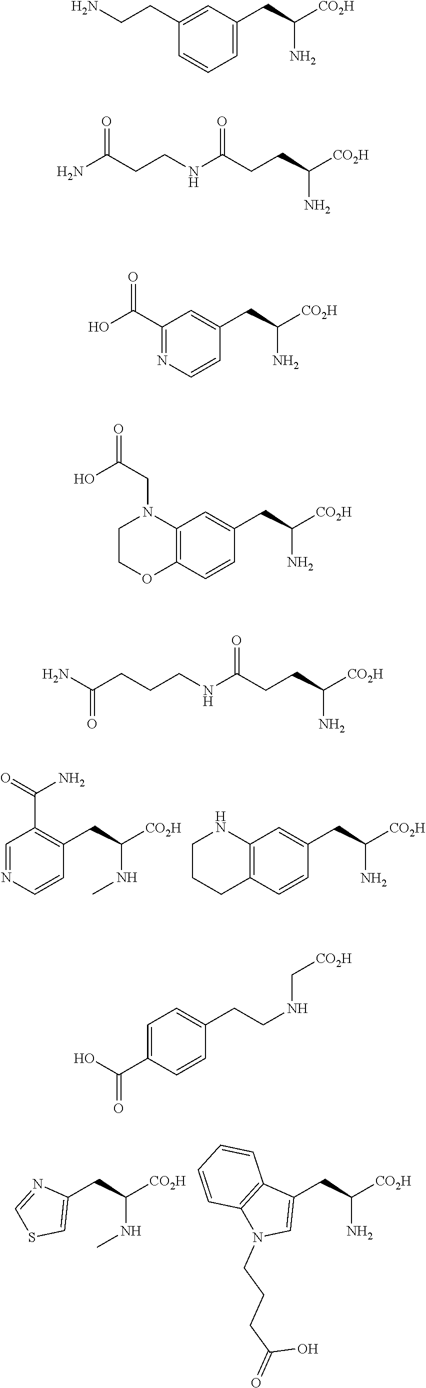

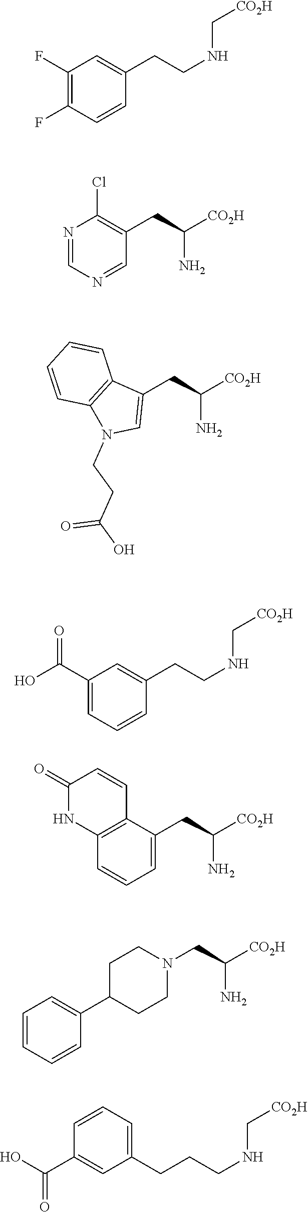

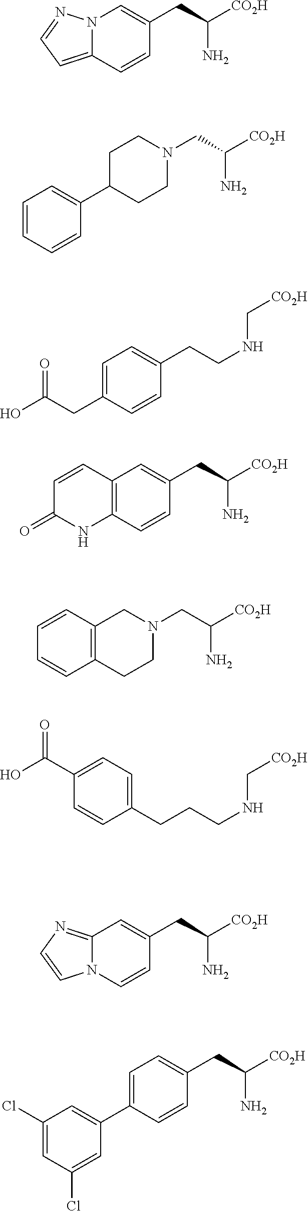

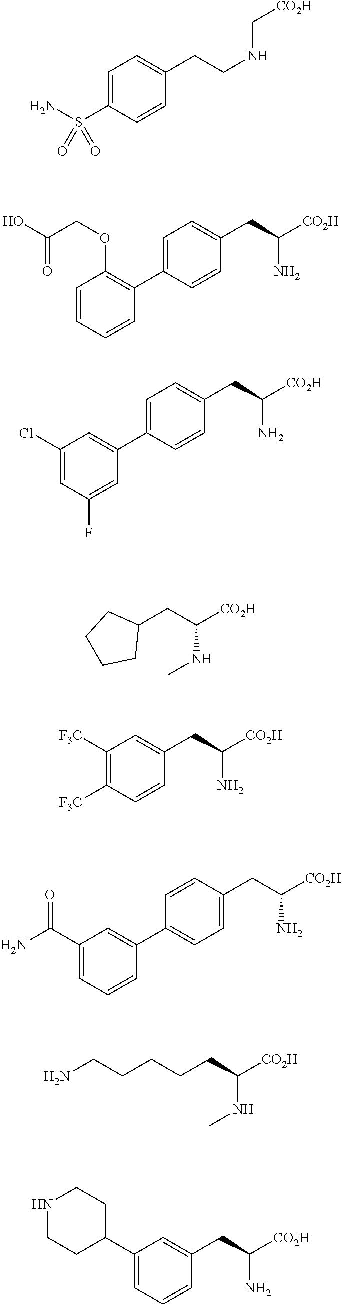

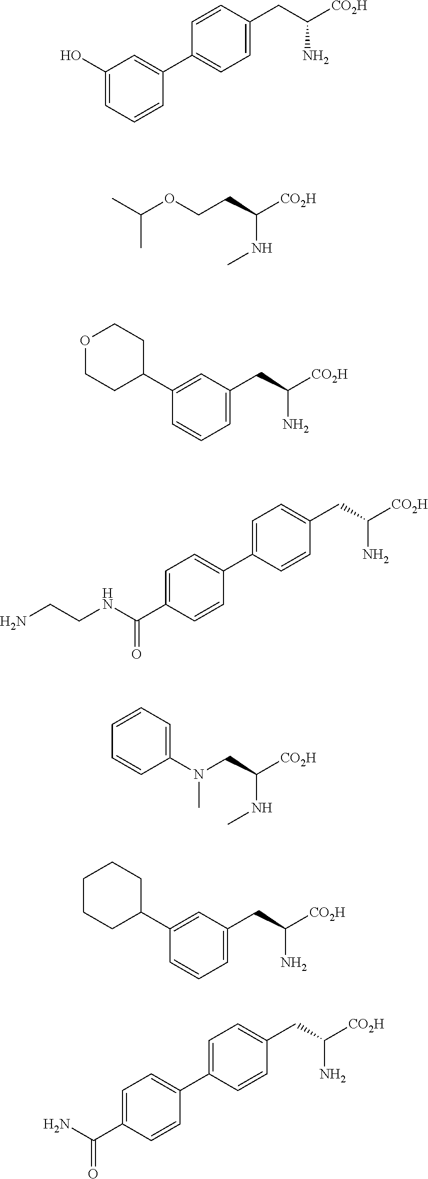

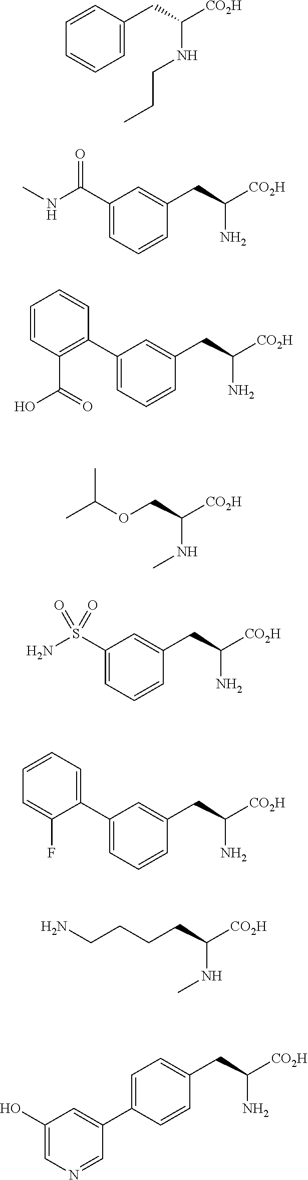

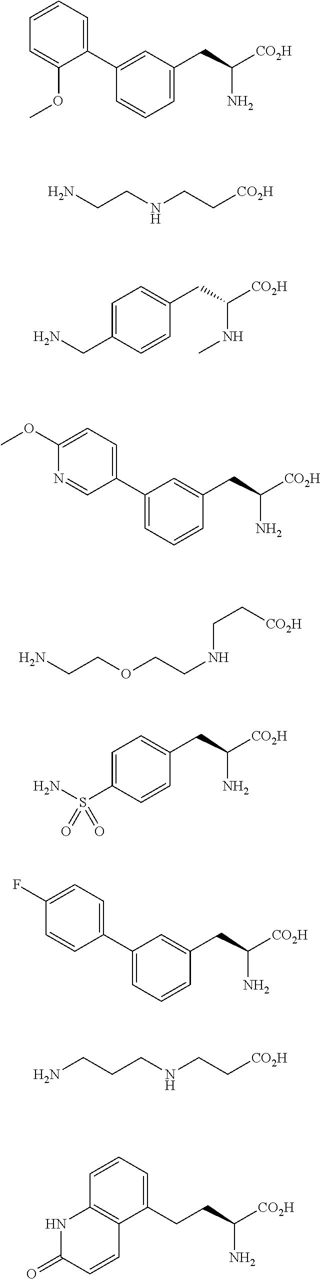

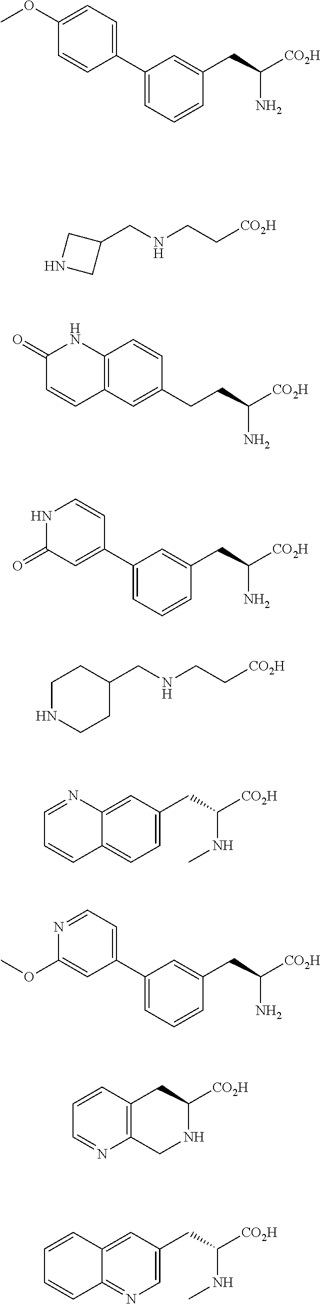

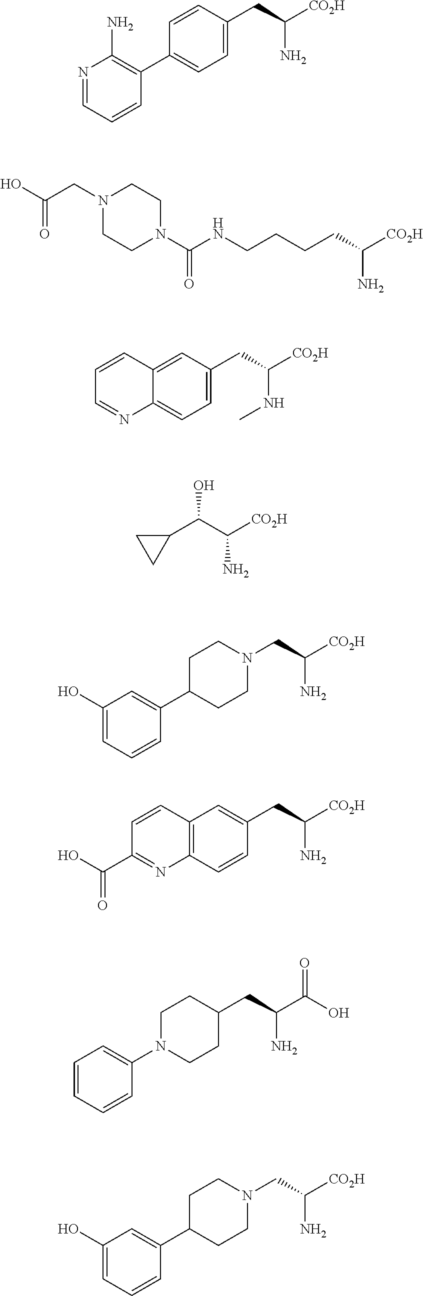

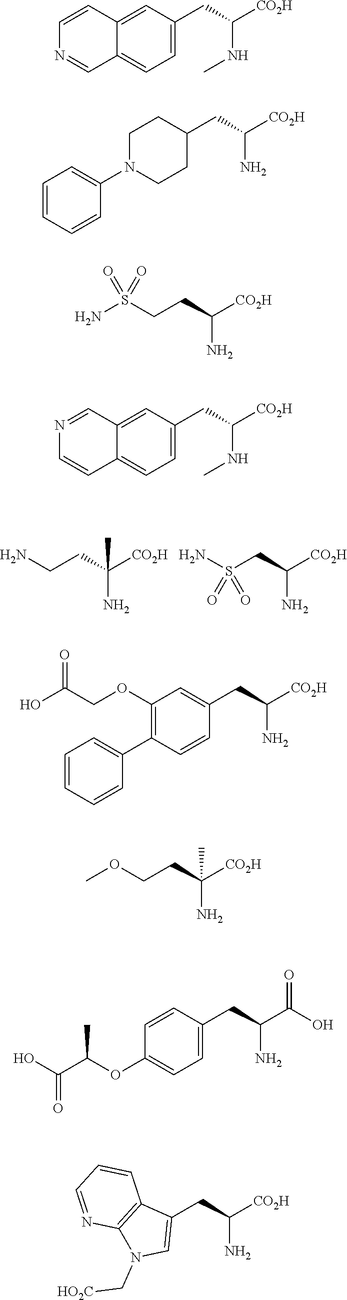

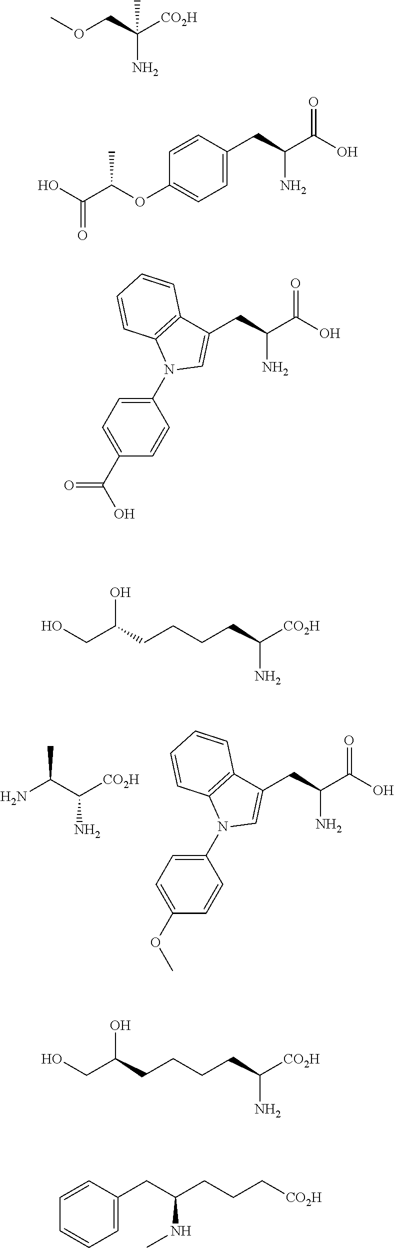

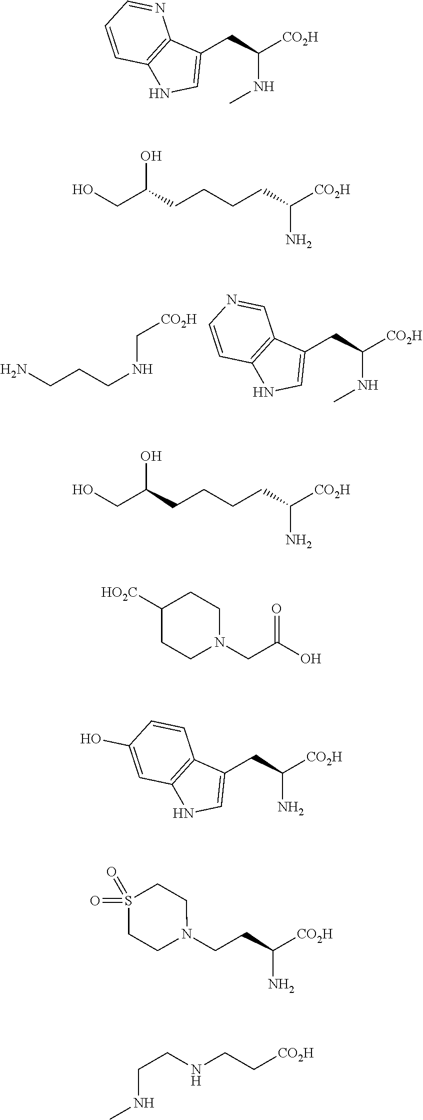

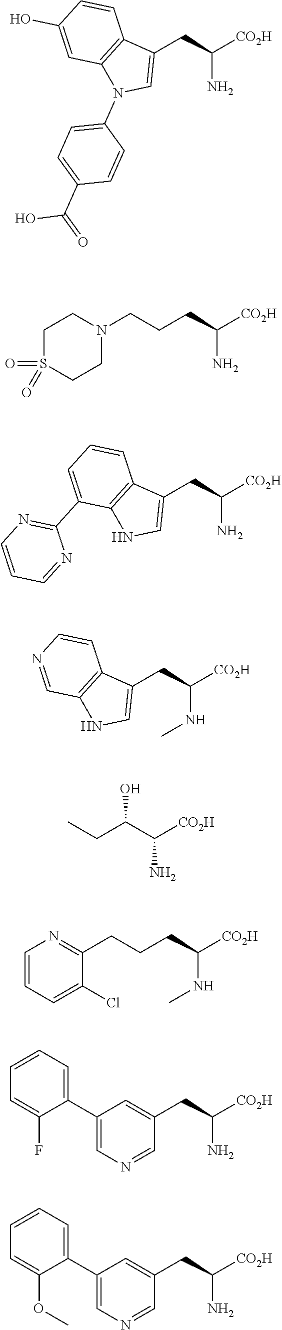

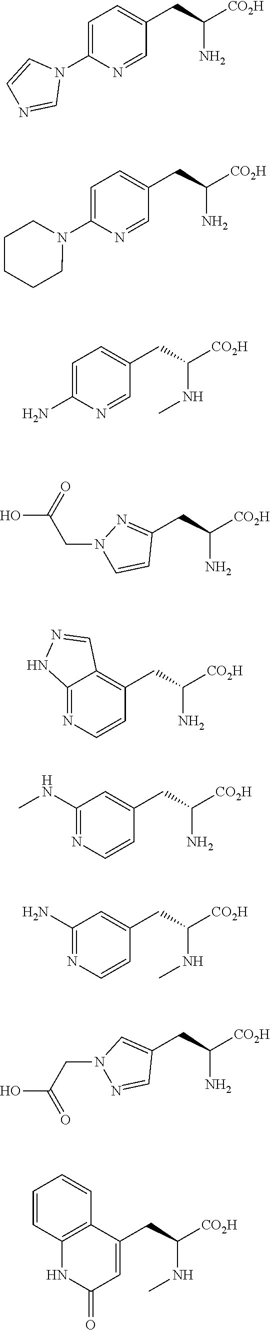

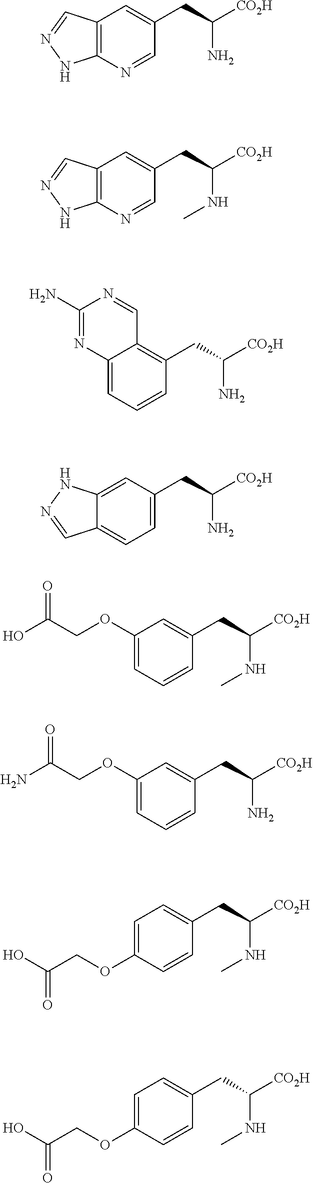

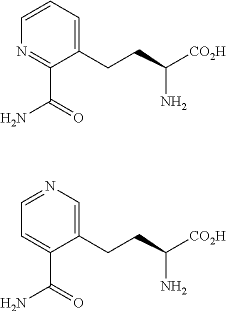 [SMILES: NC(=O)c1ncccc1CC[C@H](N)C(=O)O ; NC(=O)c1ccncc1CC[C@H](N)C(=O)O]

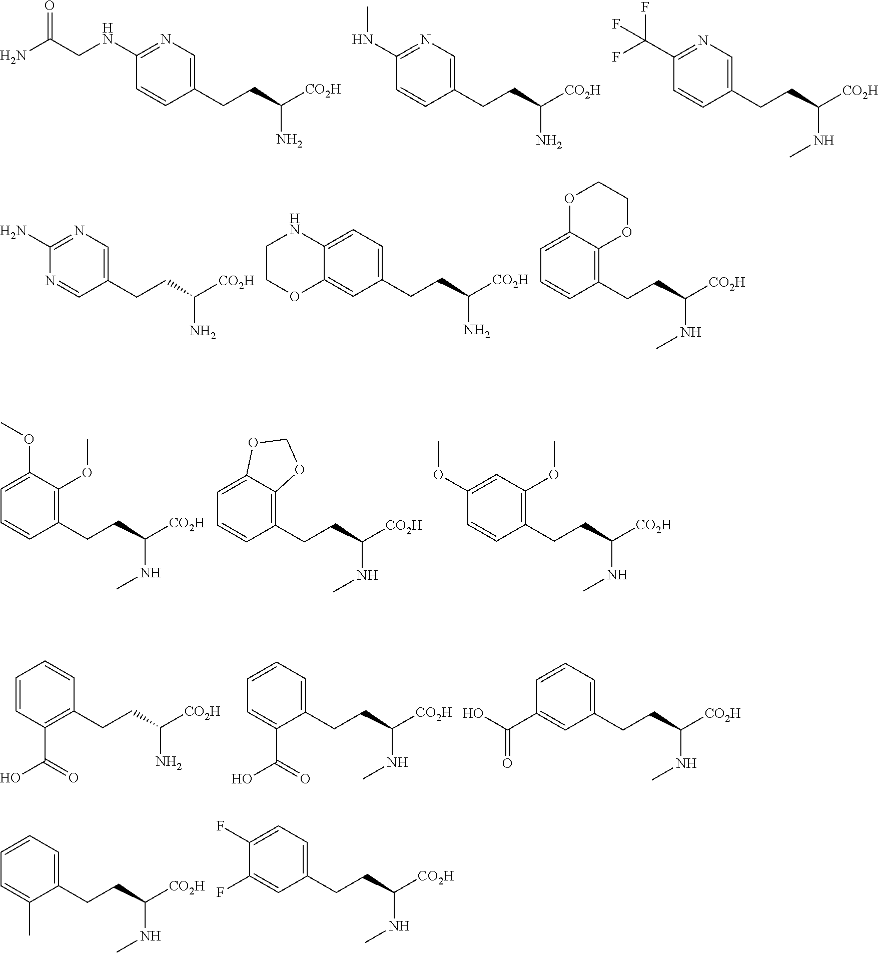

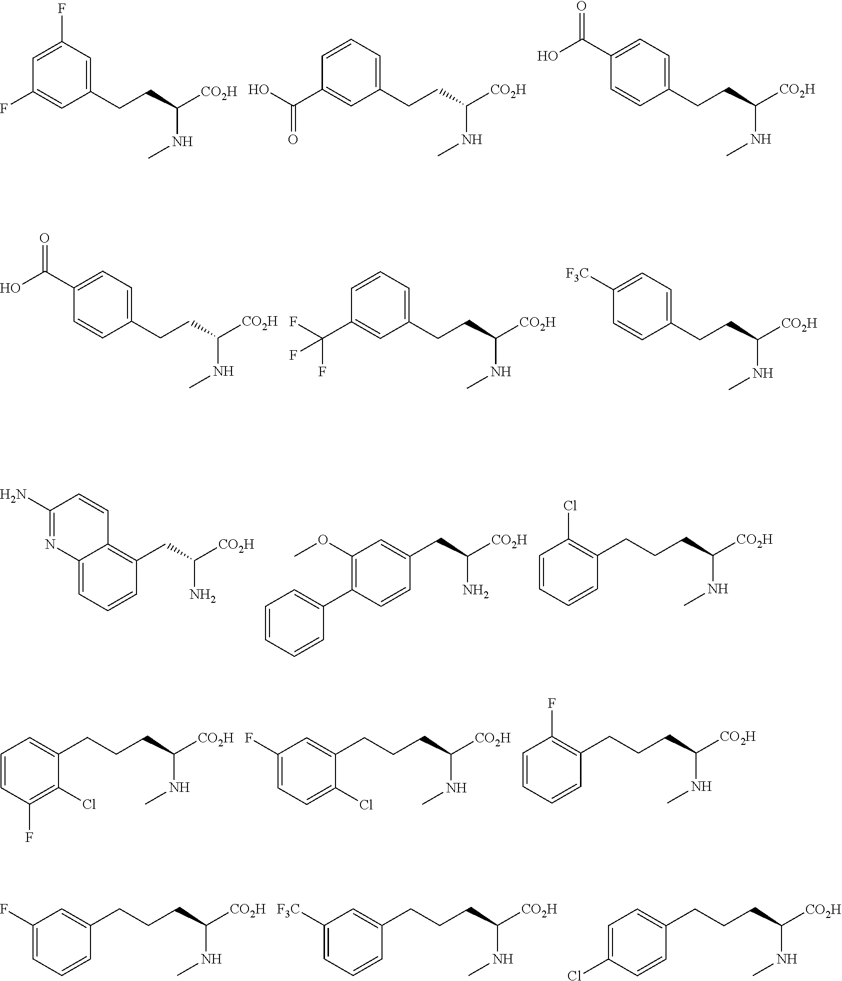

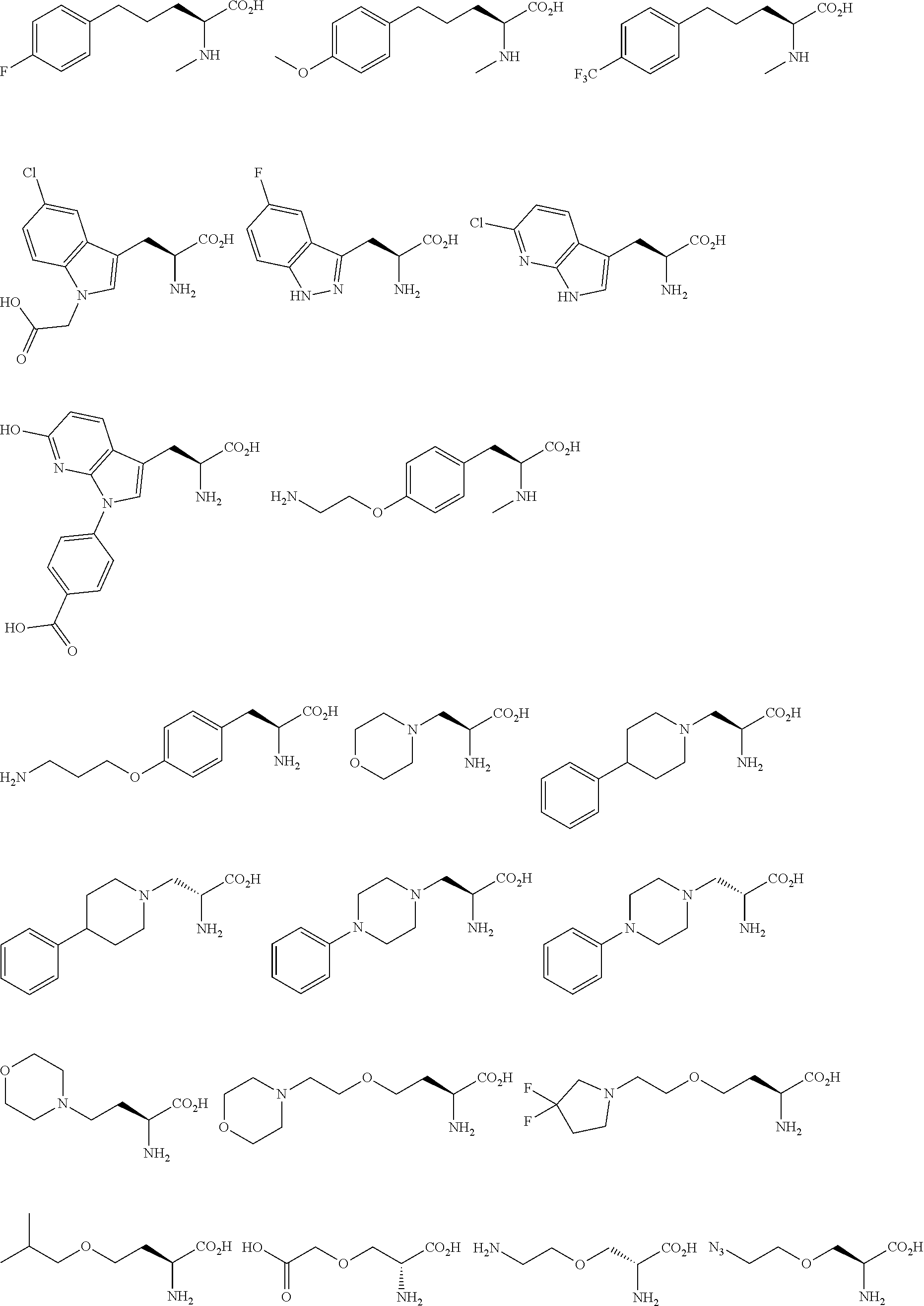

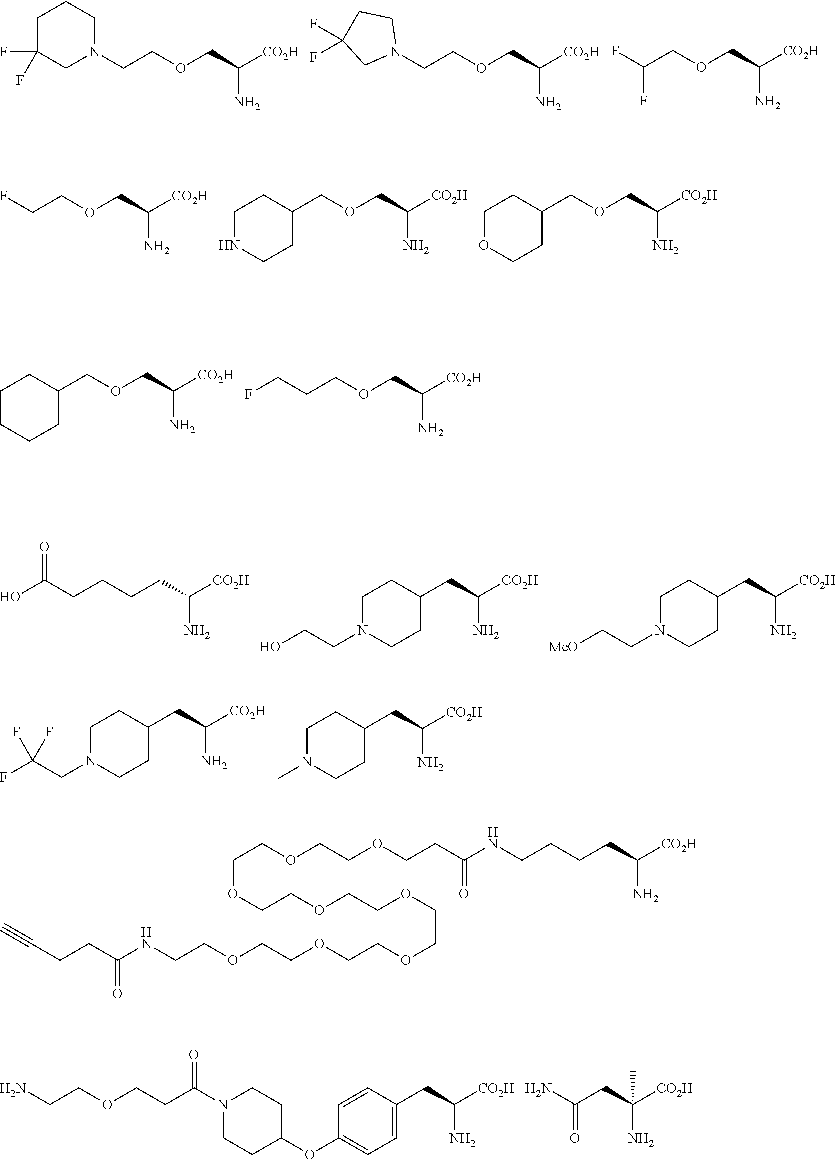

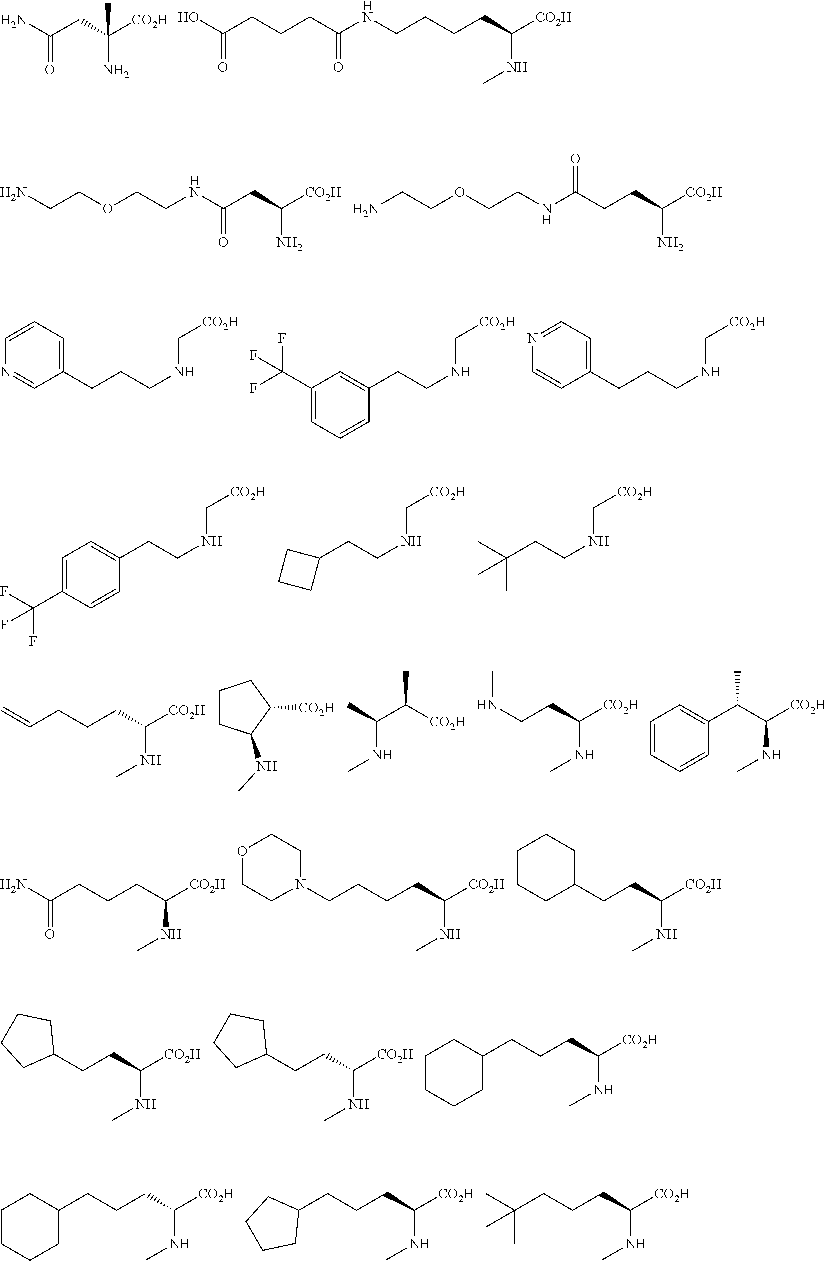

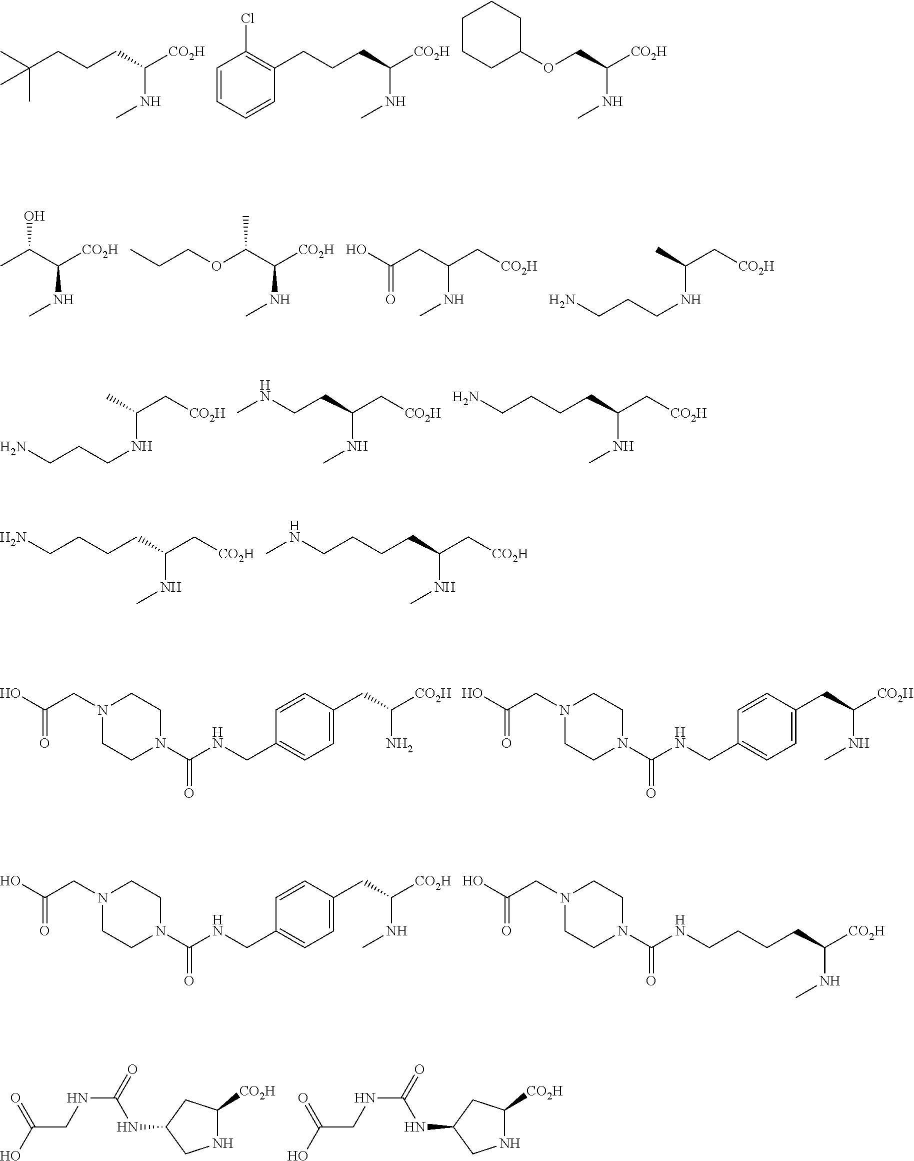

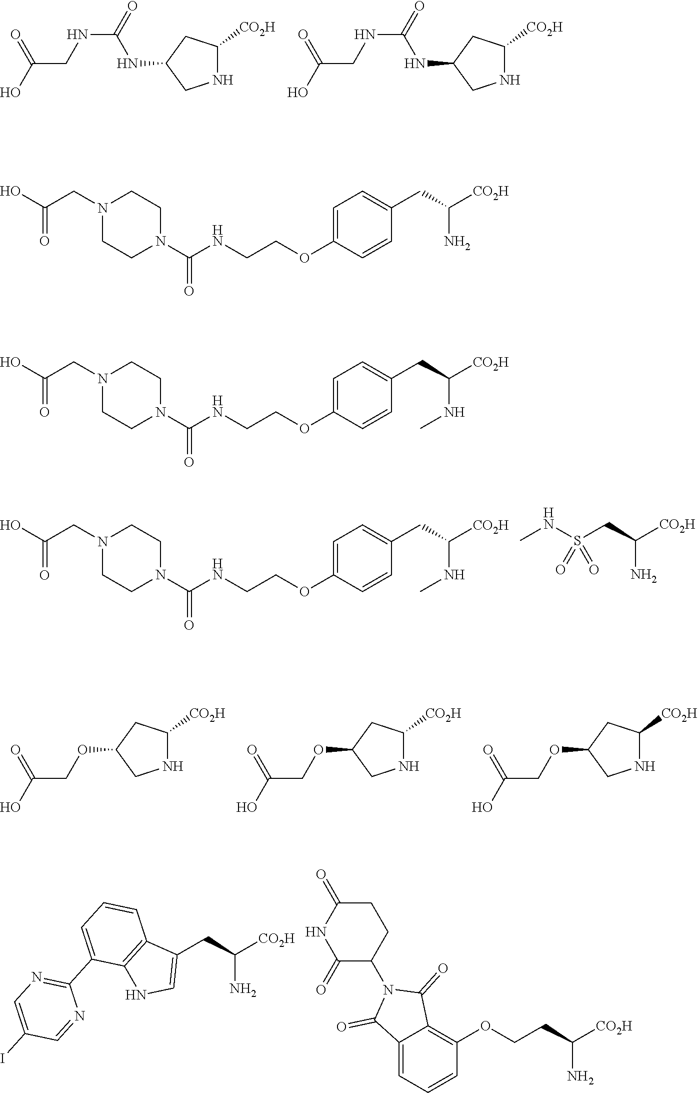

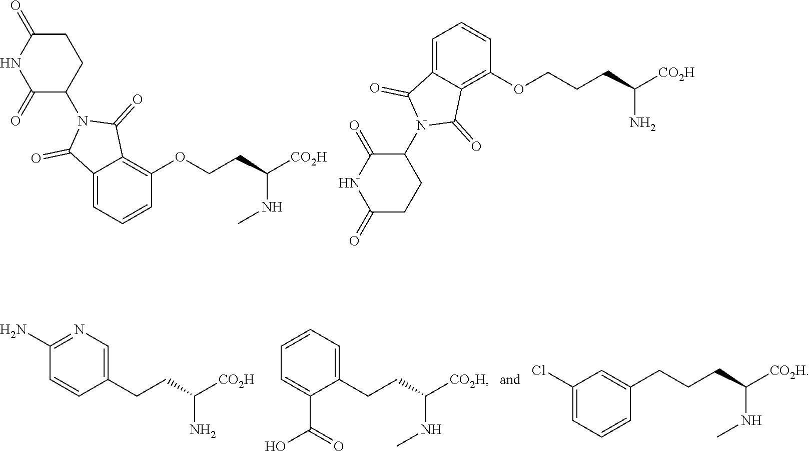 [SMILES: CN[C@@H](CCOc1cccc2c1C(=O)N(C1CCC(=O)NC1=O)C2=O)C(=O)O ; N[C@@H](CCCOc1cccc2c1C(=O)N(C1CCC(=O)NC1=O)C2=O)C(=O)O ; Nc1ccc(CC[C@@H](N)C(=O)O)cn1 ; CN[C@H](CCc1ccccc1C(=O)O)C(=O)O ; "and" ; CN[C@@H](CCCc1cccc(Cl)c1)C(=O)O]

In other words, the novel amino acid derivative of this disclosure is at least one selected from the group consisting of (S)-5-(2-amino-2-carboxyethyl)nicotinic acid, (S)-3-(4-carbamoylpyridin-3-yl)-2-(methylamino)propanoic acid, (S)-2-amino-3-(6-(methylcarbamoyl)pyridin-3-yl)propanoic acid, (S)-5-(2-amino-2-carboxyethyl)picolinic acid, (S)-2-amino-3-(6-(2-carboxyethyl)pyridin-3-yl)propanoic acid, (S)-2-amino-3-(6-ethylpyridin-3-yl)propanoic acid, (S)-2-amino-3-(6-((1-(methylsulfonyl)piperidin-4-yl)oxy)pyridin-3-yl)propanoic acid, (S)-2-amino-3-(6-((1-(carboxymethyl)piperidin-4-yl)oxy)pyridin-3-yl)propanoic acid, (S)-2-amino-3-(6-(3-chlorophenyl)pyridin-3-yl)propanoic acid, (S)-2-amino-3-(6-(3-hydroxyphenyl)pyridin-3-yl)propanoic acid, (S)-4-(2-amino-2-carboxyethyl)picolinic acid, (S)-3-(3-carbamoylpyridin-4-yl)-2-(methylamino)propanoic acid, (S)-2-(methylamino)-3-(thiazol-4-yl)propanoic acid, (S)-2-amino-3-(4-chloropyrimidin-5-yl)propanoic acid, (S)-2-amino-3-(2-oxo-1,2-dihydroquinolin-5-yl)propanoic acid, (S)-2-amino-3-(pyrazolo[1,5-a]pyridin-6-yl)propanoic acid, (S)-2-amino-3-(2-oxo-1,2-dihydroquinolin-6-yl)propanoic acid, (S)-2-amino-3-(imidazo[1,2-a]pyridin-7-yl)propanoic acid, (S)-2-amino-3-(2′-(carboxymethoxy)-[1,1′-biphenyl]-4-yl)propanoic acid, (S)-2-amino-3-(3,4-bis(trifluoromethyl)phenyl)propanoic acid, (S)-2-amino-3-(3-(piperidin-4-yl)phenyl)propanoic acid, (S)-2-amino-3-(3-(tetrahydro-2H-pyran-4-yl)phenyl)propanoic acid, (S)-2-amino-3-(3-cyclohexylphenyl)propanoic acid, (S)-2-amino-3-(3-(methylcarbamoyl)phenyl)propanoic acid, (S)-2-amino-3-(3-sulfamoyl phenyl)propanoic acid, (S)-2-amino-3-(4-(5-hydroxypyridin-3-yl)phenyl)propanoic acid, (R)-3-(4-(aminomethyl)phenyl)-2-(methylamino)propanoic acid, (S)-2-amino-3-(4-sulfamoylphenyl)propanoic acid, (S)-2-amino-4-(2-oxo-1,2-dihydroquinolin-5-yl)butanoic acid, (S)-2-amino-4-(2-oxo-1,2-dihydroquinolin-6-yl)butanoic acid, (R)-2-(methyl amino)-3-(quinolin-7-yl)propanoic acid, (R)-2-(methylamino)-3-(quinolin-3-yl)propanoic acid, (R)-2-(methylamino)-3-(quinolin-6-yl)propanoic acid, (S)-6-(2-amino-2-carb oxyethyl)quinoline-2-carboxylic acid, (R)-3-(isoquinolin-6-yl)-2-(methylamino)propanoic acid, (R)-3-(isoquinolin-7-yl)-2-(methylamino)propanoic acid, (S)-2-amino-3-(2-(carboxymethoxy)-[1,1′-biphenyl]-4-yl)propanoic acid, (S)-2-amino-3-(1-(carboxymethyl)-1H-pyrrolo[2,3-b]pyridin-3-yl)propanoic acid, 4-{3-[(2S)-2-amino-2-carboxyethyl]-1H-indol-1-yl}benzoic acid, (2S)-2-amino-3-[1-(4-methoxyphenyl)-1H-indol-3-yl]propanoic acid, (S)-2-(methylamino)-3-(1H-pyrrolo[3,2-b]pyridin-3-yl)propanoic acid, (S)-2-(methylamino)-3-(1H-pyrrolo[3,2-c]pyridin-3-yl)propanoic acid, (S)-2-amino-3-(6-hydroxy-1H-indol-3-yl)propanoic acid, (S)-4-(3-(2-amino-2-carboxyethyl)-6-hydroxy-1H-indol-1-yl)benzoic acid, (S)-2-(methylamino)-3-(1H-pyrrolo[2,3-c]pyridin-3-yl)propanoic acid, (R)-2-amino-3-(7-(pyrimidin-2-yl)-1H-indol-3-yl)propanoic acid, (S)-2-amino-3-(1H-pyrrolo[2,3-b]pyridin-3-yl)propanoic acid, (S)-2-amino-3-(7-(3-chlorophenyl)-1H-indol-3-yl)propanoic acid, (S)-2-amino-3-(4-(pyridin-2-ylmethoxy)phenyl)propanoic acid, (S)-2-amino-3-(4-(pyridin-3-ylmethoxy)phenyl)propanoic acid, (S)-2-amino-3-(7-ethoxy-1H-indol-3-yl)propanoic acid, (S)-2-amino-3-(3-((methylamino)methyl)phenyl)propanoic acid, (S)-2-amino-3-(5-(aminomethyl)pyridin-3-yl)propanoic acid, (S)-2-amino-3-((R)-3-amino-2,3-dihydro-1H-inden-5-yl)propanoic acid, (S)-2-amino-3-(3-(2-aminoethyl)phenyl)propanoic acid, (S)-2-amino-3-(4-(carboxymethyl)-3,4-dihydro-2H-benzo[b][1,4]oxazin-6-yl)propanoic acid, (S)-2-amino-3-(1,2,3,4-tetrahydroquinolin-7-yl)propanoic acid, 4-{3-[(2S)-2-amino-2-carboxyethyl]-1H-indol-1-yl}butanoic acid, (2S)-2-amino-3-[1-(2-carboxyethyl)-1H-indol-3-yl]propanoic acid, (S)-2-amino-3-(4-phenylpiperidin-1-yl)propanoic acid, (R)-2-amino-3-(4-phenylpiperidin-1-yl)propanoic acid, 2-amino-3-(3,4-dihydroisoquinolin-2(1H)-yl)propanoic acid, (S)-2-amino-3-(3′,5′-dichloro-[1,1′-biphenyl]-4-yl)propanoic acid, (S)-2-amino-3-(3′-chloro-5′-fluoro-[1,1′-biphenyl]-4-yl)propanoic acid, (R)-2-amino-3-(3′-carbamoyl-[1,1′-biphenyl]-4-yl)propanoic acid, (R)-2-amino-3-(3′-hydroxy-[1,1′-biphenyl]-4-yl)propanoic acid, (R)-2-amino-3-(4′-((2-aminoethyl)carbamoyl)-[1,1′-biphenyl]-4-yl)propanoic acid, (R)-2-amino-3-(4′-carbamoyl-[1,1′-biphenyl]-4-yl)propanoic acid, (S)-3′-(2-amino-2-carboxyethyl)-[1,1′-biphenyl]-2-carboxylic acid, (S)-2-amino-3-(2′-fluoro-[1,1′-biphenyl]-3-yl)propanoic acid, (S)-2-amino-3-(2′-methoxy-[1,1′-biphenyl]-3-yl)propanoic acid, (S)-2-amino-3-(3-(6-methoxypyridin-3-yl)phenyl)propanoic acid, (S)-2-amino-3-(4′-fluoro-[1,1′-biphenyl]-3-yl)propanoic acid, (S)-2-amino-3-(4′-methoxy-[1,1′-biphenyl]-3-yl)propanoic acid, (S)-2-amino-3-(3-(2-oxo-1,2-dihydropyridin-4-yl)phenyl)propanoic acid, (S)-2-amino-3-(3-(2-methoxypyridin-4-yl)phenyl)propanoic acid, (S)-2-amino-3-(4-(2-amino pyridin-3-yl)phenyl)propanoic acid, (2R,3S)-2-amino-3-cyclopropyl-3-hydroxypropanoic acid, (S)-2-amino-3-(1-phenylpiperidin-4-yl)propanoic acid, (R)-2-amino-3-(1-phenylpiperidin-4-yl)propanoic acid, (R)-2,4-diamino-2-methylbutanoic acid, (S)-2-amino-4-methoxy-2-methylbutanoic acid, (S)-2-amino-3-methoxy-2-methylpropanoic acid, (2S,7R)-2-amino-7,8-dihydroxyoctanoic acid, (2S,7S)-2-amino-7,8-dihydroxyoctanoic acid, (2R,7R)-2-amino-7,8-dihydroxyoctanoic acid, (2R,7S)-2-amino-7,8-dihydroxyoctanoic acid, (S)-2-amino-4-(1,1-dioxidothiomorpholino)butanoic acid, (S)-2-amino-5-(1,1-dioxidothiomorpholino)pentanoic acid, (2R,3S)-2-amino-3-hydroxypentanoic acid, (S)-2-amino-3-(4-(piperidin-4-yloxy)phenyl)propanoic acid, 4-amino-1-(2-(2-methyl-5-nitro-1H-imidazol-1-yl)ethyl)piperidine-4-carboxylic acid, (R)-2-amino-3-(1-(carboxymethyl)piperidin-4-yl)propanoic acid, (R)-2-amino-4-(methylsulfonamido)butanoic acid, (R)-2-amino-3-(methylsulfonamido)propanoic acid, (R)-2-amino-5-(methylamino) pentanoic acid, (S)-2-amino-5-(methylamino)pentanoic acid, (R)-2-amino-3-mercapto propan-1-ol, 2-(4-aminopiperidin-1-yl)acetic acid, (2S)-2-amino-4-[(2-carbamoylethyl) carbamoyl]butanoic acid, (2S)-2-amino-4-[(3-carbamoylpropyl)carbamoyl]butanoic acid, 4-(2-((carboxymethyl)amino)ethyl)benzoic acid, 2-{[2-(3,4-difluorophenyl)ethyl]amino}acetic acid, 3-(2-((carboxymethyl)amino)ethyl)benzoic acid, 3-(3-((carboxymethyl)amino)propyl)benzoic acid, 2-(4-(2-((carboxymethyl)amino)ethyl)phenyl)acetic acid, 4-(3-((carboxymethyl)amino)propyl)benzoic acid, 2-{[2-(4-sulfamoylphenyl)ethyl]amino}acetic acid, (R)-3-cyclopentyl-2-(methylamino)propanoic acid, (S)-7-amino-2-(methylamino)heptanoic acid, (2S)-2-(methylamino)-4-(propan-2-yloxy)butanoic acid, (S)-3-(methyl(phenyl)amino)-2-(methylamino)propanoic acid, (2R)-3-phenyl-2-(propylamino)propanoic acid, (2S)-2-(methylamino)-3-(propan-2-yloxy)propanoic acid, (2S)-6-amino-2-(methylamino)hexanoic acid, 3-((2-aminoethyl)amino)propanoic acid, 3-((2-(2-aminoethoxy)ethyl)amino)propanoic acid, 3-((3-aminopropyl)amino)propanoic acid, 3-((azetidin-3-ylmethyl)amino)propanoic acid, 3-((piperidin-4-ylmethyl)amino)propanoic acid, (S)-5,6,7,8-tetrahydro-1,7-naphthyridine-6-carboxylic acid, (2R)-2-amino-6-{[4-(carboxymethyl)piperazine-1-carbonyl]amino}hexanoic acid, (S)-2-amino-3-(4-(3-hydroxyphenyl)piperidin-1-yl)propanoic acid, (R)-2-amino-3-(4-(3-hydroxyphenyl) piperidin-1-yl)propanoic acid, (S)-2-amino-4-sulfamoylbutanoic acid, (2R)-2-amino-3-sulfamoylpropanoic acid, (S)-2-amino-3-(4-((R)-1-carboxyethoxy)phenyl)propanoic acid, (S)-2-amino-3-(4-((S)-1-carboxyethoxy)phenyl)propanoic acid, (2R,3S)-2,3-diaminobutanoic acid, (R)-5-(methylamino)-6-phenylhexanoic acid, 2-[(3-aminopropyl)amino]acetic acid, 1-(carboxymethyl)piperidine-4-carboxylic acid, 3-((2-(methylamino)ethyl)amino)propanoic acid, (S)-2-amino-3-(7-(pyrimidin-2-yl)-1H-indol-3-yl)propanoic acid, (S)-5-(3-chloropyridin-2-yl)-2-(methylamino)pentanoic acid, (S)-2-amino-3-(5-(2-fluorophenyl)pyridin-3-yl)propanoic acid, (S)-2-amino-3-(5-(2-methoxyphenyl)pyri din-3-yl)propanoic acid, (S)-3-(6-(1H-imidazol-1-yl)pyridin-3-yl)-2-aminopropanoic acid, (S)-2-amino-3-(6-(piperidin-1-yl)pyridin-3-yl)propanoic acid, (R)-3-(6-aminopyridin-3-yl)-2-(methylamino)propanoic acid, (S)-2-amino-3-(1-(carboxymethyl)-1H-pyrazol-3-yl)propanoic acid, (R)-2-amino-3-(1H-pyrazolo[3,4-b]pyridin-4-yl)propanoic acid, (R)-2-amino-3-(2-(methylamino)pyridin-4-yl)propanoic acid, (R)-3-(2-aminopyridin-4-yl)-2-(methylamino)propanoic acid, (S)-2-amino-3-(1-(carboxymethyl)-1H-pyrazol-4-yl)propanoic acid, (S)-2-(methylamino)-3-(2-oxo-1,2-dihydroquinolin-4-yl)propanoic acid, (S)-2-amino-3-(1H-pyrazolo[3,4-b]pyridin-5-yl)propanoic acid, (S)-2-(methylamino)-3-(1H-pyrazolo[3,4-b]pyridin-5-yl)propanoic acid, (R)-2-amino-3-(2-amino quinazolin-5-yl)propanoic acid, (S)-2-amino-3-(1H-indazol-6-yl)propanoic acid, (S)-3-(3-(carboxymethoxy)phenyl)-2-(methylamino)propanoic acid, (S)-2-amino-3-(3-(2-amino-2-oxoethoxy)phenyl)propanoic acid, (S)-3-(4-(carboxymethoxy)phenyl)-2-(meth ylamino)propanoic acid, (R)-3-(4-(carboxymethoxy)phenyl)-2-(methylamino)propanoic acid, (S)-2-amino-4-(2-carbamoylpyridin-3-yl)butanoic acid, (S)-2-amino-4-(4-carbamoylpyridin-3-yl)butanoic acid, (S)-2-amino-4-(6-((2-amino-2-oxoethyl)amino)pyrid in-3-yl)butanoic acid, (S)-2-amino-4-(6-(methylamino)pyridin-3-yl)butanoic acid, (R)-2-amino-4-(6-aminopyridin-3-yl)butanoic acid, (S)-2-(methylamino)-4-(6-(trifluoro methyl)pyridin-3-yl)butanoic acid, (R)-2-amino-4-(2-aminopyrimidin-5-yl)butanoic acid, (S)-2-amino-4-(3,4-dihydro-2H-benzo[b][1,4]oxazin-7-yl)butanoic acid, (S)-4-(2, 3-dihydrobenzo[b][1,4]dioxin-5-yl)-2-(methylamino)butanoic acid, (S)-4-(2,3-dimethoxyphenyl)-2-(methylamino)butanoic acid, (S)-4-(benzo[d][1,3]dioxol-4-yl)-2-(methylamino)butanoic acid, (S)-4-(2,4-dimethoxyphenyl)-2-(methylamino)butanoic acid, (R)-2-(3-amino-3-carboxypropyl)benzoic acid, (S)-2-(3-carboxy-3-(methylamino)propyl) benzoic acid, (R)-2-(3-carboxy-3-(methylamino)propyl)benzoic acid, (S)-3-(3-carboxy-3-(methylamino)propyl)benzoic acid, (R)-3-(3-carboxy-3-(methylamino)propyl)benzoic acid, (S)-2-(methylamino)-4-(o-tolyl)butanoic acid, (S)-4-(3,4-difluorophenyl)-2-(methylamino)butanoic acid, (S)-4-(3,5-difluorophenyl)-2-(methylamino)butanoic acid, (S)-4-(3-carboxy-3-(methylamino)propyl)benzoic acid, (R)-4-(3-carboxy-3-(methylamino)propyl)benzoic acid, (S)-2-(methylamino)-4-(3-(trifluoromethyl)phenyl)butanoic acid, (S)-2-(methylamino)-4-(4-(trifluoromethyl)phenyl)butanoic acid, (R)-2-amino-3-(2-aminoquinolin-5-yl)propanoic acid, (S)-2-amino-3-(2-methoxy-[1,1′-biphenyl]-4-yl)propanoic acid, (S)-5-(2-chlorophenyl)-2-(methylamino)pentanoic acid, (S)-5-(2-chloro-3-fluorophenyl)-2-(methylamino)pentanoic acid, (S)-5-(2-chloro-5-fluorophenyl)-2-(methylamino)pentanoic acid, (S)-5-(2-fluorophenyl)-2-(methylamino)pentanoic acid, (S)-5-(3-chlorophenyl)-2-(methylamino)pentanoic acid, (S)-5-(3-fluorophenyl)-2-(methylamino)pentanoic acid, (S)-2-(methylamino)-5-(3-(trifluoromethyl)phenyl)pentanoic acid, (S)-5-(4-chlorophenyl)-2-(methylamino)pentanoic acid, (S)-5-(4-fluorophenyl)-2-(methylamino)pentanoic acid, (S)-5-(4-methoxyphenyl)-2-(methylamino)pentanoic acid, (S)-2-(methylamino)-5-(4-(trifluoromethyl)phenyl)pentanoic acid, (S)-2-amino-3-(1-(carboxymethyl)-5-chloro-1H-indol-3-yl)propanoic acid, (S)-2-amino-3-(5-fluoro-1H-indazol-3-yl)propanoic acid, (S)-2-amino-3-(6-chloro-1H-pyrrolo[2,3-b]pyridin-3-yl)propanoic acid, (S)-4-(3-(2-amino-2-carboxyethyl)-6-hydroxy-1H-indol-1-yl)benzoic acid, (S)-3-(4-(2-aminoethoxy)phenyl)-2-(methylamino)propanoic acid, (S)-2-amino-3-(4-(3-aminopropoxy)phenyl)propanoic acid, (S)-2-amino-3-morpholinopropanoic acid, (S)-2-amino-3-(4-phenylpiperidin-1-yl)propanoic acid, (R)-2-amino-3-(4-phenylpiperidin-1-yl)propanoic acid, (S)-2-amino-3-(4-phenylpiperazin-1-yl)propanoic acid, (R)-2-amino-3-(4-phenylpiperazin-1-yl)propanoic acid, (S)-2-amino-4-morpholinobutanoic acid, (2S)-2-amino-4-[2-(morpholin-4-yl)ethoxy]butanoic acid, (2S)-2-amino-4-[2-(3,3-difluoropyrrolidin-1-yl)ethoxy]butanoic acid, (2S)-2-amino-4-(2-methylpropoxy)butanoic acid, (2R)-2-amino-3-(carboxymethoxy)propanoic acid, (2R)-2-amino-3-(2-aminoethoxy)propanoic acid, (2S)-2-amino-3-(2-azidoethoxy)propanoic acid, (2S)-2-amino-3-[2-(3,3-difluoropiperidin-1-yl)ethoxy]propanoic acid, (2S)-2-amino-3-[2-(3,3-difluoropyrrolidin-1-yl)ethoxy]propanoic acid, (2S)-2-amino-3-(2,2-difluoroethoxy)propanoic acid, (2S)-2-amino-3-(2-fluoroethoxy)propanoic acid, (2S)-2-amino-3-[(piperidin-4-yl)methoxy]propanoic acid, (2S)-2-amino-3-[(oxan-4-yl)methoxy]propanoic acid, (2S)-2-amino-3-(cyclohexylmethoxy)propanoic acid, (2S)-2-amino-3-(3-fluoropropoxy)propanoic acid, (R)-2-aminoheptanedioic acid, (S)-2-amino-3-(1-(2-hydroxyethyl)piperidin-4-yl)propanoic acid, (S)-2-amino-3-(1-(2-methoxyethyl)piperidin-4-yl)propanoic acid, (S)-2-amino-3-(1-(2,2,2-trifluoroethyl)piperidin-4-yl)propanoic acid, (S)-2-amino-3-(1-methylpiperidin-4-yl)propanoic acid, (S)-39-amino-5,33-dioxo-9,12,15,18,21,24,27,30-octaoxa-6,34-diazatetracont-1-yn-40-oic acid, (S)-2-amino-3-(4-((1-(3-(2-aminoethoxy)propanoyl)piperidin-4-yl)oxy)phenyl)propanoic acid (S)-2,4-diamino-2-methyl-4-oxobutanoic acid, (R)-2,4-diamino-2-methyl-4-oxobutanoic acid, (2S)-6-(4-carboxybutanamido)-2-(methylamino)hexanoic acid, (2S)-2-amino-3-{[2-(2-aminoethoxy)ethyl]carbamoyl}propanoic acid, (2S)-2-amino-4-{[2-(2-aminoethoxy) ethyl]carbamoyl}butanoic acid, 2-{[3-(pyridin-3-yl)propyl]amino}acetic acid, 2-({2-[3-(trifluoromethyl)phenyl]ethyl}amino)acetic acid, 2-{[3-(pyridin-4-yl)propyl]amino}acetic acid, 2-({2-[4-(trifluoromethyl)phenyl]ethyl}amino)acetic acid, 2-[(2-cyclobut ylethyl)amino]acetic acid, 2-[(3,3-dimethylbutyl)amino]acetic acid, (R)-2-(methylamino)hept-6-enoic acid, (1S,2S)-2-(methylamino)cyclopentane-1-carboxylic acid, (2R,3S)-2-methyl-3-(methylamino)butanoic acid, (S)-2,4-bis(methylamino)butanoic acid, (2S,3S)-2-(methylamino)-3-phenylbutanoic acid, (S)-6-amino-2-(methylamino)-6-oxohexanoic acid, (S)-2-(methylamino)-6-morpholinohexanoic acid, (S)-4-cyclohexyl-2-(methylamino)butanoic acid, (S)-4-cyclopentyl-2-(methylamino)butanoic acid, (R)-4-cyclopentyl-2-(methylamino)butanoic acid, (S)-5-cyclohexyl-2-(methylamino)pentanoic acid, (R)-5-cyclohexyl-2-(methylamino)pentanoic acid, (S)-5-cyclopentyl-2-(methylamino)pentanoic acid, (S)-6,6-dimethyl-2-(methylamino)heptanoic acid, (R)-6,6-dimethyl-2-(methylamino)heptanoic acid, (2S)-3-[(2-chlorophenyl)methoxy]-2-(methylamino)propanoic acid, (2S)-3-(cyclohexyloxy)-2-(methylamino)propanoic acid, (2S, 3S)-3-hydroxy-2-(methylamino)butanoic acid, (2S,3R)-2-(methylamino)-3-propoxybutanoic acid, 3-(methylamino)pentanedioic acid, (S)-3-((3-aminopropyl)amino)butanoic acid, (R)-3-((3-aminopropyl)amino)butanoic acid, (S)-3,5-bis(methylamino)pentanoic acid, (S)-7-amino-3-(methylamino)heptanoic acid, (R)-7-amino-3-(methylamino)heptanoic acid, (S)-3,7-bis(methylamino)heptanoic acid, (R)-2-amino-3-(4-((4-(carboxymethyl)piperazine-1-carboxamido)methyl)phenyl)propanoic acid, (S)-3-(4-((4-(carboxymethyl)piperazine-1-carboxamido)methyl)phenyl)-2-(methylamino)propanoic acid, (R)-3-(4-((4-(carboxymethyl)piperazine-1-carboxamido)methyl)phenyl)-2-(methylamino)propanoic acid, (2S)-6-{[4-(carboxymethyl)piperazine-1-carbonyl]amino}-2-(methyl amino)hexanoic acid, (2S,4R)-4-(3-(carboxymethyl)ureido)pyrrolidine-2-carboxylic acid, (2S,4S)-4-(3-(carboxymethyl)ureido)pyrrolidine-2-carboxylic acid, (2R,4R)-4-(3-(carboxymethyl)ureido)pyrrolidine-2-carboxylic acid, (2R,4S)-4-(3-(carboxymethyl)ureido)pyrrolidine-2-carboxylic acid, (R)-2-amino-3-(4-(2-(4-(carboxymethyl)piperazine-1-carboxamido)ethoxy)phenyl)propanoic acid, (S)-3-(4-(2-(4-(carboxymethyl)piperazine-1-carboxamido)ethoxy)phenyl)-2-(methylamino)propanoic acid, (R)-3-(4-(2-(4-(carboxymethyl)piperazine-1-carboxamido)ethoxy)phenyl)-2-(methylamino)propanoic acid, (2R)-2-amino-3-(methylsulfamoyl)propanoic acid, (2R,4R)-4-(carboxymethoxy)pyrrolidine-2-carboxylic acid, (2R,4S)-4-(carboxymethoxy)pyrrolidine-2-carboxylic acid, (2S,4S)-4-(carboxymethoxy)pyrrolidine-2-carboxylic acid, (S)-2-amino-3-(7-(5-iodo pyrimidin-2-yl)-1H-indol-3-yl)propanoic acid, (2S)-2-amino-4-{[2-(2,6-dioxopiperidin-3-yl)-1,3-dioxo-2,3-dihydro-1H-isoindol-4-yl]oxy}butanoic acid, (2S)-4-{[2-(2,6-dioxopiperidin-3-yl)-1,3-dioxo-2,3-dihydro-1H-isoindol-4-yl]oxy}-2-(methylamino)butanoic acid, and (2S)-2-amino-5-{[2-(2,6-dioxopiperidin-3-yl)-1,3-dioxo-2,3-dihydro-1H-isoindol-4-yl]oxy}pentanoic acid.

In one aspect, the novel amino acid derivative of this disclosure may contain any functional group such as carboxyl group and amino groups and any functional group in their side chains protected by conventional or any known protecting groups (e.g. Fmoc, Trt, Pdf, or Boc).

In other words, the novel amino acid derivative which side chains protected by conventional or any known protecting groups of this disclosure is at least one selected from the group consisting of: (S)-2-((((9H-fluoren-9-yl)methoxy)carbonyl)amino)-3-(5-(((3-methylpentan-3-yl)oxy)carbonyl)pyridin-3-yl)propanoic acid, (S)-2-((((9H-fluoren-9-yl)methoxy)carbonyl)(methyl)amino)-3-(4-carbamoylpyridin-3-yl)propanoic acid, (S)-2-((((9H-fluoren-9-yl)methoxy)carbonyl)amino)-3-(6-(methylcarbamoyl)pyridin-3-yl)propanoic acid, (S)-2-((((9H-fluoren-9-yl)methoxy)carbonyl)amino)-3-(6-(((3-methylpentan-3-yl)oxy)carbonyl)pyridin-3-yl)propanoic acid, (S)-2-((((9H-fluoren-9-yl)methoxy)carbonyl)amino)-3-(6-(3-(tert-butoxy)-3-oxopropyl)pyridin-3-yl)propanoic acid, (S)-2-((((9H-fluoren-9-yl)methoxy)carbonyl)amino)-3-(6-ethylpyridin-3-yl)propanoic acid, (S)-2-((((9H-fluoren-9-yl)methoxy)carbonyl)amino)-3-(6-((1-(methyl sulfonyl)piperidin-4-yl)oxy)pyridin-3-yl)propanoic acid, (S)-2-((((9H-fluoren-9-yl)methoxy)carbonyl)amino)-3-(6-((1-(2-(tert-butoxy)-2-oxoethyl)piperidin-4-yl)oxy)pyridin-3-yl)propanoic acid, (S)-2-((((9H-fluoren-9-yl)methoxy)carbonyl)amino)-3-(6-(3-chlorophenyl)pyridin-3-yl)propanoic acid, (S)-2-((((9H-fluoren-9-yl)methoxy)carbonyl)amino)-3-(6-(3-(tert-butoxy)phenyl)pyridin-3-yl)propanoic acid, (S)-2-((((9H-fluoren-9-yl)methoxy)carbonyl)amino)-3-(2-(((3-methylpentan-3-yl)oxy)carbonyl)pyridin-4-yl)propanoic acid, (S)-2-((((9H-fluoren-9-yl)methoxy)carbonyl)(methyl)amino)-3-(3-carbamoylpyridin-4-yl)propanoic acid, (S)-2-((((9H-fluoren-9-yl)methoxy)carbonyl)(methyl)amino)-3-(thiazol-4-yl)propanoic acid hydrochloride, (S)-2-((((9H-fluoren-9-yl)methoxy)carbonyl)amino)-3-(4-chloropyrimidin-5-yl)propanoic acid, (S)-2-((((9H-fluoren-9-yl)methoxy)carbonyl)amino)-3-(2-oxo-1,2-dihydroquinolin-5-yl)propanoic acid, (S)-2-((((9H-fluoren-9-yl)methoxy)carbonyl)amino)-3-(pyrazolo[1,5-a]pyridin-6-yl)propanoic acid, (S)-2-((((9H-fluoren-9-yl)methoxy)carbonyl)amino)-3-(2-oxo-1,2-dihydroquinolin-6-yl)propanoic acid, (S)-2-((((9H-fluoren-9-yl)methoxy)carbonyl)amino)-3-(imidazo [1,2-a]pyridin-7-yl)propanoic acid, (S)-2-((((9H-fluoren-9-yl)methoxy)carbonyl)amino)-3-(2′-(2-(tert-butoxy)-2-oxoethoxy)-[1,1′-biphenyl]-4-yl)propanoic acid, (S)-2-((((9H-fluoren-9-yl)methoxy)carbonyl)amino)-3-(3,4-bis(trifluoromethyl)phenyl)propanoic acid, (S)-2-((((9H-fluoren-9-yl)methoxy)carbonyl)amino)-3-(3-(1-(tert-butoxycarbonyl) piperidin-4-yl)phenyl)propanoic acid, (S)-2-((((9H-fluoren-9-yl)methoxy)carbonyl)amino)-3-(3-(tetrahydro-2H-pyran-4-yl)phenyl)propanoic acid, (S)-2-((((9H-fluoren-9-yl) methoxy)carbonyl)amino)-3-(3-cyclohexylphenyl)propanoic acid, (S)-2-((((9H-fluoren-9-yl)methoxy)carbonyl)amino)-3-(3-(methylcarbamoyl)phenyl)propanoic acid, (S)-2-((((9H-fluoren-9-yl)methoxy)carbonyl)amino)-3-(3-(N,N-bis(2,4-dimethoxybenzyl)sulfamoyl)phenyl)propanoic acid, (S)-2-((((9H-fluoren-9-yl)methoxy)carbonyl)amino)-3-(4-(5-(tert-butoxy)pyridin-3-yl)phenyl)propanoic acid, (R)-2-((((9H-fluoren-9-yl)methoxy)carbonyl)(methyl)amino)-3-(4-((((allyloxy)carbonyl)amino)methyl)phenyl)propanoic acid, (S)-2-((((9H-fluoren-9-yl)methoxy)carbonyl)amino)-3-(4-(N,N-bis(2,4-dimethoxybenzyl)sulfamoyl)phenyl)propanoic acid, (S)-2-((((9H-fluoren-9-yl)methoxy)carbonyl)amino)-4-(2-oxo-1,2-dihydroquinolin-5-yl)butanoic acid, (S)-2-((((9H-fluoren-9-yl) methoxy)carbonyl)amino)-4-(2-oxo-1,2-dihydroquinolin-6-yl)butanoic acid, (R)-2-((((9H-fluoren-9-yl)methoxy)carbonyl)(methyl)amino)-3-(quinolin-7-yl)propanoic acid, (R)-2-((((9H-fluoren-9-yl)methoxy)carbonyl)(methyl)amino)-3-(quinolin-3-yl)propanoic acid, (R)-2-((((9H-fluoren-9-yl)methoxy)carbonyl)(methyl)amino)-3-(quinolin-6-yl)propanoic acid, (S)-2-((((9H-fluoren-9-yl)methoxy)carbonyl)amino)-3-(2-(tert-butoxycarbonyl)quinolin-6-yl)propanoic acid, (R)-2-((((9H-fluoren-9-yl)methoxy)carbonyl)(methyl)amino)-3-(isoquinolin-6-yl)propanoic acid, (R)-2-((((9H-fluoren-9-yl)methoxy)carbonyl)(methyl)amino)-3-(isoquinolin-7-yl)propanoic acid, (S)-2-((((9H-fluoren-9-yl)methoxy)carbonyl)amino)-3-(2-(2-(tert-butoxy)-2-oxoethoxy)-[1,1′-biphenyl]-4-yl)propanoic acid, (S)-2-((((9H-fluoren-9-yl)methoxy)carbonyl)amino)-3-(1-(2-(tert-butoxy)-2-oxoethyl)-1H-pyrrolo[2,3-b]pyridin-3-yl)propanoic acid, (2S)-3-(1-{4-[(tert-butoxy)carbonyl]phenyl}-1H-indol-3-yl)-2-({[(9H-fluoren-9-yl)methoxy]carbonyl}amino)propanoic acid, (2S)-2-({[(9H-fluoren-9-yl)methoxy]carbonyl}amino)-3-[1-(4-methoxyphenyl)-1H-indol-3-yl]propanoic acid, (S)-2-((((9H-fluoren-9-yl)methoxy)carbonyl)(methyl)amino)-3-(1-(tert-butoxycarbonyl)-1H-pyrrolo[3,2-b]pyridin-3-yl)propanoic acid, (S)-2-((((9H-fluoren-9-yl)methoxy)carbonyl)(methyl)amino)-3-(1-(tert-butoxycarbonyl)-1H-pyrrolo[3,2-c]pyridin-3-yl)propanoic acid, (S)-2-((((9H-fluoren-9-yl)methoxy)carbonyl) amino)-3-(1-(tert-butoxycarbonyl)-6-((4-methoxybenzyl)oxy)-1H-indol-3-yl)propanoic acid, (S)-2-((((9H-fluoren-9-yl)methoxy)carbonyl)amino)-3-(1-(tert-butoxycarbonyl)-6-((tert-butoxycarbonyl)oxy)-1H-indol-3-yl)propanoic acid, (S)-2-((((9H-fluoren-9-yl) methoxy)carbonyl)amino)-3-(1-(4-(tert-butoxycarbonyl)phenyl)-6-((4-methoxybenzyl)oxy)-1H-indol-3-yl)propanoic acid, (S)-2-((((9H-fluoren-9-yl)methoxy)carbonyl)(methy 1)amino)-3-(1-(tert-butoxycarbonyl)-1H-pyrrolo[2,3-c]pyridin-3-yl)propanoic acid, (R)-2-((((9H-fluoren-9-yl)methoxy)carbonyl)amino)-3-(1-(tert-butoxycarbonyl)-7-(pyrimidin-2-yl)-1H-indol-3-yl)propanoic acid, (S)-2-((((9H-fluoren-9-yl)methoxy)carbonyl)amino)-3-(1-(2,4-dimethoxybenzyl)-1H-pyrrolo[2,3-b]pyridin-3-yl)propanoic acid, (S)-2-((((9H-fluoren-9-yl)methoxy)carbonyl)amino)-3-(1-((2,4,6-triisopropylphenyl)sulfonyl)-1H-pyrrolo[2,3-b]pyridin-3-yl)propanoic acid, (S)-2-((((9H-fluoren-9-yl)methoxy) carbonyl)amino)-3-(1-(mesitylsulfonyl)-1H-pyrrolo[2,3-b]pyridin-3-yl)propanoic acid, (S)-2-((((9H-fluoren-9-yl)methoxy)carbonyl)amino)-3-(1-(4-methoxybenzyl)-1H-pyrrolo[2,3-b]pyridin-3-yl)propanoic acid, (S)-2-((((9H-fluoren-9-yl)methoxy)carbonyl)amino)-3-(7-(3-chlorophenyl)-1H-indol-3-yl)propanoic acid, (S)-2-((((9H-fluoren-9-yl)methoxy)carbonyl)amino)-3-(4-(pyridin-2-ylmethoxy)phenyl)propanoic acid, (S)-2-((((9H-fluoren-9-yl)methoxy)carbonyl)amino)-3-(4-(pyridin-3-ylmethoxy)phenyl)propanoic acid, (S)-2-((((9H-fluoren-9-yl)methoxy)carbonyl)amino)-3-(1-(tert-butoxycarbonyl)-7-ethoxy-1H-indol-3-yl)propanoic acid, (S)-2-((((9H-fluoren-9-yl)methoxy)carbonyl)amino)-3-(3-(((tert-butoxycarbonyl)(methyl)amino)methyl)phenyl)propanoic acid, (S)-2-((((9H-fluoren-9-yl)methoxy)carbonyl)amino)-3-(5-(((tert-butoxycarbonyl)amino)methyl)pyridin-3-yl)propanoic acid, (S)-2-((((9H-fluoren-9-yl)methoxy)carbonyl)amino)-3-((R)-3-((tert-butoxycarbonyl)amino)-2,3-dihydro-1H-inden-5-yl)propanoic acid, (S)-2-((((9H-fluoren-9-yl)methoxy)carbonyl)amino)-3-(3-(2-((tert-butoxycarbonyl)amino)ethyl)phenyl)propanoic acid, (S)-2-((((9H-fluoren-9-yl)methoxy)carbonyl)amino)-3-(4-(2-(tert-butoxy)-2-oxoethyl)-3,4-dihydro-2H-benzo[b][1,4]oxazin-6-yl)propanoic acid, (S)-2-((((9H-fluoren-9-yl)methoxy)carbonyl)amino)-3-(1-(tert-butoxycarbonyl)-1,2,3,4-tetrahydroquinolin-7-yl)propanoic acid, (2S)-3-{1-[4-(tert-butoxy)-4-oxobutyl]-1H-indol-3-yl}-2-({[(9H-fluoren-9-yl)methoxy]carbonyl}amino)propanoic acid, (2S)-3-{1-[3-(tert-butoxy)-3-oxopropyl]-1H-indol-3-yl}-2-({[(9H-fluoren-9-yl)methoxy]carbonyl}amino)propanoic acid, (S)-2-((((9H-fluoren-9-yl)methoxy)carbonyl)amino)-3-(4-phenylpiperidin-1-yl)propanoic acid, (R)-2-((((9H-fluoren-9-yl)methoxy)carbonyl)amino)-3-(4-phenylpiperidin-1-yl)propanoic acid, 2-((((9H-fluoren-9-yl)methoxy)carbonyl)amino)-3-(3,4-dihydroisoquinolin-2(1H)-yl)propanoic acid, (S)-2-((((9H-fluoren-9-yl)methoxy)carbonyl)amino)-3-(3′,5′-dichloro-[1,1′-biphenyl]-4-yl)propanoic acid, (S)-2-((((9H-fluoren-9-yl)methoxy)carbonyl)amino)-3-(3′-chloro-5′-fluoro-[1,1′-biphenyl]-4-yl)propanoic acid, (R)-2-((((9H-fluoren-9-yl)methoxy)carbonyl)amino)-3-(3′-carbamoyl-[1,1′-biphenyl]-4-yl)propanoic acid, (R)-2-((((9H-fluoren-9-yl)methoxy)carbonyl)amino)-3-(3′-(tert-butoxy)-[1,1′-biphenyl]-4-yl)propanoic acid, (R)-2-((((9H-fluoren-9-yl)methoxy)carbonyl)amino)-3-(4′-((2-((tert-butoxycarbonyl)amino)ethyl)carbamoyl)-[1,1′-biphenyl]-4-yl)propanoic acid, (R)-2-((((9H-fluoren-9-yl)methoxy)carbonyl)amino)-3-(4′-carbamoyl-[1,1′-biphenyl]-4-yl)propanoic acid, (S)-2-((((9H-fluoren-9-yl)methoxy)carbon yl)amino)-3-(2′-(tert-butoxycarbonyl)-[1,1′-biphenyl]-3-yl)propanoic acid, (S)-2-((((9H-fluoren-9-yl)methoxy)carbonyl)amino)-3-(2′-fluoro-[1,1′-biphenyl]-3-yl)propanoic acid, (S)-2-((((9H-fluoren-9-yl)methoxy)carbonyl)amino)-3-(2′-methoxy-[1,1′-biphenyl]-3-yl)propanoic acid, (S)-2-((((9H-fluoren-9-yl)methoxy)carbonyl)amino)-3-(3-(6-methoxypyridin-3-yl)phenyl)propanoic acid, (S)-2-((((9H-fluoren-9-yl)methoxy)carbonyl) amino)-3-(4′-fluoro-[1,1′-biphenyl]-3-yl)propanoic acid, (S)-2-((((9H-fluoren-9-yl)methoxy)carbonyl)amino)-3-(4′-methoxy-[1,1′-biphenyl]-3-yl)propanoic acid, (S)-2-((((9H-fluoren-9-yl)methoxy)carbonyl)amino)-3-(3-(2-oxo-1,2-dihydropyridin-4-yl)phenyl)propanoic acid, (S)-2-((((9H-fluoren-9-yl)methoxy)carbonyl)amino)-3-(3-(2-methoxypyridin-4-yl)phenyl)propanoic acid, (S)-2-((((9H-fluoren-9-yl)methoxy)carbonyl)amino)-3-(4-(2-((tert-butoxycarbonyl)amino)pyridin-3-yl)phenyl)propanoic acid, (2R,3S)-2-((((9H-fluoren-9-yl)methoxy)carbonyl)amino)-3-cyclopropyl-3-hydroxypropanoic acid, (S)-2-((((9H-fluoren-9-yl)methoxy)carbonyl)amino)-3-(1-phenylpiperidin-4-yl)propanoic acid, (R)-2-((((9H-fluoren-9-yl)methoxy)carbonyl)amino)-3-(1-phenylpiperidin-4-yl) propanoic acid, (R)-2-((((9H-fluoren-9-yl)methoxy)carbonyl)amino)-4-((tert-butoxycarbonyl)amino)-2-methylbutanoic acid, (S)-2-((((9H-fluoren-9-yl)methoxy)carbonyl)amino)-4-methoxy-2-methylbutanoic acid, (S)-2-((((9H-fluoren-9-yl)methoxy)carbonyl)amino)-3-methoxy-2-methylpropanoic acid, (S)-2-((((9H-fluoren-9-yl)methoxy)carbonyl)amino)-6-((R)-2,2-dimethyl-1,3-dioxolan-4-yl)hexanoic acid, (S)-2-((((9H-fluoren-9-yl)methoxy)carbonyl)amino)-6-((S)-2,2-dimethyl-1,3-dioxolan-4-yl)hexanoic acid, (R)-2-((((9H-fluoren-9-yl)methoxy)carbonyl)amino)-6-((R)-2,2-dimethyl-1,3-dioxolan-4-yl)hexanoic acid, (R)-2-((((9H-fluoren-9-yl)methoxy)carbonyl)amino)-6-((S)-2,2-dimethyl-1,3-dioxolan-4-yl)hexanoic acid, (S)-2-((((9H-fluoren-9-yl)methoxy)carbonyl)amino)-4-(1,1-dioxidothiomorpholino)butanoic acid hydrochloride, (S)-2-((((9H-fluoren-9-yl)methoxy)carbonyl)amino)-5-(1,1-dioxidothiomorpholino)pentanoic acid hydrochloride, (2R,3S)-2-((((9H-fluoren-9-yl)methoxy)carbonyl)amino)-3-hydroxypentanoic acid, (S)-2-((((9H-fluoren-9-yl)methoxy)carbonyl)amino)-3-(4-((1-((allyloxy)carbonyl)piperidin-4-yl)oxy)phenyl)propanoic acid, 4-((((9H-fluoren-9-yl)methoxy)carbonyl)amino)-1-(2-(2-methyl-5-nitro-1H-imidazol-1-yl)ethyl)piperidine-4-carboxylic acid, (R)-2-((((9H-fluoren-9-yl)methoxy)carbonyl)amino)-3-(1-(2-(allyloxy)-2-oxoethyl)piperidin-4-yl)propanoic acid, (R)-2-((((9H-fluoren-9-yl)methoxy)carbonyl)amino)-4-(methylsulfonamido)butanoic acid, (R)-2-((((9H-fluoren-9-yl)methoxy)carbonyl)amino)-3-(methylsulfonamido)propanoic acid, (R)-2-((((9H-fluoren-9-yl)methoxy)carbonyl)amino)-5-(((allyloxy)carbonyl)(methyl)amino)pentanoic acid, (S)-2-((((9H-fluoren-9-yl)methoxy)carbonyl)amino)-5-(((allyloxy)carbonyl)(methyl)amino)pentanoic acid, (9H-fluoren-9-yl) methyl (R)-(1-(tert-butoxy)-3-mercaptopropan-2-yl)carbamate, 2-(4-((((9H-fluoren-9-yl)methoxy)carbonyl)amino)piperidin-1-yl)acetic acid hydrochloride, (2S)-2-({[(9H-fluoren-9-yl)methoxy]carbonyl}amino)-4-({2-[(9H-xanthen-9-yl)carbamoyl]ethyl}carbamoyl)butanoic acid, (2S)-2-({[(9H-fluoren-9-yl)methoxy]carbonyl}amino)-4-({3-[(9H-xanthen-9-yl)carbamoyl]propyl}carbamoyl)butanoic acid, 2-[(2-{4-[(tert-butoxy)carbonyl]phenyl}ethyl)({[(9H-fluoren-9-yl)methoxy]carbonyl})amino]acetic acid, 2-{[2-(3,4-difluorophenyl)ethyl]({[(9H-fluoren-9-yl)methoxy]carbonyl})amino}acetic acid, 2-[(2-{3-[(tert-butoxy)carbonyl]phenyl}ethyl)({[(9H-fluoren-9-yl)methoxy]carbonyl})amino]acetic acid, 2-[(3-{3-[(tert-butoxy)carbonyl]phenyl}propyl)({[(9H-fluoren-9-yl)methoxy]carbonyl})amino]acetic acid, 2-[(2-{4-[2-(tert-butoxy)-2-oxoethyl]phenyl}ethyl) ({[(9H-fluoren-9-yl)methoxy]carbonyl})amino]acetic acid, 2-[(3-{4-[(tert-butoxy)carbonyl]phenyl}propyl)({[(9H-fluoren-9-yl)methoxy]carbonyl})amino]acetic acid, 2-{[2-(4-{bis[(2,4-dimethoxyphenyl)methyl]sulfamoyl}phenyl)ethyl]({[(9H-fluoren-9-yl)methoxy]carbonyl})amino}acetic acid, (R)-2-((((9H-fluoren-9-yl)methoxy)carbonyl)(meth yl)amino)-3-cyclopentylpropanoic acid, (S)-2-((((9H-fluoren-9-yl)methoxy)carbonyl)(methyl)amino)-7-((tert-butoxycarbonyl)amino)heptanoic acid, (2S)-2-({[(9H-fluoren-9-yl)methoxy]carbonyl}(methyl)amino)-4-(propan-2-yloxy)butanoic acid, (S)-2-((((9H-fluoren-9-yl)methoxy)carbonyl)(methyl)amino)-3-(methyl(phenyl)amino)propanoic acid, (2R)-2-({[(9H-fluoren-9-yl)methoxy]carbonyl}(propyl)amino)-3-phenylpropanoic acid, (2S)-2-({[(9H-fluoren-9-yl)methoxy]carbonyl}(methyl)amino)-3-(propan-2-yloxy) propanoic acid, (2S)-6-({[(9H-fluoren-9-yl)methoxy]carbonyl}amino)-2-{methyl[(prop-2-en-1-yloxy)carbonyl]amino}hexanoic acid, 3-((((9H-fluoren-9-yl)methoxy)carbon yl)(2-((tert-butoxycarbonyl)amino)ethyl)amino)propanoic acid, 11-(((9H-fluoren-9-yl) methoxy)carbonyl)-2,2-dimethyl-4-oxo-3,8-dioxa-5,11-diazatetradecan-14-oic acid, 3-((((9H-fluoren-9-yl)methoxy)carbonyl)(3-(((allyloxy)carbonyl)amino)propyl)amino)propanoic acid, 3-((((9H-fluoren-9-yl)methoxy)carbonyl)((1-(tert-butoxycarbonyl)azetidin-3-yl)methyl)amino)propanoic acid, 3-((((9H-fluoren-9-yl)methoxy)carbonyl)((1-(tert-butoxycarbonyl)piperidin-4-yl)methyl)amino)propanoic acid, (S)-7-(((9H-fluoren-9-yl)methoxy)carbonyl)-5,6,7,8-tetrahydro-1,7-naphthyridine-6-carboxylic acid, (2R)-2-({[(9H-fluoren-9-yl)methoxy]carbonyl}amino)-6-[(4-{2-[(3-methylpentan-3-yl)oxy]-2-oxoethyl}piperazine-1-carbonyl)amino]hexanoic acid, (2S)-2-((((9H-fluoren-9-yl)methoxy)carbonyl)amino)-3-(4-(3-((tetrahydro-2H-pyran-2-yl)oxy)phenyl)piperidin-1-yl)propanoic acid, (2R)-2-((((9H-fluoren-9-yl)methoxy)carbonyl)amino)-3-(4-(3-((tetrahydro-2H-pyran-2-yl)oxy)phenyl)piperidin-1-yl)propanoic acid, (S)-2-((((9H-fluoren-9-yl)methoxy)carbonyl)amino)-4-(N-(2,4-dimethoxybenzyl)sulfamoyl)butanoic acid, (2R)-3-{bis[(2,4-dimethoxyphenyl)methyl]sulfamoyl}-2-({[(9H-fluoren-9-yl)methoxy]carbonyl}amino)propanoic acid, (S)-2-((((9H-fluoren-9-yl)methoxy)carbonyl)amino)-3-(4-(((R)-1-(tert-butoxy)-1-oxopropan-2-yl)oxy)phenyl)propanoic acid, (S)-2-((((9H-fluoren-9-yl)methoxy)carbonyl)amino)-3-(4-(((S)-1-(tert-butoxy)-1-oxopropan-2-yl)oxy)phenyl)propanoic acid, (2R,3S)-2-((((9H-fluoren-9-yl)methoxy)carbonyl)amino)-3-((tert-butoxycarbonyl)amino)butanoic acid, (R)-5-((((9H-fluoren-9-yl)methoxy)carbonyl)(methyl)amino)-6-phenylhexanoic acid, 2-{[3-({[(9H-fluoren-9-yl)methoxy]carbonyl}amino)propyl][(prop-2-en-1-yloxy)carbonyl]amino}acetic acid, 1-(2-(allyloxy)-2-oxoethyl)piperidine-4-carboxylic acid hydrochloride, 3-((2-((((9H-fluoren-9-yl)methoxy)carbonyl)(methyl)amino)ethyl)(tert-butoxycarbonyl)amino)propanoic acid, (S)-2-((((9H-fluoren-9-yl)methoxy)carbonyl)amino)-3-(1-(tert-butoxycarbonyl)-7-(pyrimidin-2-yl)-1H-indol-3-yl)propanoic acid, (S)-2-((((9H-fluoren-9-yl)methoxy)carbonyl)(methyl)amino)-5-(3-chloropyridin-2-yl)pentanoic acid, (S)-2-((((9H-fluoren-9-yl)methoxy)carbonyl)amino)-3-(5-(2-fluorophenyl)pyridin-3-yl)propanoic acid, (S)-2-((((9H-fluoren-9-yl)meth oxy)carbonyl)amino)-3-(5-(2-methoxyphenyl)pyridin-3-yl)propanoic acid, (S)-3-(6-(1H-imidazol-1-yl)pyridin-3-yl)-2-((((9H-fluoren-9-yl)methoxy)carbonyl)amino)propanoic acid, (S)-2-((((9H-fluoren-9-yl)methoxy)carbonyl)amino)-3-(6-(piperidin-1-yl)pyridin-3-yl)propanoic acid, (R)-2-((((9H-fluoren-9-yl)methoxy)carbonyl)(methyl)amino)-3-(6-((tert-butoxycarbonyl)amino)pyridin-3-yl)propanoic acid, (S)-2-((((9H-fluoren-9-yl)methoxy)carbonyl)amino)-3-(1-(2-((3-methylpentan-3-yl)oxy)-2-oxoethyl)-1H-pyrazol-3-yl)propanoic acid, (R)-2-((((9H-fluoren-9-yl)methoxy)carbonyl)amino)-3-(1H-pyrazolo[3,4-b]pyridin-4-yl)propanoic acid, (R)-2-((((9H-fluoren-9-yl)methoxy)carbonyl)amino)-3-(2-((tert-butoxycarbonyl)(methyl)amino)pyridin-4-yl)propanoic acid, (R)-2-((((9H-fluoren-9-yl)methoxy)carbonyl)(methyl)amino)-3-(2-((tert-butoxycarbonyl)amino) pyridin-4-yl)propanoic acid, (S)-2-((((9H-fluoren-9-yl)methoxy)carbonyl)amino)-3-(1-(2-((3-methylpentan-3-yl)oxy)-2-oxoethyl)-1H-pyrazol-4-yl)propanoic acid, (S)-2-((((9H-fluoren-9-yl)methoxy)carbonyl)(methyl)amino)-3-(2-oxo-1,2-dihydroquinolin-4-yl) propanoic acid, (S)-2-((((9H-fluoren-9-yl)methoxy)carbonyl)amino)-3-(1H-pyrazolo[3,4-b]pyridin-5-yl)propanoic acid, (S)-2-((((9H-fluoren-9-yl)methoxy)carbonyl)(methyl)amino)-3-(1H-pyrazolo[3,4-b]pyridin-5-yl)propanoic acid, (R)-2-((((9H-fluoren-9-yl) methoxy)carbonyl)amino)-3-(2-aminoquinazolin-5-yl)propanoic acid, (S)-2-((((9H-fluoren-9-yl)methoxy)carbonyl)amino)-3-(1H-indazol-6-yl)propanoic acid, (S)-2-((((9H-fluoren-9-yl)methoxy)carbonyl)(methyl)amino)-3-(3-(2-(tert-butoxy)-2-oxoethoxy)phenyl)propanoic acid, (S)-2-((((9H-fluoren-9-yl)methoxy)carbonyl)amino)-3-(3-(2-amino-2-oxoethoxy)phenyl)propanoic acid, (S)-2-((((9H-fluoren-9-yl)methoxy)carbonyl)(methyl)amino)-3-(4-(2-(tert-butoxy)-2-oxoethoxy)phenyl)propanoic acid, (R)-2-((((9H-fluoren-9-yl)methoxy)carbonyl)(methyl)amino)-3-(4-(2-(tert-butoxy)-2-oxoethoxy)phenyl) propanoic acid, (S)-2-((((9H-fluoren-9-yl)methoxy)carbonyl)amino)-4-(2-carbamoylpyridin-3-yl)butanoic acid, (S)-2-((((9H-fluoren-9-yl)methoxy)carbonyl)amino)-4-(4-carbamoylpyridin-3-yl)butanoic acid, (S)-2-((((9H-fluoren-9-yl)methoxy)carbonyl)amino)-4-(6-((2-amino-2-oxoethyl)amino)pyridin-3-yl)butanoic acid, (S)-2-((((9H-fluoren-9-yl)methoxy)carbonyl)amino)-4-(6-((tert-butoxycarbonyl)(methyl)amino)pyridin-3-yl)butanoic acid, (R)-2-((((9H-fluoren-9-yl)methoxy)carbonyl)amino)-4-(6-((tert-butoxycarbonyl)amino)pyridin-3-yl)butanoic acid, (S)-2-((((9H-fluoren-9-yl)methoxy)carbonyl)(methyl)amino)-4-(6-(trifluoromethyl)pyridin-3-yl)butanoic acid, (R)-2-((((9H-fluoren-9-yl)methoxy)carbonyl)amino)-4-(2-((tert-butoxycarbonyl)amino)pyrimidin-5-yl)butan oic acid, (S)-2-((((9H-fluoren-9-yl)methoxy)carbonyl)amino)-4-(4-(tert-butoxycarbonyl)-3,4-dihydro-2H-benzo[b][1,4]oxazin-7-yl)butanoic acid, (S)-2-((((9H-fluoren-9-yl) methoxy)carbonyl)(methyl)amino)-4-(2,3-dihydrobenzo[b][1,4]dioxin-5-yl)butanoic acid, (S)-2-((((9H-fluoren-9-yl)methoxy)carbonyl)(methyl)amino)-4-(2,3-dimethoxyphenyl)butanoic acid, (S)-2-((((9H-fluoren-9-yl)methoxy)carbonyl)(methyl)amino)-4-(benzo[d][1,3]dioxol-4-yl)butanoic acid, (S)-2-((((9H-fluoren-9-yl)methoxy)carbonyl)(meth yl)amino)-4-(2,4-dimethoxyphenyl)butanoic acid, (R)-2-((((9H-fluoren-9-yl)methoxy) carbonyl)amino)-4-(2-(tert-butoxycarbonyl)phenyl)butanoic acid, (S)-2-((((9H-fluoren-9-yl)methoxy)carbonyl)(methyl)amino)-4-(2-((allyloxy)carbonyl)phenyl)butanoic acid, (R)-2-((((9H-fluoren-9-yl)methoxy)carbonyl)(methyl)amino)-4-(2-((allyloxy)carbonyl)phenyl)butanoic acid, (S)-2-((((9H-fluoren-9-yl)methoxy)carbonyl)(methyl)amino)-4-(2-(tert-butoxycarbonyl)phenyl)butanoic acidm (R)-2-((((9H-fluoren-9-yl)methoxy)carbonyl)(methyl)amino)-4-(2-(tert-butoxycarbonyl)phenyl)butanoic acid, (S)-2-((((9H-fluoren-9-yl)methoxy)carbonyl)(methyl)amino)-4-(3-((allyloxy)carbonyl)phenyl)butanoic acid, (R)-2-((((9H-fluoren-9-yl)methoxy)carbonyl)(methyl)amino)-4-(3-((allyloxy)carbonyl)phenyl)butanoic acid, (S)-2-((((9H-fluoren-9-yl)methoxy)carbonyl)(methyl)amino)-4-(o-tolyl)butanoic acid, (S)-2-((((9H-fluoren-9-yl)methoxy)carbonyl)(methyl)amino)-4-(3,4-difluorophenyl)butanoic acid, (S)-2-((((9H-fluoren-9-yl)methoxy)carbonyl) (methyl)amino)-4-(3,5-difluorophenyl)butanoic acid, (S)-2-((((9H-fluoren-9-yl)methoxy)carbonyl)(methyl)amino)-4-(3-(tert-butoxycarbonyl)phenyl)butanoic acid, (R)-2-((((9H-fluoren-9-yl)methoxy)carbonyl)(methyl)amino)-4-(3-(tert-butoxycarbonyl)phenyl)butanoic acid, (S)-2-((((9H-fluoren-9-yl)methoxy)carbonyl)(methyl)amino)-4-(4-(tert-butoxycarbonyl)phenyl)butanoic acid, (R)-2-((((9H-fluoren-9-yl)methoxy)carbonyl)(methyl)amino)-4-(4-(tert-butoxycarbonyl)phenyl)butanoic acid, (S)-2-((((9H-fluoren-9-yl)methoxy)carbonyl)(methyl)amino)-4-(3-(trifluoromethyl)phenyl)butanoic acid, (S)-2-((((9H-fluoren-9-yl)methoxy)carbonyl)(methyl)amino)-4-(4-((allyloxy)carbonyl)phenyl)butanoic acid, (R)-2-((((9H-fluoren-9-yl)methoxy)carbonyl)(methyl)amino)-4-(4-((allyloxy)carbonyl)phenyl)butanoic acid, (S)-2-((((9H-fluoren-9-yl)methoxy)carbonyl)(methyl)amino)-4-(4-(trifluoromethyl)phenyl)butanoic acid, (R)-2-((((9H-fluoren-9-yl)methoxy)carbonyl)amino)-3-(2-((tert-butoxycarbonyl)amino)quinolin-5-yl)propanoic acid, (S)-2-((((9H-fluoren-9-yl)methoxy)carbonyl)amino)-3-(2-methoxy-[1,1′-biphenyl]-4-yl)propanoic acid, (S)-2-((((9H-fluoren-9-yl)methoxy)carbonyl)(methyl)amino)-5-(2-chlorophenyl)pentanoic acid, (S)-2-((((9H-fluoren-9-yl)methoxy)carbonyl)(methyl)amino)-5-(2-chloro-3-fluorophenyl)pentanoic acid, (S)-2-((((9H-fluoren-9-yl)methoxy)carbonyl)(methyl)amino)-5-(2-chloro-5-fluorophenyl)pentanoic acid, (S)-2-((((9H-fluoren-9-yl)methoxy)carbonyl)(methyl)amino)-5-(2-fluorophenyl)pentanoic acid, (S)-2-((((9H-fluoren-9-yl)methoxy)carbonyl)(methyl)amino)-5-(3-chlorophenyl)pentanoic acid, (S)-2-((((9H-fluoren-9-yl)methoxy)carbonyl)(methyl)amino)-5-(3-fluorophenyl)pentanoic acid, (S)-2-((((9H-fluoren-9-yl)methoxy)carbonyl)(methyl)amino)-5-(3-(trifluoromethyl)phenyl)pentanoic acid, (S)-2-((((9H-fluoren-9-yl)methoxy)carbonyl)(methyl)amino)-5-(4-chlorophenyl)pentanoic acid, (S)-2-((((9H-fluoren-9-yl)methoxy)carbonyl)(methyl)amino)-5-(4-fluorophenyl)pentanoic acid, (S)-2-((((9H-fluoren-9-yl)methoxy)carbonyl)(methyl)amino)-5-(4-methoxyphenyl)pentanoic acid, (S)-2-((((9H-fluoren-9-yl)methoxy)carbonyl)(methyl)amino)-5-(4-(trifluoromethyl)phenyl)pentanoic acid, (S)-2-((((9H-fluoren-9-yl)methoxy)carbonyl)amino)-3-(1-(2-(tert-butoxy)-2-oxoethyl)-5-chloro-1H-indol-3-yl)propanoic acid, (S)-2-((((9H-fluoren-9-yl)methoxy)carbonyl)amino)-3-(5-fluoro-1H-indazol-3-yl)propanoic acid, (S)-2-((((9H-fluoren-9-yl)methoxy)carbonyl)amino)-3-(6-chloro-1H-pyrrolo[2,3-b]pyridin-3-yl)propanoic acid, (S)-2-((((9H-fluoren-9-yl)methoxy)carbonyl)amino)-3-(6-((tert-butoxycarbonyl)oxy)-1-(4-(tert-butoxycarbonyl)phenyl)-1H-indol-3-yl)propanoic acid, (S)-2-((((9H-fluoren-9-yl)methoxy)carbonyl)(methyl)amino)-3-(4-(2-(((allyloxy)carbonyl)amino)ethoxy)phenyl)propanoic acid, (S)-2-((((9H-fluoren-9-yl)methoxy)carbonyl)(methyl)amino)-3-(4-(2-((tert-butoxycarbonyl)amino)ethoxy)phenyl)propanoic acid, (S)-2-((((9H-fluoren-9-yl)methoxy)carbonyl)amino)-3-(4-(3-((tert-butoxycarbonyl)amino)propoxy)phenyl)propanoic acid, (S)-2-((((9H-fluoren-9-yl)methoxy)carbonyl)amino)-3-morpholinopropanoic acid hydrochloride, (S)-2-((((9H-fluoren-9-yl)methoxy)carbonyl)amino)-3-(4-phenylpiperidin-1-yl)propanoic acid hydrochloride, (R)-2-((((9H-fluoren-9-yl)methoxy)carbonyl)amino)-3-(4-phenylpiperidin-1-yl)propanoic acid hydrochloride, (S)-2-((((9H-fluoren-9-yl)methoxy) carbonyl)amino)-3-(4-phenylpiperazin-1-yl)propanoic acid dihydrochloride, (R)-2-((((9H-fluoren-9-yl)methoxy)carbonyl)amino)-3-(4-phenylpiperazin-1-yl)propanoic acid dihydrochloride, (S)-2-((((9H-fluoren-9-yl)methoxy)carbonyl)amino)-4-morpholinobutanoic acid hydrochloride, (2S)-2-({[(9H-fluoren-9-yl)methoxy]carbonyl}amino)-4-[2-(morpholin-4-yl)ethoxy]butanoic acid, (2S)-4-[2-(3,3-difluoropyrrolidin-1-yl)ethoxy]-2-({[(9H-fluoren-9-yl)methoxy]carbonyl}amino)butanoic acid, (2S)-2-({[(9H-fluoren-9-yl)methoxy]carbonyl}amino)-4-(2-methylpropoxy)butanoic acid, (2R)-2-({[(9H-fluoren-9-yl)methoxy]carbonyl}amino)-3-[2-oxo-2-(prop-2-en-1-yloxy)ethoxy]propanoic acid, (2R)-3-(2-{[(tert-butoxy)carbonyl]amino}ethoxy)-2-({[(9H-fluoren-9-yl)methoxy]carbonyl}amino)propanoic acid, (2S)-3-(2-azidoethoxy)-2-({[(9H-fluoren-9-yl)methoxy]carbonyl}amino)propanoic acid, (2S)-3-[2-(3,3-difluoropiperidin-1-yl)ethoxy]-2-({[(9H-fluoren-9-yl)methoxy]carbonyl}amino)propanoic acid, (2S)-3-[2-(3,3-difluoropyrrolidin-1-yl)ethoxy]-2-({[(9H-fluoren-9-yl)methoxy]carbonyl}amino)propanoic acid, (2S)-3-(2,2-difluoroethoxy)-2-({[(9H-fluoren-9-yl)methoxy]carbonyl}amino)propanoic acid, (2S)-2-({[(9H-fluoren-9-yl)methoxy]carbonyl}amino)-3-(2-fluoroethoxy)propanoic acid, (2S)-3-({1-[(tert-butoxy)carbonyl]piperidin-4-yl}methoxy)-2-({[(9H-fluoren-9-yl)methoxy]carbonyl}amino)propanoic acid, (2S)-2-({[(9H-fluoren-9-yl)methoxy]carbonyl}amino)-3-[(oxan-4-yl)methoxy]propanoic acid, (2S)-3-(cyclohexylmethoxy)-2-({[(9H-fluoren-9-yl)methoxy]carbonyl}amino)propanoic acid, (2S)-2-({[(9H-fluoren-9-yl)methoxy]carbonyl}amino)-3-(3-fluoropropoxy)propanoic acid, (R)-2-((((9H-fluoren-9-yl)methoxy)carbonyl)amino)-7-(allyloxy)-7-oxoheptanoic acid, (S)-2-((((9H-fluoren-9-yl)methoxy)carbonyl)amino)-3-(1-(2-hydroxyethyl)piperidin-4-yl)propanoic acid, (S)-2-((((9H-fluoren-9-yl)methoxy)carbonyl)amino)-3-(1-(2-methoxyethyl)piperidin-4-yl)propanoic acid, (S)-2-((((9H-fluoren-9-yl)methoxy)carbonyl)amino)-3-(1-(2,2,2-trifluoroethyl)piperidin-4-yl)propanoic acid, (S)-2-((((9H-fluoren-9-yl)methoxy)carbonyl) amino)-3-(1-methylpiperidin-4-yl)propanoic acid hydrochloride, (S)-39-((((9H-fluoren-9-yl)methoxy)carbonyl)amino)-5,33-dioxo-9,12,15,18,21,24,27,30-octaoxa-6,34-diazatetracont-1-yn-40-oic acid, (S)-2-((((9H-fluoren-9-yl)methoxy)carbonyl)amino)-3-(4-((1-(3-(2-((tert-butoxycarbonyl)amino)ethoxy)propanoyl)piperidin-4-yl)oxy)phenyl)propanoic acid, (S)-2-((((9H-fluoren-9-yl)methoxy)carbonyl)amino)-4-((9H-xanthen-9-yl) amino)-2-methyl-4-oxobutanoic acid, (R)-2-((((9H-fluoren-9-yl)methoxy)carbonyl)amino)-4-((9H-xanthen-9-yl)amino)-2-methyl-4-oxobutanoic acid, (2S)-6-[5-(tert-butoxy)-5-oxopentanamido]-2-({[(9H-fluoren-9-yl)methoxy]carbonyl}(methyl)amino)hexanoic acid, (S)-14-((((9H-fluoren-9-yl)methoxy)carbonyl)amino)-2,2-dimethyl-4,12-dioxo-3,8-dioxa-5,11-diazapentadecan-15-oic acid, (S)-15-((((9H-fluoren-9-yl)methoxy)carbonyl)amino)-2,2-dimethyl-4,12-dioxo-3,8-dioxa-5,11-diazahexadecan-16-oic acid, 2-({[(9H-fluoren-9-yl)methoxy]carbonyl}[3-(pyridin-3-yl)propyl]amino)acetic acid hydrochloride, 2-({[(9H-fluoren-9-yl)methoxy]carbonyl}({2-[3-(trifluoromethyl)phenyl]ethyl})amino)acetic acid, 2-({[(9H-fluoren-9-yl)methoxy]carbonyl}[3-(pyridin-4-yl)propyl]amino)acetic acid hydrochloride, 2-({[(9H-fluoren-9-yl)methoxy]carbonyl}({2-[4-(trifluoromethyl)phenyl]ethyl})amino)acetic acid, 2-[(2-cyclobutylethyl)({[(9H-fluoren-9-yl)methoxy]carbonyl})amino]acetic acid, 2-[(3,3-dimethylbutyl)({[(9H-fluoren-9-yl)methoxy]carbonyl})amino]acetic acid, (R)-2-((((9H-fluoren-9-yl)methoxy)carbonyl)(methyl)amino)hept-6-enoic acid, (1S,2S)-2-((((9H-fluoren-9-yl)methoxy)carbonyl)(methyl)amino)cyclopentane-1-carboxylic acid, (2R,3S)-3-((((9H-fluoren-9-yl)methoxy)carbonyl)(methyl)amino)-2-methylbutanoic acid, (S)-2-((((9H-fluoren-9-yl)methoxy)carbonyl)(methyl)amino)-4-((tert-butoxycarbonyl)(methyl)amino)butanoic acid, (2S,3S)-2-((((9H-fluoren-9-yl)methoxy)carbonyl)(methyl)amino)-3-phenylbutanoic acid, (S)-2-((((9H-fluoren-9-yl)methoxy)carbonyl)(methyl)amino)-6-amino-6-oxohexanoic acid, (S)-2-((((9H-fluoren-9-yl)methoxy)carbonyl)(methyl)amino)-6-morpholinohexanoic acid hydrochloride, (S)-2-((((9H-fluoren-9-yl)methoxy)carbonyl)(methyl)amino)-4-cyclohexylbutanoic acid, (S)-2-((((9H-fluoren-9-yl)methoxy)carbonyl)(methyl)amino)-4-cyclopentylbutanoic acid, (R)-2-((((9H-fluoren-9-yl)methoxy)carbonyl)(methyl)amino)-4-cyclopentylbutanoic acid, (S)-2-((((9H-fluoren-9-yl)methoxy)carbonyl)(methyl)amino)-5-cyclohexylpentanoic acid, (R)-2-((((9H-fluoren-9-yl)methoxy)carbonyl)(methyl)amino)-5-cyclohexylpentanoic acid, (S)-2-((((9H-fluoren-9-yl)methoxy)carbonyl)(methyl)amino)-5-cyclopentylpentanoic acid, (S)-2-((((9H-fluoren-9-yl)methoxy)carbonyl)(methyl)amino)-6,6-dimethylheptanoic acid, (R)-2-((((9H-fluoren-9-yl)methoxy)carbonyl)(methyl)amino)-6,6-dimethylheptanoic acid, (2S)-3-[(2-chlorophenyl)methoxy]-2-({[(9H-fluoren-9-yl)methoxy]carbonyl}(methyl)amino)propanoic acid, (2S)-3-(cyclohexyloxy)-2-({[(9H-fluoren-9-yl)methoxy]carbonyl}(methyl)amino)propanoic acid, (2S,3S)-3-(tert-butoxy)-2-({[(9H-fluoren-9-yl)methoxy]carbonyl}(methyl)amino)butanoic acid, (2S,3R)-2-({[(9H-fluoren-9-yl)methoxy]carbonyl}(methyl)amino)-3-propoxybutanoic acid, (R)-3-((((9H-fluoren-9-yl)methoxy)carbonyl)(methyl)amino)-5-((3-methylpentan-3-yl)oxy)-5-oxopentanoic acid, (S)-3-((((9H-fluoren-9-yl)methoxy)carbonyl)(3-((tert-butoxycarbonyl)amino)propyl)amino)butanoic acid, (R)-3-((((9H-fluoren-9-yl)methoxy)carbonyl)(3-((tert-butoxycarbonyl)amino)propyl)amino)butanoic acid, (S)-3-((((9H-fluoren-9-yl)methoxy)carbonyl)(methyl)amino)-5-((tert-butoxycarbonyl)(methyl)amino)pentanoic acid, (S)-3-((((9H-fluoren-9-yl)methoxy)carbonyl)(methyl)amino)-7-((tert-butoxycarbonyl)amino)heptanoic acid, (R)-3-((((9H-fluoren-9-yl)methoxy)carbonyl)(methyl)amino)-7-((tert-butoxycarbonyl)amino)heptanoic acid, (S)-3-((((9H-fluoren-9-yl)methoxy)carbonyl)(methyl)amino)-7-((tert-butoxycarbonyl)(methyl)amino)heptanoic acid, (R)-2-((((9H-fluoren-9-yl)methoxy)carbonyl)amino)-3-(4-((4-(2-((3-methylpentan-3-yl)oxy)-2-oxoethyl)piperazine-1-carboxamido)methyl)phenyl)propanoic acid, (S)-2-((((9H-fluoren-9-yl)methoxy)carbonyl)(methyl)amino)-3-(4-((4-(2-((3-methylpentan-3-yl) oxy)-2-oxoethyl)piperazine-1-carboxamido)methyl)phenyl)propanoic acid, (R)-2-((((9H-fluoren-9-yl)methoxy)carbonyl)(methyl)amino)-3-(4-((4-(2-((3-methylpentan-3-yl)ox y)-2-oxoethyl)piperazine-1-carboxamido)methyl)phenyl)propanoic acid, (2S)-2-({[(9H-fluoren-9-yl)methoxy]carbonyl}(methyl)amino)-6-[(4-{2-[(3-methylpentan-3-yl)oxy]-2-oxoethyl}piperazine-1-carbonyl)amino]hexanoic acid, (2S,4R)-1-(((9H-fluoren-9-yl) methoxy)carbonyl)-4-(3-(2-(tert-butoxy)-2-oxoethyl)ureido)pyrrolidine-2-carboxylic acid, (2S,4S)-1-(((9H-fluoren-9-yl)methoxy)carbonyl)-4-(3-(2-(tert-butoxy)-2-oxoethyl)ureido)pyrrolidine-2-carboxylic acid, (2R,4R)-1-(((9H-fluoren-9-yl)methoxy)carbonyl)-4-(3-(2-(tert-butoxy)-2-oxoethyl)ureido)pyrrolidine-2-carboxylic acid, (2R,4S)-1-(((9H-fluoren-9-yl)methoxy)carbonyl)-4-(3-(2-(tert-butoxy)-2-oxoethyl)ureido)pyrrolidine-2-carboxylic acid, (R)-2-((((9H-fluoren-9-yl)methoxy)carbonyl)amino)-3-(4-(2-(4-(2-((3-methylpentan-3-yl)oxy)-2-oxoethyl)piperazine-1-carboxamido)ethoxy)phenyl)propanoic acid, (S)-2-((((9H-fluoren-9-yl)methoxy)carbonyl)(methyl)amino)-3-(4-(2-(4-(2-((3-methylpentan-3-yl)oxy)-2-oxoethyl)piperazine-1-carboxamido)ethoxy)phenyl)propanoic acid, (R)-2-((((9H-fluoren-9-yl)methoxy)carbonyl)(methyl)amino)-3-(4-(2-(4-(2-((3-methylpentan-3-yl)oxy)-2-oxoethyl)piperazine-1-carboxamido)ethoxy)phenyl)propanoic acid, (2R)-3-{[(2,4-dimethoxyphenyl)methyl](methyl)sulfamoyl}-2-({[(9H-fluoren-9-yl)methoxy]carbonyl}amino)propanoic acid, (2R,4R)-1-(((9H-fluoren-9-yl)methoxy)carbonyl)-4-(2-(tert-butoxy)-2-oxoethoxy)pyrrolidine-2-carboxylic acid, (2R,4S)-1-(((9H-fluoren-9-yl)methoxy)carbonyl)-4-(2-(tert-butoxy)-2-oxoethoxy)pyrrolidine-2-carboxylic acid, (2S,4S)-1-(((9H-fluoren-9-yl)methoxy)carbonyl)-4-(2-(tert-butoxy)-2-oxoethoxy)pyrrolidine-2-carboxylic acid, (S)-2-((((9H-fluoren-9-yl)methoxy)carbonyl)amino)-3-(7-(5-iodopyrimidin-2-yl)-1H-indol-3-yl)propanoic acid, (2S)-4-{[2-(2,6-dioxopiperidin-3-yl)-1,3-dioxo-2,3-dihydro-1H-isoindol-4-yl]oxy}-2-({[(9H-fluoren-9-yl)methoxy]carbonyl}amino)butanoic acid, (2S)-4-{[2-(2,6-dioxopiperidin-3-yl)-1,3-dioxo-2,3-dihydro-1H-isoindol-4-yl]oxy}-2-({[(9H-fluoren-9-yl)methoxy]carbonyl}(methyl)amino) butanoic acid, and (2S)-5-{[2-(2,6-dioxopiperidin-3-yl)-1,3-dioxo-2,3-dihydro-1H-isoindol-4-yl]oxy}-2-({[(9H-fluoren-9-yl)methoxy]carbonyl}amino)pentanoic acid.

### Exemplary Description of Synthesizing the Amino Acid Derivatives

### Reference Example-1-01: methyl (2R)-2-({[(9H-fluoren-9-yl)methoxy]carbonyl}(methyl)amino)-3-iodopropanoate

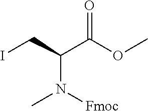

To a mixture of methyl (2S)-3-hydroxy-2-(methylamino)propanoate (CAS: 111934-24-8, 64.2 g, 482 mmol) in H₂O (700 mL) and 1,4-dioxane (700 mL) at 0° C. were added Na₂CO₃ (81.0 g, 964 mmol) and Fmoc-OSu (179 g, 531 mmol). The mixture was stirred at rt for 16 h. The reaction was quenched with H₂O. The mixture was extracted three times with EtOAc. The combined organic extracts were dried over Na₂SO₄, filtered, and then concentrated. The resulting residue was purified by silica gel flash column chromatography (isocratic, petroleum ether/EtOAc=50/50). Appropriate fractions were concentrated (73.8 g, 208 mmol). To a solution of the obtained material (60.0 g, 169 mmol) in CH₂Cl₂ (1.00 L) at 0° C. were added triphenylphosphine (66.4 g, 253 mmol), imidazole (17.2 g, 253 mmol), and iodine (64.3 g, 253 mmol). The mixture was stirred at rt for 0.25 h and then concentrated. The resulting residue was purified by silica gel flash column chromatography (isocratic, petroleum ether/EtOAc=83/17) to afford the title compound (11.0 g, 23.6 mmol). LC-MS (ESI, m/z): [M+H]⁺ 466.

The following compounds were synthesized as outlined for the preparation of Reference Example-1-01 employing appropriate starting materials in the table.

| | Structure and IUPAC name |
|---|---|
| Reference | Starting material acid |
| Example | MS data |
| 1-02 | 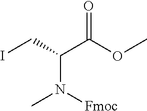 |
| | methyl (2S)-2-({[(9H-fluoren-9-yl)methoxy]carbonyl}(methyl)amino)-3- |
| | iodopropanoate |
| | methyl (2R)-3-hydroxy-2-(methylamino)propanoate (CAS: 1315054-06-8) |
| | Substantially identical to Reference Example-1-01 |

### Reference Example-1-03: methyl (2R)-2-({[(9H-fluoren-9-yl)methoxy]carbonyl}(methyl)amino)-4-iodobutanoate

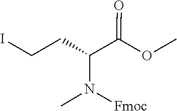

To a solution of (R)-2-((((9H-fluoren-9-yl)methoxy)carbonyl)(methyl)amino)-4-(tert-butoxy)-4-oxobutanoic acid (CAS: 1799443-40-5, 1.50 g, 3.53 mmol) in toluene (17.6 mL) and MeOH (7.13 mL) at 0° C. were added a 0.6M solution of trimethylsilyldiazomethane in hexane (7.64 mL, 4.58 mmol). The mixture was stirred at 0° C. for 10 min and then quenched with a few drops of AcOH. The resulting mixture was concentrated and azeotroped three times with toluene. To a solution of the resulting residue in DCM (17.6 mL) at 0° C. was added TFA (70.6 mL). The mixture was stirred at rt for 90 min, and then concentrated, and azeotroped three times with toluene. To a solution of the obtained material (0.2 g, 0.52 mmol) in THF (4.74 mL) at −10° C. were added 4-methylmorpholine (0.08 mL, 0.68 mmol) and ethyl carbonochloridate (0.06 mL, 0.57 mmol). The mixture was stirred at −10° C. for 30 min. To the mixture at −10° C. was added a solution of sodium borohydride (0.02 g, 0.522 mmol) in water (0.47 mL) dropwise. The mixture was stirred at −10° C. for 10 min. The mixture was poured into satd. aq. NH₄Cl. The mixture was extracted with EtOAc. The combined organic extracts were washed with brine, dried over Na₂SO₄, filtered, and concentrated. The resulting residue was purified by silica gel flash column chromatography (gradient, heptane/EtOAc=85/15 to 30/70). Appropriate fractions were concentrated. To a solution of the obtained material (0.11 g, 0.30 mmol) in DCM (2.0 mL) at 0° C. were added triphenylphosphine (0.16 g, 0.60 mmol), imidazole (0.04 g, 0.60 mmol), and iodine (0.15 g, 0.60 mmol). The mixture was stirred at rt for 1 hand then concentrated. The resulting residue was purified by silica gel flash column chromatography (gradient, heptane/EtOAc=100/0 to 50/50) to afford the title compound (0.13 g, 0.27 mmol). LC-MS (ESI, m/z): [M+H]⁺ 480.

The following compounds can be synthesized as outlined for the preparation of Reference Example-1-03 employing appropriate starting materials in the table.

| | Structure and IUPAC name |
|---|---|
| Reference | Starting material acid |
| Example | MS data |
| 1-04 | 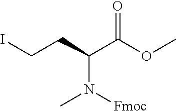 |
| | methyl (2S)-2-({[(9H-fluoren-9-yl)methoxy]carbonyl}(methyl)amino)-4- |
| | iodobutanoate |
| | (S)-2-((((9H-fluoren-9-yl)methoxy)carbonyl)(methyl)amino)-4-(tert- |
| | butoxy)-4-oxobutanoic acid (CAS: 152548-66-8) |
| | Substantially identical to Reference Example-1-03 |
| 1-05 | 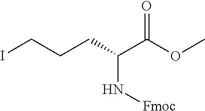 |
| | methyl (R)-2-((((9H-fluoren-9-yl)methoxy)carbonyl)amino)-5- |
| | iodopentanoate |
| | (R)-2-((((9H-fluoren-9-yl)methoxy)carbonyl)amino)-5-(tert-butoxy)-5- |
| | oxopentanoic acid (CAS: 104091-08-9) |
| | LC-MS (ESI, m/z): [M + H]⁺ 480. |
| 1-06 | 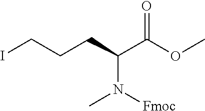 |
| | methyl (S)-2-((((9H-fluoren-9-yl)methoxy)carbonyl)(methyl)amino)-5- |
| | iodopentanoate |
| | (S)-2-((((9H-fluoren-9-yl)methoxy)carbonyl)(methyl)amino)-5-(tert- |
| | butoxy)-5-oxopentanoic acid (CAS: 200616-40-6) |
| | LC-MS (ESI, m/z): [M + H]⁺ 494. |
| 1-07 | 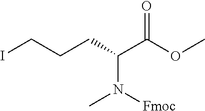 |
| | methyl (R)-2-((((9H-fluoren-9-yl)methoxy)carbonyl)(methyl)amino)-5- |
| | iodopentanoate |
| | (R)-2-((((9H-fluoren-9-yl)methoxy)carbonyl)(methyl)amino)-5-(tert- |
| | butoxy)-5-oxopentanoic acid (CAS: 1562442-35-6) |
| | Substantially identical to Reference Example-1-06 |

### Reference Example-2-01: (2R)-3-{3-[(tert-butoxy)carbonyl]phenyl}-2-({[(9H-fluoren-9-yl)methoxy]carbonyl}amino)propanoic acid

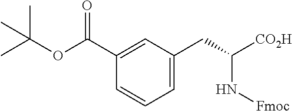

To a suspension of Zn (3.16 g, 48.4 mmol) in DMF (100 mL) was added I₂ (0.62 g, 2.43 mmol). The mixture was stirred at rt for 10 min. To the mixture at rt was added methyl (2S)-2-[[(9H-fluoren-9-ylmethoxy)carbonyl]amino]-3-iodopropanoate (CAS: 527696-77-1, 7.30 g, 16.2 mmol). The mixture was stirred at rt for an additional 1 h. To the mixture were added tert-butyl 3-bromobenzoate (CAS: 69038-74-0, 4.16 g, 16.2 mmol), Pd₂(dba)₃CHCl₃ adduct (0.50 g, 0.485 mmol), and SPhos (0.33 g, 0.81 mmol). The mixture was stirred at 50° C. for an additional 12 h, and then filtered. The filtrate was diluted with H₂O. The mixture was extracted three times with EtOAc. The combined organic extracts were washed with brine, dried over Na₂SO₄, filtered, and then concentrated. The resulting residue was purified by silica gel flash column chromatography (gradient, petroleum ether/EtOAc=99/1 to 75/25). Appropriate fractions were concentrated. To a solution of the resulting residue (5.36 g, 10.7 mmol) in ClCH₂CH₂Cl (50 mL) was added trimethyltin hydroxide (3.89 g, 21.5 mmol). The resulting mixture was stirred at 60° C. for 3 h. The reaction was quenched with aq. citric acid. The mixture was extracted three times with CH₂Cl₂. The combined organic extracts were washed with brine, dried over Na₂SO₄, filtered, and then concentrated. The resulting residue was purified by silica gel flash column chromatography (gradient, petroleum ether/EtOAc=99/1 to 50/50), and then further purified by reversed phase silica gel flash column chromatography (C18 silica gel, gradient, H₂O/CH₃CN=50/50 to 20/80) to afford the title compound (3.20 g, 6.56 mmol). LC-MS (ESI, m/z): [M+H]⁺ 486.

### Reference Example-2-02: (2S)-3-(3-carbamoyl-4-hydroxyphenyl)-2-({[(9H-fluoren-9-yl)methoxy]carbonyl}amino)propanoic acid

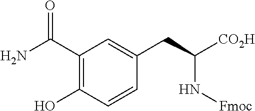

To a suspension of Zn (54.5 g, 833 mmol) in DMF (2000 mL) at rt was added I₂ (10.6 g, 41.7 mmol). The mixture was stirred at rt for 15 min. To the mixture at rt were added methyl (R)-2-((((9H-fluoren-9-yl)methoxy)carbonyl)amino)-3-iodopropanoate (CAS: 156017-42-4, 125.3 g, 278 mmol) and I₂ (10.6 g, 41.7 mmol). The mixture was stirred at rt for 1 h. To the mixture at rt were added 5-bromo-2-hydroxybenzamide (CAS: 6329-74-4, 60.0 g, 278 mmol), Pd₂(dba)₃CHCl₃ adduct (12.7 g, 13.9 mmol), and SPhos (11.4 g, 27.8 mmol). The mixture was stirred at 50° C. for 4 h. The mixture was quenched with H₂O. The mixture was extracted three times with EtOAc. The organic extracts were washed with brine, dried over Na₂SO₄, filtered, and then concentrated. The resulting residue was purified by silica gel flash column chromatography (isocratic, petroleum ether/EtOAc=25/75). Appropriate fractions were concentrated. To a mixture of the obtained material (90.0 g, 195 mmol) in 2-propanol (900 mL), THF (300 mL), and H₂O (200 mL) at 5° C. were added CaCl₂ (346 g, 3.12 mol) and LiOH·H₂O (33.0 g, 786 mmol). The mixture was stirred at rt overnight. The pH of the mixture was adjusted to pH 4 with satd. aq. NaH₂PO₄. The resulting mixture was extracted three times with EtOAc. The combined organic extracts were washed three times with H₂O, dried over Na₂SO₄, filtered, and then concentrated. The resulting residue was purified by silica gel flash column chromatography (isocratic, CH₂Cl₂/MeOH=95/5) to afford the title compound (53 g, 119 mmol). LC-MS (ESI, m/z): [M+H]⁺ 447.

### Reference Example-2-03: (2S)-3-(2-carbamoylpyridin-4-yl)-2-({[(9H-fluoren-9-yl)methoxy]carbonyl}amino)propanoic acid

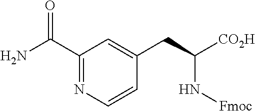

To a suspension of Zn (23.3 g, 356 mmol) in DMF (150 mL) at rt were added 1,2-dibromoethane (1.88 mL, 22.0 mmol) and chlorotrimethylsilane (0.765 mL, 6.03 mmol). The mixture was stirred at 60° C. for 30 min. To the mixture at rt was added methyl (2R)-2-[[(tert-butoxy)carbonyl]amino]-3-iodopropanoate (CAS: 93267-04-0, 32.9 g, 100 mmol) in DMF (50 mL) dropwise with stirring in 30 min. To the mixture were added a solution of 4-bromopyridine-2-carboxamide (CAS: 62150-46-3, 20.0 g, 99.0 mmol) in DMF (300 mL) and Pd(PPh₃)₂Cl₂ (3.50 g, 4.99 mmol). The resulting solution was stirred overnight at 50° C. The reaction was quenched with H₂O. The mixture was filtered. The filtrate was extracted with DCM/MeOH (10/1). The organic extract was washed with brine, dried over Na₂SO₄, filtered, and then concentrated. The resulting residue was purified by silica gel flash column chromatography (gradient, petroleum ether/EtOAc=83/17 to 0/100). Appropriate fractions were concentrated. To a mixture of the residue (15.0 g, 46.4 mmol) in MeOH (200 mL) at 0° C. was added a solution of sodium hydroxide (1.86 g, 46.5 mmol) in H₂O (200 mL) dropwise. The mixture was stirred at 0° C. for 2 h. The pH of the mixture was adjusted to pH 4 with 2M aq. HCl. The resulting mixture was concentrated. To a solution of the residue (16.0 g, 51.7 mmol) in H₂O (44 mL) at 0° C. was added aq. hydrogen chloride (22 mL) dropwise with stirring. The resulting solution was stirred for 4 h at rt. The pH of the mixture was adjusted to 7 with 1M aq. sodium hydroxide. The resulting mixture was concentrated. To a solution of the residue (13.0 g, 62.1 mmol) in 1,4-dioxane (150 mL) at 0° C. were added a solution of NaHCO₃ (10.5 g, 124 mmol) in H₂O (150 mL) and Fmoc-Cl (16.1 g, 62.1 mmol). The mixture was stirred for 2 h at rt, and the resulting mixture was concentrated. The resulting residue was purified by reversed phase silica gel flash column chromatography (C18 silica gel, gradient, H₂O/CH₃CN=80/20 to 40/60) to afford the title compound (5.00 g, 11.6 mmol). LC-MS (ESI, m/z): [M+H]⁺ 432.

### Reference Example-2-04: (2R)-3-(3-carbamoylphenyl)-2-({[(9H-fluoren-9-yl)methoxy]carbonyl}amino)propanoic acid

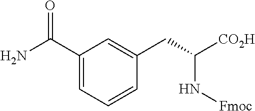

To a suspension of Zn (7.24 g, 111 mmol) in DMF (44.3 mL) at rt were added 1,2-dibromoethane (0.19 mL, 2.22 mmol) and chlorotrimethylsilane (0.29 mL, 2.22 mmol). The mixture was stirred at 60° C. for 30 min. To the mixture at rt was added methyl (2S)-2-({[(9H-fluoren-9-yl)methoxy]carbonyl}amino)-3-iodopropanoate (CAS: 527696-77-1, 10.00 g, 22.16 mmol). The mixture was stirred at rt for 1.5 h. Then the mixture of Pd₂(dba)₃CHCl₃ adduct (1.15 g, 1.11 mmol), and SPhos (1.82 g, 4.43 mmol) in DMF (22.2 mL) was stirred at 60° C. for 30 min. To the Pd complex mixture at rt were added 3-bromobenzamide (CAS: 22726-00-7, 5.32 g, 26.6 mmol) and organozinc reagent. The mixture was stirred at 50° C. for 3 h. The reaction was quenched with EtOAc and 2M aq. HCl at 0° C. The mixture was filtered. The filtrate was extracted with EtOAc. The organic extract was washed with brine, dried over Na₂SO₄, filtered, and then concentrated. The resulting residue was purified by silica gel flash column chromatography (gradient, Heptane/EtOAc=100/0 to 0/100). Appropriate fractions were concentrated. To the resulting residue (3.4 g, 7.65 mmol) in 1,4-dioxane (25.50 mL) and water (12.75 mL) at rt was added 12M HCl aq. (12.75 mL, 153 mmol). The mixture was stirred at 80° C. for 1 h, and then diluted with EtOAc and water. The mixture was extracted twice with EtOAc. The combined organic extracts were washed with H₂O and brine, dried over Na₂SO₄, filtered, and then concentrated. The resulting residue was purified by silica gel flash column chromatography (gradient, heptane/EtOAc=95/5 to 60/40) to afford the title compound (1.46 g, 3.39 mmol). LC-MS (ESI, m/z): [M+H]⁺ 431.

### Reference Example-2-05: (2S)-2-({[(9H-fluoren-9-yl)methoxy]carbonyl}amino)-3-[3-({[(prop-2-en-1-yloxy)carbonyl]amino}methyl)phenyl]propanoic acid

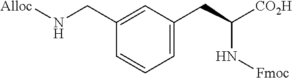

To a solution of 1-(3-bromophenyl)methanamine (CAS: 10269-01-9, 20 g, 108 mmol) in CH₂Cl₂ (200 mL) was added Et₃N (22.4 mL, 161 mmol) and Boc₂O (25.8 g, 118 mmol) at 0° C. The resulting solution was allowed to warm to rt and stirred for 1 h. The resulting mixture was washed three times with brine, dried over Na₂SO₄, filtered, and then concentrated. The resulting residue was purified by silica gel flash column chromatography (isocratic, petroleum ether/EtOAc=15/1). Appropriate fractions were concentrated. To a suspension of Zn (41.5 g, 635 mmol) in DMF (200 mL) at rt were added 1,2-dibromoethane (1.82 mL, 21.1 mmol) and chlorotrimethylsilane (1.34 mL, 10.5 mmol). The mixture was stirred at 60° C. for 30 min. To the mixture at rt was added methyl (R)-2-((((9H-fluoren-9-yl)methoxy)carbonyl)amino)-3-iodopropanoate (CAS: 156017-42-4, 57.3 g, 127 mmol). The mixture was stirred at 35° C. for 1 h. To a mixture of the obtained material in the last step (30.3 g, 106 mmol), Pd₂(dba)₃CHCl₃ adduct (4.84 g, 5.29 mmol), and tri-o-tolylphosphane (CAS: 6163-58-2, 6.45 g, 21.2 mmol) in DMF (200 mL) at 50° C. was added the organozinc reagent. The mixture was stirred overnight at 50° C. The mixture was filtered. The filtrate was diluted with H₂O and extracted with EtOAc. The organic extract was washed with brine, dried over Na₂SO₄, filtered, and then concentrated. The resulting residue was purified by silica gel flash column chromatography (isocratic, petroleum ether/EtOAc=10/1). Appropriate fractions were concentrated. To a solution of the obtained material (39.5 g, 74.4 mmol) in CH₂Cl₂ (200 mL) was added TFA (100 mL). The resulting solution was stirred overnight at rt. The resulting mixture was concentrated under vacuum. To a solution of the resulting residue (32.0 g, 60.6 mmol) in CH₂Cl₂ (200 mL) was added dropwise Et₃N (9.18 g, 90.9 mmol) and allyl chlorocarbonate (8.0 g, 66.4 mmol) at 0° C., then the mixture was allowed to warm to rt and stirred for 2 h. The resulting mixture was washed with brine, dried over dried over Na₂SO₄, filtered, and then concentrated. To a mixture of the obtained material (20.0 g, 38.8 mmol) in 2-propanol (300 mL) and H₂O (100 mL) at 0° C. were added CaCl₂ (43.1 g, 389 mmol) and NaOH (3.11 g, 77.8 mmol). The mixture was stirred at rt for 5 h. The mixture was filtered. The pH of the mixture was adjusted to 7 with aq. citric acid. The resulting mixture was extracted three times with EtOAc. The combined organic extracts were washed three times with H₂O, dried over Na₂SO₄, filtered, and then concentrated. The resulting residue was purified by silica gel flash column chromatography (isocratic, petroleum ether/EtOAc=4/1) to afford the title compound (5.67 g, 11.3 mmol). LC-MS (ESI, m/z): [M+H]⁺ 501.

### Reference Example-3-01: 4-tert-butyl 3-(9H-fluoren-9-yl)methyl (4S)-2,2-dioxo-1,2lambda6,3-oxathiazolidine-3,4-dicarboxylate

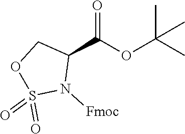

To a solution of thionyl chloride (0.474 ml, 6.50 mmol) and MeCN (15 ml) at −40° C. was added tert-butyl (2S)-2-({[(9H-fluoren-9-yl)methoxy]carbonyl}amino)-3-hydroxypropanoate (CAS: 110797-35-8, 1.92 g, 5 mmol) in MeCN (10 ml) dropwise followed by pyridine (2.02 ml, 25 mmol) while maintaining the temperature below −30° C. The mixture was stirred at rt for 1 h, and then quenched with water. The mixture was extracted with EtOAc. The organic extract was washed with H₂O, dried over Na₂SO₄, filtered, and concentrated. To a solution of the obtained residue in MeCN (30 mL) at 0° C. were added ruthenium(III) chloride (CAS: 10049-08-8, 21.0 mg, 0.100 mmol), sodium periodate (CAS: 7790-28-5, 12.8 g, 60.0 mmol) and H₂O (100 ml). The mixture was stirred at 0° C. for 2 h, and then concentrated. The mixture was extracted with Et₂O. The organic extract was washed with aq. NaHCO₃ and brine, dried over Na₂SO₄, filtered, and concentrated. The residue was purified by silica gel flash column chromatography (gradient, hexane/EtOAc=100/0 to 0/100) to afford the title compound (1.36 g, 3.05 mmol). LC-MS (ESI, m/z): [M+Na]⁺ 468.

### Reference Example-3-02: (4S)-3-{[(9H-fluoren-9-yl)methoxy]carbonyl}-2,2-dioxo-1,2lambda6,3-oxathiazolidine-4-carboxylic acid

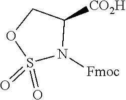

To a solution of Reference example-3-01 (5.00 g, 11.22 mmol) in CH₂Cl₂ (28.0 ml) at 0° C. was added TFA (7.00 ml, 91.0 mmol). The mixture was stirred at rt for 7 h, and then dropped into a stirring hexane (50 mL) at 0° C. The resulting suspension was stirred at rt for 1 h. The resulting solid was collected by filtration to afford the title compound (4.05 g, 10.40 mmol). LC-MS (ESI, m/z): [M+H]⁺ 389.

### Reference Example-3-03: 3-((9H-fluoren-9-yl)methyl) 4-(tert-butyl) (S)-1,2,3-oxathiazinane-3,4-dicarboxylate 2,2-dioxide

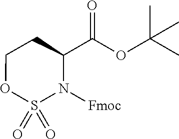

To a mixture of (S)-3-((((9H-fluoren-9-yl)methoxy)carbonyl)amino)-4-(tert-butoxy)-4-oxobutanoic acid (CAS: 129460-09-9, 15 g, 36.5 mmol) in THF (350 mL) at −10° C. were added N-methylmorpholine (CAS: 109-02-4, 5.21 mL, 47.4 mmol) and ethyl chloroformate (CAS: 541-41-3, 3.85 mL, 40.1 mmol). The resulting mixture was stirred at −10° C. for 30 min. NaBH₄ (CAS: 16940-66-2, 1.38 g, 36.5 mmol) was added and the resulting mixture was stirred at −10° C. for 30 min. A freshly prepared solution of NaBH₄ (2.76 g, 72.9 mmol) in water (80 mL) was slowly added dropwise over 15 min. The resulting mixture was stirred at −10° C. for 30 min and then diluted with EtOAc and quenched with 1M aq. KHSO₄. The resulting mixture was extracted with EtOAc. The combined organic extracts were washed with satd. aq. NH₄Cl and brine, dried over Na₂SO₄, filtered, and concentrated. To a mixture of imidazole (11.68 g, 172 mmol) and Et₃N (13.33 mL, 96 mmol) in DCM (300 mL) at −40° C. were added thionyl chloride (3.41 mL, 46.7 mmol) and a solution of the obtained material (14.51 g, 36.5 mmol) in DCM (100 mL). The resulting mixture was allowed to slowly warm up to 0° C. and stirred at 0° C. for 6 h. The mixture was poured into water. The resulting mixture was extracted twice with DCM. The combined organic extracts were washed with brine, dried over Na₂SO₄, filtered, and then concentrated. The residue was purified by flash column chromatography (gradient, heptane/EtOAc=95/5 to 70/30). Appropriate fractions were concentrated. To a solution of the obtained material (9.7 g, 21.87 mmol) in MeCN (200 mL) and Water (50 mL) at 0° C. were added ruthenium(III) chloride (CAS: 14898-67-0, 0.023 g, 0.109 mmol) and then sodium periodate (CAS: 7790-28-5, 7.48 g, 35.0 mmol) in Water (150 mL). The resulting mixture was stirred at 0° C. for 1 h. The resulting mixture was allowed to slowly warm up to rt and stirred at rt overnight. A solution of ruthenium(III) chloride (0.068 g, 0.328 mmol) and sodium periodate (4.68 g, 21.87 mmol) in water (50 mL) was added at 0° C. The resulting mixture was allowed to slowly warm up to rt and stirred at rt for 4 h. The mixture was poured into water. The resulting mixture was extracted with EtOAc. The combined organic extracts were washed with brine, dried over Na₂SO₄, filtered, and then concentrated. The residue was purified by flash column chromatography (gradient, heptane/EtOAc=90/10 to 70/30) to afford the title compound (2.25 g, 4.90 mmol). LC-MS (ESI, m/z): [M+H₂O+H]⁺ 477.

### Reference Example-3-04: 4-tert-butyl 3-(9H-fluoren-9-yl)methyl (4R)-2,2-dioxo-1,2lambda6,3-oxathiazolidine-3,4-dicarboxylate

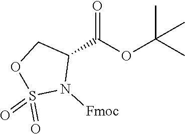

The title compound can be synthesized as outlined for the preparation of Reference Example 3-01 employing appropriate starting material tert-butyl (2R)-2-({[(9H-fluoren-9-yl)methoxy]carbonyl}amino)-3-hydroxypropanoate (CAS: 225662-91-9). LC-MS (ESI, m/z): [M−H]⁻ 444.

### Reference Example-4-01: (S)-2-((((9H-fluoren-9-yl)methoxy)carbonyl)amino)-3-((2-(tert-butoxy)-2-oxoethyl)(methyl)amino)propanoic acid

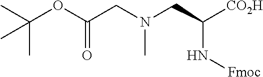

To a solution of Reference example-3-02 (931 mg, 2.39 mmol) in CH₂Cl₂ (5.2 mL) and MeCN (1.6 mL) at 0° C. were added DIPEA (626 μl, 3.59 mmol) and tert-butyl 2-(methylamino)acetate (CAS: 5616-81-9, 521 mg, 3.59 mmol) in DCM (5.2 mL). The mixture was stirred for 3 h at 0° C., and then quenched with 1M aq. KHSO₄ at 0° C. The resulting mixture was stirred for 1 h at 0° C., and then resulting solid was collected by filtration. To the solution of obtained solid in MeOH (30 mL) at rt was added 0.5 M aq. HCl (20 mL). The mixture was stirred for 24 h at rt, and then pH was adjusted to 5-7 by satd. NaHCO₃ aq. To the mixture was added EtOAc and water. The mixture was extracted three times with EtOAc. The combined organic extracts were washed with brine, dried over Na₂SO₄, and concentrated. The residue was purified by silica gel column chromatography (gradient, CH₂Cl₂/MeOH=97/3 to 92/8) to afford the title compound (749 mg, 1.648 mmol). LC-MS (ESI, m/z): [M+H]⁺ 455.

### Reference Example-4-02: (2S)-4-butoxy-2-({[(9H-fluoren-9-yl)methoxy]carbonyl}amino)butanoic acid

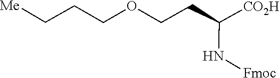

The solution of Reference example-3-03 (700 mg, 1.52 mmol) and sodium dihydrogen phosphate (CAS: 7558-80-7, 731 mg, 6.09 mmol) in 1-butanol (3.80 mL) at rt was stirred for 2 h at 40-45° C. The resulting mixture was concentrated in vacuo. The residue was dissolved with EtOAc and water. The resulting mixture was extracted with EtOAc. The combined organic extracts were washed with brine, dried over Na₂SO₄, filtered, and then concentrated. The residue was purified by flash column chromatography (gradient, heptane/EtOAc=90/10 to 60/40). Appropriate fractions were concentrated. To a solution of the obtained material (635.7 mg, 1.402 mmol) in DCM (2.63 mL) at 0° C. was added TFA (0.88 mL). The mixture was stirred at rt for 1.5 h, and then concentrated. The residue was purified by flash column chromatography (gradient, CH₂Cl₂/MeOH=100/0 to 95/5) to afford the title compound (0.52 g, 1.29 mmol). LC-MS (ESI, m/z): [M+H]⁺ 398.

### Reference Example-4-03: (2S)-3-(cyclobutylmethoxy)-2-({[(9H-fluoren-9-yl)methoxy]carbonyl}amino)propanoic acid

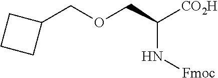

The suspension of 3-((9H-fluoren-9-yl)methyl) 4-methyl (S)-1,2,3-oxathiazolidine-3,4-dicarboxylate 2,2-dioxide (CAS: 2092899-83-5, 3.00 g, 7.44 mmol) and sodium dihydrogen phosphate (CAS: 7558-80-7, 2.68 g, 22.3 mmol) in cyclobutylmethanol (CAS: 4415-82-1, 17.5 mL) was stirred for 18 h at 70° C. The resulting mixture was diluted with AcOEt. The mixture was washed with water and brine, dried over Na₂SO₄, filtered, and then concentrated. To a mixture of the obtained material (3.05 g, 7.44 mmol) in 2-propanol (153 mL) at 0° C. were added CaCl₂ (9.91 g, 89.0 mmol) and a solution of LiOH (0.53 g, 22.3 mmol) in H₂O (65.6 mL). The mixture was stirred at rt for 18 h, and then concentrated. The resulting mixture was diluted with AcOEt and H₂O. The mixture was quenched with 1 M aq. NaH₂PO₄ at 0° C. The resulting mixture was extracted with EtOAc. The organic extract was washed with brine, dried over Na₂SO₄, filtered, and then concentrated. The residue was purified by flash column chromatography (gradient, CH₂Cl₂/MeOH=100/0 to 60/40) to afford the title compound (1.30 g, 3.29 mmol). LC-MS (ESI, m/z): [M+H]⁺ 396.

### Reference Example-5-01: (2S)-3-{4′-cyano-[1,1′-biphenyl]-3-yl}-2-({[(9H-fluoren-9-yl)methoxy]carbonyl}amino)propanoic acid

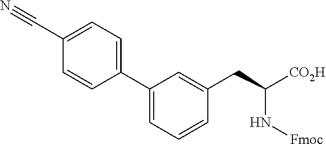

To a solution of (2S)-3-(3-bromophenyl)-2-({[(9H-fluoren-9-yl)methoxy]carbonyl}amino)propanoic acid (CAS: 220497-48-3, 15.0 g, 32.2 mmol) in THF (200 ml) were added (4-cyanophenyl)boronic acid (CAS: 126747-14-6, 7.09 g, 48.3 mmol), palladium(2+) diacetate (CAS: 3375-31-3, 1.44 g, 6.41 mmol), K₃PO₄ (20.5 g, 96.5 mmol), and 1,1′-Bis(di-tert-butylphosphino)ferrocene (CAS: 84680-95-5, 3.05 g, 6.46 mmol). The mixture was stirred at 50° C. overnight, and then filtered. The pH of the filtrate was adjusted to 7 with aq. citric acid, and then the resulting mixture was extracted three times with EtOAc. The combined organic extracts were washed three times with brine, dried over Na₂SO₄, filtered, and concentrated. The resulting residue was purified by silica gel flash column chromatography (isocratic, petroleum ether/EtOAc=1/2) to afford the title compound (7.10 g, 14.5 mmol). LC-MS (ESI, m/z): [M−H]⁻ 487.

### Reference Example-5-02: (2S)-2-({[(9H-fluoren-9-yl)methoxy]carbonyl}amino)-3-{3′-methanesulfonyl-[1,1′-biphenyl]-4-yl}propanoic acid

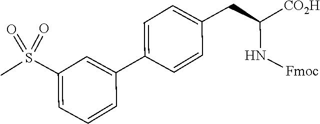

To a solution of (2S)-3-(4-bromophenyl)-2-[[(tert-butoxy)carbonyl]amino]propanoic acid (CAS: 62129-39-9, 17.0 g, 49.4 mmol) in DME/H₂O (3/1, 400 mL) were added (3-methanesulfonylphenyl)boronic acid (CAS: 373384-18-0, 10.0 g, 50.0 mmol), Pd(dppf)Cl₂ (1.1 g, 1.5 mmol), and Na₂CO₃ (9.3 g, 87.7 mmol). The resulting solution was stirred overnight at 90° C. The pH value of the solution was adjusted to 4-5 with aq. citric acid. The resulting solution was extracted with EtOAc and the combined organic layers were dried over Na₂SO₄, filtered, and then concentrated. The residue was purified by silica gel flash column chromatography (isocratic, CH₂Cl₂/MeOH=10/1). Appropriate fractions were concentrated. To a solution of the obtained material (10 g, 23.8 mmol) in CH₂Cl₂ (300 mL) was added TFA (60 mL). The resulting solution was stirred for 2 h at rt. The resulting mixture was concentrated under vacuum. To a solution of the resulting residue (15.0 g, 47.0 mmol) in 1,4-dioxane (200 mL) and H₂O (200 mL) at 0° C. was added NaHCO₃ (11.8 g, 141 mmol), followed by the addition of Fmoc-OSu (20.6 g, 61.1 mmol) in portions. The mixture was stirred overnight at rt, and then the pH of the solution was adjusted to 6 with aq. citric acid. The mixture was extracted with EtOAc. The organic extracts were washed with brine, dried over Na₂SO₄, filtered, and concentrated. The resulting residue was purified by reversed phase silica gel flash column chromatography (C18 silica gel, gradient, H₂O/CH₃CN=70/30 to 20/80) to afford the title compound (4.00 g, 7.38 mmol). LC-MS (ESI, m/z): [M−H]⁻ 540.

### Reference Example-6-01: (2S)-2-({[(9H-fluoren-9-yl)methoxy]carbonyl}amino)-7-methoxyheptanoic acid

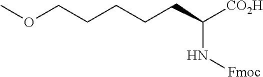

To a mixture of (15S)-9-chloro-19-[(3,4-dichlorophenyl)methyl]-6-phenyl-2-oxa-5lambda4,13,19lambda4-triaza-1-nickelapentacyclo[11.6.0.01,5.07,12.015,19]nonadeca-5,7(12),8,10-tetraene-3,14-dione (CAS: 2089661-84-5, 180 g, 300 mmol) and 1-bromo-5-methoxypentane (CAS: 14155-86-3, 108 g, 598 mmol) in DMF (1500 mL) at 0° C. was added 30% NaOMe in MeOH (64.7 g, 359 mmol) dropwise. The mixture was stirred at rt for 2 h. The reaction was quenched with H₂O. The mixture was stirred at rt for 0.5 h, and then diluted with H₂O. After 48 h, the solids formed were collected by filtration. Then the solids were washed with water and dried in an oven under reduced pressure. To the resulting solid (150 g, 214 mmol) in DME (750 mL) was added 3M aq. HCl (345 mL). The mixture was stirred at 60° C. for 3 h. The resulting mixture was concentrated under vacuum. Then the mixture was washed with H₂O (600 mL) and the solids were filtered out. To the aqueous phase were added MeCN (600 mL) and ethylenediaminetetraacetic acid disodium salt dihydrate (CAS: 6381-92-6, 79.4 g, 214 mmol). The mixture was stirred at 0° C. for 0.5 h. To the mixture were added NaOH (35.1 g, 876 mmol) in portions at 0° C. To the mixture were added Na₂CO₃ (29.5 g, 278 mmol) and Fmoc-OSu (72.1 g, 214 mmol) at 0° C. The mixture was stirred for 2 h at rt, and then pH of the solution was adjusted to 4 with 3 M aq. HCl. The mixture was diluted with water and extracted three times with EtOAc. The organic extracts were washed three times with brine, dried over Na₂SO₄, filtered, and concentrated. The resulting residue was purified by silica gel flash column chromatography (isocratic, CH₂Cl₂/MeOH=91/9) and then further purified by reversed phase silica gel flash column chromatography (C18 silica gel, gradient, H₂O/CH₃CN with 0.1% formic acid=60/40 to 40/60) to afford the title compound (49.6 g, 125 mmol). LC-MS (ESI, m/z): [M+H]⁺ 398.

### Reference Example-6-02: (2S)-2-({[(9H-fluoren-9-yl)methoxy]carbonyl}amino)-3-(1,2-oxazol-4-yl)propanoic acid

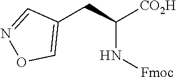

To a solution of (2R)-3,6-dimethoxy-2-(propan-2-yl)-2,5-dihydropyrazine (CAS: 109838-85-9, 4.55 g, 24.7 mmol) in THF (80 mL) at −78° C. was added n-BuLi (19.1 mL, 1.55 M in hexane) dropwise. The mixture was stirred at −78° C. for 30 min. To the reaction mixture at −78° C. was added a solution of 4-(bromomethyl)-1,2-oxazole (CAS: 6455-40-9, 4.00 g, 24.7 mmol) in THF (5.0 mL) dropwise. The resulting mixture was stirred at rt for 3 h. The reaction was then quenched with satd. aq. NH₄Cl. The mixture was extracted with EtOAc. The organic extracts were washed with brine, dried over Na₂SO₄, filtered, and concentrated. The resulting residue was purified by silica gel flash column chromatography (isocratic, hexane/EtOAc=90/10). Appropriate fractions were collected and concentrated. To a solution of the obtained material (5.17 g, 19.5 mmol) in MeOH (50 mL) at 0° C. was added 4M aq. HCl (14.7 mL). The mixture was stirred at rt for 4 h. The mixture was poured into satd. aq.

NaHCO₃. The mixture was extracted with EtOAc. The combined organic extracts were washed with brine, dried over Na₂SO₄, filtered, and concentrated. The resulting residue was purified by silica gel flash column chromatography (isocratic, hexane/EtOAc=50/50). Appropriate fractions were collected and concentrated. To a solution of the obtained material (0.80 g, 4.70 mmol) in 1,4-dioxane (30 mL) and H₂O (20 mL) at 0° C. was added NaHCO₃ (1.98 g, 23.5 mmol) and (9H-fluoren-9-yl)methyl carbonochloridate (CAS: 28920-43-6, 1.34 g, 5.17 mmol). The mixture was stirred at rt for 3 h, and then extracted with EtOAc. The organic extracts were washed with brine, dried over Na₂SO₄, filtered, and concentrated. The resulting residue was purified by silica gel flash column chromatography (gradient, hexane/EtOAc=100/0 to 50/50). Appropriate fractions were collected and concentrated. To a solution of the obtained material (1.75 g, 4.46 mmol) in ClCH₂CH₂Cl (20 mL) was added trimethyltin hydroxide (3.23 g, 17.8 mmol). The mixture was stirred at 55° C. for 3 h. The resulting mixture was concentrated. The resulting residue was purified by silica gel flash column chromatography precharged with KF powder (gradient, CH₂Cl₂/MeOH=100/0 to 85/15) to afford the title compound (1.06 g, 2.79 mmol). LC-MS (ESI, m/z): [M+H]⁺ 379.

### Reference Example-6-03: (S)-2-((((9H-fluoren-9-yl)methoxy)carbonyl)amino)-3-(4-(tert-butoxycarbonyl)-2-iodophenyl)propanoic acid

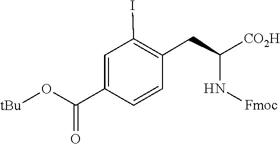

To a mixture of 4-(bromomethyl)-3-iodobenzoic acid (CAS: 496944-09-3, 2.00 g, 5.87 mmol) and tert-butyl 2,2,2-trichloroacetimidate (CAS: 98946-18-0, 2.56 g, 11.7 mmol) in THF (14.7 mL) at 0° C. was added boron trifluoride diethyl etherate (CAS: 109-63-7, 0.07 mL, 0.59 mmol). The mixture was stirred at 0° C. for 30 min. To the mixture at 0° C. was added tert-butyl 2,2,2-trichloroacetimidate (0.64 g, 2.93 mmol). The mixture was stirred at 0° C. for 30 min, and then concentrated. The residue was purified by flash column chromatography (gradient, heptane/EtOAc=100/0 to 85/15). Appropriate fractions were concentrated. To a mixture of the obtained material (1.99 g, 5.00 mmol) and (15S)-9-chloro-19-[(3,4-dichlorophenyl)methyl]-6-phenyl-2-oxa-5lambda4,13,19lambda4-triaza-1-nickelapentacyclo[11.6.0.01,5.07,12.015,19]nonadeca-5,7(12),8,10-tetraene-3,14-dione (CAS: 2089661-84-5, 3.01 g, 5.00 mmol) in DMF (33.3 mL) at rt was added 10% KOH in MeOH (3.1 mL, 5.50 mmol). The mixture was stirred at rt for 1.5 h. The reaction was quenched with H₂O. The mixture was stirred at rt for 0.5 h, and then diluted with H₂O. The mixture was stirred at rt for 2 h. The resulting solid was collected by filtration and washed with DMF/water (2/1), and then dried in an oven under reduced pressure. To the resulting solid (4.00 g, 4.36 mmol) in MeCN (69.7 mL) and water (17.4 mL) at rt was added 8-hydroxyquinoline (CAS: 148-24-3, 1.58 g, 10.9 mmol). The mixture was stirred at 60° C. for 24 h. The resulting mixture was concentrated. The resulting residue was suspended in DCM. The resulting solid was collected by filtration. To the suspension of the obtained solid in acetone (14.5 mL) and 1M aq. Na₂CO₃ (8.72 mL) at 0° C. was added Fmoc-OSu (1.12 g, 3.49 mmol). The mixture was stirred for 3 h at rt. The resulting mixture was concentrated to remove acetone, diluted with EtOAc, neutralized with 1M aq. HCl, and then filtered to remove Ni residue from previous reaction. The filtrate was extracted EtOAc. The organic extracts were washed with water and brine, dried over Na₂SO₄, filtered, and concentrated. The resulting residue was triturated with heptane/EtOAc (90/10) and the resulting solid was collected by filtration. The crude solid was purified by silica gel flash column chromatography (gradient, CH₂Cl₂/MeOH=100/0 to 90/10) to afford the title compound (1.89 g, 3.07 mmol). LC-MS (ESI, m/z): [M-tBu+H]⁺ 558.

### Reference Example-6-04: (S)-2-((9H-fluoren-9-yl)methoxy)carbonyl)amino)-2-methyl-3-(1-trityl-1H-imidazol-4-yl)propanoic acid

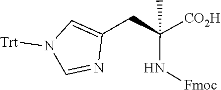

To a solution of (2R)-3,6-dimethoxy-2-(propan-2-yl)-2,5-dihydropyrazine (CAS: 109838-85-9, 10.0 g, 54.3 mmol) in THF (100 mL) at −78° C. was added n-BuLi (22.8 mL, 242.0 mmol) dropwise. The mixture was stirred at −78° C. for 30 min. To the reaction mixture at −78° C. was added a solution of iodomethane (8.10 g, 57.1 mmol) in THF (5.0 mL) dropwise. The resulting mixture was stirred at −78° C. for 1 h. The reaction was diluted with EtOAc, then the reaction was quenched with satd. aq. NH₄Cl. The mixture was extracted with EtOAc. The organic extracts we re washed with brine, dried over Na₂SO₄, filtered, and concentrated. The resulting residue was purified by silica gel flash column chromatography (isocratic, petroleum ether/EtOAc=99/1). Appropriate fractions were collected and concentrated. To a solution of the obtained material (3.5 g, 17.7 mmol) in THF (60 mL) at −78° C. was added n-BuLi (8.6 mL, 2.5M in hexane) dropwise. The mixture was stirred at −78° C. for 30 min. To the reaction mixture at −78° C. was added a solution of 4-(chloromethyl)-1-trityl-1H-imidazole (CAS: 103057-10-9, 5.4 g, 15.0 mmol) in DCM (10 mL). The mixture was stirred at rt for 1 h. The reaction was diluted with EtOAc, then the reaction was quenched with satd. aq. NH₄Cl. The mixture was extracted with EtOAc. The organic extracts were washed with brine, dried over Na₂S O₄, filtered, and concentrated. The resulting residue was purified by silica gel flash column chromatography (gradient, petroleum ether/EtOAc=99/1 to 80/20). Appropriate fractions were collected and concentrated. To a solution of the obtained material (2.0 g, 3.84 mmol) in CH₃CN (50 mL) at 0° C. were added HCl (3 mL) and H₂O (27 mL) dropwise. The mixture was stirred at rt for 6 h. The pH of the mixture was adjusted to 8 with satd. aq. NaHCO₃. The mixture was extracted with EtOAc. The organic extracts were washed with brine, dried over Na₂SO₄, filtered, and concentrated. The resulting residue was purified by silica gel flash column chromatography (isocratic, CH₂Cl₂/MeOH=10/1) to afford methyl (2S)-2-amino-2-methyl-3-[1-(triphenylmethyl)-1H-imidazol-4-yl]propanoate (Intermediate-1-1, 1.00 g, 2.35 mmol). To a solution of Intermediate-1-1 (1.60 g, 3.76 mmol) in EtOH (30 mL) at rt were added H₂O (15 mL) and NaOH (0.45 g, 11.3 mmol). The mixture was stirred at 80° C. for 2 h. The resulting mixture was concentrated. To a solution of the resulting residue (1.55 g, 3.77 mmol) and NaHCO₃ (0.63 g, 7.52 mmol) in 1,4-dioxane (30 mL) and H₂O (10 mL) at 0° C. was added a solution of F moc-OSu (1.27 g, 3.77 mmol) in 1,4-dioxane (10 mL) dropwise. The mixture was stirred at rt for 2 h. The reaction was quenched with satd. aq. citric acid. The mixture was extracted with EtOAc. The organic extracts were washed with brine, dried over Na₂SO₄, filtered, and concentrated. The resulting residue was purified by silica gel flash column chromatography (isocratic, CH₂Cl₂/MeOH=10/1), and then further purified by reversed phase silica gel flash column chromatography (C18 silica gel, gradient, H₂O/CH₃CN with 10 mmol/L NH₄HCO₃=70/30 to 50/50 to afford the title compound (1.33 g, 2.10 mmol). LC-MS (ESI, m/z): [M+H]⁺ 634

### Reference Example-6-05: (S)-2-((((9H-fluoren-9-yl)methoxy)carbonyl)(methyl)amino)-2-methyl-3-(1-trityl-1H-imidazol-4-yl)propanoic acid

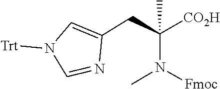

To a solution of Intermediate-1-1 (1.0 g, 2.35 mmol) in MeOH (9.0 mL) at rt were added TEA (1.0 mL, 7.21 mmol), 1M aq. NaOH (1.0 mL), and Boc₂O (1.0 g, 4.58 mmol). The mixture was stirred at rt for 16 h. The reaction was quenched with H₂O. The mixture was extracted with EtOAc. The organic extracts were washed with brine, dried over Na₂SO₄, filtered, and concentrated. The resulting residue was purified by silica gel flash column chromatography (isocratic, petroleum et her/EtOAc=50/50). Appropriate fractions were collected and concentrated. To a solution of the obtained material (0.60 g, 1.14 mmol) in DMF (5.0 mL) at 0° C. were added NaH (0.12 g 60% dispersion in mineral oil) and iodomethane (0.16 g, 1.13 mmol). The mixture was stirred at rt for 1 h. The reaction was quenched with H₂O. The mixture was extracted with EtOAc. The organic extracts were washed with brine, dried over Na₂SO₄, filtered, and concentrated. The resulting residue was purified by silica gel flash column chromatography (isocratic, petroleum ether/EtOAc=50/50). Appropriate fractions were collected and concentrated. To a solution of the obtained material (0.54 g, 1.00 mmol) in EtOH (20 mL) and H₂O (10 mL) at rt was added NaOH (0.40 g, 8.89 mmol). The mixture was stirred at 85° C. for 16 h. The reaction was quenched with aq. citric acid. The mixture was extracted with EtOAc. The organic extracts were washed with brine, dried over Na₂S O₄, filtered, and concentrated. The resulting residue was purified by silica gel flash column chromatography (isocratic, petroleum ether/EtOAc=50/50), and then further purified by reversed phase silica gel flash column chromatography (C18 silica gel, gradient, H₂O with 50 mmol/L NH₄HCO₃/CH₃CN=90/10 to 0/100). Appropriate fractions were collected and concentrated. To a solution of the obtained material (0.26 g, 0.49 mmol) in CH₃CN (7 mL) at rt was added 0.25M aq. HCl (4.0 m L). The mixture was stirred at rt for 4 h. The pH of the mixture was adjusted to 8 with satd. aq. NaHCO₃. The mixture was concentrated. To a solution of the resulting residue were added at rt 1,4-dioxane (5.0 mL), H₂O (3.0 mL), and NaHCO₃ (0.15 g, 1.79 mmol). To the mixture at 0° C. was added a solution of Fmoc-OSu (0.17 g, 0.50 mmol) in 1,4-dioxane (2.0 mL) dropwise. The mixture was stirred at rt for 5 h. The reaction was quenched with aq. citric acid. The mixture was extracted with EtOAc. The organic extracts were washed with brine, dried over Na₂S O₄, filtered, and concentrated. The resulting residue was purified by silica gel flash column chromatography (isocratic, CH₂Cl₂/MeOH=10/1), and then further purified by reversed phase silica gel flash column chromatography (C18 silica gel, gradient, H₂O with 10 mmol/L NH₄HCO₃/CH₃CN=70/30 to 30/70) to afford the title compound (0.08 g, 0.12 mmol). LC-MS (ESI, m/z): [M+H]⁺ 648.

### Reference Example-7-01: (2S)-3-[4-(2-acetamidoethoxy)phenyl]-2-({[(9H-fluoren-9-yl)methoxy]carbonyl}amino)propanoic acid

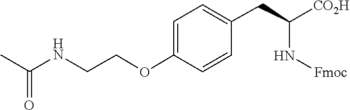

To a solution of (2S)-3-[4-(2-{[(tert-butoxy)carbonyl]amino}ethoxy)phenyl]-2-({[(9H-fluoren-9-yl)methoxy]carbonyl}amino)propanoic acid (CAS: 1013883-02-7, 50.0 g, 91.0 mmol) in DCM (100 mL) at rt was added 4M HCl in CPME (100 mL) and stirred for 3 h. The resulting solid was collected by filtration. The solid was washed by DCM and dried at 50° C. under vacuum. To a solution of the residue (17.6 g, 36.5 mmol) in 1,4-dioxane (122 mL) at 0° C. was added a solution of sodium carbonate (15.5 g, 146 mmol) in water (61.0 mL). To the suspension was transferred acetic anhydride (3.62 mL, 38.3 mmol) via a syringe and the resulting reaction mixture was stirred at 25° C. for 1 h after stirring for 10 min at 0° C. The pH of the mixture was adjusted to 3 at 0° C. with 1M aq. HCl. The remaining solid was collected by filtration and the solid was washed with water and IPE. The solid was dried overnight at 50° C. under high vacuum to afford the title compound (16.0 g, 32.8 mmol) as a white solid. LC-MS (ESI, m/z): [M+H]⁺ 489.

### Reference Example-7-02: (2S)-2-({[(9H-fluoren-9-yl)methoxy]carbonyl}amino)-4-methanesulfonamidobutanoic acid

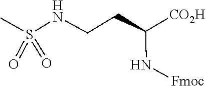

To a suspension of (2S)-4-amino-2-({[(9H-fluoren-9-yl)methoxy]carbonyl}amino)butanoic acid (CAS: 161420-87-7, 200 g, 588 mmol) in THF (2000 mL) at 0° C. was added BSA (538 g, 2.64 mol) then stirred at rt for 2 h. To the mixture at 0° C. was added dropwise methanesulfonyl chloride (CAS: 124-63-0, 80.8 g, 705 mmol). The resulting mixture was stirred at rt for 3 h then the pH of the solution was adjusted to 8 with satd. aq. NaHCO₃ at 0° C. The mixture was stirred at rt for 1 h. The pH of the solution was adjusted to 5 with 2M aq. HCl at 0° C. The mixture was extracted three times with EtOAc. The combined organic extracts were washed with brine, dried over Na₂SO₄, filtered, and concentrated. The resulting residue was purified by silica gel flash column chromatography (isocratic, CH₂Cl₂/MeOH=83/17) to afford the title compound (114 g, 272 mmol). LC-MS (ESI, m/z): [M+H]⁺ 419.

### Reference Example-7-03: (2S)-2-({[(9H-fluoren-9-yl)methoxy]carbonyl}(methyl)amino)-6-(phenylformamido)hexanoic acid

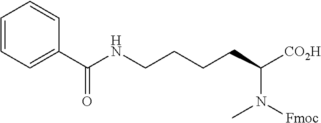

To a solution of (2S)-6-{[(tert-butoxy)carbonyl]amino}-2-({[(9H-fluoren-9-yl)methoxy]carbonyl}(methyl)amino)hexanoic acid (CAS: 197632-76-1, 20.0 g, 41.4 mmol) in CH₂Cl₂ (118 mL) was added 4M HCl in CPME (118 mL). The mixture was stirred for 2 h, and then concentrated. To the resulting residue (1.00 g, 2.39 mmol) in THF (7.96 mL) at rt was added BSA (2.43 g, 11.9 mmol). The mixture was stirred at rt for 0.5 h, and then cooled to 0° C. To the mixture was added benzoyl chloride (CAS: 98-88-4, 0.336 g, 2.39 mmol) at 0° C. The mixture was stirred at rt for 2 h, and then diluted with aq. NaHCO₃. The mixture was extracted with EtOAc. The combined organic extracts were washed with aq. HCl, dried over Na₂SO₄, filtered, and concentrated to afford the title compound (0.915 g, 1.86 mmol). LC-MS (ESI, m/z): [M+H]⁺ 487.

### Reference Example-7-04: (2S)-3-(2-acetamidoacetamido)-2-({[(9H-fluoren-9-yl)methoxy]carbonyl}(methyl)amino)propanoic acid

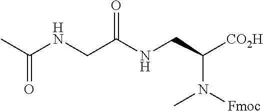

To a solution of (S)-2-((((9H-fluoren-9-yl)methoxy)carbonyl)(methyl)amino)-3-((tert-butoxycarbonyl)amino)propanoic acid (CAS: 1424972-75-7, 5 g, 11.35 mmol) in DMF (22.70 ml) were added DIPEA (2.97 ml, 17.03 mmol) and allyl bromide (CAS: 106-95-6, 1.277 ml, 14.76 mmol) at rt. The mixture was stirred at rt overnight. After diluted by EtOAc and hexane, the organic extract was washed by water, 0.25 M aq. citric acid, satd. aq. NaHCO₃, and water. The organic layer was dried over Na₂SO₄, filtered, and concentrated. The resulting residue was purified by silica gel flash column chromatography (gradient, EtOAc/hexane=0/100 to 50/50). Appropriate fractions were concentrated. To a solution of the resulting residue in CH₂Cl₂ was added 4M aq. HCl in 1,4-dioxane (20 ml, 80 mmol) at rt. After stirred for 2 h, this mixture was concentrated. To a solution of the resulting residue (2.50 g, 6.00 mmol) and 2-acetamidoacetic acid (CAS: 543-24-8, 0.843 g, 7.20 mmol) in DMF (24 ml) were added DIPEA (2.62 ml, 14.99 mmol) and HATU (2.74 g, 7.20 mmol) at 0° C. The mixture was stirred at rt for 2 h. After diluted with EtOAc and hexane, the organic extract was washed by 1M aq. HCl, satd. aq. NaHCO₃, and brine. The organic layer was dried over Na₂SO₄, filtered, and concentrated. To a solution of the resulting residue in CH₂Cl₂ (50 ml) were added phenylsilane (CAS: 694-53-1, 1.478 ml, 11.99 mmol) and Pd(PPh₃)₄ (0.173 g, 0.150 mmol) at 0° C. and the resulting mixture was stirred for 1 h at the same temperature. After concentrated, the resulting residue was purified by silica gel flash column chromatography (gradient, CH₂Cl₂/MeOH=100/0 to 70/30) to afford the title compound (1.4996 g, 3.41 mmol). LC-MS (ESI, m/z): [M+H]⁺ 440.

### Reference Example-7-05: (2S)-3-{1-[2-(tert-butoxy)-2-oxoethyl]piperidin-4-yl}-2-({[(9H-fluoren-9-yl)methoxy]carbonyl}amino)propanoic acid

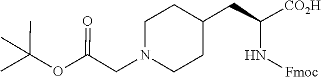

To a solution of (S)-2-((((9H-fluoren-9-yl)methoxy)carbonyl)amino)-3-(1-(tert-butoxycarbonyl)piperidin-4-yl)propanoic acid (7.5 g, 15.16 mmol) in CH₂Cl₂ (60 mL) at 0° C. was added 4M hydrogen chloride in CPME (20 mL, 80 mmol). The mixture was stirred at 0° C. for 1 h, and then concentrated. To a suspension of the residue in THF (60.0 mL) at 0° C. was added MSA (8.52 mL, 53.1 mmol). Upon stirring, the mixture turned into a clear solution. To the solution at 0° C. was added DIPEA (7.95 mL, 45.5 mmol), followed by tert-butyl bromoacetate (2.69 mL, 18.20 mmol) dropwise. The mixture was stirred at 0° C. for 1 h, and then rt for 16 h. The mixture was diluted with EtOAc and a mixture of satd. aq. NH₄Cl and H₂O. The pH was adjusted to pH 4 with 2M aq. H₂SO₄. The mixture was extracted twice with EtOAc. The combined organic extracts were washed with brine, dried over Na₂SO₄, filtered, and concentrated. The resulting residue was purified by silica gel flash column chromatography (gradient, CH₂Cl₂/MeOH=100/0 to 80/20) to afford the title compound (5.60 g, 10.76 mmol). LC-MS (ESI, m/z): [M+H]⁺ 509.

### Reference Example-8-01: (2S)-2-({[(9H-fluoren-9-yl)methoxy]carbonyl}amino)-4-{[(pyridin-4-yl)methyl]carbamoyl}butanoic acid

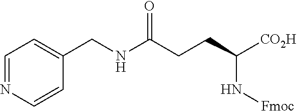

To a solution of (4S)-5-(tert-butoxy)-4-({[(9H-fluoren-9-yl)methoxy]carbonyl}amino)-5-oxopentanoic acid (CAS: 84793-07-7, 30.00 g, 70.51 mmol) in DMF (300 mL) at 0° C. were added HATU (10.25 g, 26.97 mmol), DIPEA (5.81 g, 44.95 mmol) and 1-(pyridin-4-yl)methanamine (CAS: 3731-53-1, 7.63 g, 70.51 mmol). The mixture was stirred at rt for 5 min, and then diluted with H₂O. The mixture was extracted three times with EtOAc. The combined organic extracts were washed three times with brine, dried over Na₂SO₄, filtered, and concentrated. The resulting residue was purified by silica gel flash column chromatography (isocratic, CH₂Cl₂/MeOH=10/1). Appropriate fractions were collected and concentrated (20.0 g, 38.78 mmol). To a solution of the obtained material in 4M HCl in EtOAc (210 mL) was stirred at rt for 8 h. The solids were purified by re-crystallization from EtOAc to afford the title compound (10.03 g, 21.83 mmol). LC-MS (ESI, m/z): [M+H]⁺ 460.

### Reference Example-8-02: (2S)-2-({[(9H-fluoren-9-yl)methoxy]carbonyl}amino)-4-{[(1s,3s)-3-{[(tert-butoxy)carbonyl]amino}cyclobutyl]carbamoyl}butanoic acid

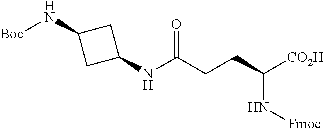

To a solution of (4S)-4-({[(9H-fluoren-9-yl)methoxy]carbonyl}amino)-5-oxo-5-(prop-2-en-1-yloxy)pentanoic acid (CAS: 144120-54-7, 12.00 g, 29.31 mmol) in DMF (120 mL) at rt were added HATU (16.72 g, 43.96 mmol), DIPEA (11.36 g, 87.93 mmol) and tert-butyl N-[(1s,3s)-3-aminocyclobutyl]carbamate (CAS: 1212395-34-0, 5.46 g, 29.31 mmol). The mixture was stirred at rt for 0.5 h, and then diluted with H₂O. The mixture was extracted three times with EtOAc. The combined organic extracts were washed with brine, dried over Na₂SO₄, filtered, and concentrated. The resulting residue was purified by silica gel flash column chromatography (gradient, petroleum ether/EtOAc=10/1 to 1/1). Appropriate fractions were concentrated (14.00 g, 24.24 mmol). To a solution of the obtained material in THF (140 mL) at rt was added Pd(PPh₃)₄ (2.80 g, 2.423 mmol). To the mixture at 0° C. was added morpholine (CAS: 110-91-8, 4.22 g, 48.470 mmol). The resulting mixture was stirred for 0.5 h at rt. The pH value of the mixture was adjusted to 5 with 3 M aq. HCl. The resulting mixture was extracted three times with EtOAc. The combined organic extracts were washed with brine, dried over Na₂SO₄, filtered, and concentrated. The resulting residue was purified by reversed phase silica gel flash column chromatography (C18 silica gel, gradient, H₂O with 0.1% TFA/CH₃CN=50/50 to 20/80) to afford the title compound (5.015 g, 9.328 mmol). LC-MS (ESI, m/z): [M+H]⁺ 538.

### Reference Example-8-03: (2S)-3-[4-(2-{4-[2-(tert-butoxy)-2-oxoethyl]piperazin-1-yl}-2-oxoethoxy)phenyl]-2-({[(9H-fluoren-9-yl)methoxy]carbonyl}amino)propanoic acid

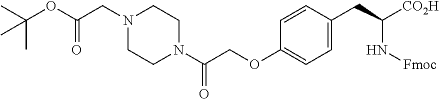

To a solution of (2S)-3-{4-[2-(tert-butoxy)-2-oxoethoxy]phenyl}-2-({[(9H-fluoren-9-yl)methoxy]carbonyl}amino)propanoic acid (CAS: 181951-92-8, 40 g, 77.28 mmol) in DMF (400 mL) at 0° C. were added NaHCO₃ (13.00 g, 154.57 mmol) and 3-bromoprop-1-ene (11.20 g, 92.74 mmol). The mixture was stirred at rt for 2 h, and then diluted with H₂O. The mixture was extracted twice with EtOAc. The combined organic extracts were washed three times with brine, dried over Na₂SO₄, filtrated, and then concentrated. The resulting residue was purified by silica gel column chromatography (isocratic, petroleum ether/EtOAc=50/50). Appropriate fractions were concentrated. To a solution of the resulting residue (31 g, 55.60 mmol) in CH₂Cl₂ (300 mL) at 0° C. was added TFA (150 mL). The mixture was stirred at rt for 2 h, and then concentrated. The resulting residue was suspended in toluene, and then concentrated. To a solution of the resulting residue in DMF (400 mL) at rt were added tert-butyl 2-(piperazin-1-yl)acetate (13.609 g, 67.983 mmol), HATU (47.00 g, 123.63 mmol), and DIPEA (31.93 g, 247.26 mmol). The mixture was stirred at rt for 3 h, and then diluted with H₂O. The mixture was extracted three times with EtOAc. The combined organic extracts were washed with three times with brine, dried over Na₂SO₄, filtered, and then concentrated. The resulting residue was purified by silica gel flash column chromatography (isocratic, petroleum ether/EtOAc=50/50). Appropriate fractions were concentrated. To a solution of the resulting residue (23 g, 33.64 mmol) in THF at rt were added morpholine (5.86 g, 67.26 mmol) and Pd(PPh₃)₄ (3.89 g, 3.36 mmol). The mixture was stirred at rt for 0.5 h. The reaction was quenched with H₂O. The pH of the mixture was adjusted to 6 with citric acid. The mixture was extracted three times with EtOAc. The combined organic extracts were washed three times with brine, dried over Na₂SO₄, filtrated, and then concentrated. The resulting residue was purified by reverse-phase flash chromatography (Column, C18 silica gel, gradient, CH₃CN/H₂O=10/90 to 50/50) to afford the title compound (11.0 g, 17.1 mmol). LC-MS (ESI, m/z): [M+H]⁺ 644.

### Reference Example-9-01: 2-({[(9H-fluoren-9-yl)methoxy]carbonyl}[4-(N-methylmethanesulfonamido)butyl]amino)acetic acid

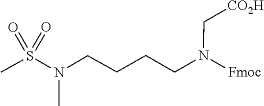

To a solution of tert-butyl N-(4-aminobutyl)-N-methylcarbamate (CAS: 144222-23-1, 15.0 g, 74.1 mmol) in CH₃CN (225 mL) at 0° C. were added tert-butyl 2-bromoacetate (CAS: 5292-43-3, 21.7 g, 111.2 mmol) and DIPEA (19.2 g, 148.3 mmol).

The mixture was stirred at rt overnight. The mixture was extracted with EtOAc. The combined organic extracts were washed with brine, dried over Na₂SO₄, filtrated, and concentrated. The resulting residue was purified by silica gel flash column chromatography (isocratic, petroleum ether/EtOAc=70/30). Appropriate fractions were concentrated. To a solution of the obtained material (14.0 g, 44.2 mmol) in 1,4-dioxane (280 mL) at 0° C. were added H₂O (280 mL), and NaHCO₃ (7.43 g, 88.5 mmol), followed by Fmoc-Cl (CAS: 28920-43-6 13.7 g, 53.1 mmol). The mixture was stirred at rt overnight. The pH of the mixture was adjusted to 5 with aq. citric acid. The mixture was extracted three times with EtOAc. The organic extracts were washed with brine, dried over Na₂SO₄, filtered, and concentrated. The resulting residue was purified by silica gel flash column chromatography (isocratic, petroleum ether/EtOAc=50/50). Appropriate fractions were concentrated. The mixture of the resulting residue (17.0 g, 31.6 mmol) and 12M aq. HCl in 1,4-dioxane (340 mL) was stirred at rt for 2 h, and then concentrated. To a solution of the resulting residue (9.00 g, 23.5 mmol) in THF (270 mL) at 0° C. was added BSA (23.9 g, 117.6 mmol). The mixture was stirred at rt overnight. To the mixture at 0° C. were added DIPEA (12.3 mL, 70.6 mmol) and methanesulfonyl chloride (3.28 mL, 42.4 mmol). The mixture was stirred at rt for 2 h. The mixture was washed three times with brine. The resulting solution was extracted with EtOAc. The organic extracts were dried over Na₂SO₄, filtered, and concentrated. The resulting residue was purified by reversed phase silica gel flash column chromatography (C18 silica gel, gradient, H₂O/CH₃CN=95/5 to 0/100) to afford the title compound (3.25 g, 7.05 mmol). LC-MS (ESI, m/z): [M+H]⁺ 439.

### Reference Example-9-02: 2-({[(9H-fluoren-9-yl)methoxy]carbonyl}(3-methanesulfonamidopropyl)amino)acetic acid

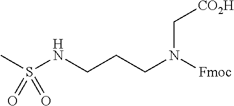

To a solution of N-(3-aminopropyl)methanesulfonamide (CAS: 88334-76-3, 0.50 g, 3.28 mmol) in AcOH (0.32 mL) and MeOH (16.1 mL) at rt were added 2-oxoacetic acid hydrate (CAS: 1152495-30-1, 0.30 mg, 3.28 mmol) and sodium cyanoborohydride (CAS: 25895-60-7, 0.14 mg, 2.20 mmol). The mixture was stirred at rt for 6 h, and then concentrated. To a solution of the resulting residue (0.69 g, 3.28 mmol) in 1,4-dioxane (16.4 mL) and H₂O (16.4 mL) at 0° C. was added NaHCO₃ (1.10 g, 13.1 mmol), followed by addition of Fmoc-OSu (1.00 g, 2.95 mmol). The mixture was stirred at rt for 2 h. The reaction mixture was acidified with 2M aq. HCl at 0° C. The mixture was extracted twice with EtOAc. The organic extracts were washed with brine, dried over Na₂SO₄, filtered, and concentrated. The resulting residue was purified by silica gel flash column chromatography (gradient, petroleum ether/EtOAc=99/1 to 50/50) to afford the title compound (0.18 g, 0.35 mmol). LC-MS (ESI, m/z): [M+H]⁺ 433.

### Reference Example-9-03: 2-[(3-{[(tert-butoxy)carbonyl](methyl)amino}propyl)({[(9H-fluoren-9-yl)methoxy]carbonyl})amino]acetic acid

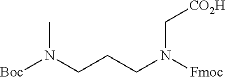

To a solution of tert-butyl N-(3-aminopropyl)-N-methylcarbamate (CAS: 150349-36-3, 1.00 g, 5.31 mmol) in CH₃CN (26.6 mL) at 0° C. were added DIPEA (1.86 mL, 10.6 mmol) and tert-butyl 2-bromoacetate (CAS: 5292-43-3, 0.78 mL, 5.31 mmol). The mixture was stirred at rt for 10 h. The reaction was quenched satd. aq. NaHCO₃. The mixture was extracted twice with EtOAc. The combined organic extracts were washed with brine, dried over Na₂SO₄, filtrated, and concentrated. To a solution of the resulting residue (1.61 g, 5.31 mmol) in H₂O (26.6 mL) at 0° C. was added NaHCO₃ (1.34 g, 15.9 mmol), followed by a solution of Fmoc-OSu (1.61 g, 4.78 mmol) in 1,4-dioxane (26.6 mL) dropwise. The mixture was stirred at rt for 20 h. The reaction mixture was acidified with 1M aq. HCl at 0° C. The mixture was extracted with EtOAc. The organic extracts were washed with brine, dried over Na₂SO₄, filtered, and concentrated. The resulting residue was purified by silica gel flash column chromatography (gradient, hexane/EtOAc=80/20 to 67/33). Appropriate fractions were concentrated. The mixture of the obtained material (0.79 g, 1.51 mmol) and 4M HCl in 1,4-dioxane (7.5 mL) was stirred at rt for 2 h, and then concentrated. To a solution of the resulting residue (0.61 g, 1.51 mmol) in THF (7.53 mL) and H₂O (7.53 mL) at 0° C. was added NaHCO₃ (0.51 g, 6.02 mmol), followed by di-tert-butyl dicarbonate (0.39 g, 1.81 mmol). The mixture was stirred at 0° C. for 2 h. The pH of the mixture was adjusted to 1 with 1M aq. HCl. The mixture was extracted with EtOAc. The organic extracts were washed with brine, dried over Na₂SO₄, filtered, and concentrated. The resulting residue was purified by silica gel flash column chromatography (isocratic, CH₂Cl₂/MeOH=91/9) to afford the title compound (0.55 g, 1.18 mmol). LC-MS (ESI, m/z): [M+H]⁺ 469.

### Reference Example-9-04: 1,3-dioxo-2,3-dihydro-1H-isoindol-2-yl-3-({[(9H-Fluoren-9-yl)methoxy]carbonyl}(2-methoxy-2-oxoethyl)amino)propanoate

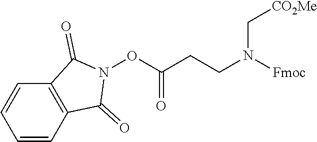

To a mixture of tert-butyl 3-((2-methoxy-2-oxoethyl)amino)propanoate (CAS: 1697106-60-7, 15 g, 69 mmol) in DCM (150 mL) at 0° C. were added DIPEA (35.7 g, 276 mmol) and Fmoc-OSu (22.1 g, 65.6 mmol). The mixture was stirred at rt for 2 h. The reaction was quenched with 1 M aq. HCl. The mixture was extracted three times with DCM. The combined organic extracts were washed with brine, dried over Na₂SO₄, filtered, and then concentrated. The resulting residue was purified by silica gel flash column chromatography (isocratic, petroleum ether/EtOAc=1/1). Appropriate fractions were concentrated. To a mixture of the obtained material (30.8 g, 70.1 mmol) in DCM (300 mL) at 0° C. were added 4 M HCl in 1,4-di oxane (300 mL, 1200 mmol). The resulting mixture was stirred at rt for 2 h, and then concentrated. To a mixture of the obtained material (25 g, 65 mmol) in DCM (300 mL) at rt were added EDCI·HCl (18.75 g, 97.8 mmol) and N-hydroxyphthalimide (CAS: 524-38-9, 11.7 g, 71.7 mmol). The mixture was stirred at rt for 2 h. The reaction was quenched by the addition of aq. NaHCO₃ at rt, and then extracted with DCM. The organic extracts were washed with brine, dried over Na₂SO₄, filtered, and then concentrated. The resulting residue was purified by silica gel flash column chromatography (isocratic, petroleum ether/EtOAc=1/1) to afford the title compound (25 g, 47 mmol).

### Reference Example-10-01: (2S)-3-(2H-1,3-benzodioxol-5-yl)-2-({[(9H-fluoren-9-yl)methoxy]carbonyl}(methyl)amino)propanoic acid

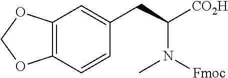

To a solution of (2S)-3-(2H-1,3-benzodioxol-5-yl)-2-[(tert-butoxycarbonyl)amino]propanoic acid (CAS: 209525-79-1, 32.0 g, 103 mmol) in THF (300 mL) at 0° C. was added sodium hydride (12.4 g, 60% dispersion in mineral oil) portionwise, followed by iodomethane (44.1 g, 310 mmol) dropwise. The mixture was stirred at rt for 24 h. The reaction was quenched with H₂O. The pH of the mixture was adjusted to 5 with citric acid. The resulting mixture was extracted three times with EtOAc. The combined extracts were washed three times with brine, dried over Na₂SO₄, filtered, and then concentrated. The resulting residue was purified by silica gel flash column chromatography (isocratic, petroleum ether/EtOAc=50/50). Appropriate fractions were concentrated. The resulting residue (27.0 g, 83.5 mmol) and 2M HCl in 1,4-dioxane (270 mL) was stirred at rt for 1 h. The resulting mixture was concentrated. To the resulting residue in (13.4 g, 51.6 mmol) in 1,4-dioxane (300 mL) and H₂O (100 mL) at 0° C. was added NaHCO₃ (8.67 g, 103 mmol), followed by Fmoc-OSu (17.4 g, 51.6 mmol) portionwise. The mixture was stirred at rt for 8 h. The pH of the solution was adjusted to 5 with citric acid. The mixture was extracted three times with EtOAc. The combined extracts were washed three times with brine, dried over Na₂SO₄, filtered, and then concentrated. The resulting residue was purified by reversed phase silica gel flash column chromatography (C18 silica gel, gradient, H₂O/CH₃CN with 0.1% NH₄HCO₃=60/40 to 30/70) to afford the title compound (5.01 g, 11.3 mmol). LC-MS (ESI, m/z): [M+H]⁺ 446.

### Reference Example-10-02: (2S)-2-({[(9H-fluoren-9-yl)methoxy]carbonyl}(methyl)amino)-6-(N-methylacetamido)hexanoic acid

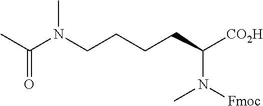

To a solution of (2S)-2-{[(benzyloxy)carbonyl]amino}-6-{[(tert-butoxy)carbonyl]amino}hexanoic acid (CAS: 2389-60-8, 10.0 g, 26.3 mmol) in THF (131 mL) at 0° C. was added sodium hydride (3.68 g, 60% dispersion in mineral oil, 92.0 mmol). The mixture was stirred at 0° C. for 0.5 h. To the mixture was added iodomethane (7.65 g, 53.9 mmol) dropwise. The mixture was stirred at rt for 12 h, and then quenched with 2M aq. H₂SO₄ at 0° C. The mixture was extracted twice with EtOAc. The organic extracts were washed with brine, dried over Na₂SO₄, filtered, and then concentrated. The resulting residue was purified by reversed phase silica gel flash column chromatography (C18 silica gel, gradient, H₂O/CH₃CN=100/0 to 40/60). Appropriate fractions were concentrated. To a solution of the resulting residue (4.49 g, 11.0 mmol) in MeOH (22.0 ml) at rt were added palladium on carbon (1.17 g, 1.10 mmol) and ammonium formate (2.77 g, 44.0 mmol). The mixture was stirred at 60° C. for 2 h, and then filtered. The filtrate was concentrated. To a solution of the resulting residue (3.02 g, 11.0 mmol) in 1,4-dioxane (27.5 mL) and H₂O (27.5 mL) at rt was added NaHCO₃ (2.33 g, 22.0 mmol), followed by Fmoc-OSu (4.45 g, 13.2 mmol). The mixture was stirred at rt overnight, and then the volatiles were removed under reduced pressure and the pH of the mixture was adjusted to 3 with 2M aq. HCl at 0° C. The mixture was extracted twice with CH₂Cl₂. The organic extracts were washed with water and brine, dried over Na₂SO₄, filtered, and concentrated. The resulting residue was purified by reversed phase silica gel flash column chromatography (C18 silica gel, gradient, H₂O/CH₃CN=100/0 to 40/60). Appropriate fractions were concentrated. The mixture of the resulting residue (5.56 g, 11.2 mmol) and 4M hydrogen chloride in CPME (56.0 mL, 224 mmol) was stirred at rt for 2 h, and then concentrated. To a solution of the resulting residue (4.85 g, 11.2 mmol) in THF (56.0 mL) and H₂O (56.0 mL) at 0° C. was added NaHCO₃ (7.53 g, 90.0 mmol), followed by acetic anhydride (2.29 g, 22.4 mmol). The mixture was stirred at rt for 2 h, and then the pH of the mixture was adjusted to 1 with 1M aq. HCl at 0° C. The mixture was extracted with EtOAc. The organic extracts were concentrated. The resulting residue was purified by silica gel flash column chromatography (isocratic, CH₂Cl₂/MeOH=91/9) to afford the title compound (4.85 g, 10.8 mmol). LC-MS (ESI, m/z): [M+H]⁺ 439.

### Reference Example-10-03: (2S)-2-({[(9H-fluoren-9-yl)methoxy]carbonyl}(methyl)amino)-3-{1-[(prop-2-en-1-yloxy)carbonyl]piperidin-4-yl}propanoic acid

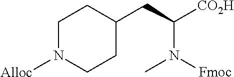

To a solution of (2S)-2-({[(9H-fluoren-9-yl)methoxy]carbonyl}amino)-3-{1-[(prop-2-en-1-yloxy)carbonyl]piperidin-4-yl}propanoic acid (CAS: 313052-03-8, 0.96 g, 2.00 mmol) and p-toluenesulfonic acid monohydrate (76 mg, 0.40 mmol) in toluene (20 mL) was added paraformaldehyde (300 mg, 10.0 mmol). The mixture was stirred at 100° C. for 3 h and then cooled to rt. The mixture was concentrated. The crude residue was purified by silica gel column chromatography (gradient, hexane/EtOAc=90/10 to 40/60). Appropriate fractions were concentrated. To a solution of the obtained compound (0.98 g, 2.00 mmol) and triisopropylsilane (3.17 g, 20 mmol) in CH₂Cl₂ (10 mL) at rt was added TFA (9.2 mL, 120 mmol). The mixture was stirred at rt for 20 h, and then concentrated. The residue was azeotroped with toluene twice.

The residue was purified by silica gel column chromatography (gradient, hexane/EtOAc=90/10 to 0/100) to afford the title compound (889 mg, 1.77 mmol). LC-MS (ESI, m/z): [M+H]⁺ 493.

### Reference Example-10-04: (2S)-3-{1-[(tert-butoxy)carbonyl]piperidin-4-yl}-2-({[(9H-fluoren-9-yl)methoxy]carbonyl}(methyl)amino)propanoic acid

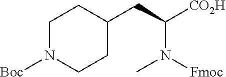

To a solution of Reference example-10-03 (16.5 g, 33.5 mmol) in THF (120 mL) at 0° C. were added PhSiH₃ (7.25 g, 67.0 mmol) and Pd(PPh₃)₄ (7.74 g, 6.7 mmol) at 0° C. The mixture was stirred at rt for 3 h at rt, and then added 1,4-dioxane (60 mL), H₂O (30 mL). To the mixture were added Boc₂O (8.77 g, 40.2 mmol) and NaHCO₃ (5.63 g, 67.0 mmol) at 0° C. The mixture was stirred at rt for 3 h at rt, and then quenched with aq. critic acid. The mixture was extracted twice with EtOAc. The combined organic extracts were washed with brine, dried over Na₂SO₄ and concentrated. The residue was purified by silica gel column chromatography (gradient, hexane/EtOAc=99/1 to 50/50) and then reverse phase C18 silica gel column chromatography (gradient, H2O/MeCN with 0.1% formic acid=45/55 to 25/75) to afford the title compound (9.43 g, 18.5 mmol). LC-MS (ESI, m/z): [M+H]⁺ 409.

### Reference Example-11-01: (2S,4R)-4-(tert-butoxy)-1-{[(9H-fluoren-9-yl)methoxy]carbonyl}pyrrolidine-2-carboxylic acid

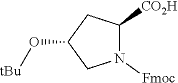

The title compound can be synthesized as outlined for the preparation methods described in WO2014/074658 A1. employing appropriate starting material 1-tert-butyl 2-methyl (2S,4R)-4-hydroxypyrrolidine-1,2-dicarboxylate (CAS: 74844-91-0) and 2-Bromo-2-methylpropane (CAS: 507-19-7). LC-MS (ESI, m/z): [M+H]⁺ 410.

### Reference Example-11-02: (2R,4S)-4-{[(tert-butoxy)carbonyl]amino}-1-{[(9H-fluoren-9-yl)methoxy]carbonyl}pyrrolidine-2-carboxylic acid

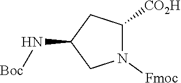

The title compound can be synthesized as outlined for the preparation methods described in Amino Acids (2014), 46(11), 2517. employing appropriate starting material 1,2-dibenzyl (2R,4R)-4-hydroxypyrrolidine-1,2-dicarboxylate (CAS: 142776-07-6). LC-MS (ESI, m/z): [M+H]⁺ 453.

### Reference Example-11-03: (2S,4R)-1-(((9H-fluoren-9-yl)methoxy)carbonyl)-4-(pyridin-3-yloxy)pyrrolidine-2-carboxylic acid

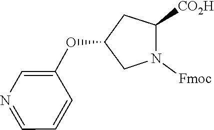

To a suspension of 1-(tert-butyl) 2-methyl (2S,4S)-4-hydroxypyrrolidine-1,2-dicarboxylate (CAS: 102195-79-9, 1.23 g, 5.00 mmol) in THF (10.0 mL) at 0° C. were added 3-Hydroxypyridine (CAS: 109-00-2, 0.52 g, 5.50 mmol), triphenylphosphane (1.57 g, 6.00 mmol), and diisopropyl azodicarboxylate (CAS: 2446-83-5, 3.29 mL, 1.9M in toluene). The mixture was stirred at rt for 18 h, and then concentrated. The resulting residue was purified by silica gel flash column chromatography (gradient, hexane/EtOAc=90/10 to 0/100). Appropriate fractions were concentrated. To the solution of the resulting residue in THF (20.0 mL) and MeOH (10.0 mL) at 0° C. was added 1M aq. sodium hydroxide (10.0 mL, 10.0 mmol). The mixture was stirred at rt for 1 h. The solution was diluted with EtOAc and organic layer was extracted twice with 1M aq. sodium hydroxide. The aqueous extracts were acidified with 1M aq. HCl, and then extracted twice with EtOAc. The combined organic extracts were washed with brine, dried over Na₂SO₄, filtered, and then concentrated. To the solution of the resulting residue in DCM (15.0 mL) at rt was added 4M hydrogen chloride in CPME (18.8 mL, 75.2 mmol). The mixture was stirred at rt for 2 h, and then concentrated. To a solution of the resulting residue in 1,4-dioxane (10.0 mL) and H₂O (10.0 mL) at 0° C. was added NaHCO₃ (1.77 g, 21.0 mmol), followed by a solution of Fmoc-OSu (2.36 g, 7.01 mmol) in 1,4-dioxane (10.0 mL) dropwise. The mixture was stirred at rt for 18 h. The mixture was acidified with 1M aq. HCl. The mixture was extracted twice with EtOAc. The combined organic extracts were washed with brine, dried over Na₂SO₄, filtered, and then concentrated. The resulting residue was purified by silica gel flash column chromatography (gradient, hexane/EtOAc=90/10 to 0/100) to afford the title compound (1.76 g, 4.09 mmol). LC-MS (ESI, m/z): [M+H]⁺ 431.

### Reference Example-11-04: (2S,4R)-4-(dimethylamino)-1-{[(9H-fluoren-9-yl)meth oxy]carbonyl}pyrrolidine-2-carboxylic acid hydrochloride

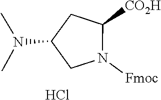

To a suspension of 1-(tert-butyl) 2-methyl (2S,4S)-4-hydroxypyrrolidine-1,2-dicarboxylate (CAS: 102195-79-9, 20.0 g, 82.0 mmol) in DCM (272 mL) at −20° C. was added DIPEA (21.4 mL, 122 mmol) and trifluoromethanesulfonyl trifluoromethanesulfonate (CAS: 358-23-6, 16.5 mL 98.0 mmol) dropwise. The mixture was stirred at −20° C. for 1 h. To the mixture at −20° C. was added dimethylamine (122 mL, 2 M in THF). The mixture was stirred at −20° C. for 16 h. The reaction mixture was washed twice with 1M aq. NaOH, brine, dried over Na₂SO₄, filtered, and then concentrated. The resulting residue was purified by silica gel flash column chromatography (gradient, CH₂Cl₂/MeOH=95/5 to 90/10). Appropriate fractions were collected and concentrated. The resulting residue was suspended with IPE/EtOAc and the resulting suspension was filtered. The filtrate was concentrated in vacuo. To a solution of the obtained material (13.5 g, 49.6 mmol) in DCM (83 mL) at rt was added 4M hydrogen chloride in MTHP (248 mL, 991 mmol). The mixture was stirred at rt for 3 h, and then concentrated. The residue was azeotroped with toluene twice. To a solution of the resulting residue in H₂O (124 mL) at 0° C. was added NaHCO₃ (16.7 g, 198 mmol), followed by a solution of Fmoc-OSu (15.9 g, 47.1 mmol) in 1,4-dioxane (124 mL) dropwise. The mixture was stirred at rt for 18 h. The mixture was extracted three times with EtOAc/THF (9/1). The combined organic extracts were washed with brine, dried over Na₂SO₄, filtered, and then concentrated. The resulting residue was purified by silica gel flash column chromatography (gradient, CH₂Cl₂/MeOH=100/0 to 60/40). Appropriate fractions were collected and concentrated. To a solution of the obtained material (1.00 g, 2.54 mmol) in 1,4-dioxane (12.7 mL) was added conc. aq. HCl (12.7 mL) and stirred at 100° C. for 1 h. The reaction mixture was concentrated. The resulting precipitate was collected by vacuum filtration to afford the title compound (0.82 g, 1.97 mmol). LC-MS (ESI, m/z): [M+H]⁺ 381.

### Reference Example-11-05: (2S,4R)-1-(((9H-fluoren-9-yl)methoxy)carbonyl)-4-cyclobutoxypyrrolidine-2-carboxylic acid

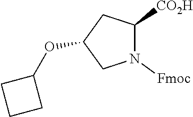

To a solution of 1-((9H-fluoren-9-yl)methyl) 2-methyl (2S,4R)-4-hydroxypyrrolidine-1,2-dicarboxylate (7.35 g, 20.0 mmol) and 2,6-lutidine (2.45 mL, 21.0 mmol) in DCM (50 mL) at 0° C. was added trifluoromethanesulfonic acid trimethylsilyl ester (CAS: 27607-77-8, 7.23 mL, 40.0 mmol) and the mixture was stirred for 1 h. Then to the solution was added cyclobutanone (CAS: 1191-95-3, 1.81 mL, 24.0 mmol) and triethylsilane (4.79 ml, 30.0 mmol). The mixture was stirred at 0° C. for 0.5 h, and at rt for 1 h. The reaction was quenched with satd. aq. NaHCO₃. The mixture was extracted with DCM. The organic extracts were dried over Na₂SO₄, filtered, and concentrated. The resulting residue was purified by silica gel flash column chromatography (gradient, Heptane/EtOAc=90/10 to 40/60). Appropriate fractions were collected and concentrated. To a solution of the obtained material (5.0 g, 11.9 mol) in 2-propanol (111 mL) and H₂O (16 mL) at 0° C. were added CaCl₂ (21.1 g, 190 mmol) and a solution of LiOH (1.14 g, 47.5 mmol) in H₂O (32 mL). The mixture was stirred at rt for 16 h, and then concentrated. The mixture was quench ed with 1M aq. NaIPO₄ at 0° C., and then filtered. The filtrate was extracted with EtOAc. The organic extracts were washed with H₂O and brine, dried over Na₂SO 4, filtered, and concentrated to afford the title compound (3.15 g, 7.73 mmol). LC-MS (ESI, m/z): [M+H]⁺ 408.

### Reference Example-11-06: (2S,5R)-1-(((9H-fluoren-9-yl)methoxy)carbonyl)-5-(naphthalen-1-yl)pyrrolidine-2-carboxylic acid

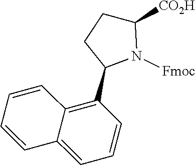

To a solution of 1-tert-butyl 2-methyl (2S)-5-oxopyrrolidine-1,2-dicarboxylate (CAS: 108963-96-8, 40.0 g, 164 mmol) in THF (400 mL) at −60° C. was added bromo(naphthalen-1-yl)magnesium (CAS: 703-55-9, 680 mL, 171 mmol, 0.25 Min THF) dropwise. The mixture was stirred at −60° C. for 3 h. The reaction was quenched with 2-methylpropane at −20° C. and added NaCl (6.34 g, 108 mmol) and NH₄Cl (3.52 g, 65.7 mmol) in water (200 mL) at rt. The mixture was extracted with EtOAc. The organic extracts were washed with brine, dried over Na₂SO₄, filtered, and concentrated The resulting residue was purified by silica gel flash column chromatography (isocratic, petroleum ether/EtOAc=83/17). Appropriate fractions were collected and concentrated. To a solution of the obtained material (34.0 g, 91.5 mmol) in DCM (340 mL) at rt was added TFA (71 mL) dropwise. The resulting solution was stirred at 30° C. for 3 h. The pH of the mixture was adjusted to 7 with aq. KHCO₃. The resulting mixture was extracted with DCM. The organic extracts were washed with brine, dried over Na₂SO₄, filtered, and concentrated. A mixture of the obtained material (21.1 g, 83.3 mmol), Boc₂O (18.2 g, 83.3 mmol), and Pd/C (4.0 g) in MeOH (220 mL) was stirred at rt for 1 day under H₂ atmosphere. The mixture was filtered and concentrated. The resulting residue was purified by silica gel flash column chromatography (isocratic, petroleum ether/EtOAc=83/17). Appropriate fractions were collected and concentrated. A mixture of the obtained material (27.4 g, 77.1 mmol) in 2-propanol (450 mL) and H₂O (150 mL) at 0° C. were added CaCl₂ (137 g, 1.23 mol) and LiOH (7.40 g, 308 mmol) was stirred at rt for 1 day. The pH of the mixture was adjusted to 4-5 with aq. NaH₂PO₄. The mixture was filtered. The filtrate was extracted with EtOAc. The combined organic extracts were washed with brine, dried over Na₂SO₄, filtered, and then concentrated. A mixture of the obtained material (24.5 g, 71.8 mmol) in DCM (74 mL) and 4M HCl in 1,4-dioxane (74 mL) was stirred at rt for 6 h and concentrated. To a mixture of the obtained material (20.0 g, 72.0 mmol) in 1,4-dioxane (450 mL) and H₂O (150 mL) at rt was added NaHCO₃ (30.3 g, 360 mmol). The mixture was stirred at rt for 2 min. To the mixture was added Fmoc-OSu (21.8 g, 64.8 mmol) at 0° C. The mixture was stirred at rt for 5 h. The pH of the mixture was adjusted to 4-5 with aq. NaH₂PO₄. The resulting mixture was extracted with EtOAc. The combined organic extracts were washed three times with brine, dried over Na₂SO₄, filtered, and then concentrated. The resulting residue was purified by silica gel flash column chromatography (isocratic, petroleum ether/EtOAc=83/17) to afford the title compound (10.6 g, 22.9 mmol). LC-MS (ESI, m/z): [M+H]⁺ 464.

### Reference Example-12-01: ((2S)-6-(carbamoylamino)-2-({[(9H-fluoren-9-yl)methoxy]carbonyl}(methyl)amino)hexanoic acid

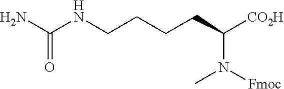

To a solution of (2S)-6-{[(tert-butoxy)carbonyl]amino}-2-({[(9H-fluoren-9-yl)methoxy]carbonyl}(methyl)amino)hexanoic acid (CAS: 197632-76-1, 20.0 g, 41.4 mmol) in DCM (120 ml) were added 4M HCl in CPME (120 mL) and stirred for 2 h. The mixture was concentrated, and dried at 50° C. under vacuum. To the obtained material (3.00 g, 7.16 mmol) in THF (21 ml) and H₂O (14 ml) was added potassium cyanate (581 mg, 7.16 mmol) at 0° C. After stirring the mixture at 23° C. overnight, to the reaction was added potassium cyanate (581 mg, 7.16 mmol) at 0° C. The mixture was stirred at 23° C. overnight, and then diluted with EtOAc. The separated organic extracts was washed with water and brine, dried over Na₂SO₄, filtered, and then concentrated to afford title compound (3.05 g, 6.09 mmol). LC-MS (ESI, m/z): [M+H]⁺ 426.

### Reference Example-13-01: (3S)-6-chloro-2-{[(9H-fluoren-9-yl)methoxy]carbonyl}-1,2,3,4-tetrahydroisoguinoline-3-carboxylic acid

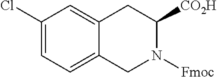

To a solution of (2S)-3-(3-chlorophenyl)-2-({[(9H-fluoren-9-yl)methoxy]carbonyl}amino)propanoic acid (CAS: 198560-44-0, 5.00 g, 25.0 mmol) in MeOH (40.0 mL) and H₂O (40.0 mL) at 0° C. were added sodium carbonate (5.36 g, 50.1 mmol) and ethyl chloroformate (CAS: 541-41-3, 4.08 g, 37.6 mmol). The mixture was stirred at rt for 2.5 h. The pH of the mixture was adjusted to pH 5 with 2 M aq. HCl. The mixture was extracted three times with EtOAc. The organic extracts were washed with brine, dried over Na₂SO₄, filtered, and then concentrated. To a solution of the obtained compound (5.00 g, 18.4 mmol) in AcOH (22.0 mL) at 0° C. was added H2SO4 (7.00 mL) and paraformaldehyde (0.78 g, 25.9 mmol). The mixture was stirred at rt for 12 h. and then cooled to rt. The reaction mixture at 0° C. was quenched with H₂O. The aqueous phase was extracted three times with EtOAc. The combined organic extracts were washed three times with brine, dried over Na₂SO₄, filtered, and then concentrated. The mixture of the obtained compound (21.2 g, 74.7 mmol) and 6M aq. HCl (600 mL) was stirred at 110° C. for 12 h and then cooled to rt. The precipitate was filtered and then dried under reduced pressure to afford the title compound (17.3 g, 69.7 mmol). LC-MS (E SI, m/z): [M+H]⁺ 434.

### Reference Example-13-02: (S)-2-(((9H-fluoren-9-yl)methoxy)carbonyl)-6,7-dichloro-1,2,3,4-tetrahydroisoquinoline-3-carboxylic acid

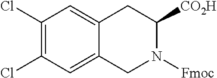

A mixture of (2S)-2-amino-3-(3,4-dichlorophenyl)propanoic acid (CAS: 52794-99-7, 15.0 g, 64.1 mmol) and SOCl₂ (38.1 g, 32.0 mmol) in MeOH (300 mL) was stirred at rt for 12 h. The mixture was concentrated. The resulting residue was purified by silica gel flash column chromatography (gradient, CH₂Cl₂/MeOH=99/1 to 91/9). Appropriate fractions were collected and concentrated. A mixture of the obtained material (18.0 g, 72.6 mmol), pyridine (11.4 g, 144 mmol), and methyl chloroformate (6.86 g, 72.6 mmol) in DCM (100 mL) was stirred at rt for 12 h. The reaction was quenched with H₂O. The mixture was extracted with DCM. The combined organic extracts were dried over Na₂SO₄, filtered, and then concentrated. The resulting residue was purified by silica gel flash column chromatography (gradient, petroleum ether/EtOAc=99/1 to 50/50). Appropriate fractions were collect ed and concentrated. A mixture of the obtained material (14.7 g, 48.0 mmol), AcOH (37.5 g, 624 mmol), H₂SO₄ (12.5 g, 127 mmol), and paraformaldehyde (3.60 g, 120 mmol) was stirred at rt for 12 h. The reaction was quenched with H₂O. The mixture was extracted with EtOAc. The combined organic extracts were concentrated. The resulting residue was purified by silica gel flash column chromatography (gradient, petroleum ether/EtOAc=99/1 to 50/50). Appropriate fractions were collected and concentrated. A mixture of the obtained material (12.0 g, 37.7 mmol), H₂O (100 mL), and conc. aq. HCl (100 mL) was stirred at 100° C. for 12 h. The mixture was filtered. The filtrate was concentrated. A mixture of the obtained material (2.00 g, 8.13 mmol), 1,4-dioxane (50 mL), H₂O (50 mL), Na₂CO₃ (1.72 g, 16.3 mmol), and Fmoc-OSu (2.74 g, 8.13 mmol) was stirred at rt for 6 h and then diluted with EtOAc. The mixture was extracted with EtOAc. The combined organic extracts were dried over Na₂SO₄, filtered, and then concentrated. The resulting residue was purified by silica gel flash column chromatography (gradient, petroleum ether/EtOAc=99/1 to 50/50) to afford the title compound (2.85 g, 6.07 mmol). LC-MS (ESI, m/z): [M+H]⁺ 468.

### Reference Example-13-03: (S)-2-(((9H-fluoren-9-yl)methoxy)carbonyl)-6-((tert-butoxycarbonyl)amino)-1,2,3,4-tetrahydroisoquinoline-3-carboxylic acid

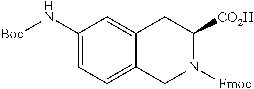

To a mixture of (2S)-2-amino-3-(3-bromophenyl)propanoic acid (CAS: 82311-69-1, 30.0 g, 122 mmol) in MeOH (200 mL) at 0° C. was added SOCl₂ (73.1 g, 614 mmol). The mixture was stirred at rt overnight. The mixture was concentrate d. The resulting residue was purified by silica gel flash column chromatography (gradient, CH₂Cl₂/MeOH=99/1 to 91/9). Appropriate fractions were collected and concentrated. To a mixture of the obtained material (30.0 g, 116 mmol), pyridine (18.4 g, 232 mmol) in DCM (100 mL) at 0° C. was added methyl chloroformate (11.0 g, 116 mmol). The mixture was stirred at rt for 12 h. The reaction was quenched with H₂O. The mixture was extracted with DCM. The combined organic extracts were dried over Na₂SO₄, filtered, and then concentrated. The resulting residue was purified by silica gel flash column chromatography (gradient, petroleum ether/EtOAc=99/1 to 50/50). Appropriate fractions were collected and concentrated. A mixture of the obtained material (29.0 g, 91.7 mmol), AcOH (71.6 g, 1.19 mol), H₂SO₄ (58.5 g, 596 mmol), and paraformaldehyde (3.30 g, 110 mmol) was stirred at 0° C. for 12 h. The reaction was diluted with EtOAc and then quenched with H₂O. The mixture was extracted with EtOAc. The combined organic extracts were dried over Na₂SO₄, filtered, and then concentrated. The resulting residue was purified by silica gel flash column chromatography (gradient, petroleum ether/EtOAc=99/1 to 50/50). Appropriate fractions were collected and concentrated. A mixture of the obtained material (25.0 g, 76.2 mmol), BocNH₂ (CAS: 4248-19-5, 8.84 g, 75.4 mmol), Pd(OAc)₂ (0.17 g, 0.762 mmol), and Xphos (3.63 g, 7.62 mmol) in 1,4-dioxane (200 mL) was stirred at 100° C. for 4 h under N₂ atmosphere. The mixture was concentrated. The resulting residue was purified by silica gel flash column chromatography (gradient, petroleum ether/EtOAc=99/1 to 50/50). Appropriate fractions were collected and concentrated. A mixture of the obtained material (20.0 g, 54.9 mmol) in 6M aq. HCl (100 mL) was stirred at 100° C. for 1 day. The mixture was concentrated. A mixture of the obtained material (22.0 g, 114 mmol), 1,4-dioxane (100 mL), H₂O (100 mL), Na₂CO₃ (24.3 g, 229 mmol), and Fmoc-OSu (38.6 g, 114 mmol) was stirred at rt for 12 h. The mixture was extracted with EtOAc. The combined organic extracts were washed with brine, dried over Na₂SO₄, filtered, and then concentrated. The resulting residue was purified by silica gel flash column chromatography (gradient, petroleum ether/EtOAc=99/1 to 50/50). Appropriate fractions were collected and concentrated. A mixture of the obtained material (7.00 g, 16.9 mmol), Na₂CO₃ (3.58 g, 33.8 mmol), and Boc₂O (4.42 g, 20.3 mmol) in DCM (100 mL) was stirred at rt for 12 h. The mixture was concentrated. The resulting residue was purified by silica gel flash column chromatography (gradient, petroleum ether/EtOAc=99/1 to 50/50) to afford the title compound (1.95 g, 3.79 mmol). LC-MS (ESI, m/z): [M+Na]⁺ 537.

### Reference Example-14-01: (S,Z)-2-((((9H-fluoren-9-yl)methoxy)carbonyl)amino)-3-(4-(2-methyl-3-((2,2,4,6,7-pentamethyl-2,3-dihydrobenzofuran-5-yl)sulfonyl)guanidino)phenyl)propanoic acid

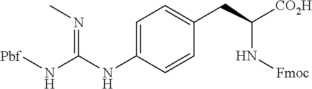

To a solution of 2,2,4,6,7-pentamethyl-2,3-dihydrobenzofuran-5-sulfonamide (CAS: 378230-81-0, 1.70 g, 6.31 mmol) in DMF (25.0 mL) at 0° C. was added sodium hydride (0.27 g, 60% dispersion in mineral oil, 6.63 mmol). The mixture was stirred at rt for 15 min and then isothiocyanatomethane (CAS: 556-61-6, 0.46 g, 6.31 mmol) was added and stirred at 50° C. for 30 min. After cooled to rt, reaction mixture was diluted with water. The mixture was acidified with 2M aq. HCl. The aqueous phase was extracted with hexane/ethyl acetate (1/1). The combined organic extracts were washed four times with water, brine, dried over Na₂SO₄, filtered, and then concentrated. To a solution of the obtained compound (2.20 g, 6.42 mmol) and methyl (S)-2-((((9H-fluoren-9-yl)methoxy)carbonyl)amino)-3-(4-aminophenyl)propanoate (CAS: 125218-63-5, 2.43 g, 5.84 mmol) in DMF (30.0 mL) at rt was added EDCI·HCl. The resulting mixture was stirred at rt for 1 h. The reaction mixture at 0° C. was quenched with satd. aq. NaHCO₃. The aqueous phase was extracted with hexane/ethyl acetate (1/1).

The combined organic extracts were washed four times with H₂O, brine, dried over Na₂SO₄, filtered, and then concentrated. The resulting residue was purified by silica gel flash column chromatography (gradient, hexane/EtOAc=100/0 to 30/70). Appropriate fractions were concentrated. To a solution of the resulting residue (4.50 g, 6.21 mmol) in ClCH₂CH₂Cl (35.0 mL) at rt was added Me₃SnOH (4.49 g, 24.8 mmol). The resulting mixture was stirred at 50° C. for 6 h. The reaction mixture was concentrated. The resulting residue was purified by silica gel flash column chromatography (gradient, CH₂Cl₂/MeOH=100/0 to 90/10) to afford the title compound (3.75 g, 5.28 mmol). LC-MS (ESI, m/z): [M+H]⁺ 711.

### Reference Example-14-02: (2S)-2-({[(9H-fluoren-9-yl)methoxy]carbonyl}(methyl)amino)-5-[(E)-N′-methyl-N′-[(2,2,4,6,7-pentamethyl-2,3-dihydro-1-benzofuran-5-yl)sulfonyl]carbamimidamido]pentanoic acid

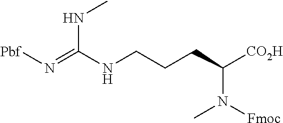

To a solution of 2,2,4,6,7-pentamethyl-2,3-dihydrobenzofuran-5-sulfonamide (CAS: 378230-81-0, 3.00 g, 11.1 mmol) in DMF (30 mL) at 0° C. was added NaH (0.47 g, 11.7 mmol) and stirred at rt for 15 min. Then isothiocyanatomethane (CAS: 556-61-6, 0.80 g, 10.9 mmol) was added and stirred at 50° C. for 30 min. The mixture was cooled at rt then diluted with water. The mixture was acidified with 2 M aq. HCl. The mixture was extracted with EtOAc/hexane. The organic extracts were washed with brine, dried over Na₂SO₄, filtered, and then concentrated to afford 3.05 g of 2,2,4,6,7-pentamethyl-N-(methylcarbamothioyl)-2,3-dihydrobenzofuran-5-sulfonamide (Intermediate-2-1). To a solution of (S)-2-((((9H-fluoren-9-yl)methoxy)carbonyl)(methyl)amino)-5-((tert-butoxycarbonyl)amino)pentanoic acid (CAS: 1793105-28-8, 4.00 g, 8.54 mmol) in DCM (42.7 mL) at rt was added 4M HCl in CPME (42.7 mL). The mixture was stirred at rt for 1 h then concentrated. To a suspension of the obtained compound (0.50 g, 1.24 mmol) in THF (7.5 mL) at rt was added MSA (0.59 mL, 3.70 mmol) and stirred at rt for 30 min. To the mixture at rt were added Intermediate-2-1 (0.42 g, 1.24 mmol) and EDCI·HCl (0.25 g, 1.30 mmol). The mixture was stirred at rt for 1 h. The reaction was quenched with 10% aq. citric acid. The organic extracts were washed with brine, dried over Na₂SO₄, filtered, and then concentrated. The residue was purified by silica gel column chromatography (gradient, CH₂Cl₂/MeOH=100/0 to 0/100) to afford the title compound (0.44 g, 1.24 mmol). LC-MS (ESI, m/z): [M+H]⁺ 677.

### Reference Example-14-03: (S,E)-2-((((9H-fluoren-9-yl)methoxy)carbonyl)amino)-3-(1-(N-methyl-N′-((2,2,4,6,7-pentamethyl-2,3-dihydrobenzofuran-5-yl)sulfonyl)carbamimidoyl)piperidin-4-yl)propanoic acid

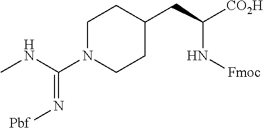

To a solution of 2,2,4,6,7-pentamethyl-2,3-dihydrobenzofuran-5-sulfonamide (CAS: 378230-81-0, 12.0 g, 44.6 mmol) in DMF (80 mL) at 0° C. was added NaH (1.12 g, 44.6 mmol) and stirred at 0° C. for 15 min. To the mixture was added isothiocyanatomethane (3.19 g, 43.7 mmol) at 0° C. The mixture was stirred at 50° C. for 30 min. The mixture was quenched with H₂O. The pH of the mixture was adjusted to 4 with 2M aq. HCl. The mixture was extracted with EtOAc. The organic extracts were washed with H₂O and brine, dried over Na₂SO₄, filtered, and concentrated. A mixture of (2S)-2-[[(9H-fluoren-9-ylmethoxy)carbonyl]amino]-3-(piperidin-4-yl)propanoic acid (CAS: 368866-16-4, 5.50 g, 13.9 mmol) and MSA (5.06 g, 34.86 mmol) in THF (160 mL) was stirred at rt for 30 min. To the mixture were add ed the obtained material above (4.77 g, 13.9 mmol) and EDCI·HCl (2.81 g, 14.6 m mol). The mixture was stirred at rt for 1 h. The mixture was quenched with H₂O. The mixture was extracted with EtOAc. The organic extracts were washed with brine, dried over Na₂SO₄, filtered, and concentrated. The resulting residue was purified by silica gel flash column chromatography (gradient, CH₂Cl₂/MeOH=99/1 to 90/10), and then further purified by reversed phase silica gel flash column chromatography (C18 silica gel, gradient, H₂O/CH₃CN with 0.1% formic acid=55/45 to 30/70) to afford the title compound (3.00 g, 4.28 mmol). LC-MS (ESI, m/z): [M+H]⁺ 703.

### Reference Example-14-04: (2S)-6-[(Z)—N′,N′-dimethyl-N″-[(2,2,4,6,7-pentamethyl-2,3-dihydro-1-benzofuran-5-yl)sulfonyl]carbamimidamido]-2-({[(9H-fluoren-9-yl)methoxy]carbonyl}amino)hexanoic acid

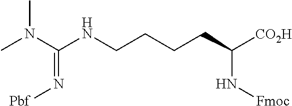

To a solution of prop-2-en-1-yl (2S)-6-amino-2-{[(9H-fluoren-9-ylmethoxy)carbonyl]amino}hexanoate hydrochloride (CAS: 815619-80-8, 132 g, 297 mmol) and DIEA (78.3 mL, 450 mmol) in DCM (1500 mL) at 0° C. was added 2,2,4,6,7-pentamethyl-3H-1-benzofuran-5-sulfonyl isothiocyanate (CAS: 690665-11-3, 101 g, 326 mmol). The mixture was stirred at rt for 30 min. To the mixture were added EDCI·HCl (160 g, 593 mmol) and dimethylamine (2M in THF, 296 mL, 593 mmol) at rt. The mixture was stirred at rt for 0.5 h. The mixture was washed with brine, dried over Na₂SO₄, filtered, and concentrated. The resulting residue was purified by silica gel flash column chromatography (isocratic, petroleum ether/EtOAc=33/67). Appropriate fractions were collected and concentrated. To a solution of the obtained material (140 g, 191 mmol) and N-methylaniline (61.6 g, 575 mmol) in THF (2000 mL) at rt was added Pd(PPh₃)₄ (11.1 g, 9.58 mmol) under N₂ atmosphere. The mixture was stirred at rt for 0.5 h. The mixture was diluted with H₂O and extracted with EtOAc. The organic extracts were washed with brine, dried over Na₂SO₄, filtered, and concentrated. The resulting residue was purified by silica gel flash column chromatography (isocratic, CH₂Cl₂/MeOH=91/9) to afford the title compound (111 g, 160 mmol). LC-MS (ESI, m/z): [M+H]⁺ 691.

### Reference Example-14-05: (2S)-2-({[(9H-fluoren-9-yl)methoxy]carbonyl}amino)-5-[(Z)—N′-[(2,2,4,6,7-pentamethyl-2,3-dihydro-1-benzofuran-5-yl)sulfonyl]-N″-(2-phenylethyl)carbamimidamido]pentanoic acid

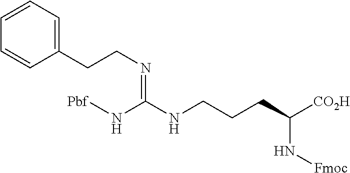

To a solution of 2,2,4,6,7-pentamethyl-2,3-dihydrobenzofuran-5-sulfonamide (CAS: 378230-81-0, 2.69 g, 10.0 mmol) in DMF (33.3 mL) at 0° C. was added NaH (0.42 g, 60% dispersion in mineral oil) and stirred at rt for 15 min. To the mixture was added (2-isothiocyanatoethyl)benzene (CAS: 2257-09-2, 1.47 mL, 10.0 mmol) and stirred at 50° C. for 30 min. The reaction was quenched with 1M aq. HCl at 0° C. The resulting mixture was extracted with hexane/EtOAc. The combined extracts were washed with H₂O and brine, dried over Na₂SO₄, filtered, and then concentrated. The resulting residue was triturated with hexane/EtOAc and the resulting solid was collected by filtration. To a suspension of (S)-2-((((9H-fluoren-9-yl)meth oxy)carbonyl)amino)-5-aminopentanoic acid hydrochloride (CAS: 201046-57-3, 3.13 g, 8.00 mmol) in THF (40.0 mL) at rt was added MSA (3.85 mL, 24.0 mmol). The mixture was stirred at rt for 30 min. To the mixture were added the obtained solid in the last step (3.46 g, 8.00 mmol) and EDCI·HCl (1.61 g, 8.40 mmol) at rt. The mixture was stirred at rt for 1 h. The reaction was quenched with 10% aq. citric acid. The resulting mixture was extracted with EtOAc. The combined extracts were washed with H₂O and brine, dried over Na₂SO₄, filtered, and then concentrated. The resulting residue was purified by silica gel flash column chromatography (gradient, DCM/EtOAc=80/20 to 40/60, then DCM/MeOH 100/0 to 80/20), and then further purified by silica gel flash column chromatography (gradient, DCM/Me OH 98/2 to 80/20). Appropriate fractions were collected and concentrated. The resulting residue was triturated with hexane and the resulting solid was collected by filtration to afford the title compound (2.65 g, 3.53 mmol). LC-MS (ESI, m/z): [M+H]⁺ 753.

### Reference Example-15-01: (2S)-2-({[(9H-fluoren-9-yl)methoxy]carbonyl}amino)-6-[(4-{2-oxo-2-[(2-phenylpropan-2-yl)oxy]ethyl}piperazine-1-carbonyl)amino]hexanoic acid

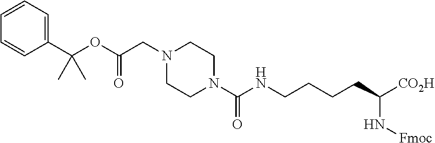

To a solution of (2S)-6-{[(tert-butoxy)carbonyl]amino}-2-({[(9H-fluoren-9-yl)methoxy]carbonyl}amino)hexanoic acid (CAS: 71989-26-9, 50 g, 107 mmol) in DCM (300 ml) at rt was added 4M HCL in CPME (200 mL) and stirred for 4 h. Formed solid was collected by filtration. The solid was triturated with hexane and the resulting solid was collected by filtration. To a suspension of the obtained material (1.00 g, 2.47 mmol) in THF (12.4 mL) at rt was added MSA (CAS: 7449-74-3, 0.79 g, 5.43 mmol). The mixture was stirred at rt for 0.5 h. To the solution at 0° C. were added DIPEA (0.52 mL, 2.96 mmol) and 4-nitrophenyl chloroformate (CAS: 7693-46-1, 0.597 g, 2.96 mmol). The mixture was stirred at 0° C. for 2 h. The reaction was quenched with 2M aq. HCl. The mixture was extracted twice with EtOAc. The combined organic extracts were washed with H₂O, brine, dried over Na₂SO₄, filtered, and concentrated. The resulting residue was purified by silica gel flash chromatography (gradient, CH₂Cl₂/MeOH=98/2 to 60/40) to afford 0.77 g of (2S)-2-({[(9H-fluoren-9-yl)methoxy]carbonyl}amino)-6-{[(4-nitrophenoxy)carbonyl]amino}hexanoic acid (Intermediate-3-1). To a refluxed solution of piperazine (7.42 g, 86 mmol) in THF (48 mL) was added dropwise a solution of 2-phenylpropan-2-yl 2-bromo acetate (CAS: 153698-47-6, 3.69 g, 14.35 mmol) in THF (24 mL) over 5 min. The mixture was stirred for 2 h under the reflux, and then cooled to rt, and then filter ed. The filtrate was concentrated. The resulting residue was purified by silica gel flash column chromatography (gradient, CH₂Cl₂/MeOH=100/0 to 80/20, and then CH₂Cl₂/MeOH with 1% Et₃N=100/0 to 80/20). Appropriate fractions were concentrated. To a solution of Intermediate-3-1 (4.98 g, 9.34 mmol) in THF (28 mL) at 0° C. was added dropwise a solution of the obtained material in the last step (2.57 g, 9.81 mmol) and DIPEA (1.80 mL, 10.27 mmol) in DMF (3.1 mL). The mixture was stirred at 30° C. for 3 h. The reaction at rt was quenched with 2M aq. NaH₂PO₄. The mixture was extracted with EtOAc/THF (ca. 5/1). The combined organic extracts were washed with brine, dried over Na₂SO₄, filtrated, and concentrated. The resulting residue was purified by silica gel flash chromatography (gradient, CH₂Cl₂/MeOH=100/0 to 92/8) to afford the title compound (3.32 g, 5.06 mmol). LC-MS (ESI, m/z): [M+H]⁺ 658.

### Reference Example-16-01: (2S)-3-[1-(3-{[(tert-butoxy)carbonyl]amino}propyl)-1H-indol-3-yl]-2-({[(9H-fluoren-9-yl)methoxy]carbonyl}amino)propanoic acid

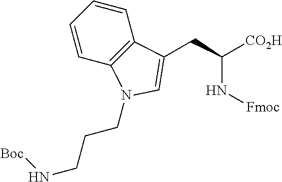

To a solution of (2S)-2-[[(benzyloxy)carbonyl]amino]-3-(1H-indol-3-yl)propanoic acid (CAS: 7432-21-5, 20.0 g, 59.1 mmol) in DMF (100 mL) and THF (100 mL) at 0° C. were added potassium tert-butoxide (13.3 g, 118 mmol). The mixture was stirred at rt for 30 min. To the mixture was added tert-butyl (3-bromopropyl)carbamate (CAS: 83948-53-2, 14.8 g, 62.1 mmol) at 0° C. The mixture was stirred at rt for 3 h. The reaction mixture at 0° C. was quenched with H₂O. The aqueous phase was extracted three times with EtOAc. The combined organic extracts were washed three times with brine, dried over Na₂SO₄, filtered, and then concentrated. The resulting residue was purified by silica gel flash column chromatography (isocratic, petroleum ether/EtOAc=25/75). Appropriate fractions were concentrated. To a solution of the obtained material (10.0 g, 20.2 mmol) in MeOH (100 mL) at rt was added Pd/C (4.5 g, wet). The mixture was stirred at rt for 2 h under H₂ atmosphere, and then filtered. The filtrate was concentrated. To a solution of the obtained material (10.0 g, 27.7 mmol) in 1,4-dioxane (90.0 mL) and H₂O (16.0 mL) at 0° C. was added NaHCO₃ (4.65 g, 55.3 mmol), followed by Fmoc-OSu (9.33 g, 27.7 mmol). The mixture was stirred at rt for 3 h. The pH of the mixture was adjusted to pH 5-6 with 20% aq. citric acid. The mixture was extracted three times with EtOAc. The organic extracts were washed with brine, dried over Na₂SO₄, filtered, and then concentrated. The resulting residue was purified by silica gel flash column chromatography (isocratic, petroleum ether/EtOAc=20/80) to afford the title compound (10.6 g, 18.1 mmol). LC-MS (ESI, m/z): [M-Boc+H]⁺ 484.

### Reference Example-17-01: 3-((((9H-fluoren-9-yl)methoxy)carbonyl)(3-((tert-butoxycarbonyl)amino)-2,2-dimethylpropyl)amino)propanoic acid

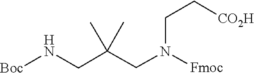

To a solution of tert-butyl (3-amino-2,2-dimethylpropyl)carbamate (CAS: 292606-35-0, 5.00 g, 24.7 mmol) in MeCN (55 mL) at rt was added acrylic acid (CAS: 79-10-7, 1.78 g, 24.7 mmol). The mixture was stirred at 80° C. for 8 h. To the mixture at 0° C. were added H₂O (55 mL), NaHCO₃ (8.31 g, 99.0 mmol), and Fmoc-OSu (7.92 g, 23.5 mmol) in 1,4-dioxane (55 mL). The mixture was stirred at rt for 15 h. The mixture was concentrated. The resulting residue was washed with Et OAc, then the pH of the aqueous layer was adjusted to 4 with citric acid. The mixture was extracted with EtOAc. The organic extracts were concentrated to afford the title compound (5.10 g, 103 mmol). LC-MS (ESI, m/z): [M+H]⁺ 497.

### Reference Example-18-01: 3-((((9H-fluoren-9-yl)methoxy)carbonyl)amino)bicyclo[3.1.0]hexane-3-carboxylic acid

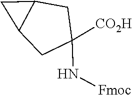

To a solution of bicyclo[3.1.0]hexan-3-one (5.00 g, 52.0 mmol) in H2O/EtOH (40 mL) at rt were added (NH₄)₂CO₃ (12.5 g, 130.0 mmol) and KCN (3.70 g, 57.2 mmol). The mixture was stirred at 50° C. overnight. The resulting mixture was concentrated. The mixture was extracted with EtOAc. The organic extracts were washed with brine, dried over Na₂SO₄, filtered, and concentrated. To a solution of the obtained material (0.50 g, 3.01 mmol) in H₂O at rt was added KOH (0.84 g, 15.0 mmol). The mixture was stirred at 100° C. overnight. The pH of the mixture was adjusted to 7 with aq. HCl. The mixture was concentrated. To a solution of the obtained material (3.00 g, 21.3 mmol) in 1,4-dioxane (30 mL) and H₂O (30 mL) at r t were added NaHCO₃ (3.60 g, 42.5 mmol) and Fmoc-OSu (7.10 g, 21.3 mmol). The mixture was stirred at rt for 2 h. The pH of the mixture was adjusted to 5 with conc. aq. HCl. The mixture was extracted with EtOAc. The organic extracts were dried over Na₂SO₄, filtered, and concentrated. The resulting residue was purified by reversed phase silica gel flash column chromatography (C18 silica gel, gradient, H₂O/CH₃CN=90/10 to 50/50) to afford the title compound (1.48 g, 4.07 mmol) LC-MS (ESI, m/z): [M+H]⁺ 364.

### Reference Example 19-01: (2R)-3-{[(2,4-dimethoxyphenyl)methyl]sulfamoyl}-2-({[(9H-fluoren-9-yl)methoxy]carbonyl}amino)propanoic acid

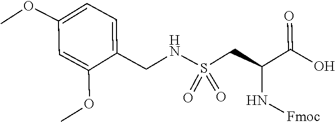

To a solution of (2R)-2-({[(9H-fluoren-9-yl)methoxy]carbonyl}amino)-3-[(triphenylmethyl)sulfanyl]propanoic acid (CAS: 103213-32-7, 20.0 g, 34.1 mmol) in DMF (200 mL) at 0° C. were added potassium carbonate (14.2 g, 102 mmol) and a solution of iodomethane (14.5 g, 102 mmol) in DMF (50.0 mL). The mixture was stirred at rt for 3 h. The reaction was then quenched with H₂O. The mixture was extracted with EtOAc. The organic extracts were washed with brine, dried over Na₂SO₄, filtered, and concentrated. To a solution of the obtained material (7.0 g, 11.7 mmol) in MeCN (70 mL) at rt was added H₂O (0.63 g, 35.0 mmol). To the mixture at 0° C. was added tert-butyl hypochlorite (CAS: 507-40-4, 3.80 g, 35.0 mmol) dropwise. The mixture was stirred at 0° C. for 15 min. To the mixture at 0° C. was added 1-(2,4-dimethoxyphenyl) methanamine (CAS: 20781-20-8, 7.81 g, 46.7 mmol). The mixture was stirred at rt for 1 h. The mixture was concentrated. The resulting residue was purified by silica gel flash column chromatography (isocratic, petroleum ether/EtOAc=67/33). Appropriate fractions were collected and concentrated. To a solution of the obtained material (4.40 g, 7.93 mmol) in ClCH₂CH₂Cl (40.0 mL) was added trimethyltin hydroxide (2.87 g, 15.9 mmol). The resulting mixture was stirred at 60° C. for 16 h. The mixture was concentrated. The resulting residue was dissolved in EtOAc. The mixture was washed with 1M aq. HCl, brine, dried over Na₂SO₄, filtered, and then concentrated. The resulting residue was purified by silica gel flash column chromatography (isocratic, CH₂Cl₂/MeOH=97/3) to afford the title compound (2.33 g, 4.22 mmol). LC-MS (ESI, m/z): [M−H]⁻ 539.

### Reference Example-20-01: (2S)-3-[4-(2-{[2-(tert-butoxy)-2-oxoethyl][(tert-butoxy)carbonyl]amino}ethoxy)phenyl]-2-({[(9H-fluoren-9-yl)methoxy]carbonyl}amino)propanoic acid

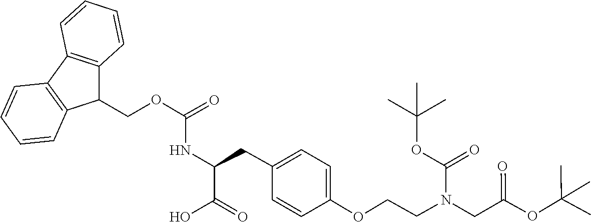 [SMILES: CC(C)(C)OC(=O)CN(CCOc1ccc(C[C@H](NC(=O)OCC2c3ccccc3-c3ccccc32)C(=O)O)cc1)C(=O)OC(C)(C)C]

To a solution of benzyl (2S)-2-{[(benzyloxy)carbonyl]amino}-3-(4-hydroxyphenyl)propanoate (CAS: 5513-40-6, 10.43 g, 25.72 mmol) and tert-butyl 2-{[(tert-butoxy)carbonyl](2-hydroxyethyl)amino}acetate (CAS: 1314371-47-5, 4.72 g, 17.14 mmol) in toluene (29 mL) and THF (29 mL) were added triphenylphosphine (6.74 g, 25.70 mmol) and Di-2-methoxyethyl azodicarboxylate (6.02 g, 25.70 mmol) at 0° C. The mixture was stirred at rt for 1 h and then quenched with H₂O. The mixture was extracted with toluene. The combined organic extracts were washed with H₂O, brine, dried over Na₂SO₄, filtered and concentrated. The resulting residue was purified by silica gel flash column chromatography (gradient, heptane/EtOAc=90/10 to 50/50). Appropriate fractions were concentrated. To a solution of the obtained compound (24.0 g, 36.2 mmol) in trifluoroethanol (45 ml) and CH₂Cl₂ (136 mL) at rt was added triethylsilane (23.14 mL, 144.8 mmol) and palladium on carbon (5.78 g, 5.43 mmol). The mixture was stirred at rt for 6 h. The mixture was filtered and the filtrate was concentrated. To a solution of the resulting residue in 1,4-dioxane (145 mL) and H₂O (145 mL) at 0° C. was added NaHCO₃ (12.18 g, 145.0 mmol), followed by a solution of Fmoc-OSu (11.60 g, 34.4 mmol) in 1,4-dioxane (72 mL) dropwise. The mixture was stirred for 3 h at rt, and then pH of the solution was adjusted to 4-5 with 2 M aq. HCl at 0° C. The mixture was extracted twice with EtOAc. The organic extracts were washed with brine, dried over Na₂SO₄, filtered, and concentrated. The resulting residue was purified by silica gel flash column chromatography (gradient, CH₂Cl₂/MeOH=100/0 to 85/15) to afford the title compound (21.82 g, 32.7 mmol). LC-MS (ESI, m/z): [M+H]⁺ 661.

### Reference Example-21-01: (R)-4-((((9H-fluoren-9-yl)methoxy)carbonyl)amino)-5-(4-chlorophenyl)pentanoic acid

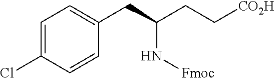

To a solution of tert-butyl (S)-(1-(4-chlorophenyl)-3-oxopropan-5 2-yl)carbamate (CAS: 322392-25-6, 6.00 g, 21.1 mmol) in DCM (60 mL) at rt was added ethyl 2-(triphenyl-lambda5-phosphanylidene)acetate (CAS: 1099-45-2, 8.84 g, 25.4 mmol). The mixture was stirred at rt for 2 h. The mixture was concentrated. The resulting residue was purified by silica gel flash column chromatography (gradient, petroleum ether/EtOAc=99/1 to 75/25). Appropriate fractions were collected and concentrated. A mixture of the obtained material (6.00 g, 17.0 mmol) and Rh/C (6.00 g) in EtOAc (60 mL) was stirred at rt for 2 h under H₂ atmosphere. The mixture was filtered and concentrated. To a solution of the obtained material (3.00 g, 8.43 mmol) in THF (20 mL) and H2O (20 mL) at rt was added LiOH (605 mg, 25.3 mmol). The mixture was stirred at rt for 4 h. The mixture was concentrated. The resulting residue was purified by silica gel flash column chromatography (gradient, petroleum ether/EtOAc=99/1 to 91/9). Appropriate fractions were collected and concentrated. To the resulting residue (2.70 g, 8.24 5 mmol) at 0° C. was added HCl in 1,4-dioxane (20 mL). The mixture was stirred at rt for 2 h. The mixture was concentrated. To a solution of the resulting residue (1.80 g, 7.91 mmol) in 1,4-dioxane (30 mL) and H2O (15 mL) at 0° C. were added NaHCO₃ (1.99 g, 23.7 mmol) and Fmoc-OSu (2.67 g, 7.91 mmol). The mixture was stirred at rt for 3 h. The mixture was diluted with H₂O and extracted with EtOAc. The organic extracts were washed with brine, dried over Na₂SO₄, filtered, and concentrated. The resulting residue was purified by reversed phase silica gel flash column chromatography (C18 silica gel, gradient, H₂O/CH3CN with 0.1% formic acid=40/60 to 25/75) to afford the title compound (1.92 g, 4.27 mmol). LC-MS (ESI, m/z): [M+Na]⁺ 472.

### Example-1-01: (2S)-2-((((9H-fluoren-9-yl)methoxy)carbonyl)amino)-3-(5-(((3-m ethylpentan-3-yl)oxy)carbonyl)pyridin-3-yl)propanoic acid

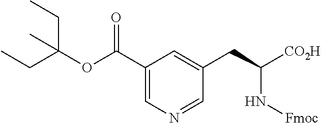

To a solution of 5-bromopyridine-3-carboxylic acid (CAS: 20826-04-4, 50 g, 248 mmol) in DCM (1000 mL) at 0° C. were added 3-methyl-3-pentanol (CAS: 77-74-7, 253 g, 2475 mmol), EDCI·HCl (94.9 g, 495 mmol) and DMAP (60.5 g, 495 mmol). The mixture was stirred at rt for 1 h. The reaction was quenched by the addition of aq. NaHCO₃ at rt and extracted with DCM. The organic extracts were washed with brine, dried over Na₂SO₄, filtered, and then concentrated. The resulting residue was purified by silica gel flash column chromatography (isocratic, petroleum ether/DCM=5/1). Appropriate fractions were concentrated. To a suspension of Zn (41.1 g, 629 mmol) in DMF (1000 mL) was added I₂ (16.0 g, 62.9 mmol). The mixture was stirred at rt for 10 min. To the mixture at rt was added methyl (2R)-2-({[(9H-fluoren-9-yl)methoxy]carbonyl}amino)-3-iodopropanoate (CAS: 156017-42-4, 114 g, 252 mmol). The mixture was stirred at rt for an additional 1 h. To the mixture at rt were added the obtained material in the last step (60 g, 210 mmol), SPhos (17.2 g, 41.9 mmol), and Pd₂(dba)₃CHCl₃ adduct (10.7 g, 10.5 mmol). The mixture was stirred at 50° C. for an additional 1 h. The reaction was quenched by the addition of water at rt. The mixture was filtered. The filtrate was extracted three times with EtOAc. The combined organic extracts were washed with brine, dried over Na₂SO₄, filtered, and then concentrated. The resulting residue was purified by silica gel flash column chromatography (isocratic, petroleum ether/EtOAc=5/1). Appropriate fractions were concentrated. To a mixture of the obtained material (85 g, 160 mmol) in 2-propanol (1500 mL) at 0° C. was added a solution of CaCl₂ (284 g, 2563 mmol) in H₂O (400 mL). The resulting mixture was stirred at 0° C. for an additional 10 min. To the mixture at 0° C. was added a solution of LiOH (15.4 g, 641 mmol) in H₂O (100 mL) dropwise. The mixture was stirred at rt for 2 days. The pH of the mixture was adjusted to pH 5 with 1 M aq. HCl. The resulting mixture was extracted three times with EtOAc. The combined organic extracts were washed with brine, dried over Na₂SO₄, filtered, and then concentrated. The resulting residue was purified by silica gel flash column chromatography (isocratic, DCM/MeOH=6/1) to afford the title compound (52.2 g, 101 mmol). ¹H NMR (500 MHz, DMSO-d₆) δ 13.00 (br, 1H), 8.96-8.85 (m, 1H), 8.74-8.65 (m, 1H), 8.21-8.12 (m, 1H), 7.94-7.76 (m, 3H), 7.63-7.54 (m, 2H), 7.44-7.34 (m, 2H), 7.32-7.19 (m, 2H), 4.32-4.07 (m, 4H), 3.26-3.17 (m, 1H), 3.04-2.94 (m, 1H), 2.02-1.86 (m, 2H), 1.86-1.73 (m, 2H), 1.49-1.39 (m, 3H), 0.87-0.81 (m, 6H). LC-MS (ESI, m/z): [M+H]⁺ 517.

### Example-1

The following compounds can be synthesized as outlined for the preparation of Reference example-2-01, Reference example-2-02, Reference example-2-03, Reference example-2-04, Reference example-2-05, or Example-1-01 employing appropriate starting materials in the table.

| | Structure and IUPAC name |
|---|---|
| | Starting material-A |
| | Starting material-B |
| Example | NMR and MS |
| 1-02 | 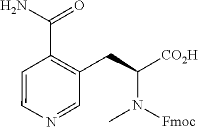 |
| | (S)-2-((((9H-fluoren-9-yl)methoxy)carbonyl)(methyl)amino)-3-(4- |
| | carbamoylpyridin-3-yl)propanoic acid |
| | methyl (2R)-2-({[(9H-fluoren-9-yl)methoxy]carbonyl}(methyl)amino)-3- |
| | iodopropanoate (CAS: 2973756-19-1) |
| | 3-Bromo-4-pyridinecarboxamide (CAS: 13958-99-1) |
| | ¹H NMR (500 MHz, DMSO-d₆) δ 12.97 (br, 1H), 8.53-8.47 (m, 1H), 8.42- |
| | 8.23 (m, 1H), 8.15-7.99 (m, 1H), 7.94-7.81 (m, 2H), 7.72 (s, 1H), 7.63- |
| | 7.46 (m, 2H), 7.45-7.21 (m, 5H), 5.06-4.70 (m, 1H), 4.21 (s, 2H), 4.14- |
| | 4.03 (m, 1H), 3.48-3.36 (m, 1H), 3.23-2.96 (m, 1H), 2.79-2.65 (m, 3H). |
| | LC-MS (ESI, m/z): [M + H]⁺ 446. |
| 1-03 | 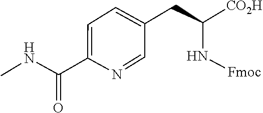 |
| | (S)-2-((((9H-fluoren-9-yl)methoxy)carbonyl)amino)-3-(6- |
| | (methylcarbamoyl)pyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | 5-bromo-N-methylpyridine-2-carboxamide (CAS: 845305-87-5) |
| | ¹H NMR (500 MHz, DMSO-d₆) δ 12.65 (br, 1H), 8.79-8.68 (m, 1H), 8.59- |
| | 8.50 (m, 1H), 8.00-7.79 (m, 5H), 7.67-7.35 (m, 4H), 7.35-7.22 (m, 2H), |
| | 4.33-4.10 (m, 4H), 3.26-2.91 (m, 2H), 2.81 (d, J = 4.7 Hz, 3H). LC-MS |
| | (ESI, m/z): [M + H]⁺ 446. |
| 1-04 | 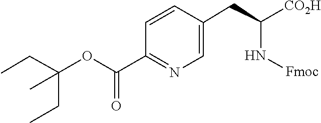 |
| | (S)-2-((((9H-fluoren-9-yl)methoxy)carbonyl)amino)-3-(6-(((3- |
| | methylpentan-3-yl)oxy)carbonyl)pyridin-3-yl)propanoic acid |
| | (2R)-2-({[(9H-fluoren-9-yl)methoxy]carbonyl}amino)-3- |
| | methyl (2R)-2-({[(9H-fluoren-9-yl)methoxy]carbonyl}amino)-3- |
| | iodopropanoate (CAS: 156017-42-4) |
| | 5-bromopyridine-2-carboxylic acid (CAS: 30766-11-1) |
| | ¹H NMR (500 MHz, DMSO-d₆) δ 13.23 (br, 1H), 8.70-8.41 (m, 1H), 7.95- |
| | 7.77 (m, 4H), 7.67-7.54 (m, 3H), 7.48-7.20 (m, 4H), 4.31-4.08 (m, 4H), |
| | 3.25-3.17 (m, 1H), 3.03-2.90 (m, 1H), 2.03-1.89 (m, 2H), 1.87-1.74 (m, |
| | 2H), 1.44 (s, 3H), 0.95-0.75 (m, 6H). LC-MS (ESI, m/z): [M + H]⁺ 517. |
| 1-05 | 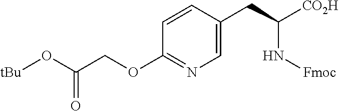 |
| | (S)-2-((((9H-fluoren-9-yl)methoxy)carbonyl)amino)-3-(6-(3-(tert-butoxy)- |
| | 3-oxopropyl)pyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 3-(5-bromopyridin-2-yl)propanoate (CAS: 1997699-66-7) |
| | ¹H NMR (500 MHZ, DMSO-d₆) δ 12.81 (br, 1H), 8.38-8.36 (m, 1H), 7.88 |
| | (d, J = 7.5 Hz, 2H), 7.76 (d, J = 8.5 Hz, 1H), 7.66-7.61 (m, 2H), 7.60-7.56 |
| | (m, 1H), 7.44-7.38 (m, 2H), 7.34-7.27 (m, 2H), 7.17 (d, J = 7.9 Hz, 1H), |
| | 4.23-4.13 (m, 4H), 3.10-3.04 (m, 1H), 2.94-2.82 (m, 3H), 2.61-2.54 (m, |
| | 2H), 1.34 (s, 9H). LC-MS (ESI, m/z): [M + H]⁺ 517. |
| 1-06 | 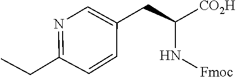 |
| | (S)-2-((((9H-fluoren-9-yl)methoxy)carbonyl)amino)-3-(6-ethylpyridin-3- |
| | yl)propanoic acid |
| | methyl (2R)-2-({[(9H-fluoren-9-yl)methoxy]carbonyl}amino)-3- |
| | iodopropanoate (CAS: 156017-42-4) |
| | 5-Bromo-2-ethylpyridine (CAS: 38749-90-5) |
| | ¹H NMR (500 MHz, DMSO-d₆) δ 12.86 (br, 1H), 8.45 (s, 1H), 7.91-7.86 |
| | (m, 2H), 7.84-7.78 (m, 1 H), 7.66-7.59 (m, 2H), 7.44-7.38 (m, 2H), |
| | 7.36-7.25 (m, 3H), 4.25-4.13 (m, 4H), 3.20-3.04 (m, 1 H), 2.93-2.85 |
| | (m, 1H), 2.80-2.69 (m, 2H), 1.24-1.14 (m, 3H). LC-MS (ESI, m/z): |
| | [M + H]⁺ 417. |
| 1-07 | 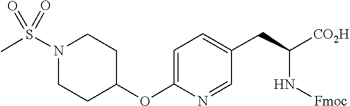 |
| | (S)-2-((((9H-fluoren-9-yl)methoxy)carbonyl)amino)-3-(6-((1- |
| | (methylsulfonyl)piperidin-4-yl)oxy)pyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | 5-Bromo-2-[[1-(methylsulfonyl)-4-piperidinyl]oxy]pyridine |
| | (CAS: 494771-87-8) |
| | ¹H NMR (500 MHz, DMSO-d₆) δ 12.80 (br, 1H), 8.03 (d, J = 2.1 Hz, 1H), |
| | 7.89 (d, J = 7.5 Hz, 2H), 7.78 (d, J = 8.5 Hz, 1H), 7.67-7.58 (m, 3H), 7.44- |
| | 7.39 (m, 2H), 7.33-7.26 (m, 2H), 6.70 (d, J = 8.3 Hz, 1H), 5.12-5.02 (m, |
| | 1H), 4.28-4.11 (m, 4H), 3.40-3.25 (m, 2H), 3.12-2.98 (m, 3H), 2.93- |
| | 2.86 (m, 3H), 2.83-2.75 (m, 1H), 2.12-1.84 (m, 2H), 1.74-1.59 (m, 2H). |
| | LC-MS (ESI, m/z): [M + H]⁺ 566. |
| 1-08 | 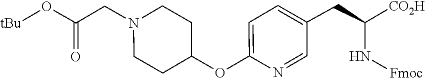 |
| | (S)-2-((((9H-fluoren-9-yl)methoxy)carbonyl)amino)-3-(6-((1-(2-(tert- |
| | butoxy)-2-oxoethyl)piperidin-4-yl)oxy)pyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 2-(4-((5-bromopyridin-2-yl)oxy)piperidin-1-yl)acetate |
| | ¹H NMR (500 MHz, DMSO-d₆) δ 12.81 (br, 1H), 8.03 (s, 1H), 7.91-7.86 |
| | (m, 2H), 7.82-7.74 (m, 1H), 7.68-7.58 (m, 3H), 7.44-7.38 (m, 2H), 7.35- |
| | 7.25 (m, 2H), 6.73-6.68 (m, 1H), 5.22-4.92 (m, 1H), 4.33-4.05 (m, 5H), |
| | 3.62-3.55 (m, 1H), 3.08-3.01 (m, 1H), 2.94 (s, 2H), 2.85-2.80 (m, 1H), |
| | 2.78 (s, 2H), 2.20-1.99 (m, 2H), 1.91-1.71 (m, 2H), 1.46 (s, 9H). LC-MS |
| | (ESI, m/z): [M + H]⁺ 602. |
| 1-09 | 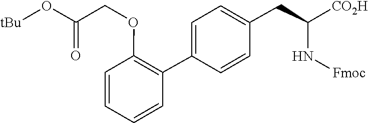 |
| | (S)-2-((((9H-fluoren-9-yl)methoxy)carbonyl)amino)-3-(6-(3- |
| | chlorophenyl)pyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | 5-bromo-2-(3-chlorophenyl)pyridine (CAS: 1374665-29-8) |
| | ¹H NMR (500 MHZ, DMSO-d₆) δ 12.92 (br, 1H), 8.61-8.56 (m, 1H), 8.09 |
| | (s, 1H), 8.07-8.00 (m, 1H), 7.94 (d, J = 8.2 Hz, 1H), 7.87 (d, J = 7.5 Hz, |
| | 2H), 7.83-7.73 (m, 2H), 7.68-7.59 (m, 2H), 7.55-7.45 (m, 2H), 7.42- |
| | 7.35 (m, 2H), 7.33-7.24 (m, 2H), 4.31-4.08 (m, 4H), 3.27-3.07 (m, 1H), |
| | 2.99-2.78 (m, 1H). LC-MS (ESI, m/z): [M + H]⁺ 499. |
| 1-10 | 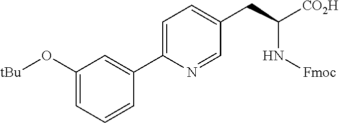 |
| | (S)-2-((((9H-fluoren-9-yl)methoxy)carbonyl)amino)-3-(6-(3-(tert- |
| | butoxy)phenyl)pyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | 5-bromo-2-(3-(tert-butoxy)phenyl)pyridine |
| | ¹H NMR (500 MHz, DMSO-d₆) δ 12.82 (s, 1H), 8.56 (m, 1H), 7.86 (t, J = |
| | 7.8 Hz, 3H), 7.70-7.81 (m, 3H), 7.57-7.68 (m, 3H), 7.43-7.33 (m, 3H), |
| | 7.33-7.18 (m, 2H), 7.03 (dd, J = 8.0, 1.8 Hz, 1H), 4.08-4.29 (m, 4H), 3.11- |
| | 3.22 (m, 1H), 2.86-2.98 (m, 1H), 1.32 (s, 9H). LC-MS (ESI, m/z): [M + H]⁺ |
| | 537. |
| 1-11 | 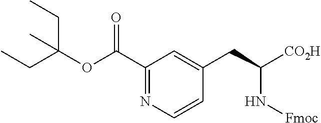 |
| | (S)-2-((((9H-fluoren-9-yl)methoxy)carbonyl)amino)-3-(2-(((3- |
| | methylpentan-3-yl)oxy)carbonyl)pyridin-4-yl)propanoic acid |
| | methyl (2R)-2-({[(9H-fluoren-9-yl)methoxy]carbonyl}amino)-3- |
| | iodopropanoate (CAS: 156017-42-4) |
| | 4-bromopyridine-2-carboxylic acid (CAS: 30766-03-1) |
| | ¹H NMR (500 MHZ, DMSO-d₆) δ 13.39-11.99 (br, 1H), 8.57 (d, J = 4.9 |
| | Hz, 1H), 7.92 (s, 1H), 7.87 (d, J = 7.6 Hz, 2H), 7.72 (d, J = 7.3 Hz, 1H), |
| | 7.64-7.52 (m, 2H), 7.50 (d, J = 5.3 Hz, 1H), 7.42-7.37 (m, 2H), 7.33-7.21 |
| | (m,2 H), 4.28-4.12 (m, 4H), 3.25-3.18 (m, 1H), 3.03-2.94 (m, 1H), 1.99- |
| | 1.90 (m, 2H), 1.87-1.75 (m, 2H), 1.43 (s, 3H), 0.84 (t, J = 7.5 Hz, 6H). LC- |
| | MS (ESI, m/z): [M + H]⁺ 517. |
| 1-12 | 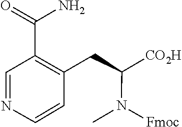 |
| | (S)-2-((((9H-fluoren-9-yl)methoxy)carbonyl)(methyl)amino)-3-(3- |
| | carbamoylpyridin-4-yl)propanoic acid |
| | methyl (2R)-2-({[(9H-fluoren-9-yl)methoxy]carbonyl}(methyl)amino)-3- |
| | iodopropanoate (CAS: 2973756-19-1) |
| | 4-Bromo-3-pyridinecarboxamide (CAS: 89581-36-2) |
| | ¹H NMR (500 MHZ, DMSO-d₆) δ 13.31-12.64 (br, 1H), 8.57 (d, J = 4.7 |
| | Hz, 1H), 8.45-8.41 (m, 1H), 8.04 (s, 1H), 7.92-7.84 (m, 2H), 7.63 (s, 1H), |
| | 7.60-7.52 (m, 2H), 7.44-7.38 (m, 2H), 7.35-7.28 (m, 2H), 7.18-6.88 (m, |
| | 1H), 4.97-4.80 (m, 1H), 4.33-4.19 (m, 2H), 4.15 (s, 1H), 3.49-3.41 (m, |
| | 1H), 3.24-2.95 (m, 1H), 2.68-2.64 (m, 3H). LC-MS (ESI, m/z): [M + H]⁺ |
| | 446. |
| 1-13 | 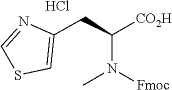 |
| | (S)-2-((((9H-fluoren-9-yl)methoxy)carbonyl)(methyl)amino)-3-(thiazol-4- |
| | yl)propanoic acid hydrochloride |
| | methyl (2R)-2-({[(9H-fluoren-9-yl)methoxy]carbonyl}(methyl)amino)-3- |
| | iodopropanoate (CAS: 2973756-19-1) |
| | 4-iodo-1,3-thiazole (CAS: 108306-60-1) |
| | ¹H NMR (500 MHz, DMSO-d₆) δ 13.80-12.00 (m, 1H), 9.14-9.02 (m, |
| | 1H), 7.89 (d, J = 7.5 Hz, 2H), 7.64-7.53 (m, 2H), 7.44-7.37 (m, 3H), 7.35- |
| | 7.24 (m, 3H), 5.04-4.85 (m, 1H), 4.32-4.14 (m, 3H), 3.34-3.16 (m, 2H), |
| | 2.76 (s, 3H). LC-MS (ESI, m/z): [M + H]⁺ 409. |
| 1-14 | 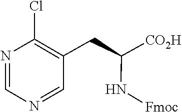 |
| | (S)-2-((((9H-fluoren-9-yl)methoxy)carbonyl)amino)-3-(4-chloropyrimidin- |
| | 5-yl)propanoic acid |
| | methyl (2R)-2-({[(9H-fluoren-9-yl)methoxy]carbonyl}amino)-3- |
| | iodopropanoate (CAS: 156017-42-4) |
| | 5-Bromo-4-chloropyrimidine (CAS: 56181-39-6) |
| | ¹H NMR (500 MHZ, DMSO-d₆) δ 13.08 (s, 1H), 8.93 (s, 1H), 8.72 (s, 1H), |
| | 7.92-7.83 (m, 3H), 7.67-7.58 (m, 2H), 7.45-7.37 (m, 2H), 7.35-7.25 (m, |
| | 2H), 4.40-4.11 (m, 4H), 3.30-3.23 (m, 1H), 3.05-2.96 (m, 1H). LC-MS |
| | (ESI, m/z): [M + H]⁺ 424. |
| 1-15 | 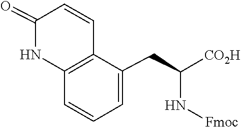 |
| | (S)-2-((((9H-fluoren-9-yl)methoxy)carbonyl)amino)-3-(2-oxo-1,2- |
| | dihydroquinolin-5-yl)propanoic acid |
| | methyl (2R)-2-({[(9H-fluoren-9-yl)methoxy]carbonyl}amino)-3- |
| | iodopropanoate (CAS: 156017-42-4) |
| | 5-Bromo-2-methoxyquinoline (CAS: 99455-06-8) |
| | ¹H NMR (500 MHZ, DMSO-d₆) δ 12.90 (s, 1H), 11.75 (s, 1H), 8.10 (d, J = |
| | 9.8 Hz, 1H), 7.88 (d, J = 7.6 Hz, 2H), 7.80 (d, J = 8.5 Hz, 1H), 7.63 (d, J = |
| | 7.5 Hz, 1H), 7.60 (d, J = 7.5 Hz, 1H), 7.44-7.35 (m, 3H), 7.34-7.24 (m, |
| | 2H), 7.20 (d, J = 8.2 Hz, 1H), 7.09 (d, J = 7.3 Hz, 1H), 6.55 (d, J = 9.8 Hz, |
| | 1H), 4.24-4.00 (m, 4H), 3.47-3.38 (m, 1H), 3.18-3.10 (m, 1H). LC-MS |
| | (ESI, m/z): [M + H]⁺ 455. |
| 1-16 | 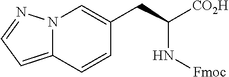 |
| | (S)-2-((((9H-fluoren-9-yl)methoxy)carbonyl)amino)-3-(pyrazolo[1,5- |
| | alpyridin-6-yl)propanoic acid |
| | methyl (2R)-2-({[(9H-fluoren-9-yl)methoxy]carbonyl}amino)-3- |
| | iodopropanoate (CAS: 156017-42-4) |
| | 6-Bromopyrazolo[1,5-a]pyridine (CAS: 1264193-11-4) |
| | ¹H NMR (500 MHZ, DMSO-d₆) δ 12.86 (br, 1 H), 8.57 (s, 1 H), 7.95 (d, |
| | J = 2.1 Hz, 1 H), 7.90-7.78 (m, 3 H), 7.65-7.55 (m, 3 H), 7.42-7.33 |
| | (m, 2 H), 7.30-7.13 (m, 2 H), 6.59-6.54 (m, 1 H), 4.27-4.12 (m, 4 H), |
| | 3.16-3.08 (m, 1 H), 2.93-2.83 (m, 1 H). LC-MS (ESI, m/z): [M + H]⁺ 428. |
| 1-17 | 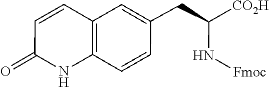 |
| | (S)-2-((((9H-fluoren-9-yl)methoxy)carbonyl)amino)-3-(2-oxo-1,2- |
| | dihydroquinolin-6-yl)propanoic acid |
| | methyl (2R)-2-({[(9H-fluoren-9-yl)methoxy]carbonyl}amino)-3- |
| | iodopropanoate (CAS: 156017-42-4) |
| | 6-Bromo-2-methoxyquinoline (CAS: 99455-05-7) |
| | ¹H NMR (500 MHz, DMSO-d₆) δ 11.70 (s, 1H), 7.92-7.84 (m, 2H), 7.82- |
| | 7.78 (m, 1H), 7.78-7.71 (m, 1H), 7.66-7.57 (m, 2H), 7.53 (s, 1H), 7.44- |
| | 7.36 (m, 3H), 7.33-7.14 (m, 4H), 6.48-6.43 (m, 1H), 4.24-4.13 (m, 4H), |
| | 3.16-3.10 (m, 1H), 2.95-2.88 (m, 1H). LC-MS (ESI, m/z): [M + H]⁺ 455. |
| 1-18 | 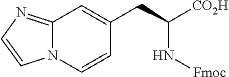 |
| | (S)-2-((((9H-fluoren-9-yl)methoxy)carbonyl)amino)-3-(imidazo[1,2- |
| | a]pyridin-7-yl)propanoic acid |
| | methyl (2R)-2-({[(9H-fluoren-9-yl)methoxy]carbonyl}amino)-3- |
| | iodopropanoate (CAS: 156017-42-4) |
| | 7-Bromoimidazo[1,2-a]pyridine (CAS: 808744-34-5) |
| | ¹H NMR (500 MHZ, DMSO-d₆) δ 12.83 (br, 1H), 8.52-8.42 (m, 1H), 7.93- |
| | 7.76 (m, 4H), 7.65-7.57 (m, 2H), 7.57-7.49 (m, 1H), 7.49-7.42 (m, 1H), |
| | 7.42-7.31 (m, 2H), 7.31-7.13 (m, 2H), 6.90-6.78 (m, 1H), 4.33-4.24 (m, |
| | 1H), 4.24-4.10 (m, 3H), 3.18-3.04 (m, 1H), 2.98-2.76 (m, 1H). LC-MS |
| | (ESI, m/z): [M + H]⁺ 428. |
| 1-19 | 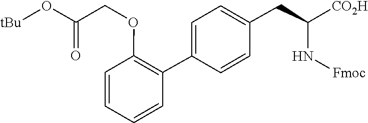 |
| | (S)-2-((((9H-fluoren-9-yl)methoxy)carbonyl)amino)-3-(2′-(2-(tert-butoxy)- |
| | 2-oxoethoxy)-[1,1′-biphenyl]-4-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 2-((4′-bromo-[1,1′-biphenyl]-2-yl)oxy)acetate |
| | ¹H NMR (500 MHZ, DMSO-d₆) δ 12.81 (br, 1H), 7.88 (d, J = 7.6 Hz, 2H), |
| | 7.81 (d, J = 8.4 Hz, 1H), 7.69-7.66 (m, 2H), 7.51-7.46 (m, 2H), 7.43-7.36 |
| | (m, 2H), 7.34-7.23 (m, 6H), 7.04-6.99 (m, 1H), 6.96-6.91 (m, 1H), 4.69- |
| | 4.61 (m, 2H), 4.25-4.12 (m, 4H), 3.16-3.08 (m, 1H), 2.96-2.88 (m, 1H), |
| | 1.40 (s, 9H). LC-MS (ESI, m/z): [M − tBu + H]⁺ 538. |
| 1-20 | 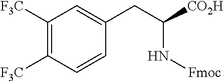 |
| | (S)-2-((((9H-fluoren-9-yl)methoxy)carbonyl)amino)-3-(3,4- |
| | bis(trifluoromethyl)phenyl)propanoic acid |
| | (2R)-2-({[(9H-fluoren-9-yl)methoxy]carbonyl}amino)-3- |
| | methyl (2R)-2-({[(9H-fluoren-9-yl)methoxy]carbonyl}amino)-3- |
| | iodopropanoate (CAS: 156017-42-4) |
| | 4-Bromo-1,2-bis(trifluoromethyl)benzene (CAS: 320-29-6) |
| | ¹H NMR (500 MHz, DMSO-d₆) δ 12.95 (br, 1H), 8.02-7.77 (m, 6H), 7.60 |
| | (d, J = 7.3 Hz, 2H), 7.44-7.21 (m, 4H), 4.33-4.13 (m, 4H), 3.33-3.29 (m, |
| | 1H), 3.06 (dd, J = 13.6, 11.0 Hz, 1H). LC-MS (ESI, m/z): [M + H]⁺ 524. |
| 1-21 | 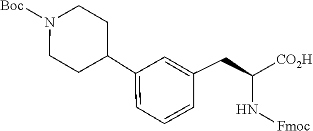 |
| | (S)-2-((((9H-fluoren-9-yl)methoxy)carbonyl)amino)-3-(3-(1-(tert- |
| | butoxycarbonyl)piperidin-4-yl)phenyl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 4-(3-bromophenyl)piperidine-1-carboxylate (CAS: 886362-62-5) |
| | ¹H NMR (500 MHZ, DMSO-d₆) δ 12.77 (br, 1H), 7.88 (d, J = 7.5 Hz, 2H), |
| | 7.80-7.35 (m, 5H), 7.35-6.98 (m, 6H), 4.29-3.90 (m, 6H), 3.19-2.56 (m, |
| | 5H), 1.80-1.26 (m, 13H). LC-MS (ESI, m/z): [M − Boc + H]⁺ 471. |
| 1-22 | 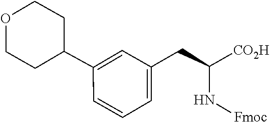 |
| | (S)-2-((((9H-fluoren-9-yl)methoxy)carbonyl)amino)-3-(3-(tetrahydro-2H- |
| | pyran-4-yl)phenyl)propanoic acid |
| | methyl (2R)-2-({[(9H-fluoren-9-yl)methoxy]carbonyl}amino)-3- |
| | iodopropanoate (CAS: 156017-42-4) |
| | 4-(3-Bromophenyl)tetrahydro-2H-pyran (CAS: 1353855-35-2) |
| | ¹H NMR (500 MHZ, DMSO-d₆) 812.75 (br, 1H), 7.88 (d, J = 7.5 Hz, 2H), |
| | 7.74 (d, J = 8.5 Hz, 1H), 7.68-7.61 (m, 2H), 7.44-7.38 (m, 2H), 7.33-7.25 |
| | (m, 2H), 7.23-7.14 (m, 2H), 7.12-7.06 (m, 2H), 4.24-4.11 (m, 4H), 3.94- |
| | 3.85 (m, 2H), 3.44-3.35 (m, 2H), 3.12-3.02 (m, 1H), 2.93-2.80 (m, 1H), |
| | 2.74-2.63 (m, 1H), 1.69-1.57 (m, 4H). LC-MS (ESI, m/z): [M + H]⁺ 472. |
| 1-23 | 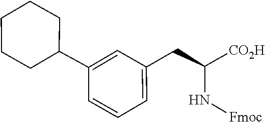 |
| | (S)-2-((((9H-fluoren-9-yl)methoxy)carbonyl)amino)-3-(3- |
| | cyclohexylphenyl)propanoic acid |
| | methyl (2R)-2-({[(9H-fluoren-9-yl)methoxy]carbonyl}amino)-3- |
| | iodopropanoate (CAS: 156017-42-4) |
| | 1-Bromo-3-cyclohexylbenzene (CAS: 19920-84-4) |
| | ¹H NMR (500 MHZ, DMSO-d₆) 812.74 (br, 1H), 7.88 (d, J = 7.5 Hz, 2H), |
| | 7.76-7.60 (m, 3H), 7.44-7.37 (m, 2H), 7.34-7.24 (m, 2H), 7.21-6.99 (m, |
| | 4H), 4.26-4.09 (m, 4H), 3.11-3.00 (m, 1H), 2.90-2.80 (m, 1H), 2.47- |
| | 2.37 (m, 1H), 1.77-1.70 (m, 4H), 1.70-1.63 (m, 1H), 1.41-1.26 (m, 4H), |
| | 1.23-1.12 (m, 1H). LC-MS (ESI, m/z): [M + H]⁺ 470. |
| 1-24 | 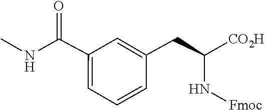 |
| | (S)-2-((((9H-fluoren-9-yl)methoxy)carbonyl)amino)-3-(3- |
| | (methylcarbamoyl)phenyl)propanoic acid |
| | methyl (2R)-2-({[(9H-fluoren-9-yl)methoxy]carbonyl}amino)-3- |
| | iodopropanoate (CAS: 156017-42-4) |
| | 3-Bromo-N-methylbenzamide (CAS: 49834-22-2) |
| | ¹H NMR (500 MHz, DMSO-d₆) 812.77 (br, 1H), 8.44-8.34 (m, 1H), 7.87 |
| | (d, J = 7.6 Hz, 2H), 7.83-7.71 (m, 2H), 7.71-7.46 (m, 3H), 7.46-7.24 (m, |
| | 6H), 4.29-4.07 (m, 4H), 3.18-3.06 (m, 1H), 2.97-2.81 (m, 1H), 2.81- |
| | 2.72 (m, 3H). LC-MS (ESI, m/z): [M + H]⁺ 445. |
| 1-25 | 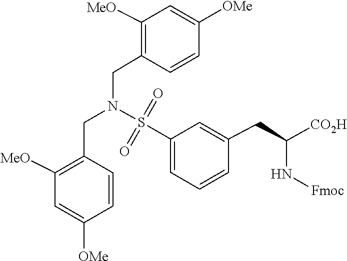 |
| | (S)-2-((((9H-fluoren-9-yl)methoxy)carbonyl)amino)-3-(3-(N,N-bis(2,4- |
| | dimethoxybenzyl)sulfamoyl)phenyl)propanoic acid |
| | methyl (2R)-2-({[(9H-fluoren-9-yl)methoxy]carbonyl}amino)-3- |
| | iodopropanoate (CAS: 156017-42-4) |
| | 3-Bromo-N,N-bis[(2,4-dimethoxyphenyl)methyl]benzenesulfonamide |
| | (CAS: 2417373-09-0) |
| | ¹H NMR (500 MHZ, DMSO-d₆) δ 12.86 (br, 1H), 7.94-7.75 (m, 3H), 7.67 |
| | (s, 1H), 7.64-7.33 (m, 7H), 7.33-7.19 (m, 2H), 6.95 (d, J = 8.5 Hz, 2H), |
| | 6.42-6.29 (m, 4H), 4.37-3.87 (m, 8H), 3.68 (s, 6H), 3.51 (s, 6H), 3.23- |
| | 3.03 (m, 1H), 3.03-2.82 (m, 1H). LC-MS (ESI, m/z): [M + Na]⁺ 789. |
| 1-26 | 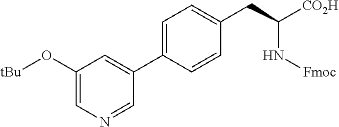 |
| | (S)-2-((((9H-fluoren-9-yl)methoxy)carbonyl)amino)-3-(4-(5-(tert- |
| | butoxy)pyridin-3-yl)phenyl)propanoic acid |
| | methyl (2R)-2-({[(9H-fluoren-9-yl)methoxy]carbonyl}amino)-3- |
| | iodopropanoate (CAS: 156017-42-4) |
| | 3-(4-bromophenyl)-5-(tert-butoxy)pyridine |
| | ¹H NMR (500 MHZ, DMSO-d₆) δ 12.73 (br, 1H), 8.64-8.53 (m, 1H), 8.25 |
| | (s, 1H), 7.87 (d, J = 7.0 Hz, 2H), 7.82-7.15 (m, 12H), 4.28-4.09 (m, 4H), |
| | 3.19-2.87 (m, 2H), 1.34 (s, 9H). LC-MS (ESI, m/z): [M + H]⁺ 537. |
| 1-27 | 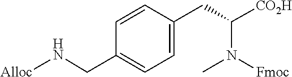 |
| | (R)-2-((((9H-fluoren-9-yl)methoxy)carbonyl)(methyl)amino)-3-(4- |
| | ((((allyloxy)carbonyl)amino)methyl)phenyl)propanoic acid |
| | methyl (2S)-2-({[(9H-fluoren-9-yl)methoxy]carbonyl}(methyl)amino)-3- |
| | iodopropanoate (CAS: 2973756-21-5) |
| | 1-(4-bromophenyl)methanamine (CAS: 3959-07-7) |
| | ¹H NMR (300 MHz, DMSO-d₆) δ 12.92 (br, 1H), 7.88 (d, J = 7.5 Hz, 2H), |
| | 7.84-7.66 (m, 1H), 7.66-7.48 (m, 2H), 7.48-7.24 (m, 4H), 7.24-6.84 (m, |
| | 4H), 6.02-5.77 (m, 1H), 5.40-4.99 (m, 2H), 4.85-4.59 (m, 1H), 4.59- |
| | 4.42 (m, 2H), 4.42-4.05 (m, 5H), 3.25-2.75 (m, 2H), 2.75-2.59 (m, 3H). |
| | LC-MS (ESI, m/z): [M + H]⁺ 515. |
| 1-28 | 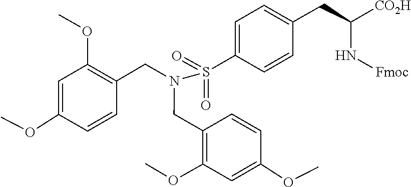 |
| | (S)-2-((((9H-fluoren-9-yl)methoxy)carbonyl)amino)-3-(4-(N,N-bis(2,4- |
| | dimethoxybenzyl)sulfamoyl)phenyl)propanoic acid |
| | methyl (2R)-2-({[(9H-fluoren-9-yl)methoxy]carbonyl}amino)-3- |
| | iodopropanoate (CAS: 156017-42-4) |
| | 4-Bromo-N,N-bis[(2,4-dimethoxyphenyl)methyl]benzenesulfonamide |
| | (CAS: 1440421-56-6) |
| | ¹H NMR (500 MHZ, DMSO-d₆) δ 12.95 (br, 1H), 7.91-7.69 (m, 3H), 7.69- |
| | 7.50 (m, 4H), 7.50-7.24 (m, 6H), 6.99-6.86 (m, 2H), 6.47-6.26 (m, 4H), |
| | 4.30-4.08 (m, 8H), 3.69 (s, 6H), 3.52 (s, 6H), 3.23-3.12 (m, 1H), 3.03- |
| | 2.90 (m, 1H). LC-MS (ESI, m/z): [M − H]⁻ 765. |
| 1-29 | 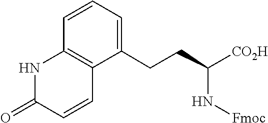 |
| | (S)-2-((((9H-fluoren-9-yl)methoxy)carbonyl)amino)-4-(2-oxo-1,2- |
| | dihydroquinolin-5-yl)butanoic acid |
| | methyl (2S)-2-({[(9H-fluoren-9-yl)methoxy]carbonyl}amino)-4- |
| | iodobutanoate (CAS: 1354921-71-3) |
| | 5-Bromo-2-methoxyquinoline (CAS: 99455-06-8) |
| | ¹H NMR (500 MHZ, DMSO-d₆) δ 12.65 (br, 1H), 11.74 (br, 1H), 8.10 (d, |
| | J = 9.9 Hz, 1H), 7.98-7.83 (m, 3H), 7.81-7.69 (m, 2H), 7.46-7.30 (m, 5H), |
| | 7.19 (d, J = 8.2 Hz, 1H), 7.01 (d, J = 7.3 Hz, 1H), 6.46 (d, J = 9.9 Hz, 1H), |
| | 4.42-4.10 (m, 3H), 4.07-3.88 (m, 1H), 3.06-2.79 (m, 2H), 2.04-1.79 |
| | (m, 2H). LC-MS (ESI, m/z): [M + H]⁺ 469. |
| 1-30 | 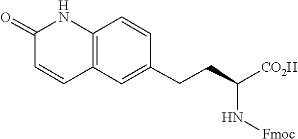 |
| | (S)-2-((((9H-fluoren-9-yl)methoxy)carbonyl)amino)-4-(2-oxo-1,2- |
| | dihydroquinolin-6-yl)butanoic acid |
| | methyl (2S)-2-({[(9H-fluoren-9-yl)methoxy]carbonyl}amino)-4- |
| | iodobutanoate (CAS: 1354921-71-3) |
| | 6-Bromo-2-methoxyquinoline (CAS: 99455-05-7) |
| | ¹H NMR (500 MHZ, DMSO-d₆) δ 12.91-12.45 (m, 1H), 11.68 (s, 1H), 7.91 |
| | (d, J = 7.5 Hz, 2H), 7.82 (d, J = 9.5 Hz, 1H), 7.79-7.72 (m, 3H), 7.50-7.40 |
| | (m, 3H), 7.38-7.32 (m, 3H), 7.26-7.20 (m, 1H), 6.47 (d, J = 9.5 Hz, 1H), |
| | 4.37-4.10 (m, 3H), 3.95-3.85 (m, 1H), 2.76-2.61 (m, 2H), 2.05-1.87 (m, |
| | 2H). LC-MS (ESI, m/z): [M + H]⁺ 469. |
| 1-31 | 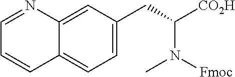 |
| | (R)-2-((((9H-fluoren-9-yl)methoxy)carbonyl)(methyl)amino)-3-(quinolin- |
| | 7-yl)propanoic acid |
| | methyl (2S)-2-({[(9H-fluoren-9-yl)methoxy]carbonyl}(methyl)amino)-3- |
| | iodopropanoate (CAS: 2973756-21-5) |
| | 7-Bromoquinoline (CAS: 4965-36-0) |
| | ¹H NMR (500 MHZ, METHANOL-d₄) δ 8.80-8.70 (m, 1H), 8.34-8.21 (m, |
| | 1H), 7.93-7.77 (m, 2H), 7.76-7.68 (m, 2H), 7.60-7.49 (m, 1H), 7.48- |
| | 7.22 (m, 5H), 7.19-6.99 (m, 2H), 5.21-4.87 (m, 1H), 4.31-4.22 (m, 1H), |
| | 4.13-3.96 (m, 2H), 3.68-3.42 (m, 1H), 3.35-3.09 (m, 1H), 2.86-2.83 (m, |
| | 3H). LC-MS (ESI, m/z): [M + H]⁺ 453. |
| 1-32 | 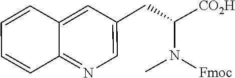 |
| | (R)-2-((((9H-fluoren-9-yl)methoxy)carbonyl)(methyl)amino)-3-(quinolin- |
| | 3-yl)propanoic acid |
| | methyl (2S)-2-({[(9H-fluoren-9-yl)methoxy]carbonyl}(methyl)amino)-3- |
| | iodopropanoate (CAS: 2973756-21-5) |
| | 3-Bromoquinoline (CAS: 5332-24-1) |
| | ¹H NMR (500 MHZ, METHANOL-d₄) δ 8.87-8.55 (m, 1H), 8.27-7.87 (m, |
| | 2H), 7.84-7.78 (m, 1H), 7.75-7.65 (m, 3H), 7.60-7.17 (m, 6H), 7.15- |
| | 7.07 (m, 1H), 5.12-4.67 (m, 1H), 4.47-4.21 (m, 1H), 4.16-4.09 (m, 1H), |
| | 4.07-4.00 (m, 1H), 3.61-2.91 (m, 2H), 2.85-2.75 (m, 3H). LC-MS (ESI, |
| | m/z): [M + H]⁺ 453. |
| 1-33 | 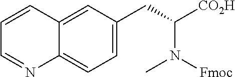 |
| | (R)-2-((((9H-fluoren-9-yl)methoxy)carbonyl)(methyl)amino)-3-(quinolin- |
| | 6-yl)propanoic acid |
| | methyl (2S)-2-({[(9H-fluoren-9-yl)methoxy]carbonyl}(methyl)amino)-3- |
| | iodopropanoate (CAS: 2973756-21-5) |
| | 6-Bromoquinoline (CAS: 5332-25-2) |
| | ¹H NMR (500 MHZ, METHANOL-d₄) δ 8.81-8.66 (m, 1H), 8.24-8.19 (m, |
| | 1H), 7.98-7.83 (m, 1H), 7.80-7.64 (m, 3H), 7.60-7.19 (m, 7H), 7.15- |
| | 6.94 (m, 1H), 5.11-4.67 (m, 1H), 4.55-4.24 (m, 1H), 4.15-3.94 (m, 2H), |
| | 3.64-2.89 (m, 2H), 2.81-2.72 (m, 3H). LC-MS (ESI, m/z): [M + H]⁺ 453. |
| 1-34 | 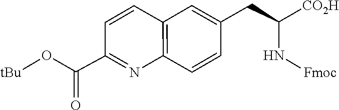 |
| | (S)-2-((((9H-fluoren-9-yl)methoxy)carbonyl)amino)-3-(2-(tert- |
| | butoxycarbonyl)quinolin-6-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 6-bromoquinoline-2-carboxylate (CAS: 2732996-01-7) |
| | ¹H NMR (500 MHZ, DMSO-d₆) δ 12.90 (br, 1H), 8.41 (d, J = 8.5 Hz, 1H), |
| | 8.18-7.96 (m, 2H), 7.96-7.64 (m, 5H), 7.64-7.29 (m, 4H), 7.29-7.08 (m, |
| | 2H), 4.51-3.97 (m, 4H), 3.50-2.98 (m, 2H), 1.61 (s, 9H). LC-MS (ESI, |
| | m/z): [M + H]⁺ 539. |
| 1-35 | 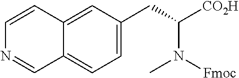 |
| | (R)-2-((((9H-fluoren-9-yl)methoxy)carbonyl)(methyl)amino)-3- |
| | (isoquinolin-6-yl)propanoic acid |
| | methyl (2S)-2-({[(9H-fluoren-9-yl)methoxy]carbonyl}(methyl)amino)-3- |
| | iodopropanoate (CAS: 2973756-21-5) |
| | 6-Bromoisoquinoline (CAS: 34784-05-9) |
| | ¹H NMR (500 MHZ, METHANOL-d₄) δ 9.17-9.07 (m, 1H), 8.38-8.30 (m, |
| | 1H), 8.01-7.89 (m, 1H), 7.81-7.59 (m, 4H), 7.54-7.19 (m, 6H), 7.16- |
| | 7.01 (m, 1H), 5.20-4.62 (m, 1H), 4.55-3.98 (m, 3H), 3.65-2.85 (m, 2H), |
| | 2.77 (s, 3H). LC-MS (ESI, m/z): [M + H]⁺ 453. |
| 1-36 | 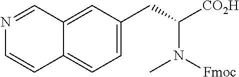 |
| | (R)-2-((((9H-fluoren-9-yl)methoxy)carbonyl)(methyl)amino)-3- |
| | (isoquinolin-7-yl)propanoic acid |
| | methyl (2S)-2-({[(9H-fluoren-9-yl)methoxy]carbonyl}(methyl)amino)-3- |
| | iodopropanoate (CAS: 2973756-21-5) |
| | 7-Bromoisoquinoline (CAS: 58794-09-5) |
| | ¹H NMR (500 MHZ, METHANOL-d₄) δ 9.13-9.01 (m, 1H), 8.43-8.28 (m, |
| | 1H), 7.94 (s, 1H), 7.84-7.74 (m, 2H), 7.74-7.61 (m, 3H), 7.53-7.43 (m, |
| | 1H), 7.41-7.20 (m, 4H), 7.15-7.03 (m, 1H), 5.16-4.62 (m, 1H), 4.55- |
| | 3.95 (m, 3H), 3.62-3.33 (m, 1H), 3.18-2.88 (m, 1H), 2.81-2.68 (m, 3H), |
| | LC-MS: [M + H]⁺ 453. |
| 1-37 | 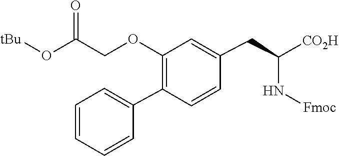 |
| | (S)-2-((((9H-fluoren-9-yl)methoxy)carbonyl)amino)-3-(2-(2-(tert-butoxy)- |
| | 2-oxoethoxy)-[1,1′-biphenyl]-4-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 2-((4-bromo-[1,1′-biphenyl]-2-yl)oxy)acetate |
| | ¹H NMR (500 MHz, DMSO-d₆) δ 12.79 (br, 1H), 7.90-7.80 (m, 2H), 7.80- |
| | 7.75 (m, 1H), 7.70-7.60 (m, 2H), 7.55-7.45 (m, 2H), 7.45-7.25 (m, 8H), |
| | 7.10-7.00 (m, 1H), 7.00-6.90 (m, 1H), 4.64 (s, 2H), 4.30-4.15 (m, 4H), |
| | 3.20-3.10 (m, 1H), 3.00-2.90 (m, 1H), 1.40 (s, 9H). LC-MS (ESI, m/z): |
| | [M − tBu + H]⁺ 538. |
| 1-38 | 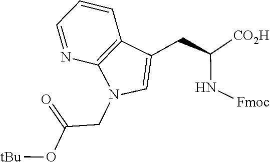 |
| | (S)-2-((((9H-fluoren-9-yl)methoxy)carbonyl)amino)-3-(1-(2-(tert-butoxy)- |
| | 2-oxoethyl)-1H-pyrrolo[2,3-b]pyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 2-(3-iodo-1H-pyrrolo[2,3-b]pyridin-1-yl)acetate |
| | ¹H NMR (500 MHZ, DMSO-d₆) δ 12.73 (br, 1H), 8.26-8.15 (m, 1H), 8.07- |
| | 7.95 (m, 1H), 7.88 (d, J = 7.6 Hz, 2H), 7.77-7.61 (m, 3H), 7.43-7.23 (m, |
| | 5H), 7.12-7.01 (m, 1H), 4.93 (s, 2H), 4.26-4.14 (m, 4H), 3.24-3.12 (m, |
| | 1H), 3.10-2.97 (m, 1H), 1.38 (s, 9H). LC-MS (ESI, m/z): [M + H]⁺ 542. |
| 1-39 | 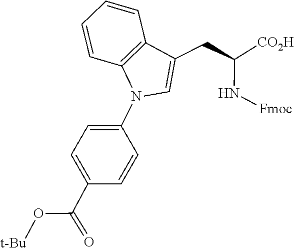 |
| | (2S)-3-(1-{4-[(tert-butoxy)carbonyl]phenyl}-1H-indol-3-yl)-2-({[(9H- |
| | fluoren-9-yl)methoxy]carbonyl}amino)propanoic acid |
| | methyl (2R)-2-({[(9H-fluoren-9-yl)methoxy]carbonyl}amino)-3- |
| | iodopropanoate (CAS: 156017-42-4) |
| | tert-butyl 4-(3-iodo-1H-indol-1-yl)benzoate |
| | ¹H NMR (500 MHZ, DMSO-d₆) δ 12.80 (br, 1H), 8.02 (d, J = 8.5 Hz, 2H), |
| | 7.85 (d, J = 7.6 Hz, 3H), 7.72 (d, J = 7.9 Hz, 1H), 7.69-7.61 (m, 5H), 7.57 |
| | (s, 1H), 7.39-7.33 (m, 2H), 7.27-7.21 (m, 2H), 7.20-7.15 (m, 2H), 4.37- |
| | 4.27 (m, 1H), 4.23-4.11 (m, 3H), 3.31-3.26 (m, 1H), 3.20-3.03 (m, 1H), |
| | 1.58 (s, 9H). LC-MS (ESI, m/z): [M + H]⁺ 603. |
| 1-40 | 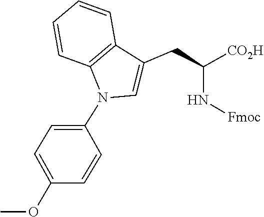 |
| | (2S)-2-({[(9H-fluoren-9-yl)methoxy]carbonyl}amino)-3-[1-(4- |
| | methoxyphenyl)-1H-indol-3-yl]propanoic acid |
| | methyl (2R)-2-({[(9H-fluoren-9-yl)methoxy]carbonyl}amino)-3- |
| | iodopropanoate (CAS: 156017-42-4) |
| | 3-iodo-1-(4-methoxyphenyl)-1H-indole |
| | ¹H NMR (500 MHZ, DMSO-d₆) δ 12.78 (br, 1H), 7.91-7.79 (m, 3H), 7.70- |
| | 7.61 (m, 3H), 7.45-7.35 (m, 6H), 7.28-7.21 (m, 1H), 7.20-7.15 (m, 2H), |
| | 7.14-7.07 (m, 3H), 4.34-4.25 (m, 1H), 4.24-4.08 (m, 3H), 3.82 (s, 3H), |
| | 3.30-3.22 (m, 1H), 3.19-3.02 (m, 1H). LC-MS (ESI, m/z): [M + H]⁺ 533. |
| 1-41 | 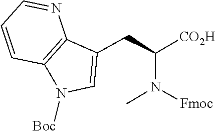 |
| | (S)-2-((((9H-fluoren-9-yl)methoxy)carbonyl)(methyl)amino)-3-(1-(tert- |
| | butoxycarbonyl)-1H-pyrrolo[3,2-b]pyridin-3-yl)propanoic acid |
| | methyl (2R)-2-({[(9H-fluoren-9-yl)methoxy]carbonyl}(methyl)amino)-3- |
| | iodopropanoate (CAS: 2973756-19-1) |
| | tert-butyl 3-iodo-1H-pyrrolo[3,2-b]pyridine-1-carboxylate |
| | (CAS: 1316228-21-3) |
| | ¹H NMR (500 MHz, DMSO-d₆) δ 13.10 (br, 1H), 9.25-9.06 (m, 1H), 8.40- |
| | 8.30 (m, 1H), 7.95-7.78 (m, 2H), 7.78-7.10 (m, 8H), 5.05-4.92 (m, 1H), |
| | 4.31-3.93 (m, 3H), 3.50-2.95 (m, 2H), 2.80-2.70 (m, 3H), 1.63-1.43 (m, |
| | 9H). LC-MS (ESI, m/z): [M + H]⁺ 542. |
| 1-42 | 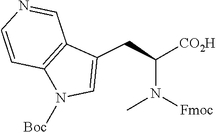 |
| | (S)-2-((((9H-fluoren-9-yl)methoxy)carbonyl)(methyl)amino)-3-(1-(tert- |
| | butoxycarbonyl)-1H-pyrrolo[3,2-c]pyridin-3-yl)propanoic acid |
| | methyl (2R)-2-({[(9H-fluoren-9-yl)methoxy]carbonyl}(methyl)amino)-3- |
| | iodopropanoate (CAS: 2973756-19-1) |
| | tert-butyl 3-iodo-1H-pyrrolo[3,2-c]pyridine-1-carboxylate |
| | (CAS: 877060-48-5) |
| | ¹H NMR (500 MHz, DMSO-d₆) δ 13.10 (br, 1H), 9.01 (s, 1H), 8.48-8.40 |
| | (m, 1H), 7.92-7.70 (m, 3H), 7.70-7.09 (m, 7H), 5.15-4.97 (m, 1H), 4.32- |
| | 3.90 (m, 3H), 3.50-3.05 (m, 2H), 2.84-2.67 (m, 3H), 1.62-1.39 (m, 9H). |
| | LC-MS (ESI, m/z): [M + H]⁺ 542. |
| 1-43 | 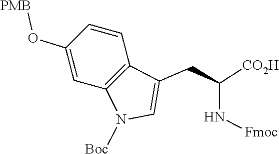 |
| | (S)-2-((((9H-fluoren-9-yl)methoxy)carbonyl)amino)-3-(1-(tert- |
| | butoxycarbonyl)-6-((4-methoxybenzyl)oxy)-1H-indol-3-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 3-iodo-6-((4-methoxybenzyl)oxy)-1H-indole-1-carboxylate |
| | ¹H NMR (500 MHZ, DMSO-d₆) δ 12.89 (br, 1 H), 7.87 (d, J-7.6 Hz, 2 H), |
| | 7.78-7.59 (m, 4 H), 7.53 (d, J = 8.5 Hz, 1 H), 7.42-7.35 (m, 5 H), 7.30- |
| | 7.20 (m, 2 H), 6.95-6.92 (m, 3 H), 5.04 (s, 2 H), 4.28-4.12 (m, 4 H), 3.75 |
| | (s, 3 H), 3.18-3.11 (m, 1 H), 3.03-2.94 (m, 1 H), 1.63-1.51 (m, 9 H). LC- |
| | MS (ESI, m/z): [M + H]⁺ 663. |
| 1-44 | 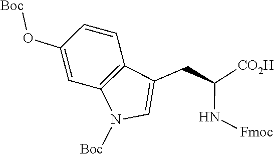 |
| | (S)-2-((((9H-fluoren-9-yl)methoxy)carbonyl)amino)-3-(1-(tert- |
| | butoxycarbonyl)-6-((tert-butoxycarbonyl)oxy)-1H-indol-3-yl)propanoic |
| | acid |
| | methyl (2R)-2-({[(9H-fluoren-9-yl)methoxy]carbonyl}amino)-3- |
| | iodopropanoate (CAS: 156017-42-4) |
| | tert-butyl 6-((tert-butoxycarbonyl)oxy)-3-iodo-1H-indole-1-carboxylate |
| | ¹H NMR (500 MHz, DMSO-d₆) δ 12.87 (br, 1H), 7.90-7.85 (m, 2H), 7.84- |
| | 7.77 (m, 2H), 7.69-7.65 (m, 1H), 7.64-7.57 (m, 3H), 7.42-7.31 (m, 2H), |
| | 7.30-7.20 (m, 2H), 7.10-7.06 (m, 1H), 4.33-4.27 (m, 1H), 4.23-4.06 (m, |
| | 3H), 3.24-3.12 (m, 1H), 3.08-2.99 (m, 1H), 1.62-1.45 (m, 18H). LC- |
| | MS (ESI, m/z): [M − 2tBu + H]⁺ 531. |
| 1-45 | 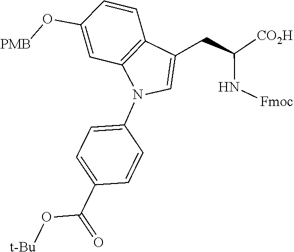 |
| | (S)-2-((((9H-fluoren-9-yl)methoxy)carbonyl)amino)-3-(1-(4-(tert- |
| | butoxycarbonyl)phenyl)-6-((4-methoxybenzyl)oxy)-1H-indol-3- |
| | yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 4-(3-iodo-6-((4-methoxybenzyl)oxy)-1H-indol-1-yl)benzoate |
| | ¹H NMR (500 MHZ, DMSO-d₆) δ 12.78 (br, 1H), 8.05-7.97 (m, 2H), 7.89- |
| | 7.77 (m, 3H), 7.68-7.55 (m, 5H), 7.44-7.33 (m, 5H), 7.27-7.15 (m, |
| | 3H), 6.97-6.90 (m, 2H), 6.90-6.85 (m, 1H), 5.03 (s, 2H), 4.36-4.26 (m, |
| | 1H), 4.22-4.07 (m, 3H), 3.75 (s, 3H), 3.28-3.00 (m, 2H), 1.58 (s, 9H). LC- |
| | MS (ESI, m/z): [M + H]⁺ 739. |
| 1-46 | 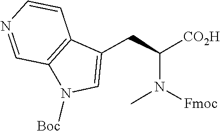 |
| | (S)-2-((((9H-fluoren-9-yl)methoxy)carbonyl)(methyl)amino)-3-(1-(tert- |
| | butoxycarbonyl)-1H-pyrrolo[2,3-c]pyridin-3-yl)propanoic acid |
| | methyl (2R)-2-({[(9H-fluoren-9-yl)methoxy]carbonyl}(methyl)amino)-3- |
| | iodopropanoate (CAS: 2973756-19-1) |
| | tert-butyl 3-iodo-1H-pyrrolo[2,3-c]pyridine-1-carboxylate |
| | (CAS: 1174038-59-5) |
| | ¹H NMR (500 MHZ, DMSO-d₆) δ 13.10 (br, 1H), 9.25-9.13 (m, 1H), 8.41- |
| | 8.28 (m, 1H), 7.95-7.78 (m, 2H), 7.78-7.57 (m, 2H), 7.57-7.10 (m, 6H), |
| | 5.05-4.94 (m, 1H), 4.31-3.91 (m, 3H), 3.45-3.05 (m, 2H), 2.80-2.70 (m, |
| | 3H), 1.62-1.44 (m, 9H). LC-MS (ESI, m/z): [M + H]⁺ 542. |
| 1-47 | 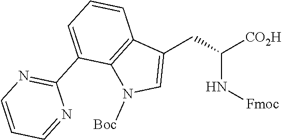 |
| | (R)-2-((((9H-fluoren-9-yl)methoxy)carbonyl)amino)-3-(1-(tert- |
| | butoxycarbonyl)-7-(pyrimidin-2-yl)-1H-indol-3-yl)propanoic acid |
| | methyl (2S)-2-({[(9H-fluoren-9-yl)methoxy]carbonyl}amino)-3- |
| | iodopropanoate (CAS: 527696-77-1) |
| | tert-butyl 3-iodo-7-(pyrimidin-2-yl)-1H-indole-1-carboxylate |
| | ¹H NMR (500 MHZ, DMSO-d₆) δ 12.88 (br, 1H), 8.90-8.81 (m, 2H), 7.97- |
| | 7.08 (m, 14H), 4.40-4.28 (m, 1H), 4.27-4.00 (m, 3H), 3.28-3.18 (m, |
| | 1H), 3.14-3.03 (m, 1H), 1.37-1.21 (m, 9H). LC-MS (ESI, m/z): [M + H]⁺ |
| | 605. |
| 1-48 | 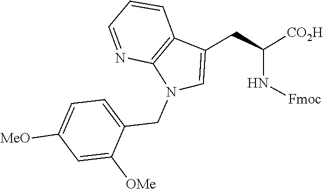 |
| | (S)-2-((((9H-fluoren-9-yl)methoxy)carbonyl)amino)-3-(1-(2,4- |
| | dimethoxybenzyl)-1H-pyrrolo[2,3-b]pyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | 1-(2,4-dimethoxybenzyl)-3-iodo-1H-pyrrolo[2,3-b]pyridine |
| | ¹H NMR (500 MHZ, DMSO-d₆) δ 12.64 (br, 1H), 8.27-8.18 (m, 1H), 8.05- |
| | 7.97 (m, 1H), 7.91-7.85 (m, 2H), 7.76-7.69 (m, 1H), 7.67-7.59 (m, 2H), |
| | 7.45-7.36 (m, 2H), 7.32-7.27 (m, 2H), 7.27-7.21 (m, 1H), 7.08-7.02 (m, |
| | 1H), 6.74-6.69 (m, 1H), 6.54 (d, J = 1.0 Hz, 1H), 6.34-6.25 (m, 1H), 5.32 |
| | -5.22 (m, 2H), 4.28-4.10 (m, 4H), 3.80 (s, 3H), 3.64 (s, 3H), 3.22-3.12 |
| | (m, 1H), 3.06-2.96 (m, 1H). LC-MS (ESI, m/z): [M + H]⁺ 578. |
| 1-49 | 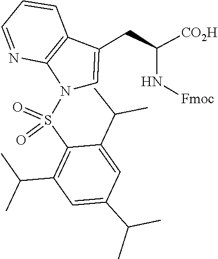 |
| | (S)-2-((((9H-fluoren-9-yl)methoxy)carbonyl)amino)-3-(1-((2,4,6- |
| | triisopropylphenyl)sulfonyl)-1H-pyrrolo[2,3-b]pyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | 3-iodo-1-((2,4,6-triisopropylphenyl)sulfonyl)-1H-pyrrolo[2,3-b]pyridine |
| | ¹H NMR (500 MHZ, DMSO-d₆) δ 12.93 (br, 1H), 8.20-8.14 (m, 1H), 8.13- |
| | 8.08 (m, 1H), 7.93-7.82 (m, 3H), 7.80-7.69 (m, 1H), 7.68-7.60 (m, 2H), |
| | 7.43-7.17 (m, 7H), 4.37-4.21 (m, 4H), 4.21-4.13 (m, 2H), 3.29-3.22 (m, |
| | 1H), 3.07 (dd, J = 14.6, 9.9 Hz, 1H), 2.99-2.86 (m, 1H), 1.20-1.15 (m, |
| | 6H), 1.12-1.01 (m, 12H). LC-MS (ESI, m/z): [M + H]⁺ 694. |
| 1-50 | 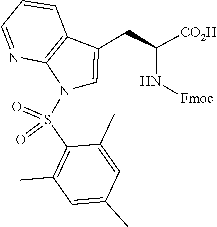 |
| | (S)-2-((((9H-fluoren-9-yl)methoxy)carbonyl)amino)-3-(1- |
| | (mesitylsulfonyl)-1H-pyrrolo[2,3-b]pyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | 3-iodo-1-(mesitylsulfonyl)-1H-pyrrolo[2,3-b]pyridine |
| | ¹H NMR (500 MHz, DMSO-d₆) δ 12.91 (br, 1H), 8.22-8.15 (m, 1H), 8.14- |
| | 8.03 (m, 1H), 7.92-7.80 (m, 3H), 7.74 (br, 1H), 7.65 (dd, J = 7.3, 2.6 Hz, |
| | 2H), 7.44-7.19 (m, 2H), 7.32-7.19 (m, 3H), 7.02 (s, 2H), 4.30-4.07 (m, |
| | 4H), 3.28-3.20 (m, 1H), 3.10-3.02 (m, 1H), 2.58-2.52 (m, 6H), 2.22 (s, |
| | 3H). LC-MS (ESI, m/z): [M + H]⁺ 610. |
| 1-51 | 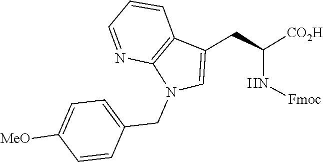 |
| | (S)-2-((((9H-fluoren-9-yl)methoxy)carbonyl)amino)-3-(1-(4- |
| | methoxybenzyl)-1H-pyrrolo[2,3-b]pyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | 3-Iodo-1-[(4-methoxyphenyl)methyl]-1H-pyrrolo[2,3-b]pyridine |
| | (CAS: 2122311-81-1) |
| | ¹H NMR (500 MHz, DMSO-d₆) 812.78 (br, 1H), 8.29-8.20 (m, 1H), 8.07- |
| | 7.98 (m, 1H), 7.92-7.81 (m, 2H), 7.79-7.71 (m, 1H), 7.68-7.60 (m, 2H), |
| | 7.47-7.34 (m, 3H), 7.33-7.21 (m, 2H), 7.14 (d, J = 8.5 Hz, 2H), 7.09-7.02 |
| | (m, 1H), 6.77 (d, J = 8.7 Hz, 2H), 5.37-5.27 (m, 2H), 4.29-4.06 (m, 4H), |
| | 3.62 (s, 3H), 3.23-3.10 (m, 1H), 3.07-2.92 (m, 1H). LC-MS (ESI, m/z): |
| | [M + H]⁺ 548. |
| 1-52 | 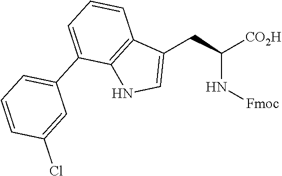 |
| | (S)-2-((((9H-fluoren-9-yl)methoxy)carbonyl)amino)-3-(7-(3- |
| | chlorophenyl)-1H-indol-3-yl)propanoic acid |
| | methyl (2R)-2-({[(9H-fluoren-9-yl)methoxy]carbonyl}amino)-3- |
| | iodopropanoate (CAS: 156017-42-4) |
| | 7-(3-chlorophenyl)-3-iodo-1H-indole |
| | ¹H NMR (500 MHZ, DMSO-d₆) δ 12.75 (br, 1H), 10.90 (s, 1H), 7.95-7.80 |
| | (m, 2H), 7.79-7.21 (m, 13H), 7.21-7.07 (m, 2H), 4.40-4.05 (m, 4H), 3.29 |
| | -2.97 (m, 2H). LC-MS (ESI, m/z): [M + H]⁺ 537. |
| 1-53 | 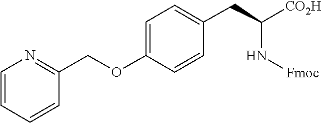 |
| | (S)-2-((((9H-fluoren-9-yl)methoxy)carbonyl)amino)-3-(4-(pyridin-2- |
| | ylmethoxy)phenyl)propanoic acid |
| | (2R)-2-({[(9H-fluoren-9-yl)methoxy]carbonyl}amino)-3- |
| | methyl (2R)-2-({[(9H-fluoren-9-yl)methoxy]carbonyl}amino)-3- |
| | iodopropanoate (CAS: 156017-42-4) |
| | 2-((4-iodophenoxy)methyl)pyridine (CAS: 1249538-55-3) |
| | ¹H NMR (500 MHZ, DMSO-d₆) δ 12.73 (br, 1H), 8.57 (d, J = 4.4 Hz, 1H), |
| | 7.88 (d, J = 7.5 Hz, 2H), 7.83-7.77 (m, 1H), 7.73-7.61 (m, 3H), 7.47 (d, |
| | J = 7.9 Hz, 1H), 7.43-7.37 (m, 2H), 7.35-7.28 (m, 3H), 7.23-7.14 (m, 2H), |
| | 6.99-6.89 (m, 2H), 5.18-5.09 (m, 2H), 4.22-4.07 (m, 4H), 3.05-2.97 (m, |
| | 1H), 2.84-2.77 (m, 1H). LC-MS (ESI, m/z): [M + H]⁺ 495. |
| 1-54 | 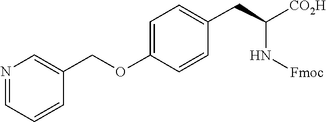 |
| | (S)-2-((((9H-fluoren-9-yl)methoxy)carbonyl)amino)-3-(4-(pyridin-3- |
| | ylmethoxy)phenyl)propanoic acid |
| | methyl (2R)-2-({[(9H-fluoren-9-yl)methoxy]carbonyl}amino)-3- |
| | iodopropanoate (CAS: 156017-42-4) |
| | 3-((4-iodophenoxy)methyl)pyridine (CAS: 1247924-89-5) |
| | ¹H NMR (500 MHz, DMSO-d₆) δ 12.72 (br, 1H), 8.66-8.62 (m, 1H), 8.55- |
| | 8.52 (m, 1H), 7.88 (d, J = 7.6 Hz, 2H), 7.85-7.80 (m, 1H), 7.70 (d, J = 8.4 |
| | Hz, 1H), 7.65 (dd, J = 7.5, 7.5 Hz, 2H), 7.43-7.38 (m, 3H), 7.34-7.27 (m, |
| | 2H), 7.24-7.16 (m, 2H), 6.95-6.90 (m, 2H), 5.08 (s, 2H), 4.22-4.11 (m, |
| | 4H), 3.06-2.96 (m, 1H), 2.84-2.78 (m, 1H). LC-MS (ESI, m/z): [M + H]⁺ |
| | 495. |
| 1-55 | 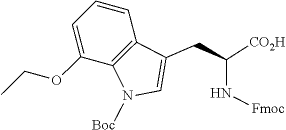 |
| | (S)-2-((((9H-fluoren-9-yl)methoxy)carbonyl)amino)-3-(1-(tert- |
| | butoxycarbonyl)-7-ethoxy-1H-indol-3-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 7-ethoxy-3-iodo-1H-indole-1-carboxylate |
| | ¹H NMR (500 MHZ, DMSO-d₆) δ 12.57 (br, 1H), 7.90-7.79 (m, 2H), 7.72- |
| | 7.48 (m, 4H), 7.45-7.34 (m, 3H), 7.32-7.25 (m, 1H), 7.24-7.18 (m, 2H), |
| | 6.83-6.73 (m, 1H), 4.64-4.36 (m, 1H), 4.22-3.90 (m, 5H), 3.42-3.36 (m, |
| | 1H), 3.01-2.74 (m, 1H), 1.64-1.51 (m, 9H), 1.47-1.36 (m, 3H). LC-MS |
| | (ESI, m/z): [M + H]⁺ 571. |
| 1-56 | 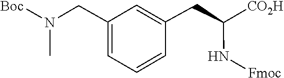 |
| | (S)-2-((((9H-fluoren-9-yl)methoxy)carbonyl)amino)-3-(3-(((tert- |
| | butoxycarbonyl)(methyl)amino)methyl)phenyl)propanoic acid |
| | methyl (2R)-2-({[(9H-fluoren-9-yl)methoxy]carbonyl}amino)-3- |
| | iodopropanoate (CAS: 156017-42-4) |
| | tert-butyl N-[(3-bromophenyl)methyl]-N-methylcarbamate |
| | (CAS: 317358-61-5) |
| | ¹H NMR (500 MHz, DMSO-d₆) δ 12.71 (br, 1H), 7.88 (d, J = 7.6 Hz, 2H), |
| | 7.71 (d, J = 8.4 Hz, 1H), 7.68-7.37 (m, 4H), 7.35-7.22 (m, 3H), 7.21-7.00 |
| | (m, 3H), 4.38-4.27 (m, 2H), 4.23-4.09 (m, 4H), 3.12-2.99 (m, 1H), 2.92- |
| | 2.75 (m, 1H), 2.75-2.65 (m, 3H), 1.48-1.30 (m, 9H). LC-MS (ESI, m/z): |
| | [M − Boc + H]⁺ 431. |
| 1-57 | 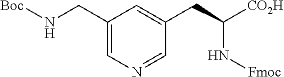 |
| | (S)-2-((((9H-fluoren-9-yl)methoxy)carbonyl)amino)-3-(5-(((tert- |
| | butoxycarbonyl)amino)methyl)pyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl N-[(5-bromopyridin-3-yl)methyl]carbamate (CAS: 943722-24-5) |
| | ¹H NMR (500 MHZ, DMSO-d₆) δ 12.85 (br, 1H), 8.40-8.29 (m, 2H), 7.90- |
| | 7.77 (m, 3H), 7.69-7.53 (m, 3H), 7.45-7.38 (m, 3H), 7.34-7.27 (m, 2H), |
| | 4.23-4.05 (m, 6H), 3.09 (dd, J = 14.0, 4.4 Hz, 1H), 2.89 (dd, J = 13.8, 10.6 |
| | Hz, 1H), 1.38 (s, 9H). LC-MS (ESI, m/z): [M + H]⁺ 518. |
| 1-58 | 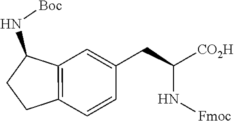 |
| | (S)-2-((((9H-fluoren-9-yl)methoxy)carbonyl)amino)-3-((R)-3-((tert- |
| | butoxycarbonyl)amino)-2,3-dihydro-1H-inden-5-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl N-[(1R)-6-bromo-2,3-dihydro-1H-inden-1-yl]carbamate |
| | (CAS: 1246509-78-3) |
| | ¹H NMR (500 MHZ, DMSO-d₆) δ 12.63 (br, 1H), 7.93-7.82 (m, 2H), 7.78- |
| | 7.48 (m, 3H), 7.48-7.37 (m, 2H), 7.37-7.21 (m, 2H), 7.21-7.01 (m, 4H), |
| | 5.04-4.76 (m, 1H), 4.29-4.06 (m, 4H), 3.09-2.93 (m, 1H), 2.93-2.76 (m, |
| | 2H), 2.74-2.60 (m, 1H), 2.39-2.24 (m, 1H), 1.85-1.69 (m, 1H), 1.42 (s, |
| | 9H). LC-MS (ESI, m/z): [M − Boc + H]⁺ 443. |
| 1-59 | 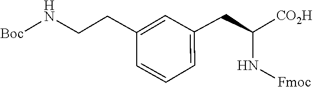 |
| | (S)-2-((((9H-fluoren-9-yl)methoxy)carbonyl)amino)-3-(3-(2-((tert- |
| | butoxycarbonyl)amino)ethyl)phenyl)propanoic acid |
| | methyl (2R)-2-({[(9H-fluoren-9-yl)methoxy]carbonyl}amino)-3- |
| | iodopropanoate (CAS: 156017-42-4) |
| | tert-butyl N-[2-(3-bromophenyl)ethyl]carbamate (CAS: 153732-25-3) |
| | ¹H NMR (500 MHZ, DMSO-d₆) δ 12.69 (br, 1H), 7.88 (d, J = 7.6 Hz, 2H), |
| | 7.75-7.16 (m, 8H), 7.14-6.99 (m, 3H), 6.96-6.47 (m, 1H), 4.25-4.10 (m, |
| | 4H), 3.16-2.96 (m, 3H), 2.89-2.69 (m, 1H), 2.69-2.60 (m, 2H), 1.41- |
| | 1.28 (m, 9H). LC-MS (ESI, m/z): [M + H]⁺ 531. |
| 1-60 | 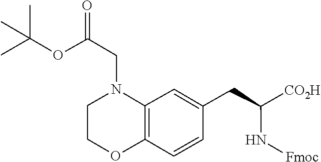 |
| | (S)-2-((((9H-fluoren-9-yl)methoxy)carbonyl)amino)-3-(4-(2-(tert-butoxy)- |
| | 2-oxoethyl)-3,4-dihydro-2H-benzo[b][1,4]oxazin-6-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 2-(6-bromo-2,3-dihydro-4H-benzo[b][1,4]oxazin-4-yl)acetate |
| | ¹H NMR (500 MHz, DMSO-d₆) δ 12.74 (br, 1H), 7.88 (d, J = 7.5 Hz, 2H), |
| | 7.71-7.11 (m, 7H), 6.64-6.54 (m, 1H), 6.54-6.42 (m, 2H), 4.24-3.92 (m, |
| | 8H), 3.44-3.18 (m, 2H), 2.93-2.86 (m, 1H), 2.73-2.59 (m, 1H), 1.41- |
| | 1.31 (m, 9H). LC-MS (ESI, m/z): [M + H]⁺ 559. |
| 1-61 | 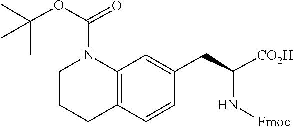 |
| | (S)-2-((((9H-fluoren-9-yl)methoxy)carbonyl)amino)-3-(1-(tert- |
| | butoxycarbonyl)-1,2,3,4-tetrahydroquinolin-7-yl)propanoic acid |
| | methyl (2R)-2-({[(9H-fluoren-9-yl)methoxy]carbonyl}amino)-3- |
| | iodopropanoate (CAS: 156017-42-4) |
| | tert-butyl 7-bromo-1,2,3,4-tetrahydroquinoline-1-carboxylate |
| | (CAS: 1187932-64-4) |
| | ¹H NMR (500 MHZ, DMSO-d₆) δ 12.86-12.64 (m, 1H), 7.91-7.85 (m, |
| | 2H), 7.74-7.69 (m, 1H), 7.69-7.61 (m, 2H), 7.57-7.46 (m, 1H), 7.44- |
| | 7.38 (m, 2H), 7.35-7.24 (m, 2H), 7.04-6.96 (m, 1H), 6.92-6.88 (m, 1H), |
| | 4.23-4.10 (m, 4H), 3.68-3.60 (m, 1H), 3.51-3.41 (m, 1H), 3.05-2.98 (m, |
| | 1H), 2.86-2.78 (m, 1H), 2.70-2.62 (m, 2H), 1.82-1.72 (m, 2H), 1.45 (s, |
| | 9H). LC-MS (ESI, m/z): [M + H]⁺ 543. |
| 1-62 | 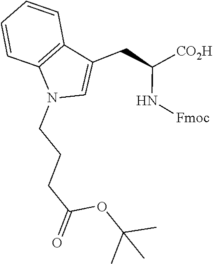 |
| | (2S)-3-{1-[4-(tert-butoxy)-4-oxobutyl]-1H-indol-3-yl}-2-({[(9H-fluoren-9- |
| | yl)methoxy]carbonyl}amino)propanoic acid |
| | methyl (2R)-2-({[(9H-fluoren-9-yl)methoxy]carbonyl}amino)-3- |
| | iodopropanoate (CAS: 156017-42-4) |
| | tert-butyl 4-(3-iodo-1H-indol-1-yl)butanoate |
| | ¹H NMR (500 MHz, DMSO-d₆) δ 12.88 (br, 1H), 7.93-7.82 (m, 2H), 7.74- |
| | 6.99 (m, 12H), 4.36-3.99 (m, 6H), 3.28-3.16 (m, 1H), 3.11-2.94 (m, |
| | 1H), 2.19-2.07 (m, 2H), 1.97-1.82 (m, 2H), 1.34 (s, 9H). LC-MS (ESI, |
| | m/z): [M + H]⁺ 569. |
| 1-63 | 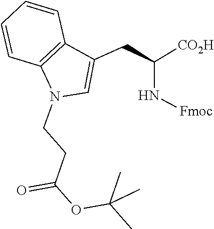 |
| | (2S)-3-{1-[3-(tert-butoxy)-3-oxopropyl]-1H-indol-3-yl}-2-({[(9H-fluoren- |
| | 9-yl)methoxy]carbonyl}amino)propanoic acid |
| | (2R)-2-({[(9H-fluoren-9-yl)methoxy]carbonyl}amino)-3- |
| | methyl (2R)-2-({[(9H-fluoren-9-yl)methoxy]carbonyl}amino)-3- |
| | iodopropanoate (CAS: 156017-42-4) |
| | tert-butyl 3-(3-iodo-1H-indol-1-yl)propanoate |
| | ¹H NMR (500 MHZ, DMSO-d₆) δ 12.78-12.61 (m, 1H), 7.91-7.82 (m, |
| | 2H), 7.70-7.63 (m, 3H), 7.57 (d, J = 7.8 Hz, 1H), 7.50-7.23 (m, 5H), 7.21- |
| | 6.99 (m, 3H), 4.37-3.99 (m, 6H), 3.21-3.12 (m, 1H), 3.07-2.95 (m, 1H), |
| | 2.65 (t, J = 6.6 Hz, 2H), 1.30 (s, 9H). LC-MS (ESI, m/z): [M + H]⁺ 555. |

### Example-2

The following compounds can be synthesized as outlined for the preparation of Reference example-4-01, Reference example-4-02, or Reference example-4-03 employing appropriate starting materials in the table.

| | Structure and IUPAC name |
|---|---|
| | Starting material-A |
| | Starting material-B |
| Example | NMR and MS |
| 2-01 | 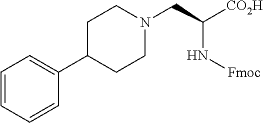 |
| | (S)-2-((((9H-fluoren-9-yl)methoxy)carbonyl) |
| | amino)-3-(4-phenylpiperidin- |
| | 1-yl)propanoic acid |
| | 3-((9H-fluoren-9-yl)methyl) 4-(tert-butyl) |
| | (S)-1,2,3-oxathiazolidine-3,4- |
| | dicarboxylate 2,2-dioxide (CAS: 2417280-85-2) |
| | 4-Phenylpiperidine (CAS: 771-99-3) |
| | ¹H NMR (500 MHz, METHANOL-d₄) δ 7.89 − |
| | 7.75 (m, 2H), 7.71 − 7.61 (m, 2H), 7.44 − 7.18 (m, 9H), |
| | 4.49 − 4.31 (m, 3H), 4.27 − 4.20 (m, 1H), 3.80 − |
| | 3.66 (m, 2H), 3.40 − 3.30 (m, 2H), 3.23 − 3.08 (m, 2H), |
| | 2.93 − 2.82 (m, 1H), 2.15 − 1.92 (m, 4H). LC-MS |
| | (ESI, m/z): [M + H]⁺ 471. |
| 2-02 | 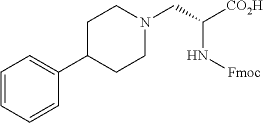 |
| | (R)-2-((((9H-fluoren-9-yl)methoxy)carbonyl) |
| | amino)-3-(4-phenylpiperidin-1-yl)propanoic acid |
| | Reference example-3-04 |
| | 4-Phenylpiperidine (CAS: 771-99-3) |
| | Substantially identical to Example 2-01 |
| 2-03 | 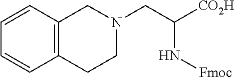 |
| | 2-((((9H-fluoren-9-yl)methoxy)carbonyl)amino)-3-(3,4- |
| | dihydroisoquinolin-2(1H)-yl)propanoic acid |
| | 4-tert-butyl 3-(9H-fluoren-9-yl)methyl 2,2- |
| | dioxo-1,2lambda6,3-oxathiazolidine-3,4-dicarboxylate |
| | 1,2,3,4-Tetrahydroisoquinoline (CAS: 91-21-4) |
| | ¹H NMR (300 MHz, METHANOL-d₄) δ 7.80 (d, J = 7.5 |
| | Hz, 2H), 7.73 − 7.60 (m, 2H), 7.51 − 7.06 (m, 8H), 4.68 − |
| | 4.34 (m, 4H), 4.29 − 4.17 (m, 1H), 3.72 − 3.40 (m, 3H), |
| | 3.25 − 3.00 (m, 2H). LC-MS (ESI, m/z): [M + H]⁺ 443. |

### Example-3

The following compounds can be synthesized as outlined for the preparation of Reference example-5-01 or Reference example-5-02 employing appropriate starting materials in the table.

| | Structure and IUPAC name |
|---|---|
| | Starting material-A |
| | Starting material-B |
| Example | NMR and MS |
| 3-01 | 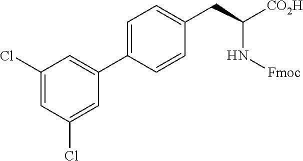 |
| | (S)-2-((((9H-fluoren-9-yl)methoxy)carbonyl)amino)-3- |
| | (3′,5′-dichloro-[1,1′-biphenyl]-4-yl)propanoic acid |
| | (2S)-3-(4-bromophenyl)-2-({[(9H-fluoren-9- |
| | yl)methoxy]carbonyl} amino)propanoic acid |
| | (CAS: 198561-04-5) |
| | (3,5-Dichlorophenyl)boronic acid (CAS: 67492-50-6) |
| | ¹H NMR (500 MHz, DMSO-d₆) δ 12.81 |
| | (br, 1H), 7.88 (d, J = 7.5 Hz, 2H), |
| | 7.78 (d, J = 8.5 Hz, 1H), 7.74 − 7.45 (m, |
| | 7H), 7.45 − 7.11 (m, 6H), 4.28 − 4.10 |
| | (m, 4H), 3.21 − 2.85 (m, 2H). LC-MS (ESI, m/z): |
| | [M + H]⁺ 532. |
| 3-02 | 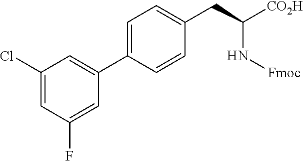 |
| | (S)-2-((((9H-fluoren-9-yl)methoxy) |
| | carbonyl)amino)-3-(3′-chloro-5′- |
| | fluoro-[1,1′-biphenyl]-4-yl)propanoic acid |
| | (2S)-3-(4-bromophenyl)-2-({[(9H-fluoren-9- |
| | yl)methoxy]carbonyl}amino)propanoic acid |
| | (CAS: 198561-04-5) |
| | B-(3-Chloro-5-fluorophenyl)boronic acid (CAS: 328956-61-2) |
| | ¹H NMR (500 MHz, DMSO-d₆) δ 12.75 (br, 1H), |
| | 7.88 (d, J = 7.6 Hz, 2H), 7.78 (d, J = 8.7 Hz, 1H), 7.74 − |
| | 7.45 (m, 6H), 7.45 − 7.18 (m, 7H), 4.29 − 4.10 (m, 4H), |
| | 3.19 − 2.87 (m, 2H). LC-MS (ESI, m/z): [M + H]⁺ 516. |
| 3-03 | 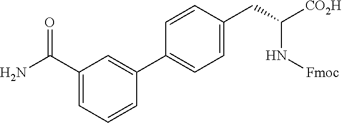 |
| | (R)-2-((((9H-fluoren-9-yl)methoxy)carbonyl)amino)-3- |
| | (3′-carbamoyl-[1,1′-biphenyl]-4-yl)propanoic acid |
| | (2R)-3-(4-bromophenyl)-2-({[(9H-fluoren-9- |
| | yl)methoxy\|carbonyl }amino)propanoic acid |
| | (CAS: 198545-76-5) |
| | 3-carbamoylphenylboronic acid (CAS: 351422-73-6) |
| | ¹H NMR (500 MHz, DMSO-d₆) δ 12.87 (br, 1H), |
| | 8.14 (br, 1H), 8.09 (br, 1H), 7.92 − 7.59 (m, 9H), |
| | 7.59 − 7.18 (m, 8H), 4.29 − 4.10 (m, 4H), 3.18 − |
| | 2.87 (m, 2H). LC-MS (ESI, m/z): [M + H]⁺ 507. |
| 3-04 | 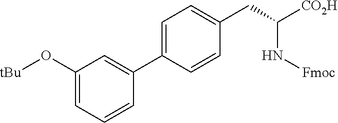 |
| | (R)-2-((((9H-fluoren-9-yl)methoxy)carbonyl)amino)- |
| | 3-(3′-(tert-butoxy)-[1,1′-biphenyl]-4-yl)propanoic acid |
| | (2R)-3-(4-bromophenyl)-2-({[(9H-fluoren-9- |
| | yl)methoxy]carbonyl}amino)propanoic acid |
| | (CAS: 198545-76-5) |
| | [3-(tert-butoxy)phenyl]boronic acid (CAS: 312931-06-9) |
| | ¹H NMR (500 MHz, DMSO-d₆) δ 12.80 (br, 1H), 7.87 |
| | (d, J = 7.6 Hz, 2H), 7.77 (d, J = 8.4 Hz, 1H), 7.66 (d, J = |
| | 7.5 Hz, 1H), 7.63 (d, J = 7.6 Hz, 1H), 7.54 (d, J = 8.2 Hz, 2H), |
| | 7.42 − 7.24 (m, 8H), 7.16 (s, 1H), 6.96 (d, J = 7.4 Hz, 1H), |
| | 4.27 − 4.12 (m, 4H), 3.16 − 3.09 (m, 1H), 2.95 − 2.87 (m, |
| | 1H), 1.32 (s, 9H). LC-MS (ESI, m/z): [M + H]⁺ 536. |
| 3-05 | 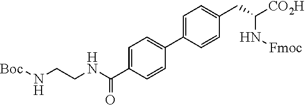 |
| | (R)-2-((((9H-fluoren-9-yl)methoxy)carbonyl)amino)- |
| | 3-(4′-((2-((tert-butoxycarbonyl)amino)ethyl)carbamoyl)- |
| | [1,1′-biphenyl]-4-yl)propanoic acid |
| | (2R)-3-(4-bromophenyl)-2-({[(9H-fluoren-9- |
| | yl)methoxy]carbonyl}amino)propanoic acid |
| | (CAS: 198545-76-5) |
| | (4-((2-((tert-butoxycarbonyl)amino)ethyl) |
| | carbamoyl)phenyl)boronic acid |
| | (CAS: 860626-05-7) |
| | ¹H NMR (500 MHz, METHANOL-d₄) δ 7.83 (d, J = 8.2 |
| | Hz, 2H), 7.76 (d, J = 7.6 Hz, 2H), 7.62 − 7.54 (m, 4H), |
| | 7.49 (d, J = 8.1 Hz, 2H), 7.38 − 7.16 (m, 6H), 4.40 − 4.01 |
| | (m, 4H), 3.49 − 3.43 (m, 2H), 3.34 − 3.22 (m, 3H), 3.02 − |
| | 2.92 (m, 1H), 1.41 (s, 9H). LC-MS (ESI, m/z): [M + H]⁺ 650. |
| 3-06 | 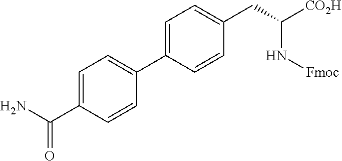 |
| | (R)-2-((((9H-fluoren-9-yl)methoxy)carbonyl)amino)- |
| | 3-(4′-carbamoyl-[1,1′-biphenyl]-4-yl)propanoic acid |
| | (2R)-3-(4-bromophenyl)-2-({[(9H-fluoren-9- |
| | yl)methoxy\|carbonyl }amino)propanoic acid |
| | (CAS: 198545-76-5) |
| | 4-Carbamoylphenylboronic acid (CAS: 123088-59-5) |
| | ¹H NMR (500 MHz, DMSO-d₆) δ 12.89 (br, 1H), 8.01 (s, |
| | 1H), 7.94 (d, J = 8.2 Hz, 2H), 7.88 (d, J = 7.5 Hz, 2H), |
| | 7.76 − 7.48 (m, 7H), 7.45 − 7.25 (m, 7H), 4.25 − 4.14 |
| | (m, 4H), 3.19 − 3.01 (m, 1H), 2.98 − 2.75 (m, 1H). LC-MS |
| | (ESI, m/z): [M + H]⁺ 507. |
| 3-07 | 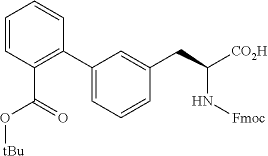 |
| | (S)-2-((((9H-fluoren-9-yl)methoxy) |
| | carbonyl)amino)-3-(2′-(tert- |
| | butoxycarbonyl)-[1,1′-biphenyl]-3-yl)propanoic acid |
| | (2S)-3-(3-bromophenyl)-2-({[(9H-fluoren-9- |
| | yl)methoxy]carbonyl}amino)propanoic acid |
| | (CAS: 220497-48-3) |
| | tert-butyl 2-(4,4,5,5-tetramethyl-1,3,2- |
| | dioxaborolan-2-yl)benzoate (CAS:956229-69-9) |
| | ¹H NMR (500 MHz, DMSO-d₆) δ 12.78 (br, 1H), |
| | 7.87 (d, J = 7.5 Hz, 2H), 7.75 (d, J = 8.4 Hz, 1H), |
| | 7.69 − 7.61 (m, 3H), 7.56 − 7.20 (m, 10H), 7.13 (d, |
| | J = 7.3 Hz, 1H), 4.25 − 4.11 (m, 4H), 3.12 (dd, J = |
| | 13.7, 4.1 Hz, 1H), 2.96 − 2.87 (m, 1H), 1.18 |
| | (s, 9H). LC-MS (ESI, m/z): [M − tBu + H]⁺ 508. |
| 3-08 | 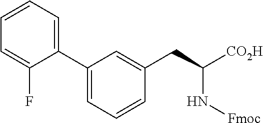 |
| | (S)-2-((((9H-fluoren-9-yl)methoxy)carbonyl)amino)-3-(2′- |
| | fluoro-[1,1′-biphenyl]-3-yl)propanoic acid |
| | (2S)-3-(3-bromophenyl)-2-({[(9H-fluoren-9- |
| | yl)methoxy\|carbonyl}amino)propanoic acid |
| | (CAS: 220497-48-3) |
| | 2-Fluorophenylboronic acid (CAS: 1993-03-9) |
| | ¹H NMR (500 MHz, DMSO-d₆) δ 12.80 (br, 1H), 7.87 |
| | (d, J = 7.5 Hz, 2H), 7.81 (d, J = 8.5 Hz, 1H), 7.63 (dd, |
| | J = 10.2, 7.7 Hz, 2H), 7.56 − 7.46 (m, 2H), 7.44 − 7.36 |
| | (m, 5H), 7.34 − 7.21 (m, 5H), 4.27 − 4.12 (m, 4H), 3.20 − |
| | 3.09 (m, 1H), 2.95 (dd, J = 13.8, 10.6 Hz, 1H). LC-MS |
| | (ESI, m/z): [M + H]⁺ 482. |
| 3-09 | 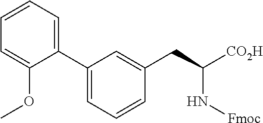 |
| | (S)-2-((((9H-fluoren-9-yl)methoxy)carbonyl)amino)-3- |
| | (2′-methoxy-[1,1′-biphenyl]-3-yl)propanoic acid |
| | (2S)-3-(3-bromophenyl)-2-({[(9H-fluoren-9- |
| | yl)methoxy]carbonyl}amino)propanoic acid |
| | (CAS: 220497-48-3) |
| | 2-Methoxyphenylboronic acid (CAS: 5720-06-9) |
| | ¹H NMR (500 MHz, DMSO-d₆) δ 12.77 (br, 1H), 7.89 − |
| | 7.83 (m, 2H), 7.76 (d, J = 8.5 Hz, 1H), 7.67 − 7.62 (m, 2H), |
| | 7.41 − 7.21 (m, 10H), 7.08 (d, J = 8.1 Hz, 1H), 7.01 − 6.97 |
| | (m, 1H), 4.26 − 4.13 (m, 4H), 3.73 (s, 3H), 3.20 − 3.06 (m, |
| | 1H), 2.97 − 2.87 (m, 1H). LC-MS (ESI, m/z): [M + H]⁺ 494. |
| 3-10 | 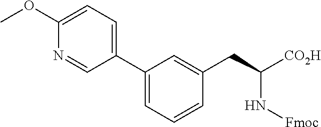 |
| | (S)-2-((((9H-fluoren-9-yl)methoxy)carbonyl)amino)- |
| | 3-(3-(6-methoxypyridin-3-yl)phenyl)propanoic acid |
| | (2S)-3-(3-bromophenyl)-2-({[(9H-fluoren-9- |
| | yl)methoxy]carbonyl}amino)propanoic acid |
| | (CAS: 220497-48-3) |
| | (6-Methoxypyridin-3-yl)boronic acid (CAS: 163105-89-3) |
| | ¹H NMR (500 MHz, DMSO-d₆) δ 12.82 (br, 1H), 8.47 (d, J = |
| | 2.5 Hz, 1H), 8.02 − 7.96 (m, 1H), 7.86 (d, J = 7.5 Hz, 2H), |
| | 7.79 (d, J = 8.5 Hz, 1H), 7.66 − 7.55 (m, 3H), 7.55 − 7.46 |
| | (m, 1H), 7.43 − 7.32 (m, 3H), 7.32 − 7.17 (m, 3H), 6.94 − |
| | 6.84 (m, 1H), 4.30 − 4.09 (m, 4H), 3.89 (s, 3H), 3.21 − |
| | 3.08 (m, 1H), 2.99 − 2.78 (m, 1H). LC-MS (ESI, m/z): |
| | [M + H]⁺ 495. |
| 3-11 | 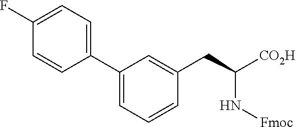 |
| | (S)-2-((((9H-fluoren-9-yl)methoxy)carbonyl)amino)- |
| | 3-(4′-fluoro-[1,1′-biphenyl]-3-yl)propanoic acid |
| | (2S)-3-(3-bromophenyl)-2-({[(9H-fluoren-9- |
| | yl)methoxy]carbonyl }amino)propanoic acid |
| | (CAS: 220497-48-3) |
| | (4-Fluorophenyl)boronic acid (CAS: 1765-93-1) |
| | ¹H NMR (500 MHz, DMSO-d₆) 8 12.79 (br, 1 H), 7.89 − |
| | 7.81 (m, 3 H), 7.72 − 7.66 (m, 2 H), 7.65 − 7.58 (m, 3 H), |
| | 7.49 (d, J = 7.78 Hz, 1 H), 7.41 − 7.35 (m, 3 H), 7.29 − |
| | 7.19 (m, 5 H), 4.28 − 4.11 (m, 4 H), 3.17 (dd, J = 13.7, 4.1 |
| | Hz, 1 H), 2.94 (dd, J = 13.7, 10.8 Hz, 1 H). LC-MS |
| | (ESI, m/z): [M + H]⁺ 482. |
| 3-12 | 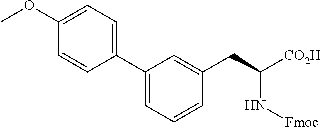 |
| | (S)-2-((((9H-fluoren-9-yl)methoxy)carbonyl)amino)- |
| | 3-(4′-methoxy-[1,1′-biphenyl]-3-yl)propanoic acid |
| | (2S)-3-(3-bromophenyl)-2-({[(9H-fluoren-9- |
| | yl)methoxy]carbonyl}amino)propanoic acid |
| | (CAS: 220497-48-3) |
| | (4-Methoxyphenyl)boronic acid (CAS: 5720-07-0) |
| | ¹H NMR (500 MHz, DMSO-d₆) δ 13.0 (br, 1 H), 7.87 |
| | (d, J = 7.5 Hz, 2 H), 7.65 − 7.54 (m, 5H), 7.47 − 7.48 |
| | (m, 8H), 6.97 (d, J = 8.5 Hz, 2 H), 4.19 − 4.13 |
| | (m, 3 H), 3.82 − 3.69 (m, 4 H), 3.20 − 3.12 (m, 1 H), |
| | 2.99 − 2.89 (m, 1 H). LC-MS (ESI, m/z): [M + H]⁺ 494. |
| 3-13 | 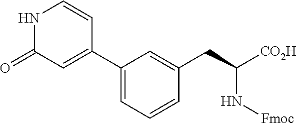 |
| | (S)-2-((((9H-fluoren-9-yl)methoxy)carbonyl)amino)-3- |
| | (3-(2-oxo-1,2-dihydropyridin-4-yl)phenyl)propanoic acid |
| | (2S)-3-(3-bromophenyl)-2-({[(9H-fluoren-9- |
| | yl)methoxy]carbonyl}amino)propanoic acid |
| | (CAS: 220497-48-3) |
| | (6-oxo-1,6-dihydropyridin-3-yl)boronic acid |
| | (CAS: 903899-13-8) |
| | ¹H NMR (500 MHz, DMSO-d₆) δ 12.77 (br, 1H), 11.64 |
| | (br, 1H), 7.91 − 7.79 (m, 3H), 7.70 − 7.65 (m, 1H), 7.65 − |
| | 7.58 (m, 2H), 7.58 − 7.50 (m, 1H), 7.45 − 7.33 (m, 5H), |
| | 7.31 − 7.18 (m, 2H), 6.63 (d, J = 1.5 Hz, 1H), 6.55 − 5.98 (m, |
| | 1H), 4.29 − 4.20 (m, 1H), 4.20 − 4.09 (m, 3H), 3.21 − |
| | 3.14 (m, 1H), 2.97 − 2.88 (m, 1H). LC-MS (ESI, m/z): |
| | [M + H]⁺ 481. |
| 3-14 | 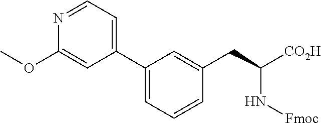 |
| | (S)-2-((((9H-fluoren-9-yl)methoxy)carbonyl)amino)- |
| | 3-(3-(2-methoxypyridin-4-yl)phenyl)propanoic acid |
| | (2S)-3-(3-bromophenyl)-2-({[(9H-fluoren-9- |
| | yl)methoxy]carbonyl}amino)propanoic acid |
| | (CAS: 220497-48-3) |
| | (2-methoxypyridin-4-yl)boronic acid (CAS: 762262-09-9) |
| | ¹H NMR (500 MHz, DMSO-d₆) δ 12.83 (br, 1H), 8.25 − 8.17 |
| | (m, 1H), 7.90 − 7.81 (m, 3H), 7.77 − 7.71 (m, 1H), 7.67 − |
| | 7.57 (m, 3H), 7.49 − 7.33 (m, 4H), 7.33 − 7.28 (m, 1H), |
| | 7.28 − 7.22 (m, 1H), 7.22 − 7.17 (m, 1H), 7.11 (s, 1H), 4.31 − |
| | 4.22 (m, 1H), 4.21 − 4.10 (m, 3H), 3.88 (s, 3H), 3.23 − |
| | 3.16 (m, 1H), 3.00 − 2.91 (m, 1H). LC-MS (ESI, m/z): |
| | [M + H]⁺ 495. |
| 3-15 | 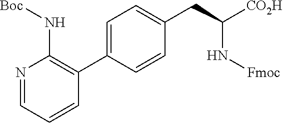 |
| | (S)-2-((((9H-fluoren-9-yl)methoxy)carbonyl)amino)-3-(4-(2- |
| | ((tert-butoxycarbonyl)amino)pyridin-3-yl)phenyl) |
| | propanoic acid |
| | (2S)-3-(4-bromophenyl)-2-({[(9H-fluoren-9- |
| | yl)methoxy]carbonyl }amino)propanoic acid |
| | (CAS: 198561-04-5) |
| | (2-((tert-butoxycarbonyl)amino)pyridin-3-yl)boronic acid |
| | (CAS: 863753-35-9) |
| | ¹H NMR (500 MHz, METHANOL-d₄) δ 8.35 − 8.29 (m, 1H), |
| | 7.80 (d, J = 7.6 Hz, 2H), 7.68 − 7.58 (m, 3H), 7.41 − 7.26 |
| | (m, 9H), 4.42 − 4.29 (m, 2H), 4.26 − 4.12 (m, 2H), 3.31 − |
| | 3.26 (m, 1H), 3.06 − 2.96 (m, 1H), 1.34 − 1.30 (m, |
| | 9H). LC-MS (ESI, m/z): [M + H]⁺ 580. |

### Example-4-01: (2R,3S)-2-((((9H-fluoren-9-yl)methoxy)carbonyl)amino)-3-cyclopropyl-3-hydroxypropanoic acid

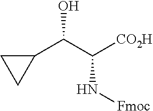

To a solution of (5R)-5-phenylmorpholin-2-one (CAS: 121269-45-2, 3 g, 17 mmol) in toluene (30 mL) were added Molecular Sieve 4A (CAS: 70955-01-0, 1.5 g) and cyclopropanecarbaldehyde (CAS: 1489-69-6, 11.9 g, 169 mmol). The mixture was stirred at 110° C. for 24 h, and then concentrated. The resulting residue was purified by silica gel flash column chromatography (isocratic, petroleum ether/Et₂O=5/1). Appropriate fractions were concentrated. To a mixture of the resulting residue (2.8 g, 9.4 mmol) in MeOH (40 mL) was added 1 M aq. HCl (10 mL, 10 mmol). The resulting mixture was stirred at 80° C. for 3 h, and then concentrated. The resulting residue was purified by reversed phase silica gel flash column chromatography (C18 silica gel, gradient, 10 mM aq. NH₄HCO₃/CH₃CN=90/10 to 50/50). Appropriate fractions were concentrated. To a mixture of the resulting residue (1 g, 3.6 mmol) in MeOH (5 mL) and water (1 mL) were added Pd(OH)₂/C (0.5 g) and TFA (0.41 g, 3.6 mmol). The resulting mixture was stirred at rt overnight under hydrogen atmosphere, and then concentrated. To a solution of the resulting residue (0.56 g, 3.52 mmol) in 1,4-dioxane (5 mL) and H₂O (2.5 mL) at 0° C. was added NaHCO₃ (0.89 g, 10.6 mmol), followed by a solution of Fmoc-OSu (1.13 g, 3.35 mmol) in 1,4-dioxane (3 mL) dropwise. The mixture was stirred for 2 h at rt, and then pH of the solution was adjusted to 5 with satd. aq. NaH₂PO₄. The mixture was extracted three times with EtOAc. The organic extracts were washed with brine, dried over Na₂SO₄, filtered, and concentrated. The resulting residue was purified by silica gel flash column chromatography (isocratic, petroleum ether/EtOAc=1/1). Appropriate fractions were concentrated. To a mixture of the resulting residue (0.5 g, 1.3 mmol) in 1,4-dioxane (10 mL) was added 6 M aq. HCl (5 mL, 30 mmol). The resulting mixture was stirred at 80° C. for 2 h, and then concentrated. The resulting residue was purified by silica gel flash column chromatography (isocratic, petroleum ether/EtOAc=1/1) to afford the title compound (0.30 g, 0.82 mmol). ¹H NMR (500 MHz, DMSO-d₆) δ 12.62 (br, 1H), 7.93-7.87 (m, 2H), 7.79-7.61 (m, 2H), 7.45-7.39 (m, 2H), 7.37-7.29 (m, 2H), 7.20-7.11 (m, 1H), 5.21-4.72 (m, 1H), 4.35-4.22 (m, 3H), 4.17-4.08 (m, 1H), 3.30-3.12 (m, 1H), 0.97-0.88 (m, 1H), 0.49-0.32 (m, 3H), 0.19-0.01 (m, 1H). LC-MS (ESI, m/z): [M+H]⁺ 368.

### Example-4

The following compounds can be synthesized as outlined for the preparation of Reference example-6-01, Reference example-6-02, Reference example-6-03, Reference example-6-04, Reference example-6-05, Example-4-01 or the methods described in Tetrahedron: Asymmetry 2001, 12, 481. employing appropriate starting materials in the table.

| | Structure and IUPAC name |
|---|---|
| | Starting material-A |
| | Starting material-B |
| Example | NMR and MS |
| 4-02 | 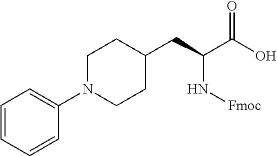 |
| | (S)-2-((((9H-fluoren-9-yl)methoxy)carbonyl)amino)-3-(1-phenylpiperidin- |
| | 4-yl)propanoic acid |
| | (15S)-9-chloro-19-[(3,4-dichlorophenyl)methyl]-6-phenyl-2-oxa- |
| | 5lambda4,13,19lambda4-triaza-1- |
| | nickleapentacyclo[11.6.0.01,5.07,12.015,19]nonadeca-5,7(12),8,10- |
| | tetraene-3,14-dione (CAS: 2089661-84-5) |
| | 4-(Bromomethyl)-1-phenylpiperidine (CAS: 1337606-49-1) |
| | ¹H NMR (500 MHz, DMSO-d₆) δ 12.6 (br, 1H), 7.90 (d, J = 7.5 Hz, 2H), |
| | 7.79-7.58 (m, 3H), 7.48-7.38 (m, 2H), 7.37-7.26 (m, 2H), 7.25-7.09 (m, |
| | 2H), 6.92 (d, J = 8.2 Hz, 2H), 6.79-6.67 (m, 1H), 4.42-4.16 (m, 3H), 4.11- |
| | 3.88 (m, 1H), 3.74-3.57 (m, 2H), 2.68-2.52 (m, 2H), 1.83-1.40 (m, 5H), |
| | 1.34-1.07 (m, 2H). LC-MS (ESI, m/z): [M + H]⁺ 471. |
| 4-03 | 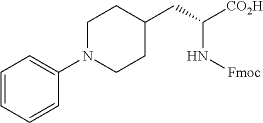 |
| | (R)-2-((((9H-fluoren-9-yl)methoxy)carbonyl)amino)-3-(1-phenylpiperidin- |
| | 4-yl)propanoic acid |
| | (15R)-9-chloro-19-[(3,4-dichlorophenyl)methyl]-6-phenyl-2-oxa- |
| | 5lambda4,13,19lambda4-triaza-1- |
| | nickelapentacyclo[11.6.0.01,5.07,12.015,19]nonadeca-5,7(12),8,10- |
| | tetraene-3,14-dione (CAS: 2408623-29-8) |
| | 4-(Bromomethyl)-1-phenylpiperidine (CAS: 1337606-49-1) |
| | Substantially identical to Example 4-02 |
| 4-04 | 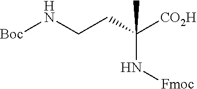 |
| | (R)-2-((((9H-fluoren-9-yl)methoxy)carbonyl)amino)-4-((tert-butoxycarbonyl)amino)- |
| | 2-methylbutanoic acid |
| | (2S)-3,6-dimethoxy-2-(propan-2-yl)-2,5-dihydropyrazine (CAS: 78342-42-4) |
| | tert-butyl (2-bromoethyl)carbamate (CAS: 39684-80-5) |
| | ¹H NMR (500 MHz, DMSO-d₆) δ 12.46 (br, 1H), 7.89 (d, J = 7.5 Hz, 2H), |
| | 7.72 (d, J = 7.5 Hz, 2H), 7.52 (s, 1H), 7.42 (dd, J = 7.5, 6.5 Hz, 2H), 7.33 |
| | (dd, J = 7.5, 7.0 Hz, 2H), 6.82-6.35 (m, 1H), 4.29-4.16 (m, 3H), 3.01- |
| | 2.78 (m, 2H), 2.04-1.76 (m, 2H), 1.37 (s, 9H), 1.34 (s, 3H). LC-MS (ESI, |
| | m/z): [M − Boc + H]⁺ 355. |
| 4-05 | 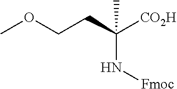 |
| | (S)-2-((((9H-fluoren-9-yl)methoxy)carbonyl)amino)-4-methoxy-2-methylbutanoic acid |
| | (2R)-3,6-dimethoxy-2-(propan-2-yl)-2,5-dihydropyrazine (CAS: 109838-85-9) |
| | 1-bromo-2-methoxyethane (CAS: 6482-24-2) |
| | ¹H NMR (500 MHz, DMSO-d₆) δ 12.47 (br, 1H), 7.89 (d, J = 7.5 Hz, 2H), |
| | 7.71 (d, J = 7.5 Hz, 2H), 7.42 (dd, J = 7.5, 7.0 Hz, 2H), 7.38 (s, 1H), 7.36- |
| | 7.29 (m, 2H), 4.33-4.18 (m, 3H), 3.41-3.23 (m, 2H), 3.18 (s, 3H), 2.13- |
| | 1.90 (m, 2H), 1.35 (s, 3H). LC-MS (ESI, m/z): [M + H]⁺ 370. |
| 4-06 | 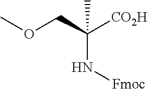 |
| | (S)-2-((((9H-fluoren-9-yl)methoxy)carbonyl)amino)-3-methoxy-2- |
| | methylpropanoic acid |
| | (2R)-3,6-dimethoxy-2-(propan-2-yl)-2,5-dihydropyrazine (CAS: 109838-85-9) |
| | bromo(methoxy)methane (CAS: 13057-17-5) |
| | ¹H NMR (500 MHz, DMSO-d₆) δ 12.58 (br, 1H), 7.89 (d, J = 7.5 Hz, 2H), |
| | 7.72 (d, J = 7.5 Hz, 2H), 7.45-7.36 (m, 3H), 7.36-7.30 (m, 2H), 4.36-4.16 |
| | (m, 3H), 3.63 (d, J = 9.0 Hz, 1H), 3.51 (d, J = 9.0 Hz, 1H), 3.24 (s, 3H), 1.35 |
| | (s, 3H). LC-MS (ESI, m/z): [M + H]⁺ 356. |
| 4-07 | 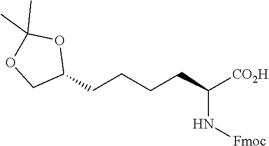 |
| | (S)-2-((((9H-fluoren-9-yl)methoxy)carbonyl)amino)-6-((R)-2,2-dimethyl- |
| | 1,3-dioxolan-4-yl)hexanoic acid |
| | (15S)-9-chloro-19-[(3,4-dichlorophenyl)methyl]-6-phenyl-2-oxa- |
| | 5lambda4,13,19lambda4-triaza-1- |
| | nickelapentacyclo[11.6.0.01,5.07,12.015,19]nonadeca-5,7(12),8,10- |
| | tetraene-3,14-dione (CAS: 2089661-84-5) |
| | 4-[(4R)-2,2-dimethyl-1,3-dioxolan-4-yl]butyl 4-methylbenzene-1-sulfonate |
| | (CAS: 266318-68-7) |
| | ¹H NMR (500 MHz, DMSO-d₆) δ 12.51 (br, 1H), 7.89 (d, J = 7.5 Hz, 2H), |
| | 7.78-7.58 (m, 3H), 7.42 (dd, J = 7.5, 7.5 Hz, 2H), 7.33 (dd, J = 7.5, 7.5 Hz, |
| | 2H), 4.35-4.17 (m, 3H), 4.05-3.86 (m, 3H), 3.51-3.38 (m, 1H), 1.75- |
| | 1.55 (m, 2H), 1.55-1.21 (m, 12H). LC-MS (ESI, m/z): [M + H]⁺ 454. |
| 4-08 | 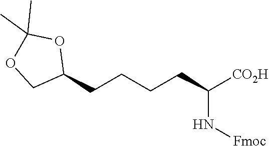 |
| | (S)-2-((((9H-fluoren-9-yl)methoxy)carbonyl)amino)-6-((S)-2,2-dimethyl- |
| | 1,3-dioxolan-4-yl)hexanoic acid |
| | (15S)-9-chloro-19-[(3,4-dichlorophenyl)methyl]-6-phenyl-2-oxa- |
| | 5lambda4,13,19lambda4-triaza-1- |
| | nickelapentacyclo[11.6.0.01,5.07,12.015,19]nonadeca-5,7(12),8,10- |
| | tetraene-3,14-dione (CAS: 2089661-84-5) |
| | 4-[(4S)-2,2-dimethyl-1,3-dioxolan-4-yl]butyl 4-methylbenzene-1-sulfonate |
| | (CAS: 136963-50-3) |
| | ¹H NMR (500 MHz, DMSO-d₆) δ 12.52 (br, 1H), 7.90 (d, J = 7.5 Hz, 2H), |
| | 7.78-7.55 (m, 3H), 7.42 (dd, J = 7.5, 7.5 Hz, 2H), 7.33 (dd, J = 7.5, 7.5 Hz, |
| | 2H), 4.34-4.18 (m, 3H), 4.04-3.87 (m, 3H), 3.42-3.38 (m, 1H), 1.75- |
| | 1.55 (m, 2H), 1.55-1.18 (m, 12H). LC-MS (ESI, m/z): [M + H]⁺ 454. |
| 4-09 | 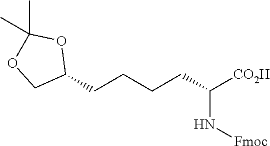 |
| | (R)-2-((((9H-fluoren-9-yl)methoxy)carbonyl)amino)-6-((R)-2,2-dimethyl- |
| | 1,3-dioxolan-4-yl)hexanoic acid |
| | (15R)-9-chloro-19-[(3,4-dichlorophenyl)methyl]-6-phenyl-2-oxa- |
| | 5lambda4,13,19lambda4-triaza-1- |
| | nickelapentacyclo[11.6.0.01,5.07,12.015,19]nonadeca-5,7(12),8,10- |
| | tetraene-3,14-dione (CAS: 2408623-29-8) |
| | 4-[(4R)-2,2-dimethyl-1,3-dioxolan-4-yl]butyl 4-methylbenzene-1-sulfonate |
| | (CAS: 266318-68-7) |
| | ¹H NMR (500 MHz, DMSO-d₆) δ 12.48 (br, 1H), 7.90 (d, J = 7.5 Hz, 2H), |
| | 7.77-7.56 (m, 3H), 7.42 (dd, J = 7.5, 7.5 Hz, 2H), 7.33 (dd, J = 7.5, 7.0 |
| | Hz, 2H), 4.35-4.16 (m, 3H), 4.05-3.86 (m, 3H), 3.45-3.37 (m, 1H), 1.75- |
| | 1.55 (m, 2H), 1.55-1.18 (m, 12H). LC-MS (ESI, m/z): [M + H]⁺ 454. |
| 4-10 | 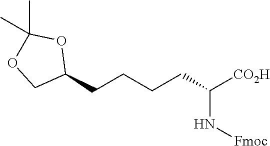 |
| | (R)-2-((((9H-fluoren-9-yl)methoxy)carbonyl)amino)-6-((S)-2,2-dimethyl- |
| | 1,3-dioxolan-4-yl)hexanoic acid |
| | (15R)-9-chloro-19-[(3,4-dichlorophenyl)methyl]-6-phenyl-2-oxa- |
| | 5lambda4,13,19lambda4-triaza-1- |
| | nickelapentacyclo[11.6.0.01,5.07,12.015,19]nonadeca-5,7(12),8,10- |
| | tetraene-3,14-dione (CAS: 2408623-29-8) |
| | 4-[(4S)-2,2-dimethyl-1,3-dioxolan-4-yl]butyl 4-methylbenzene-1-sulfonate |
| | (CAS: 136963-50-3) |
| | ¹H NMR (500 MHz, DMSO-d₆) δ 12.45 (br, 1H), 7.90 (d, J = 7.5 Hz, 2H), |
| | 7.76-7.56 (m, 3H), 7.42 (dd, J = 7.5, 7.0 Hz, 2H), 7.33 (dd, J = 7.5, 7.0 Hz, |
| | 2H), 4.33-4.18 (m, 3H), 4.04-3.86 (m, 3H), 3.42-3.38 (m, 1H), 1.74- |
| | 1.55 (m, 2H), 1.55-1.12 (m, 12H). LC-MS (ESI, m/z): [M + H]⁺ 454. |
| 4-11 | 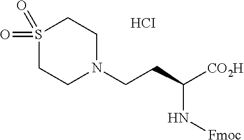 |
| | (S)-2-((((9H-fluoren-9-yl)methoxy)carbonyl)amino)-4-(1,1- |
| | dioxidothiomorpholino)butanoic acid hydrochloride |
| | (15S)-9-chloro-19-[(3,4-dichlorophenyl)methyl]-6-phenyl-2-oxa- |
| | 5lambda4,13,19lambda4-triaza-1- |
| | nickelapentacyclo[11.6.0.01,5.07,12.015,19]nonadeca-5,7(12),8,10- |
| | tetraene-3,14-dione (CAS: 2089661-84-5) |
| | 4-(2-chloroethyl)thiomorpholine 1,1-dioxide (CAS: 55160-66-2) |
| | ¹H NMR (500 MHz, DMSO-d₆) δ 12.52 (br, 1H), 7.90 (d, J = 7.5 Hz, 2H), |
| | 7.76-7.68 (m, 2H), 7.64 (d, J = 8.1 Hz, 1H), 7.46-7.38 (m, 2H), 7.37-7.30 |
| | (m, 2H), 4.30 (d, J = 7.0 Hz, 2H), 4.27-4.19 (m, 1H), 4.10-3.99 (m, 1H), |
| | 3.13-2.99 (m, 4H), 2.94-2.81 (m, 4H), 2.07 (s, 2H), 1.93-1.82 (m, 1H), |
| | 1.82-1.71 (m, 1H). LC-MS (ESI, m/z): [M + H]⁺ 459. |
| 4-12 | 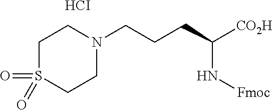 |
| | (S)-2-((((9H-fluroren-9-yl)methoxy)carbonyl)amino)-5-(1,1- |
| | dioxidothiomorpholino)pentanoic acid hydrochloride |
| | (15S)-9-chloro-19-[(3,4-dichlorophenyl)methyl]-6-phenyl-2-oxa- |
| | 5lambda4,13,19lambda4-triaza-1- |
| | nickelapentacyclo[11.6.0.01,5.07,12.015,19]nonadeca-5,7(12),8,10- |
| | tetraene-3,14-dione (CAS: 2089661-84-5) |
| | 4-(3-chloropropyl)thiomorpholine 1,1-dioxide (CAS: 903567-74-8) |
| | ¹H NMR (500 MHz, DMSO-d₆) δ 12.67 (br, 1H), 11.60 (br, 1H), 7.90 (d, |
| | J = 7.5 Hz, 2H), 7.78-7.60 (m, 3H), 7.47-7.38 (m, 2H), 7.38-7.27 (m, 2H), |
| | 4.38-4.15 (m, 3H), 4.05-3.93 (m, 1H), 3.93-2.70 (m, 10H), 1.87-1.44 |
| | (m, 4H). LC-MS (ESI, m/z): [M + H]⁺ 473. |
| 4-13 | 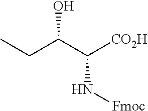 |
| | (2R,3S)-2-((((9H-fluoren-9-yl)methoxy)carbonyl)amino)-3- |
| | hydroxypentanoic acid |
| | (5R)-5-phenylmorpholin-2-one (CAS: 121269-45-2) |
| | Propanal (CAS: 123-38-6) |
| | ¹H NMR (500 MHz, DMSO-d₆) δ 11.62 (br, 1H), 7.92-7.88 (m, 1H), 7.84- |
| | 7.79 (m, 1H), 7.78-7.58 (m, 2H), 7.48-7.37 (m, 2H), 7.36-7.27 (m, 2H), |
| | 7.12-6.99 (m, 1H), 5.12-4.69 (m, 1H), 4.58-4.17 (m, 3H), 4.10-3.93 (m, |
| | 1H), 3.84-3.71 (m, 1H), 1.45-1.28 (m, 2H), 0.89-0.78 (m, 3H). |
| | LC-MS (ESI, m/z): [M + H]⁺ 356. |
| 4-14 | 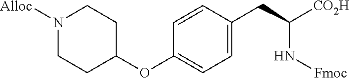 |
| | (S)-2-((((9H-fluoren-9-yl)methoxy)carbonyl)amino)-3-(4-((1- |
| | ((allyloxy)carbonyl)piperidin-4-yl)oxy)phenyl)propanoic acid |
| | (15S)-9-chloro-19-[(3,4-dichlorophenyl)methyl]-6-phenyl-2-oxa- |
| | 5lambda4,13,19lambda4-triaza-1- |
| | nickelapentacyclo[11.6.0.01,5.07,12.015,19]nonadeca-5,7(12),8,10- |
| | tetraene-3,14-dione (CAS: 2089661-84-5) |
| | allyl 4-(4-(chloromethyl)phenoxy)piperidine-1-carboxylate (CAS: |
| | 2803493-36-7) |
| | ¹H NMR (500 MHz, DMSO-d₆) δ 12.73 (br, 1H), 7.89 (d, J = 7.5 Hz, |
| | 2H), 7.82-7.59 (m, 3H), 7.46-7.21 (m, 4H), 7.18 (d, J = 8.5 Hz, 2H), 6.95- |
| | 6.80 (m, 2H), 5.97-5.87 (m, 1H), 5.30-5.15 (m, 2H), 4.55-4.40 (m, 3H), |
| | 4.40-4.09 (m, 4H), 3.72-3.61 (m, 2H), 3.30-3.14 (m, 2H), 3.14-2.86 (m, |
| | 1H), 2.86-2.71 (m, 1H), 1.93-1.79 (m, 2H), 1.56-1.40 (m, 2H). |
| | LC-MS (ESI, m/z): [M + H]⁺ 571. |

### Example-5

The following compounds can be synthesized as outlined for the preparation of Reference example-7-01, Reference example-7-02, Reference example-7-03, Reference example-7-04, or Reference example-7-05 employing appropriate starting materials in the table.

| | Structure and IUPAC name |
|---|---|
| | Starting material-A |
| | Starting material-B |
| Example | NMR and MS |
| 5-01 | 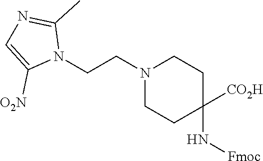 |
| | 4-((((9H-fluoren-9-yl)methoxy)carbonyl)amino)-1-(2-(2- |
| | methyl-5-nitro-1H-imidazol-1-yl)ethyl)piperidine-4-carboxylic |
| | acid4-((((9H-fluoren-9-yl)methoxy)carbonyl)amino)piperidine- |
| | 4-carboxylic acid (CAS: 436867-70-8) |
| | 1-(2-bromoethyl)-2-methyl-5-nitro-imidazole (CAS: 6058-57-7) |
| | ¹H NMR (500 MHz, DMSO-d₆) δ 12.39 (br, 1 H), 8.01 (s, 1 H), |
| | 7.93-7.86 (m, 2 H), 7.77-7.69 (m, 2 H), 7.56 (br, 1 H), 7.45- |
| | 7.38 (m, 2 H), 7.37-7.30 (m, 2 H), 4.43-4.32 (m, 2 H), 4.28- |
| | 4.19 (m, 3 H), 2.65-2.53 (m, 4 H), 2.48-2.45 (m, 3 H), |
| | 2.33-2.18 (m, 2 H), 2.00-1.74 (m, 4 H). |
| | LC-MS (ESI, m/z): [M + H]⁺ 520. |
| 5-02 | 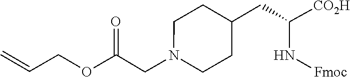 |
| | (R)-2-((((9H-fluoren-9-yl)methoxy)carbonyl)amino)-3-(1-(2- |
| | (allyloxy)-2-oxoethyl)piperidin-4-yl)propanoic acid |
| | (R)-2-((((9H-fluoren-9-yl)methoxy)carbonyl)amino)-3-(1-(tert- |
| | butoxycarbonyl)piperidin-4-yl)propanoic acid (CAS: 313052- |
| | 00-5) |
| | 2-Propen-1-yl 2-bromoacetate (CAS: 40630-84-0) |
| | ¹H NMR (500 MHz, DMSO-d₆) δ 12.49 (br, 1H), 7.98-7.81 |
| | (m, 2H), 7.79-7.56 (m, 3H), 7.50-7.23 (m, 4H), 5.99-5.82 |
| | (m, 1H), 5.41-5.16 (m, 2H), 4.67-4.51 (m, 2H), 4.40-4.16 (m, |
| | 3H), 4.06-3.84 (m, 1H), 3.25 (s, 2H), 2.93-2.71 (m, 2H), 2.27- |
| | 1.98 (m, 2H), 1.72-1.42 (m, 4H), 1.42-0.98 (m, |
| | 3H). LC-MS (ESI, m/z): [M + H]⁺ 493. |
| 5-03 | 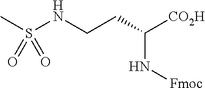 |
| | (R)-2-(((9H-fluoren-9-yl)methoxy)carbonyl)amino)- |
| | 4-(methylsulfonamido)butanoic acid |
| | (2R)-4-Amino-2-[[(9H-fluoren-9-ylmethoxy)carbonyl] |
| | amino]butanoic acid (CAS: 201484-12-0) |
| | Methansulfonyl Chloride (CAS: 124-63-0) |
| | ¹H NMR (400 MHz, DMSO-d₆) δ 12.64 (s, 1H), 7.89 |
| | (d, J = 7.4 Hz, 2H), 7.79-7.58 (m, 3H), 7.51-7.24 |
| | (m, 4H), 7.15-6.97 (m, 1H), 4.43-3.98 (m, |
| | 4H), 3.15-2.95 (m, 2H), 2.88 (s, 3H), 2.02-1.69 (m, 2H). |
| | LC-MS (ESI, m/z): [M + H]⁺ 419. |
| 5-04 | 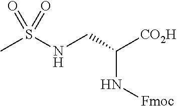 |
| | (R)-2-((((9H-fluoren-9-yl)methoxy)carbonyl)amino)- |
| | 3-(methylsulfonanmido)propanoic acid |
| | (2R)-3-amino-2-({[(9H-fluoren-9-yl)methoxy]carbonyl} |
| | amino)propanoic acid (CAS: 251317-00-7) |
| | Methansulfonyl Chloride (CAS: 124-63-0) |
| | ¹H NMR (400 MHz, DMSO-d₆) δ 12.85 (br, 1H), 7.90 |
| | (d, J = 7.5 Hz, 2H), 7.72 (d, J = 7.5 Hz, 2H), 7.54 (d, |
| | J = 8.5 Hz, 1H), 7.45-7.40 (m, 2H), 7.38-7.25 (m, |
| | 2H), 7.14 (s, 1H), 4.32 (d, J = 6.9 Hz, 2H), 4.25-4.22 |
| | (m, 1H), 4.18-4.05 (m, 1H), 3.31-3.17 (m, 2H), |
| | 2.89 (s, 3H). LC-MS (ESI, m/z): |
| | [M − tBu + H]⁺ 405. |
| 5-05 | 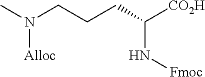 |
| | (R)-2-((((9H-fluoren-9-yl)methoxy)carbonyl)amino)- |
| | 5-(((allyloxy)carbonyl)(methyl)amino)pentanoic acid |
| | (R)-2-((((9H-fluoren-9-yl)methoxy)carbonyl)amino)-5- |
| | (methylamino)pentanoic acid (CAS: 2415918-40-8) |
| | Allyl Chloroformate (CAS: 2937-50-0) |
| | ¹H NMR (500 MHz, DMSO-d₆) δ 12.60 (br, 1H), |
| | 7.89 (d, J = 7.5 Hz, 2H), 7.75-7.70 (m, 2H), 7.69- |
| | 7.61 (m, 1H), 7.45-7.38 (m, 2H), 7.35-7.30 (m, |
| | 2H), 5.97-5.86 (m, 1H), 5.32-5.21 (m, 1H), 5.20- |
| | 5.12 (m, 1H), 4.54-4.46 (m, 2H), 4.33-4.20 (m, 3H), |
| | 4.00-3.92 (m, 1H), 3.29-3.15 (m, 2H), 2.88-2.76 (m, 3H), |
| | 1.68-1.47 (m, 4H). LC-MS (ESI, m/z): [M + H]⁺ 453. |
| 5-06 | 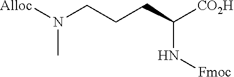 |
| | (S)-2-((((9H-fluoren-9-yl)methoxy)carbonyl)amino)-5- |
| | (((allyloxy)carbonyl)(methyl)amino)pentanoic acid |
| | (S)-2-((((9H-fluoren-9-yl)methoxy)carbonyl)amino)-5- |
| | (methylamino)pentanoic acid (CAS: 1659291-26-5) |
| | Allyl Chloroformate (CAS: 2937-50-0) |
| | Substantially identical to Example 5-05 |
| 5-07 | 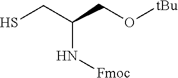 |
| | (9H-fluoren-9-yl)methyl (R)-(1-(tert-butoxy)-3- |
| | mercaptopropan-2-yl)carbamate |
| | (R)-2-amino-3-(tert-butoxy)propane-1-thiol (CAS: 2350294- |
| | 34-5) |
| | N-(9-Fluorenylmethoxycarbonyloxy)succinimide (CAS: |
| | 82911-69-1) |
| | ¹H NMR (300 MHz, METHANOL-d₄) δ 7.82 (d, J = 7.4 Hz, |
| | 2H), 7.68 (d, J = 7.4 Hz, 2H), 7.47-7.27 (m, 4H), |
| | 4.50-4.13 (m, 3H), 3.77-3.69 (m, 1H), 3.78-3.63 (m, |
| | 1H), 3.59-3.36 (m, 2H), 2.85-2.35 (m, 2H), 1.20 (s, 9H). |
| | LC-MS (ESI, m/z): [M + H]⁺ 386. |
| 5-08 | 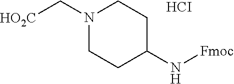 |
| | 2-(4-((((9H-fluoren-9-yl)methoxy)carbonyl)amino)piperidin- |
| | 1-yl)acetic acid hydrochloride |
| | 2-(4-aminopiperidin-1-yl)acetic acid (CAS: 805179-70-8) |
| | N-[(9H-Fluoren-9-ylmethoxy)carbonyloxy]succinimide (CAS: |
| | 82911-69-1) |
| | ¹H NMR (500 MHz, DMSO-d₆) δ 14.01 (br, 1H), 9.99 (br, 1H), |
| | 7.90 (d, J = 7.5 Hz, 2H), 7.69 (d, J = 7.3 Hz, 2H), |
| | 7.51 (d, J = 6.6 Hz, 1H), 7.47-7.40 (m, 2H), 7.35-7.19 (m, 2H), |
| | 4.52-4.16 (m, 3H), 4.06 (s, 2H), 3.65-3.28 (m, 5H), 2.03- |
| | 1.86 (m, 2H), 1.84-1.69 (m, 2H). LC-MS (ESI, m/z): |
| | [M + H]⁺ 381. |

### Example-6

The following compounds can be synthesized as outlined for the preparation of Reference example-8-01, Reference example-8-02, or Reference example-8-03 employing appropriate starting materials in the table.

| | Structure and IUPAC name |
|---|---|
| | Starting material-A |
| | Starting material-B |
| Example | NMR and MS |
| 6-01 | 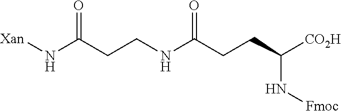 |
| | (2S)-2-({[(9H-fluoren-9-yl)methoxy]carbonyl}amino)-4-({2-[(9H-xanthen-9-yl) |
| | carbamoyl]ethyl}carbamoyl)butanoic acid |
| | (4S)-4-({[(9H-fluoren-9-yl)methoxy]carbonyl}amino)-5-oxo-5-(prop-2-en-1-yloxy) |
| | pentanoic acid (CAS: 144120-54-7) |
| | 3-Amino-N-9H-xanthen-9-ylpropanamide (CAS: 1606262-06-9) |
| | ¹H NMR (500 MHz, DMSO-d₆) δ 12.65 (br, 1H), 8.89-8.83 (m, 1H), 7.99- |
| | 7.92 (m, 1H), 7.92-7.86 (m, 2H), 7.77-7.69 (m, 2H), 7.66-7.56 (m, 1H), |
| | 7.45-7.39 (m, 2H), 7.39-7.36 (m, 2H), 7.35-7.29 (m, 4H), 7.16-7.10 (m, |
| | 4H), 6.32-6.28 (m, 1H), 4.31-4.20 (m, 3H), 3.98-3.89 (m, 1H), 3.31- |
| | 3.23 (m, 2H), 2.35-2.28 (m, 2H), 2.21-2.10 (m, 2H), 2.01-1.91 (m, 1H), |
| | 1.83-1.74 (m, 1H). LC-MS (ESI, m/z): [M + H]⁺ 620. |
| 6-02 | 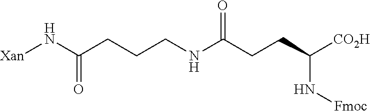 |
| | (2S)-2-({[(9H-fluoren-9-yl)methoxy]carbonyl}amino)-4-({3-[(9H- |
| | xanthen-9-yl)carbamoyl]propyl}carbamoyl)butanoic acid |
| | (4S)-4-({[(9H-fluoren-9-yl)methoxy]carbonyl}amino)-5-oxo-5-(prop-2-en- |
| | 1-yloxy)pentanoic acid (CAS: 144120-54-7) |
| | 4-Amino-N-9H-xanthen-9-ylbutanamide (CAS: 1583447-19-1) |
| | ¹H NMR (500 MHz, DMSO-d₆) δ 12.78 (br, 1H), 8.79 (d, J = 8.8 Hz, 1H), |
| | 7.94-7.83 (m, 3H), 7.71 (d, J = 7.5 Hz, 2H), 7.46-7.27 (m, 9H), 7.18-7.08 |
| | (m, 4H), 6.28 (d, J = 8.7 Hz, 1H), 4.33-4.16 (m, 3H), 3.97-3.80 (m, 1H), |
| | 3.09-2.99 (m, 2H), 2.20-2.08 (m, 4H), 2.05-1.89 (m, 1H), 1.86-1.63 (m, |
| | 3H). LC-MS (ESI, m/z): [M + H]⁺ 634. |

### Example-7-01: 2-[(2-{4-[(tert-butoxy)carbonyl]phenyl}ethyl)({[(9H-fluoren-9-yl) methoxy]carbonyl})amino]acetic acid

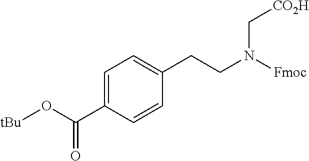

To a suspension of dibromonickel trihydrate (CAS: 7789-49-3, 4.64 g, 17.0 mmol) in DMA (300 mL) at rt was added 4,4′-di-tert-butyl-2,2′-bipyridine (CAS: 72914-19-3, 4.57 g, 17.0 mmol). The mixture was stirred at rt for 15 min. To the mixture at rt were added tert-butyl 4-bromobenzoate (CAS: 59247-47-1, 29.2 g, 114 mmol), Reference Example-9-04 (30 g, 56.8 mmol), and Zn (18.6 g, 283 mmol). The mixture was stirred at rt for an additional 0.5 h. The reaction was quenched by the addition of water at rt. The mixture was filtered. The filtrate was extracted three times with EtOAc. The combined organic extracts were washed with brine, dried over Na₂SO₄, filtered, and then concentrated. The resulting residue was purified by silica gel flash column chromatography (isocratic, petroleum ether/EtOAc=1/1). Appropriate fractions were concentrated. To a mixture of the obtained material (20 g, 38.8 mmol) in 2-propanol (600 mL) at 0° C. were added a solution of CaCl₂ (68.9 g, 620 mmol) in H₂O (150 mL) and a solution of LiOH (3.72 g, 155 mmol) in H₂O (50 mL). The mixture was stirred at rt for 16 h. The pH of the mixture was adjusted to pH 5 with satd. aq. NaH₂PO₄. The resulting mixture was extracted three times with EtOAc. The combined organic extracts were washed with brine, dried over Na₂SO₄, filtered, and then concentrated. The resulting residue was purified by reversed phase silica gel flash column chromatography (C18 silica gel, gradient, 10 mM aq. NH₄HCO₃/CH₃CN=95/5 to 0/100) to afford the title compound (10.2 g, 20.3 mmol). ¹H NMR (500 MHz, DMSO-d₆) δ 12.68 (br, 1H), 7.92-7.86 (m, 2H), 7.86-7.55 (m, 4H), 7.47-7.00 (m, 6H), 4.52-4.45 (m, 1H), 4.28-4.14 (m, 2H), 4.00-3.69 (m, 2H), 3.56-3.07 (m, 2H), 2.91-2.41 (m, 2H), 1.55-1.51 (m, 9H). LC-MS (ESI, m/z): [M+Na]⁺ 524.

### Example-7

The following compounds can be synthesized as outlined for the preparation of Reference example-9-01, Reference example-9-02, Reference example-9-03, or Example-7-01 employing appropriate starting materials in the table.

| | Structure and IUPAC name |
|---|---|
| | Starting material-A |
| | Starting material-B |
| Example | NMR and MS |
| 7-02 | 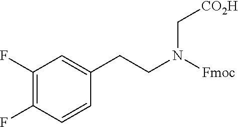 |
| | 2-{[2-(3,4-difluorophenyl)ethyl]({[(9H-fluoren-9-yl)methoxy]carbonyl}) |
| | amino}acetic acid |
| | 2-(3,4-difluorophenyl)ethan-1-amine (CAS: 220362-31-2) |
| | 2-oxoacetic acid hydrate (CAS: 1152495-30-1) |
| | ¹H NMR (500 MHz, DMSO-d₆) δ 12.67 (br, 1H), 7.89 (d, J = 7.6 Hz, 2H), |
| | 7.68-7.55 (m, 2H), 7.45-7.38 (m, 2H), 7.37-6.64 (m, 5H), 4.56 (d, J = |
| | 5.0 Hz, 1H), 4.31-4.15 (m, 2H), 3.98-3.71 (m, 2H), 3.50-3.00 (m, 2H), |
| | 2.84-2.28 (m, 2H). LC-MS (ESI, m/z): [M + H]⁺ 438. |
| 7-03 | 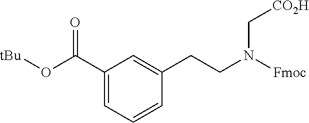 |
| | 2-[(2-{3-[(tert-butoxy)carbonyl]phenyl}ethyl)({[(9H-fluoren-9-yl)methoxy]carbonyl}) |
| | amino]acetic acid |
| | Reference example 9-04 |
| | tert-Butyl 3-bromobenzoate (CAS: 69038-74-0) |
| | ¹H NMR (500 MHz, DMSO-d₆) δ 12.70 (br, 1H), 7.92-7.82 (m, 2H), 7.81- |
| | 7.55 (m, 4H), 7.52-7.10 (m, 6H), 4.51-4.32 (m, 1H), 4.32-4.09 (m, 2H), |
| | 4.09-3.74 (m, 2H), 3.53-3.12 (m, 2H), 2.92-2.46 (m, 2H), 1.55-1.47 (m, |
| | 9H). LC-MS (ESI, m/z): [M − tBu + H]⁺ 446. |
| 7-04 | 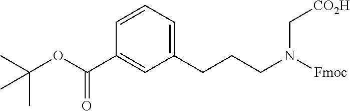 |
| | 2-[(3-{3-[(tert-butoxy)carbonyl]phenyl}propyl)({[(9H-fluoren-9-yl)methoxy] |
| | carbonyl})amino]acetic acid |
| | tert-butyl 3-(3-aminopropyl)benzoate (CAS: 2639434-60-7) |
| | 2-oxoacetic acid hydrate (CAS: 1152495-30-1) |
| | ¹H NMR (500 MHz, DMSO-d₆) δ 12.72 (br, 1H), 7.94-7.83 (m, 2H), 7.77- |
| | 7.55 (m, 4H), 7.52-7.20 (m, 6H), 4.45 (d, J = 5.5 Hz, 1H), 4.33-4.15 (m, |
| | 2H), 4.01-3.77 (m, 2H), 3.34-2.95 (m, 2H), 2.68-2.27 (m, 2H), 1.85- |
| | 1.44 (m, 11H). LC-MS (ESI, m/z): [M − tBu + H]⁺ 460. |
| 7-05 | 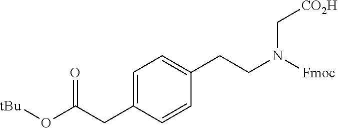 |
| | 2-[(2-{4-[2-(tert-butoxy)-2-oxoethyl]phenyl}ethyl)({[(9H-fluoren-9- |
| | yl)methoxy]carbonyl})amino]acetic acid |
| | Reference example 9-04 |
| | tert-butyl 2-(4-bromophenyl)acetate (CAS: 33155-58-7) |
| | ¹H NMR (500 MHz, DMSO-d₆) δ 12.73 (br, 1H), 7.99-6.81 (m, 12H), 4.57- |
| | 4.48 (m, 1H), 4.35-4.15 (m, 2H), 3.93-3.72 (m, 2H), 3.65-3.41 (m, 3H), |
| | 3.16-3.04 (m, 1H), 3.00-2.70 (m, 1H), 2.43-2.33 (m, 1H), 1.46-1.06 (m, |
| | 9H). LC-MS (ESI, m/z): [M − tBu + H]⁺ 460. |
| 7-06 | 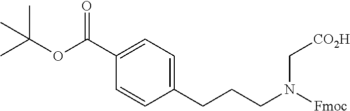 |
| | 2-[(3-{4-[(tert-butoxy)carbonyl]phenyl}propyl)({[(9H-fluoren-9- |
| | yl)methoxy]carbonyl})amino]acetic acid |
| | tert-butyl 4-(3-aminopropyl)benzoate (CAS: 2639426-54-1) |
| | 2-oxoacetic acid hydrate (CAS: 1152495-30-1) |
| | ¹H NMR (500 MHz, DMSO-d₆) δ 13.00-12.48 (br, 1H), 7.91-7.85 (m, |
| | 2H), 7.85-7.76 (m, 2H), 7.64-7.58 (m, 2H), 7.44-7.35 (m, 2H), 7.35- |
| | 7.28 (m, 3H), 7.21 (d, J = 8.1 Hz, 1H), 4.45 (d, J = 5.5 Hz, 1H), 4.30-4.17 |
| | (m, 2H), 3.95 (s, 1H), 3.81 (s, 1H), 3.32-3.25 (m, 2H), 2.99 (t, J = 7.3 Hz, |
| | 1H), 2.62 (t, J = 7.7 Hz, 1H), 2.33 (t, J = 7.9 Hz, 1H), 1.78 (t, J = 7.3 Hz, |
| | 1H), 1.56-1.49 (m, 9H). LC-MS (ESI, m/z): [M − tBu + H]⁺ 460. |
| 7-07 | 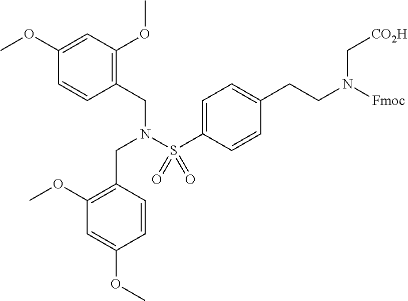 |
| | 2-{[2-(4-{bis[(2,4- |
| | dimethoxyphenyl)methyl]sulfamoyl}phenyl)ethyl]({[(9H-fluoren-9- |
| | yl)methoxy]carbonyl})amino}acetic acid |
| | Reference example 9-04 |
| | 4-Bromo-N,N-bis[(2,4-dimethoxyphenyl)methyl]benzenesulfonamide |
| | (CAS: 1440421-56-6) |
| | ¹H NMR (500 MHz, DMSO-d₆) δ 12.96-12.53 (br, 1H), 7.93-7.85 (m, |
| | 2H), 7.70-7.55 (m, 4H), 7.44-7.30 (m, 5H), 7.07-6.93 (m, 3H), 6.40- |
| | 6.34 (m, 4H), 4.58 (d, J = 4.9 Hz, 1H), 4.33-4.16 (m, 6H), 4.01-3.79 (m, |
| | 2H), 3.72-3.67 (m, 6H), 3.60-3.54 (m, 6H), 3.49 (t, J = 7.6 Hz, 1H), 3.14- |
| | 3.02 (m, 1H), 2.88 (t, J = 7.6 Hz, 1H), 2.47-2.39 (m, 1H). LC-MS (ESI, |
| | m/z): Not Found. |

### Example-8

The following compounds can be synthesized as outlined for the preparation of Reference example-10-01, Reference example-10-02, Reference example-10-03, Reference example-10-04, or Reference example-6-05 employing appropriate starting materials in the table.

| | Structure and IUPAC name |
|---|---|
| | Starting material-A |
| | Starting material-B |
| Example | NMR and MS |
| 8-01 | 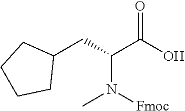 |
| | (R)-2-((((9H-fluoren-9-yl)methoxy)carbonyl)(methyl)amino)-3- |
| | cyclopentylpropanoic acid |
| | (2R)-3-cyclopentyl-2-({[(9H-fluoren-9- |
| | yl)methoxy]carbonyl}amino)propanoic acid (CAS: 1262802-59-4) |
| | Paraformaldehyde (CAS: 30525-89-4) |
| | ¹H NMR (500 MHz, DMSO-d₆) δ 12.73 (br, 1H), 7.93-7.84 (m, 2H), 7.68- |
| | 7.60 (m, 2H), 7.45-7.37 (m, 2H), 7.35-7.28 (m, 2H), 4.60-4.39 (m, 2H), |
| | 4.37-4.22 (m, 2H), 2.74-2.68 (m, 3H), 1.85-1.33 (m, 9H), 1.13-0.93 (m, |
| | 2H). LC-MS (ESI, m/z): [M + H]⁺ 394. |
| 8-02 | 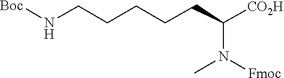 |
| | (S)-2-((((9H-fluoren-9-yl)methoxy)carbonyl)(methyl)amino)-7-((tert- |
| | butoxycarbonyl)amino)heptanoic acid |
| | (2S)-2-({[(9H-fluoren-9-yl)methoxy]carbonyl}amino)-7-{[(prop-2-en-1- |
| | yloxy)carbonyl]amino}heptanoic acid (CAS: 281655-70-7) |
| | Paraformaldehyde (CAS: 30525-89-4) |
| | ¹H NMR (500 MHz, DMSO-d₆) δ 12.75 (br, 1H), 7.94-7.85 (m, 2H), 7.70- |
| | 7.58 (m, 2H), 7.45-7.37 (m, 2H), 7.36-7.28 (m, 2H), 6.77 (br, 1H), 4.53- |
| | 4.23 (m, 4H), 2.94-2.84 (m, 2H), 2.75-2.68 (m, 3H), 1.81-1.48 (m, 2H), |
| | 1.37 (s, 15H). LC-MS (ESI, m/z): [M − Boc + H]⁺ 397. |
| 8-03 | 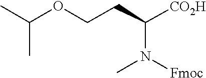 |
| | (2S)-2-({[(9H-fluoren-9-yl)methoxy]carbonyl}(methyl)amino)-4-(propan- |
| | 2-yloxy)butanoic acid |
| | (2S)-2-({[(9H-fluoren-9-yl)methoxy]carbonyl}amino)-4-(propan-2- |
| | yloxy)butanoic acid (CAS: 2350000-84-7) |
| | Paraformaldehyde (CAS: 30525-89-4) |
| | ¹H NMR (500 MHz, DMSO-d₆) δ 12.79 (br, 1H), 7.94-7.87 (m, 2H), 7.68- |
| | 7.61 (m, 2H), 7.46-7.38 (m, 2H), 7.37-7.28 (m, 2H), 4.62-4.52 (m, 1H), |
| | 4.41-4.17 (m, 3H), 3.50-3.12 (m, 3H), 2.79-2.72 (m, 3H), 2.08-1.75 (m, |
| | 2H), 1.10-0.97 (m, 6H). LC-MS (ESI, m/z): [M + H]⁺ 398. |
| 8-04 | 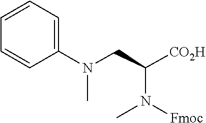 |
| | (S)-2-((((9H-fluoren-9-yl)methoxy)carbonyl)(methyl)amino)-3- |
| | (methyl(phenyl)amino)propanoic acid |
| | (2S)-2-{[(tert-butoxy)carbonyl]amino}-3-[methyl(phenyl)amino]propanoic |
| | acid (CAS: 2680587-49-7) |
| | iodomethane (CAS: 74-88-4) |
| | ¹H NMR (500 MHz, DMSO-d₆) δ 13.02 (br, 1H), 7.93-7.82 (m, 2H), 7.60- |
| | 7.12 (m, 8H), 6.72-6.55 (m, 3H), 4.75-4.60 (m, 1H), 4.38-4.14 (m, 3H), |
| | 3.95-3.71 (m, 2H), 2.82-2.70 (m, 6H). LC-MS (ESI, m/z): [M + H]⁺ 431. |
| 8-05 | 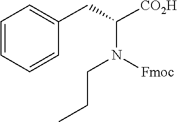 |
| | (2R)-2-({[(9H-fluoren-9-yl)methoxy]carbonyl}(propyl)amino)-3- |
| | phenylpropanoic acid |
| | (2R)-2-({[(9H-fluoren-9-yl)methoxy]carbonyl}amino)-3-phenylpropanoic |
| | acid (CAS: 86123-10-6) |
| | Propanal (CAS: 123-38-6) |
| | ¹H NMR (500 MHz, DMSO-d₆) δ 12.59 (br, 1H), 7.90-7.84 (m, 2H), 7.71- |
| | 7.58 (m, 2H), 7.44-7.36 (m, 2H), 7.36-7.30 (m, 2H), 7.23-6.85 (m, 5H), |
| | 4.62-4.51 (m, 1H), 4.44-4.30 (m, 1H), 4.29-4.15 (m, 2H), 3.17-2.89 (m, |
| | 2H), 2.71-2.56 (m, 1H), 2.37-2.27 (m, 1H), 1.21-1.06 (m, 1H), 0.83-0.76 |
| | (m, 1H), 0.64-0.56 (m, 1H), 0.44-0.33 (m, 2H). LC-MS (ESI, m/z): [M + H]⁺ 430. |
| 8-06 | 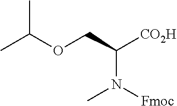 |
| | (2S)-2-({[(9H-fluoren-9-yl)methoxy]carbonyl}(methyl)amino)-3-(propan- |
| | 2-yloxy)propanoic acid |
| | (2S)-2-({[(9H-fluoren-9-yl)methoxy]carbonyl}amino)-3-(propan-2- |
| | yloxy)propanoic acid (CAS: 2137146-08-6) |
| | Paraformaldehyde (CAS: 30525-89-4) |
| | ¹H NMR (500 MHz, DMSO-d₆) δ 12.91 (br, 1H), 7.95-7.85 (m, 2H), 7.69- |
| | 7.61 (m, 2H), 7.46-7.38 (m, 2H), 7.37-7.27 (m, 2H), 4.71-4.63 (m, 1H), |
| | 4.38-4.20 (m, 3H), 3.81-3.64 (m, 2H), 3.60-3.46 (m, 1H), 2.89-2.80 (m, |
| | 3H), 1.08-1.03 (m, 6H). LC-MS (ESI, m/z): [M + H]⁺ 384. |
| 8-07 | 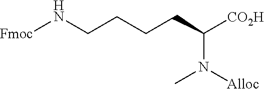 |
| | (2S)-6-({[(9H-fluoren-9-yl)methoxy]carbonyl}amino)-2-{methyl[(prop-2- |
| | en-1-yloxy)carbonyl]amino}hexanoic acid |
| | (2S)-6-({[(9H-fluoren-9-yl)methoxy]carbonyl}amino)-2-{[(prop-2-en-1- |
| | yloxy)carbonyl]amino}hexanoic acid (CAS: 186305-56-1) |
| | Paraformaldehyde (CAS: 30525-89-4) |
| | ¹H NMR (400 MHz, DMSO-d₆) δ 12.80 (br, 1H), 7.89 (d, J = 7.6 Hz, 2H), |
| | 7.68 (d, J = 7.5 Hz, 2H), 7.45-7.38 (m, 2H), 7.36-7.28 (m, 3H), 5.97-5.83 |
| | (m, 1H), 5.30-5.12 (m, 2H), 4.59-4.38 (m, 3H), 4.33-4.24 (m, 2H), 4.23- |
| | 4.18 (m, 1H), 3.04-2.88 (m, 2H), 2.80-2.73 (m, 3H), 1.86-1.76 (m, 1H), |
| | 1.76-1.65 (m, 1H), 1.50-1.32 (m, 2H), 1.29-1.11 (m, 2H). LC-MS (ESI, m/z): |
| | [M + H]⁺ 467. |

### Example-9

The following compounds can be synthesized as outlined for the preparation of Reference example-17-01 employing appropriate starting materials in the table.

| | Structure and IUPAC name |
|---|---|
| | Starting material-A |
| | Starting material-B |
| Example | NMR and MS |
| 9-01 | 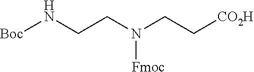 |
| | 3-((((9H-fluoren-9-yl)methoxy)carbonyl)(2-((tert-butoxycarbonyl)amino)ethyl)amino) |
| | propanoic acid |
| | tert-butyl (2-aminoethyl)carbamate (CAS: 57260-73-8) |
| | Acrylic acid (CAS: 79-10-7) |
| | ¹H NMR (500 MHz, DMSO-d₆) δ 12.23 (br, 1H), 7.94-7.82 (m, 2H), 7.71- |
| | 7.57 (m, 2H), 7.46-7.35 (m, 2H), 7.35-7.26 (m, 2H), 6.88-6.39 (m, 1H), |
| | 4.43-4.23 (m, 3H), 3.43-3.30 (m, 1H), 3.30-3.05 (m, 3H), 3.05-2.83 (m, |
| | 2H), 2.48-2.12 (m, 2H), 1.45-1.20 (m, 9H). LC-MS (ESI, m/z): [M + H]⁺ 455. |
| 9-02 | 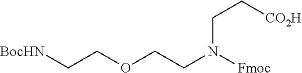 |
| | 11-(((9H-fluoren-9-yl)methoxy)carbonyl)-2,2-dimethyl-4-oxo-3,8-dioxa- |
| | 5,11-diazatetradecan-14-oic acid |
| | tert-butyl (2-(2-aminoethoxy)ethyl)carbamate (CAS: 127838-22-2) |
| | Acrylic acid (CAS: 79-10-7) |
| | ¹H NMR (500 MHz, DMSO-d₆) δ 12.26 (br, 1H), 7.88 (dd, J = 7.5, 7.5 Hz, |
| | 2H), 7.63 (d, J = 7.5 Hz, 2H), 7.41 (dd, J = 7.5, 7.0 Hz, 2H), 7.37-7.29 |
| | (m, 2H), 6.75 (t, J = 5.5 Hz, 1H), 4.49-4.34 (m, 2H), 4.34-4.23 (m, 1H), |
| | 3.46-3.15 (m, 6H), 3.14-2.97 (m, 4H), 2.38 (dd, J = 7.5, 7.0 Hz, 1H), 2.20 |
| | (dd, J = 7.5, 7.0 Hz, 1H), 1.41-1.30 (m, 9 H). LC-MS (ESI, m/z): [M + H]⁺ 499. |
| 9-03 | 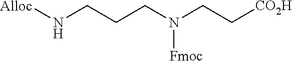 |
| | 3-((((9H-fluoren-9-yl)methoxy)carbonyl)(3- |
| | (((allyloxy)carbonyl)amino)propyl)amino)propanoic acid |
| | 2-Propen-1-yl N-(3-aminopropyl)carbamate (CAS: 503817-47-8) |
| | Acrylic acid (CAS: 79-10-7) |
| | ¹H NMR (500 MHz, DMSO-d₆) δ 12.24 (br, 1H), 7.95-6.72 (m, 9H), 5.96- |
| | 5.80 (m, 1H), 5.30-5.06 (m, 2H), 4.51-4.20 (m, 5H), 3.31-2.67 (m, 6H), |
| | 2.43-2.15 (m, 2H), 1.62-1.23 (m, 2H). LC-MS (ESI, m/z): [M + H]⁺ 453. |
| 9-04 | 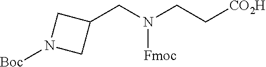 |
| | 3-((((9H-fluoren-9-yl)methoxy)carbonyl)((1-(tert-butoxycarbonyl)azetidin- |
| | 3-yl)methyl)amino)propanoic acid |
| | tert-Butyl 3-(aminomethyl)azetidine-1-carboxylate (CAS: 325775-44-8) |
| | Acrylic acid (CAS: 79-10-7) |
| | ¹H NMR (500 MHz, DMSO-d₆) δ 12.25 (br, 1H), 7.95-7.85 (m, 2H), 7.63 |
| | (d, J = 7.5 Hz, 2H), 7.44-7.36 (m, 2H), 7.35-7.27 (m, 2H), 4.60-4.39 (m, |
| | 2H), 4.31-4.24 (m, 1H), 3.86-3.37 (m, 4H), 3.31-3.07 (m, 4H), 2.75- |
| | 2.52 (m, 1H), 2.41-2.25 (m, 1H), 2.20-2.12 (m, 1H), 1.37 (s, 9H). |
| | LC-MS (ESI, m/z): [M + H]⁺ 481. |
| 9-05 | 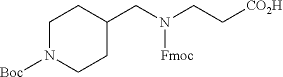 |
| | 3-((((9H-fluoren-9-yl)methoxy)carbonyl)((1-(tert- |
| | butoxycarbonyl)piperidin-4-yl)methyl)amino)propanoic acid |
| | tert-butyl 4-(aminomethyl)piperidine-1-carboxylate (CAS: 144222-22-0) |
| | Acrylic acid (CAS: 79-10-7) |
| | ¹H NMR (500 MHz, DMSO-d₆) δ 12.22 (br, 1H), 7.86 (d, J = 7.5 Hz, 2H), |
| | 7.63 (d, J = 7.5 Hz, 2 H), 7.39 (t, J = 7.1 Hz, 2H), 7.35-7.29 (m, 2H), 4.60 (d, |
| | J = 3.5 Hz, 1H), 4.26 (br, 1H), 3.87 (br, 1H), 3.78 (br, 1H), 3.26-3.13 (m, |
| | 2H), 3.01 (d, J = 6.4 Hz, 1H), 2.71-2.52 (m, 2H), 2.37 (br, 3H), 2.22- |
| | 2.10 (m, 1H), 1.48-1.31 (m, 10H), 1.22-1.10 (m, 1H), 1.05 (d, J = 11.1 Hz, |
| | 1H), 0.95-0.79 (m, 1H), 0.52-0.39 (m, 1H). LC-MS (ESI, m/z): [M + H]⁺ 509. |

### Example-10

The following compounds can be synthesized as outlined for the preparation of Reference example-13-01, Reference example-13-02, or Reference example-13-30 employing appropriate starting materials in the table.

| | Structure and IUPAC name |
|---|---|
| | Starting material-A |
| | Starting material-B |
| Example | NMR and MS |
| 10-01 | 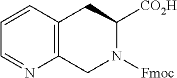 |
| | (S)-7-(((9H-fluoren-9-yl)methoxy)carbonyl)-5,6,7,8- |
| | tetrahydro-1,7-naphthyridine-6-carboxylic acid |
| | (2S)-2-amino-3-(pyridin-3-yl)propanoic acid |
| | (CAS: 64090-98-8) |
| | ethyl carbonochloridate (CAS: 541-41-3) |
| | ¹H NMR (500 MHz, DMSO-d₆) δ 13.00 (br, 1H), 8.44- |
| | 8.39 (m, 1H), 7.957.86 (m, 2H), 7.72-7.63- |
| | (m, 3H), 7.46-7.38 (m, 2H), 7.37-7.29 (m, 2H), 7.27-7.21 |
| | (m, 1H), 5.02-4.96 (m, 1H), 4.70-4.60 (m, 1H), 4.50-4.29 (m, |
| | 4H), 3.25-3.12 (m, 2H). LC-MS (ESI, m/z): [M + H]⁺ 401. |

### Example-11

The following compounds can be synthesized as outlined for the preparation of Reference example-15-01 employing appropriate starting materials in the table.

| | Structure and IUPAC name |
|---|---|
| | Starting material-A |
| | Starting material-B |
| Example | NMR and MS |
| 11-01 | 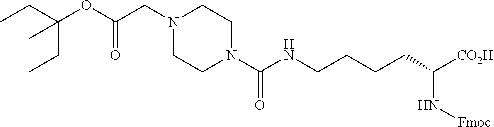 |
| | (2R)-2-({[(9H-fluoren-9-yl)methoxy]carbonyl}amino)-6-[(4-{2-[(3- |
| | methylpentan-3-yl)oxy]-2-oxoethyl}piperazine-1- |
| | carbonyl)amino]hexanoic acid |
| | (2R)-6-amino-2-({[(9H-fluoren-9-yl)methoxy]carbonyl}amino)hexanoic |
| | acid hydrochloride (CAS: 201002-47-3) |
| | 3-methylpentan-3-yl 2-(piperazin-1-yl)acetate (CAS: 2973756-40-8) |
| | ¹H NMR (500 MHz, DMSO-d₆) δ 12.58 (br, 1H), 7.90 (d, J = 7.5 Hz, 2H), |
| | 7.72 (d, J = 7.6 Hz, 2H), 7.57-7.45 (m, 1H), 7.42 (t, J = 7.5 Hz, 2H), 7.33 |
| | (t, J = 7.4 Hz, 2H), 6.52-6.41 (m, 1H), 4.33-4.17 (m, 3H), 3.92-3.79 (m, |
| | 1H), 3.27-3.22 (m, 4H), 3.12 (s, 2H), 3.04-2.93 (m, 2H), 2.46-2.38 (m, |
| | 4H), 1.86-1.75 (m, 2H), 1.74-1.53 (m, 4H), 1.44-1.21 (m, 7H), 0.79 (t, J = |
| | 7.5 Hz, 6H). LC-MS (ESI, m/z): [M + H]⁺ 623. |

### Example-12-01: (2S)-2-((((9H-fluoren-9-yl)methoxy)carbonyl)amino)-3-(4-(3-((tetrahydro-2H-pyran-2-yl)oxy)phenyl)piperidin-1l-yl)propanoic acid

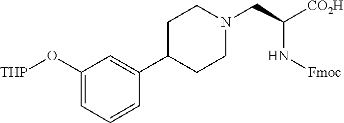

To a solution of 3-(4-Piperidinyl)phenol (CAS: 110878-71-2, 1.75 g, 9.88 mmol) in DCM (19 mL) at rt was added 2,2,2-trifluoro-N-methyl-N-(trimethylsilyl)acetamide (CAS: 24589-78-4, 4.14 ml, 22.5 mmol). To a solution of Reference example-3-02 (4 g, 8.98 mmol) in DCM (19 ml) and acetonitrile (6 ml) at 0° C. was added a solution of the obtained material as above in DCM, and then stirred at 15° C. for 7 h. To the resulting mixture at 0° C. was added heptane (60 mL), and then stirred at 0° C. for 1 h. The resulting solid was corrected. To a mixture of the obtained material (2.66 g, 4.27 mmol) in DCM (14 mL) at 0° C. was added TFA (7.1 mL). The resulting mixture was stirred at rt overnight, and then concentrated. The resulting mixture was dissolved with DCM. The pH of the mixture was adjusted to pH 4 with satd. aq. NaHCO₃. The resulting mixture was extracted three times with DCM. The combined organic extracts were washed with brine, dried over Na₂SO₄, filtered, and then concentrated. To a mixture of the obtained material (1.30 g, 2.67 mmol) in DCE (6.67 ml) at rt were added pyridinium p-toluenesulfonate (CAS: 24057-28-1, 0.34 g, 1.33 mmol) and 3,4-dihydro-2H-pyran (CAS: 110-87-2, 4.88 ml, 53.4 mmol). The resulting mixture was stirred at 75° C. for 13 h. The reaction was quenched with satd. aq. NaHCO₃. The pH of the mixture was adjusted to pH 4 with 1 M aq. HCl and satd. aq. NaHCO₃. The resulting mixture was extracted three times with DCM. The combined organic extracts were washed with brine, dried over Na₂SO₄, filtered, and then concentrated. The resulting residue was purified by silica gel flash column chromatography (gradient, DCM/MeOH=100/0 to 85/15) to afford the title compound (891 mg, 1.56 mmol). ¹H NMR (500 MHz, METHANOL-d₄) δ 7.88-7.76 (m, 2H), 7.72-7.63 (m, 2H), 7.46-7.26 (m, 4H), 7.26-7.17 (m, 1H), 6.99-6.85 (m, 3H), 5.41 (br, 1H), 4.50-4.31 (m, 3H), 4.27-4.20 (m, 1H), 3.93-3.81 (m, 1H), 3.80-3.63 (m, 2H), 3.62-3.52 (m, 1H), 3.38-3.23 (m, 2H), 3.38-3.23 (m, 2H), 3.21-3.03 (m, 2H), 2.91-2.73 (m, 1H), 2.13-1.73 (m, 4H), 1.73-1.45 (m, 4H). LC-MS (ESI, m/z): [M+H]⁺ 571.

### Example-12

The following compounds can be synthesized as outlined for the preparation of Example-12-01 employing appropriate starting materials in the table.

| | Structure and IUPAC name |
|---|---|
| | Starting material-A |
| | Starting material-B |
| Example | NMR and MS |
| 12-02 | 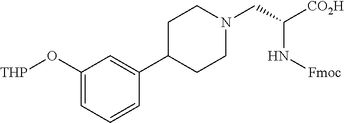 |
| | (2R)-2-((((9H-fluoren-9-yl)methoxy)carbonyl)amino)- |
| | 3-(4-(3-((tetrahydro- |
| | 2H-pyran-2-yl)oxy)phenyl)piperidin-1-yl)propanoic acid |
| | Reference example-3-04 |
| | 3-(4-Piperidinyl)phenol (CAS: 110878-71-2) |
| | Substantially identical to Example 12-01 |

### Example-13

The following compounds can be synthesized as outlined for the preparation of Reference example-19-01 employing appropriate starting materials in the table.

| | Structure and IUPAC name |
|---|---|
| | Starting material-A |
| | Starting material-B |
| Example | NMR and MS |
| 13-01 | 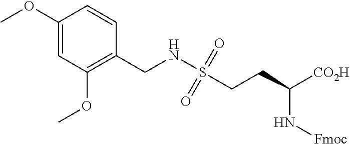 |
| | (S)-2-((((9H-fluoren-9-yl)methoxy)carbonyl)amino)-4-(N-(2,4-dimethoxybenzyl) |
| | sulfamoyl)butanoic acid |
| | (2S)-2-({[(9H-fluoren-9-yl)methoxy]carbonyl}amino)-4- |
| | [(triphenylmethyl)sulfanyl]butanoic acid (CAS: 167015-23-8) |
| | 2,4-Dimethoxybenzylamine (CAS: 20781-20-8) |
| | ¹H NMR (500 MHz, DMSO-d₆) δ 12.84 (br, 1H), 7.90 (d, J = 7.5 Hz, 2H), |
| | 7.77-7.59 (m, 3H), 7.46-7.36 (m, 3H), 7.36-7.30 (m, 2H), 7.19 (d, J = |
| | 8.2 Hz, 1H), 6.53 (d, J = 2.3 Hz, 1H), 6.50-6.47 (m, 1H), 4.41-4.17 (m, |
| | 3H), 4.03 (d, J = 6.0 Hz, 3H), 3.85-3.61 (m, 6H), 3.08-2.88 (m, 2H), 2.15- |
| | 2.04 (m, 1H), 2.02-1.90 (m, 1H). LC-MS (ESI, m/z): Not Found. |
| 13-02 | 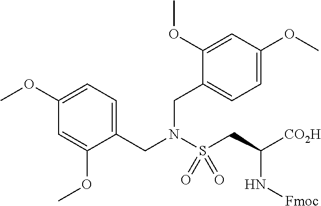 |
| | (2R)-3-{bis[(2,4-dimethoxyphenyl)methyl]sulfamoyl}-2-({[(9H-fluoren-9- |
| | yl)methoxy]carbonyl}amino)propanoic acid |
| | (2R)-2-({[(9H-fluoren-9-yl)methoxy]carbonyl}amino)-3- |
| | [(triphenylmethyl)sulfanyl]propanoic acid (CAS: 103213-32-7) |
| | bis[(2,4-dimethoxyphenyl)methyl]amine (CAS: 20781-23-1) |
| | ¹H NMR (500 MHz, DMSO-d₆) δ 13.11 (br, 1H), 7.94-7.81 (m, 3H), 7.76- |
| | 7.61 (m, 2H), 7.48-7.36 (m, 2H), 7.33-7.23 (m, 2H), 7.04 (d, J = 8.4 Hz, |
| | 2H), 6.55-6.38 (m, 4H), 4.44-4.34 (m, 1H), 4.33-4.15 (m, 7H), 3.73 (s, |
| | 6H), 3.67 (s, 6H), 3.55-3.41 (m, 2H). LC-MS (ESI, m/z): Not Found. |

### Example-14

The following compounds can be synthesized as outlined for the preparation of Reference example-20-01 employing appropriate starting materials in the table.

| | Structure and IUPAC name |
|---|---|
| | Starting material-A |
| | Starting material-B |
| Example | NMR and MS |
| 14-01 | 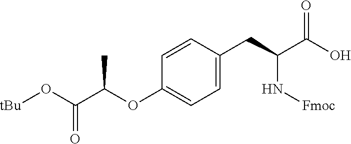 |
| | (S)-2-((((9H-fluoren-9-yl)methoxy)carbonyl)amino)-3-(4-(((R)-1-(tert-butoxy)-1- |
| | oxopropan-2-yl)oxy)phenyl)propanoic acid |
| | benzyl (2S)-2-{[(benzyloxy)carbonyl]amino}-3-(4- |
| | hydroxyphenyl)propanoate (CAS: 5513-40-6) |
| | tert-butyl (2S)-2-hydroxypropanoate (CAS: 13650-70-9) |
| | ¹H NMR (500 MHz, DMSO-d₆) δ 12.79 (br, 1H), 7.89 (d, J = 7.5 Hz, 2H), |
| | 7.69-7.48 (m, 3H), 7.44-7.38 (m, 2H), 7.35-7.27 (m, 2H), 7.19-7.09 (m, |
| | 2H), 6.79-6.70 (m, 2H), 4.68-4.62 (m, 1H), 4.22-4.06 (m, 4H), 3.00 (dd, |
| | J = 13.8, 4.2 Hz, 1H), 2.79 (dd, J = 13.7, 10.4 Hz, 1H), 1.43 (d, J = 6.7 Hz, |
| | 3H), 1.36 (s, 9H). LC-MS (ESI, m/z): [M − tBu + H]⁺ 476. |
| 14-02 | 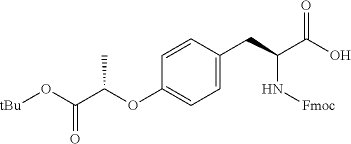 |
| | (S)-2-((((9H-fluoren-9-yl)methoxy)carbonyl)amino)-3-(4-(((S)-1-(tert-butoxy)-1- |
| | oxopropan-2-yl)oxy)phenyl)propanoic acid |
| | benzyl (2S)-2-{[(benzyloxy)carbonyl]amino}-3-(4- |
| | hydroxyphenyl)propanoate (CAS: 5513-40-6) |
| | tert-butyl (2R)-2-hydroxypropanoate (CAS: 68166-83-6) |
| | ¹H NMR (500 MHz, DMSO-d₆) δ 12.78 (br, 1H), 7.88 (d, J = 7.5 Hz, 2H), |
| | 7.68-7.63 (m, 2H), 7.58-7.50 (m, 1H), 7.44-7.38 (m, 2H), 7.35-7.26 (m, |
| | 2H), 7.17-7.05 (m, 2H), 6.74 (d, J = 8.5 Hz, 2H), 4.68 (q, J = 6.7 Hz, 1H), |
| | 4.22-4.02 (m, 4H), 3.00 (dd, J = 13.9, 4.3 Hz, 1H), 2.80 (dd, J = 13.8, 10.1 Hz, 1H), |
| | 1.44 (d, J = 6.7 Hz, 3H), 1.35 (s, 9H). LC-MS (ESI, m/z): [M − tBu + H]⁺ 476. |

### Example-15: (2R,3S)-2-((((9H-fluoren-9-yl)methoxy)carbonyl)amino)-3-((tert-butoxycarbonyl)amino)butanoic acid

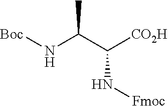

To a solution of (2R,3S)-3-azido-2-({[(9H-fluoren-9-yl)methoxy]carbonyl}amino)butanoic acid (CAS: 1932349-21-7, 1.2 g, 3.3 mmol) in iPrOH (12 mL) were added Pd/C (10%, 0.5 g) and 2 M aq. HCl (3.3 mL, 6.6 mmol). The resulting mixture was stirred at rt for 2 h under hydrogen atmosphere. The mixture was filtered and the filtrate was concentrated. To a solution of the resulting residue in 1,4-dioxane (15 mL) and H₂O (5 mL) at 0° C. was added NaHCO₃ (1.1 g, 13 mmol), followed by the addition of Boc₂O (0.86 g, 3.9 mmol). The mixture was stirred at rt overnight, and then the pH of the solution was adjusted to 4 with 1 M aq. HCl. The mixture was extracted three times with EtOAc. The organic extracts were washed with brine, dried over Na₂SO₄, filtered, and concentrated. The resulting residue was triturated with Et₂O. The resulting solid was collected by filtration to afford the title compound (0.60 g, 1.36 mmol). ¹H NMR (500 MHz, DMSO-d₆) δ 12.74 (br, 1H), 7.99-7.85 (m, 2H), 7.79-7.64 (m, 2H), 7.48-7.12 (m, 5H), 6.97-6.73 (m, 1H), 4.40-4.18 (m, 3H), 4.15-3.88 (m, 2H), 1.37 (s, 9H), 1.08-0.91 (m, 3H). LC-MS (ESI, m/z): [M-Boc+H]⁺ 341.

### Example-16: (R)-4-((((9H-fluoren-9-yl)methoxy)carbonyl)amino)-5-phenylpentanoic acid

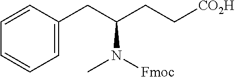

The title compound can be synthesized as outlined for the preparation of Reference example-21-01 employing appropriate starting material (9H-fluoren-9-yl)methyl N-methyl-N-[(2S)-4-oxo-1-phenylbutan-2-yl]carbamate (CAS: 950843-92-2) and ethyl 2-(triphenyl-lambda5-phosphanylidene)acetate (CAS: 1099-45-2). ¹H NMR (500 MHz, DMSO-d₆) δ 11.96 (br, 1H), 7.93-7.83 (m, 2H), 7.75-7.51 (m, 2H), 7.48-7.08 (m, 8H), 6.87-6.74 (m, 1H), 4.51-4.10 (m, 4H), 3.92-3.14 (m, 1H), 2.78-2.52 (m, 3H), 2.30-1.91 (m, 2H), 1.71-0.88 (m, 5H). LC-MS (ESI, m/z): [M+H]⁺ 444.

### Example-17: 2-{[3-({[(9H-fluoren-9-yl)methoxy]carbonyl}amino)propyl][(prop-2-en-1-yloxy)carbonyl]amino}acetic acid

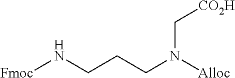

To a suspension of 2-[(3-{[(tert-butoxy)carbonyl]amino}propyl)({[(9H-fluoren-9-yl)methoxy]carbonyl})amino]acetic acid (CAS: 143192-31-8, 14.2 g, 31.3 mmol) in MeCN (104 mL) at 0° C. was added diethylamine (26.2 ml, 250 mmol). The mixture was stirred at rt for 2 h, and then concentrated. The resulting residue was azeotroped three times with toluene. To a solution of the resulting residue in 1,4-dioxane (100 mL) at 0° C. were added satd. aq. NaHCO₃ (30 ml) and allyl chloroformate (CAS: 2937-50-0, 5.01 mL, 46.9 mmol). The mixture was stirred at rt for 19 h, and then concentrated. The resulting mixture was washed with Et₂O. The pH of the aqueous layer was acidified with 6 N aq. HCl. The resulting mixture was extracted twice with EtOAc. The combined organic extracts were washed with brine, dried over Na₂SO₄, filtered, and then concentrated. The resulting residue was purified by silica gel flash column chromatography (gradient, DCM/MeOH=100/0 to 80/20). Appropriate fractions were collected and concentrated. To the resulting residue (9.72 g, 30.7 mmol) in CH₂Cl₂ (102 mL) and water (1.66 mL, 92 mmol) at 0° C. was added TFA (23.7 mL, 307 mmol). The resulting solution was stirred at rt for 3 h, and then concentrated. The resulting residue was azeotroped three times with toluene. To a solution of the residue (9.91 g, 30 mmol) in 1,4-dioxane (45 mL) at 0° C. were added a solution of NaHCO₃ (10.1 g, 120 mmol) in H₂O (75 mL) and a solution of Fmoc-OSu (9.61 g, 28.5 mmol) in 1,4-dioxane (30 mL). The mixture was stirred at rt for 18 h. The reaction was then quenched with 1 M aq. HCl. The mixture was extracted twice with EtOAc. The organic extracts were washed with brine, dried over Na₂SO₄, filtered, and concentrated. The resulting residue was purified by silica gel flash column chromatography (gradient, DCM/MeOH=100/0 to 84/16). Appropriate fractions were collected and concentrated. The resulting residue was triturated with DCM/heptane (1/5). The resulting solid was collected by filtration, then rinsed with heptane to afford the title compound (10.8 g, 24.1 mmol). ¹H NMR (400 MHz, DMSO-d₆) δ 12.66 (br, 1H), 7.89 (d, J=7.5 Hz, 2H), 7.68 (d, J=7.4 Hz, 2H), 7.49-7.23 (m, 5H), 5.95-5.81 (m, 1H), 5.31-5.19 (m, 1H), 5.17-5.12 (m, 1H), 4.58-4.43 (m, 2H), 4.38-4.15 (m, 3H), 4.00-3.82 (m, 2H), 3.29-3.21 (m, 2H), 3.08-2.90 (m, 2H), 1.75-1.57 (m, 1H). LC-MS (ESI, m/z): [M+H]⁺ 439.

### Example-18: 1-(2-(allyloxy)-2-oxoethyl)piperidine-4-carboxylic acid hydrochloride

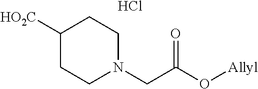

To a solution of tert-butyl piperidine-4-carboxylate hydrochloride (CAS: 892493-65-1, 4.43 g, 20 mmol) in DMF (50 mL) at 0° C. were added potassium carbonate (8.29 g, 60.0 mmol) and prop-2-en-1-yl 2-chloroacetate (CAS: 2916-14-5, 2.32 mL, 20.0 mmol). The mixture was stirred at rt for 18 h. The reaction was then quenched with H₂O. The mixture was extracted twice with EtOAc. The organic extracts were washed with water and brine, dried over Na₂SO₄, filtered, and concentrated. The resulting residue was purified by silica gel flash column chromatography (gradient, heptane/EtOAc=100/0 to 60/40). Appropriate fractions were collected and concentrated. To a mixture of the resulting residue (4.5 g, 15.9 mmol) in DCM (32 mL) at 0° C. was added 4 M HCl in 4-methyltetrahydropyran (15.9 mL, 63.5 mmol). The resulting mixture was stirred at rt for 6 h. The resulting solid was collected by filtration, then rinsed with DCM to afford the title compound (2.82 g, 10.7 mmol). ¹H NMR (500 MHz, DMSO-d₆) δ 12.60 (b, 1H), 10.72 (br, 1H), 5.99-5.90 (m, 1H), 5.39 (dd, J=1.2, 17.2 Hz, 1H), 5.28 (d, J=10.5 Hz, 1H), 4.71 (d, J=5.3 Hz, 2H), 4.26 (br s, 2H), 3.64-3.46 (m, 2H), 3.19-2.95 (m, 2H), 2.11-1.99 (m, 3H), 1.97-1.77 (m, 2H). LC-MS (ESI, m/z): Not Found.

### Example-19: 3-((2-((((9H-fluoren-9-yl)methoxy)carbonyl)(methyl)amino)ethyl)(tert-butoxycarbonyl)amino)propanoic acid

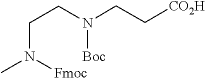

To a suspension of phenylmethyl N-(2-aminoethyl)-N-methylcarbamate (CAS: 19023-94-0, 5.1 g, 24.5 mmol) and cesium carbonate (16.0 g, 49.0 mmol) in t-BuOH (122 ml) was added ethyl acrylate (CAS: 140-88-5, 53.4 ml, 490 mmol). The mixture was stirred at rt overnight, then warmed to 40° C. and stirred for 3 h. The reaction mixture was diluted with EtOAc. The mixture was extracted with EtOAc. The combined organic extracts were washed with water and brine, dried over Na₂SO₄, filtered, and concentrated. The resulting residue was purified by silica gel flash column chromatography (gradient, DCM/MeOH=100/0 to 90/10). Appropriate fractions were collected and concentrated. To a solution of the resulting residue (4.91 g, 15.9 mmol) in DCM (53 mL) were added a solution of Boc₂O (3.82 g, 17.5 mmol) in DCM (27 mL) and Et₃N (3.33 ml, 23.9 mmol). The mixture was stirred at rt for 1 h. The reaction mixture was washed with 1 M aq. HCl·, H₂O and brine, dried over Na₂SO₄, filtered and concentrated. To a mixture of the obtained material (6.5 g, 15.9 mmol) in THF (27 mL) at 0° C. was added a solution of LiOH (1.53 g, 63.7 mmol) in H₂O (27 mL) dropwise. The mixture was stirred at rt for 5 h. The pH of the mixture was adjusted to pH 2 with 2 M aq. H₂SO₄ at 0° C. The resulting mixture was extracted twice with EtOAc. The combined organic extracts were washed with water and brine, dried over Na₂SO₄, filtered, and then concentrated. To a solution of the resulting residue (6.06 g, 15.9 mmol) in Trifluoroethanol (43 ml) and DCM (11 ml) at rt were added triethylsilane (CAS: 617-86-7, 12.7 ml, 80 mmol) and Pd/C (1.69 g, 1.59 mmol). The mixture was stirred at rt for 4 h. The reaction mixture was filtered and the filtrate was concentrated. The resulting residue was triturated with IPE. To a solution of the obtained material (3.92 g, 15.9 mmol) in THF (27 mL) at 0° C. were added a solution of NaHCO₃ (5.35 g, 63.7 mmol) in H₂O (27 mL), followed by the addition of Fmoc-OSu (5.37 g, 15.9 mmol). The mixture was stirred at rt for 1 h. The pH of the mixture was adjusted to pH 3 with 1 M aq. HCl at 0° C. The mixture was extracted twice with EtOAc. The organic extracts were washed with water and brine, dried over Na₂SO₄, filtered, and concentrated. The resulting residue was purified by silica gel flash column chromatography (gradient, DCM/MeOH=100/0 to 90/10) to afford the title compound (5.71 g, 12.2 mmol). ¹H NMR (500 MHz, DMSO-d₆) δ 12.25 (br, 1H), 7.89 (d, J=6.5 Hz, 2H), 7.69-7.59 (m, 2H), 7.41 (dd, J=7.5, 7.5 Hz, 2H), 7.34 (dd, J=7.5, 7.5 Hz, 2H), 4.43-4.22 (m, 3H), 3.39-3.21 (m, 3H), 3.21-3.01 (m, 3H), 2.85-2.72 (m, 3H), 2.47-2.38 (m, 1H), 2.38-2.27 (m, 1H), 1.49-1.18 (m, 9H). LC-MS (ESI, m/z): [M+H]⁺ 469.

### Example-20: (S)-2-((((9H-fluoren-9-yl)methoxy)carbonyl)amino)-3-(1-(tert-butoxycarbonyl)-7-(pyrimidin-2-yl)-1H-indol-3-yl)propanoic acid

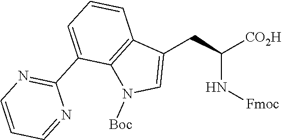

To a solution of 7-(pyrimidin-2-yl)-1H-indole (CAS: 2135521-47-8, 15.0 g, 76.8 mmol) in DMF (500 mL) at rt was added KOH (CAS: 1310-58-3, 8.62 g, 154 mmol). The mixture was stirred at rt for 30 min. To the mixture at rt was added I₂ (21.5 g, 84.5 mmol). The mixture was stirred at rt for 1 h. The mixture was extracted three times with EtOAc. The combined organic extracts were washed three times with brine, dried over Na₂SO₄, filtered, and then concentrated. To a solution of the resulting residue (21.0 g, 65.4 mmol) in CH₂Cl₂ (1000 mL) was added 4-dimethylaminopyridine (CAS: 1122-58-3, 0.80 mg, 6.54 mmol) and di-tert-butyl decarbonate (CAS: 24424-99-5, 21.4 g, 98.1 mmol). The mixture was stirred at rt for 2 h, and then concentrated. The resulting residue was purified by silica gel flash column chromatography (isocratic, petroleum ether/EtOAc=80/20). Appropriate fractions were concentrated. To a suspension of Zn (9.32 g, 142 mmol) in DMF (600 mL) was added I₂ (1.81 g, 7.12 mmol). The mixture was stirred at rt for 10 min. To the mixture at rt was added methyl (2R)-2-[[(9H-fluoren-9-ylmethoxy)carbonyl]amino]-3-iodopropanoate (CAS: 156017-42-4, 25.7 g, 57.0 mmol). The mixture was stirred at rt for an additional 1 h. To the mixture were added the obtained material in the last step (20.0 g, 47.5 mmol), Pd₂(dba)₃ (0.87 g, 0.95 mmol), and SPhos (0.97 g, 2.37 mmol). The mixture was stirred at 60° C. for an additional 3 h, and then filtered. The mixture was extracted three times with EtOAc. The combined organic extracts were washed three times with brine, dried over Na₂SO₄, filtered, and then concentrated. The resulting residue was purified by silica gel flash column chromatography (isocratic, petroleum ether/EtOAc=50/50). Appropriate fractions were concentrated. To a mixture of the resulting residue (20.0 g, 32.3 mmol) in 2-propanol (900 mL) and H₂O (300 mL) at 0° C. were added CaCl₂ (57.4 g, 517 mmol) and LiOH·H₂O (5.43 g, 129 mmol). The mixture was stirred at rt for 2 h. The pH of the mixture was adjusted to pH 7 with aq. citric acid. The resulting mixture was extracted twice with EtOAc. The combined organic extracts were washed twice with brine, dried over Na₂SO₄, filtered, and then concentrated. The resulting residue was purified by reversed phase silica gel flash column chromatography (C18 silica gel, gradient, H₂O/CH₃CN=100/0 to 0/100) to afford the title compound (10.4 g, 17.2 mmol). ¹H NMR (400 MHz, DMSO-d₆) δ 13.05 (br, 1H), 8.85 (d, J=5.2 Hz, 2H), 7.93-7.76 (m, 4H), 7.74-7.54 (m, 4H), 7.46-7.24 (m, 6H), 4.39-4.15 (m, 4H), 3.31-3.03 (m, 2H), 1.30 (s, 9H). LC-MS (ESI, m/z): [M+H]⁺ 605.

### Example-21

The following compounds can be synthesized as outlined for the preparation of Reference example-2-01, Reference example-2-02, Reference example-2-03, Reference example-2-04, Reference example-2-05, or Example-1-01 employing appropriate starting materials in the table.

| | Structure and IUPAC name |
|---|---|
| | Starting material-A |
| Ex- | Starting material-B |
| ample | NMR and MS |
| 21-01 | 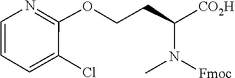 |
| | (S)-2-((((9H-fluoren-9-yl)methoxy)carbonyl)(methyl)amino)-5-(3- |
| | chloropyridin-2-yl)pentanoic acid |
| | Reference example-1-06 |
| | 2-bromo-3-chloropyridine (CAS: 96424-68-9) |
| | ¹H NMR (500 MHz, DMSO-d₆) δ 12.80 (br, 1H), 8.53-8.42 (m, |
| | 1H), 7.92-7.17 (m, 10H), 4.64-4.19 (m, 4H), 2.99-2.67 (m, 5H), |
| | 1.95-1.44 (m, 4H). LC-MS (ESI, m/z): [M + H]⁺ 465. |
| 21-02 | 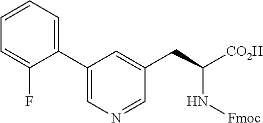 |
| | (S)-2-((((9H-fluoren-9-yl)methoxy)carbonyl)amino)-3-(5-(2- |
| | fluorophenyl)pyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | 3-bromo-5-(2-fluorophenyl)pyridine (CAS: 1070882-68-6) |
| | ¹H NMR (500 MHz, DMSO-d₆) δ 12.87 (br, 1H), 8.65-8.61 (m, |
| | 1H), 8.55-8.51 (m, 1H), 7.95-7.80 (m, 4H), 7.66-7.16 (m, 10H), |
| | 4.33-4.24(m, 1H), 4.24-4.10 (m, 3H), 3.25-3.20 (m, 1H), 3.00- |
| | 2.95 (m, 1H). LC-MS (ESI, m/z): [M + H]⁺ 483. |
| 21-03 | 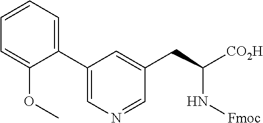 |
| | (S)-2-((((9H-fluoren-9-yl)methoxy)carbonyl)amino)-3-(5-(2- |
| | methoxyphenyl)pyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | 3-bromo-5-(2-methoxyphenyl)pyridine (CAS: 1276123-25-1) |
| | ¹H NMR (500 MHz, DMSO-d₆) δ 12.88 (br, 1H), 8.58-8.54 (m, |
| | 1H), 8.45-8.41 (m, 1H), 7.90-7.76 (m, 4H), 7.64-7.60 (m, 2H), |
| | 7.44-7.17 (m, 6H), 7.17-6.99 (m, 2H), 4.30-4.06 (m, 4H), 3.74 |
| | (s, 3H), 3.20-3.15 (m, 1H), 2.97-2.92 (m, 1H). LC-MS (ESI, |
| | m/z): [M + H]⁺ 495. |
| 21-04 | 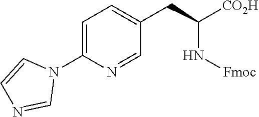 |
| | (S)-3-(6-(1H-imidazol-1-yl)pyridin-3-yl)-2-((((9H-fluoren-9- |
| | yl)methoxy)carbonyl)amino)propanoic acid |
| | methyl (2R)-2-({[(9H-fluoren-9-yl)methoxy]carbonyl}amino)-3- |
| | iodopropanoate (CAS: 156017-42-4) |
| | 5-bromo-2-(1H-imidazol-1-yl)pyridine (CAS: 380381-18-0) |
| | ¹H NMR (500 MHz, DMSO-d₆) δ 12.88 (br, 1H), 8.56-8.52 (m, |
| | 1H), 8.42-8.38 (m, 1H), 7.96-7.95 (m, 1H), 7.94-7.91 (m, 1H), |
| | 7.88-7.85 (m, 2H), 7.85-7.81 (m, 1H), 7.79-7.73 (m, 1H), 7.64- |
| | 7.59 (m, 2H), 7.41-7.35 (m, 2H), 7.31-7.24 (m, 2H), 7.14 (s, |
| | 1H), 4.29-4.14 (m, 4H), 3.26-3.08 (m, 1H), 2.97-2.89 (m, |
| | 1H). LC-MS (ESI, m/z): [M + H]⁺ 455. |
| 21-05 | 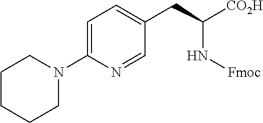 |
| | (S)-2-((((9H-fluoren-9-yl)methoxy)carbonyl)amino)-3-(6- |
| | (piperidin-1-yl)pyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | 5-bromo-2-(piperidin-1-yl)pyridine (CAS: 24255-95-6) |
| | ¹H NMR (500 MHz, DMSO-d₆) δ 12.74 (br, 1H), 7.98 (d, J = |
| | 2.3 Hz, 1H), 7.88 (d, J = 7.5 Hz, 2H), 7.72 (d, J = 8.4 Hz, 1H), |
| | 7.69-7.60 (m, 2H), 7.43-7.38 (m, 3H), 7.34-7.24 (m, 2H), |
| | 6.69 (d, J = 8.7 Hz, 1H), 4.22-4.01 (m, 4H), 3.45-3.40 (m, 4H), |
| | 2.98-2.89 (m, 1H), 2.77-2.67 (m, 1H), 1.60-1.43 (m, 6H). |
| | LC-MS (ESI, m/z): [M + H]⁺ 472. |
| 21-06 | 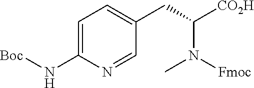 |
| | (R)-2-((((9H-fluoren-9-yl)methoxy)carbonyl)(methyl)amino)-3-(6- |
| | ((tert-butoxycarbonyl)amino)pyridin-3-yl)propanoic acid |
| | methyl (2S)-2-({[(9H-fluoren-9-yl)methoxy]carbonyl}(methyl) |
| | amino)-3-iodopropanoate (CAS: 2973756-21-5) |
| | tert-Butyl (5-iodopyridin-2-yl)carbamate (CAS: 375853-79-5) |
| | ¹H NMR (500 MHz, DMSO-d₆) δ 12.97 (br, 1H), 9.73-9.59 (m, |
| | 1H), 8.16-7.98 (m, 1H), 7.94-7.83 (m, 2H), 7.76-7.63 (m, |
| | 1H), 7.61-7.25 (m, 7H), 4.87-4.59 (m, 1H), 4.37-4.11 (m, |
| | 3H), 3.21-2.77 (m, 2H), 2.77-2.64 (m, 3H), 1.52-1.41 (m, 9H). |
| | LC-MS (ESI, m/z): [M + H]⁺ 518. |
| 21-07 | 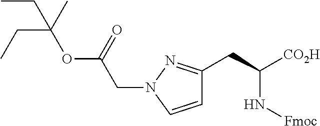 |
| | (S)-2-((((9H-fluoren-9-yl)methoxy)carbonyl)amino)-3-(1-(2-((3- |
| | methylpentan-3-yl)oxy)-2-oxoethyl)-1H-pyrazol-3-yl)propanoic |
| | acid methyl (2R)-2-({[(9H-fluoren-9-yl)methoxy]carbonyl} |
| | amino)-3-iodopropanoate (CAS: 156017-42-4) |
| | 3-methylpentan-3-yl 2-(3-bromo-1H-pyrazol-1-yl)acetate |
| | ¹H NMR (500 MHZ, DMSO-d₆) δ 12.69 (br, 1H), 7.89 (d, J = |
| | 7.5 Hz, 2H), 7.69 (d, J = 6.9 Hz, 2H), 7.65-7.56 (m, 2H), 7.44- |
| | 7.39 (m, 2H), 7.35-7.29 (m, 2H), 6.11 (d, J = 2.1 Hz, 1H), 4.87 |
| | (s, 2H), 4.31-4.11 (m, 4H), 3.03-2.98 (m, 1H), 2.91-2.82 (m, |
| | 1H), 1.81-1.72 (m, 2H), 1.70-1.61 (m, 2H), 1.30 (s, 3H), |
| | 0.79-0.75 (m, 6H).LCMS (m/z): [M + H]⁺ 520 |
| 21-08 | 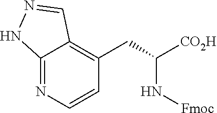 |
| | (R)-2-((((9H-fluoren-9-yl)methoxy)carbonyl)amino)-3-(1H- |
| | pyrazolo[3,4-b]pyridin-4-yl)propanoic acid |
| | methyl (2S)-2-({[(9H-fluoren-9-yl)methoxy]carbonyl}amino)-3- |
| | iodopropanoate (CAS: 527696-77-1) |
| | 4-bromo-1H-pyrazolo[3,4-b]pyridine (CAS: 856859-49-9) |
| | ¹H NMR (500 MHz, DMSO-d₆) δ 13.60 (br, 1H), 12.95 (br, 1H), |
| | 8.39 (d, J = 4.6 Hz, 1H), 8.28 (s, 1H), 7.90-7.83 (m, 3H), 7.61- |
| | 7.53 (m, 2H), 7.45-7.35 (m, 2H), 7.33-7.18 (m, 2H), 7.06 (d, |
| | J = 4.7 Hz, 1H), 4.45-4.39 (m, 1H), 4.20-4.09 (m, 3H), 3.48- |
| | 3.42 (m, 1H), 3.31-3.23 (m, 1H). LCMS (m/z): [M + H]⁺ 429 |
| 21-09 | 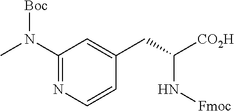 |
| | (R)-2-((((9H-fluoren-9-yl)methoxy)carbonyl)amino)-3-(2-((tert- |
| | butoxycarbonyl)(methyl)amino)pyridin-4-yl)propanoic acid |
| | methyl (2S)-2-({[(9H-fluoren-9-yl)methoxy]carbonyl} amino)-3- |
| | iodopropanoate (CAS: 527696-77-1) |
| | tert-butyl (4-bromopyridin-2-yl)(methyl)carbamate |
| | (CAS: 946000-13-1) |
| | ¹H NMR (500 MHz, DMSO-d₆) δ 12.87 (br, 1H), 8.25 (d, J = |
| | 5.0 Hz, 1H), 7.88 (d, J = 7.6 Hz, 2H), 7.79 (d, J = 8.5 Hz, 1H), |
| | 7.64-7.60 (m, 2H), 7.53(s, 1H), 7.45-7.22 (m, 4H), 7.07-7.03 (m, |
| | 1H), 4.28-4.07 (m, 4H), 3.21 (s, 3H), 3.15-2.86 (m, 2H), 1.43 (s, |
| | 9H). LC-MS (ESI, m/z): [M + H]⁺ 518. |
| 21-10 | 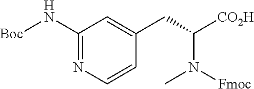 |
| | (R)-2-((((9H-fluoren-9-yl)methoxy)carbonyl)(methyl)amino)-3-(2- |
| | ((tert-butoxycarbonyl)amino)pyridin-4-yl)propanoic acid |
| | methyl (2S)-2-({[(9H-fluoren-9-yl)methoxy]carbonyl}(methyl) |
| | amino)-3-iodopropanoate (CAS: 2973756-21-5) |
| | tert-butyl N-(4-iodopyridin-2-yl)carbamate (CAS: 405939-28-8) |
| | ¹H NMR (500 MHz, DMSO-d₆) δ 13.04 (br, 1H), 9.79-9.54 (m, |
| | 1H), 8.16-8.05 (m, 1H), 7.94-7.82 (m, 2H), 7.77-7.69 (m, 1H), |
| | 7.64-7.46 (m, 2H), 7.44-7.34 (m, 2H), 7.34-7.21 (m, 2H), 6.93- |
| | 6.64 (m, 1H), 4.88-4.75 (m, 1H), 4.32-4.12 (m, 3H), 3.25- |
| | 2.90 (m, 2H), 2.80-2.67 (m, 3H), 1.46-1.34 (m, 9H). |
| | LC-MS (ESI, m/z): [M + H]⁺ 518. |
| 21-11 | 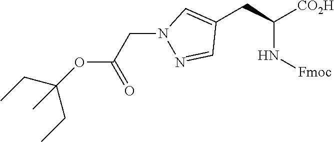 |
| | (S)-2-((((9H-fluoren-9-yl)methoxy)carbonyl)amino)-3-(1-(2-((3- |
| | methylpentan-3-yl)oxy)-2-oxoethyl)-1H-pyrazol-4-y1)propanoic |
| | acid |
| | methyl (2R)-2-({[(9H-fluoren-9-yl)methoxy] |
| | carbonyl}amino)-3-iodopropanoate (CAS: 156017-42-4) |
| | 3-methylpentan-3-yl 2-(4-bromo-1H-pyrazol-1-yl)acetate |
| | ¹H NMR (500 MHz, DMSO-d₆) δ 12.72 (br, 1H), 7.89 (d, J = |
| | 7.6 Hz, 2H), 7.74-7.55 (m, 3H), 7.53 (s, 1H), 7.42 (t, J = 7.0 Hz, |
| | 2H), 7.35-7.20 (m, 3H), 4.90-4.83 (m, 2H), 4.27-4.14 (m, 3H), |
| | 4.08-4.01 (m, 1H), 2.94-2.88 (m, 1H), 2.78-2.63 (m, 1H), |
| | 1.80-1.72 (m, 2H), 1.69-1.60 (m, 2H),1.29 (s, 3H), |
| | 0.80-0.73 (m, 6H). LC-MS (ESI, m/z): [M + H]⁺ 520. |
| 21-12 | 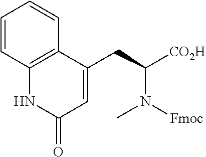 |
| | (S)-2-((((9H-fluoren-9-yl)methoxy)carbonyl)(methyl)amino)-3-(2- |
| | oxo-1,2-dihydroquinolin-4-yl)propanoic acid |
| | methyl (2R)-2-({[(9H-fluoren-9-yl)methoxy]carbonyl }(methyl) |
| | amino)-3-iodopropanoate (CAS: 2973756-19-1) |
| | 4-Bromo-2(1H)-quinolinone (CAS: 938-39-6) |
| | ¹H NMR (500 MHz, DMSO-d₆) δ 11.77-11.63 (m, 1H), |
| | 7.95-7.76 (m, 3H), 7.59-7.25 (m, 8H), 7.24-7.16 (m, 2H), |
| | 6.45-6.33 (m, 1H), 5.02-4.82 (m, 1H), 4.29-4.12 (m, |
| | 2H), 4.02-3.84 (m, 1H), 3.71-3.03 (m, 2H), |
| | 2.82 (s, 3H). LC-MS (ESI, m/z): [M + H]⁺ 469. |
| 21-13 | 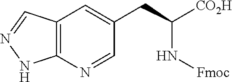 |
| | (S)-2-((((9H-fluoren-9-yl)methoxy)carbonyl)amino)-3-(1H- |
| | pyrazolo[3,4-b]pyridin-5-yl)propanoic acid |
| | methyl (2R)-2-({[(9H-fluoren-9-yl)methoxy]carbonyl}amino)-3- |
| | iodopropanoate (CAS: 156017-42-4) |
| | 5-bromo-1H-pyrazolo[3,4-b]pyridine (CAS: 875781-17-2) |
| | ¹H NMR (500 MHz, DMSO-d₆) δ 13.55 (s, 1H), 13.03-12.60 |
| | (m, 1H), 8.46 (d, J = 1.8 Hz, 1H), 8.09 (s, 1H), 8.04 (s, 1H), |
| | 7.90-7.81 (m, 3H), 7.60 (dd, J = 7.6, 18.4 Hz, 2H), 7.42-7.36 |
| | (m, 2H), 7.31-7.19 (m, 2H), 4.28-4.08 (m, 4H), 3.28-3.19 (m, |
| | 1H), 3.05-2.97 (m, 1H). LCMS (ESI, m/z): [M + H]⁺ 429. |
| 21-14 | 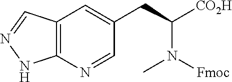 |
| | (S)-2-((((9H-fluoren-9-yl)methoxy)carbonyl)(methyl)amino)-3- |
| | (1H-pyrazolo[3,4-b]pyridin-5-yl)propanoic acid |
| | methyl (2R)-2-({[(9H-fluoren-9-yl)methoxy]carbonyl}(methyl) |
| | amino)-3-iodopropanoate (CAS: 2973756-19-1) |
| | 5-bromo-1H-pyrazolo[3,4-b]pyridine (CAS: 875781-17-2) |
| | ¹H NMR (500 MHz, DMSO-d₆) δ 13.59-13.44 (m, 1H), 8.46- |
| | 8.34 (m, 1H), 8.06-7.98 (m, 2H), 7.92-7.77 (m, 3H), 7.55-7.17 |
| | (m, 7H), 4.91-4.72 (m, 1H), 4.21-4.05 (m, 3H), 3.18-2.93 (m, |
| | 1H), 2.83-2.72 (m, 3H). LCMS (ESI, m/z): [M + H]⁺ 443. |
| 21-15 | 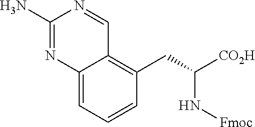 |
| | (R)-2-((((9H-fluoren-9-yl)methoxy)carbonyl)amino)-3-(2- |
| | aminoquinazolin-5-yl)propanoic acid |
| | methyl (2S)-2-({[(9H-fluoren-9-yl)methoxy]carbonyl}amino)-3- |
| | iodopropanoate (CAS: 527696-77-1) |
| | 5-bromoquinazolin-2-amine (CAS: 181871-83-0) |
| | ¹H NMR (500 MHz, DMSO-d₆) δ 7.95-7.60 (m, 5H), 7.48-6.90 |
| | (m, 9H), 6.59-6.27 (m, 1H), 5.99-5.85 (m, 1H), 4.92-4.74 (m, |
| | 1H), 4.67-4.29 (m, 3H), 3.70-3.50 (m, 1H), 2.94-2.73 (m, 1H). |
| | LC-MS (ESI, m/z): [M + H]⁺ 455. |
| 21-16 | 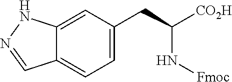 |
| | (S)-2-((((9H-fluoren-9-yl)methoxy)carbonyl)amino)-3-(1H- |
| | indazol-6-yl)propanoic acid |
| | methyl (2R)-2-({[(9H-fluoren-9-yl)methoxy]carbonyl}amino)-3- |
| | iodopropanoate (CAS: 156017-42-4) |
| | 6-bromo-1H-indazole (CAS: 79762-54-2) |
| | ¹H NMR (500 MHz, DMSO-d₆) δ 12.96 (s, 1H), 8.00 (s, 1H), |
| | 7.86 (d, J = 7.5 Hz, 2H), 7.78-7.55 (m, 4H), |
| | 7.50-7.35 (m, 4H), 7.30-7.11 (m, 2H), 7.04 (d, J = |
| | 8.2 Hz, 1H), 4.27-4.04 (m, 4H), 3.25-3.19 (m, 1H), |
| | 3.04-2.96 (m, 1H). LCMS (ESI, m/z): [M + H]⁺ 428. |
| 21-17 | 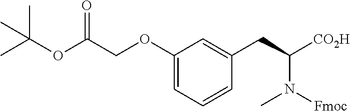 |
| | (S)-2-((((9H-fluoren-9-yl)methoxy)carbonyl)(methyl)amino)-3-(3- |
| | (2-(tert-butoxy)-2-oxoethoxy)phenyl)propanoic acid |
| | methyl (2R)-2-({[(9H-fluoren-9-yl)methoxy]carbonyl }(methyl) |
| | amino)-3-iodopropanoate (CAS: 2973756-19-1) |
| | tert-butyl 2-(3-bromophenoxy)acetate (CAS: 277331-38-1) |
| | ¹H NMR (500 MHz, DMSO-d₆) δ 12.51 (br, 1 H) |
| | 7.88 (d, J = 7.6 Hz, 2 H) 7.05-7.68 (m, 7 H) 6.60-6.91 (m, |
| | 3 H) 4.70-4.93 (m, 1 H) 4.45-4.65 (m, 2 H) |
| | 4.06-4.35 (m, 3 H) 2.79-3.24 (m, 2 H) 2.65-2.77 (m, 3 H) |
| | 1.32-1.45 (m, 9 H). LC-MS (ESI, m/z): [M − tBu + H]⁺ 476. |
| 21-18 | 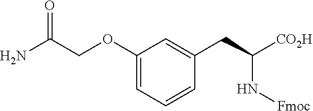 |
| | (S)-2-((((9H-fluoren-9-yl)methoxy)carbonyl)amino)-3-(3-(2- |
| | amino-2-oxoethoxy)phenyl)propanoic acid |
| | methyl (2R)-2-({[(9H-fluoren-9-y1)methoxy]carbonyl}amino)-3- |
| | iodopropanoate (CAS: 156017-42-4) |
| | 2-(3-bromophenoxy)acetamide (CAS: 214210-03-4) |
| | ¹H NMR (500 MHz, DMSO-d₆) δ 12.77 (br, 1H), 7.88 (d, J = |
| | 7.5 Hz, 2H), 7.80-7.73 (m, 1H), 7.70-7.59 (m, 2H), 7.55-7.48 (m, |
| | 1H), 7.45-7.36 (m, 3H), 7.35-7.15 (m, 3H), 6.95-6.76 (m, |
| | 3H), 4.39 (s, 2H), 4.24-4.12 (m, 4H), 3.12-3.00 (m, 1H), |
| | 2.89-2.80 (m, 1H). LC-MS (ESI, m/z): [M + H]⁺ 461 |
| 21-19 | 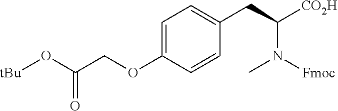 |
| | (S)-2-((((9H-fluoren-9-yl)methoxy)carbonyl)(methyl)amino)-3-(4- |
| | (2-(tert-butoxy)-2-oxoethoxy)phenyl)propanoic acid |
| | methyl (2R)-2-({[(9H-fluoren-9-y1)methoxy]carbonyl}(methyl) |
| | amino)-3-iodopropanoate (CAS: 2973756-19-1) |
| | tert-butyl 2-(4-bromophenoxy)acetate (CAS: 282116-88-5) |
| | ¹H NMR (500 MHz, DMSO-d₆) δ 12.88 (br, 1H), 7.89 (dd, J = |
| | 2.8, 7.6 Hz, 2H), 7.59-7.48 (m, 2H), 7.44-7.38 (m, 2H), 7.35- |
| | 7.27 (m, 2H), 7.11 (d, J = 8.5 Hz, 1H), 6.94 (d, J = 8.4 Hz, 1H), |
| | 6.79-6.73 (m, 2H), 4.76-4.49 (m, 3H), 4.34-4.16 (m, 3H), |
| | 3.17-3.10 (m, 1H), 3.05-2.73 (m, 1H), 2.71-2.61 (m, 3H), |
| | 1.42-1.37 (m, 9H). LCMS (m/z): [M − tBu + H]⁺ 476 |
| 21-20 | 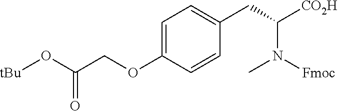 |
| | (R)-2-((((9H-fluoren-9-yl)methoxy)carbonyl)(methyl)amino)-3-(4- |
| | (2-(tert-butoxy)-2-oxoethoxy)phenyl)propanoic acid |
| | methyl (2S)-2-({[(9H-fluoren-9-yl)methoxy]carbonyl}(methyl) |
| | amino)-3-iodopropanoate (CAS: 2973756-21-5) |
| | tert-butyl 2-(4-bromophenoxy)acetate (CAS: 282116-88-5) |
| | Substantially identical to Example 21-19 |
| 21-21 | 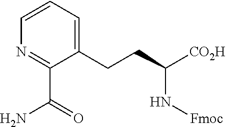 |
| | (S)-2-((((9H-fluoren-9-yl)methoxy)carbonyl)amino)-4-(2- |
| | carbamoylpyridin-3-yl)butanoic acid |
| | methyl (2S)-2-({[(9H-fluoren-9-yl)methoxy]carbonyl}amino)-4- |
| | iodobutanoate (CAS: 1354921-71-3) |
| | 3-Iodo-2-pyridinecarboxamide (CAS: 2089325-58-4) |
| | ¹H NMR (500 MHz, DMSO-d₆) δ 12.51 (br, 1H), 8.50-8.42 (m, |
| | 1H), 8.08-7.96 (m, 1H), 7.94-7.85 (m, 2H), 7.79-7.72 (m, |
| | 2H), 7.72-7.58 (m, 2H), 7.58-7.38 (m, 4H), |
| | 7.38-7.25 (m, 2H), 4.39-4.13 (m, 3H), |
| | 4.03-3.83 (m, 1H), 3.12-2.91 (m, 2H), 2.17-2.00 (m, 1H), |
| | 1.96-1.79 (m, 1H). LC-MS (ESI, m/z): [M + H]⁺ 446. |
| 21-22 | 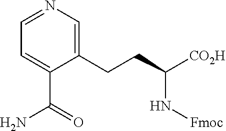 |
| | (S)-2-((((9H-fluoren-9-yl)methoxy)carbonyl)amino)-4-(4- |
| | carbamoylpyridin-3-yl)butanoic acid |
| | methyl (2S)-2-({[(9H-fluoren-9-yl)methoxy]carbonyl} amino)-4- |
| | iodobutanoate (CAS: 1354921-71-3) |
| | 3-Iodo-4-pyridinecarboxamide (CAS: 4502-19-6) |
| | ¹H NMR (500 MHz, DMSO-d₆) δ 12.55 (br, 1H), 8.47 (br, |
| | 2H), 8.01(s, 1H), 7.90 (d, J = 7.5 Hz, 2H), |
| | 7.80-7.56 (m, 4H), 7.47-7.24 (m, 5H), 4.38-4.13 (m, |
| | 3H), 3.99-3.82 (m, 1H), 2.89-2.70 (m, 2H), 2.12-1.98 (m, |
| | 1H), 1.94-1.79 (m, 1H). LC-MS (ESI, m/z): [M + H]⁺ 446. |
| 21-23 | 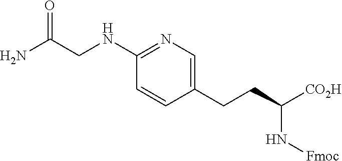 |
| | (S)-2-((((9H-fluoren-9-yl)methoxy)carbonyl)amino)-4-(6-((2- |
| | amino-2-oxoethyl)amino)pyridin-3-yl)butanoic acid |
| | methyl (2S)-2-({[(9H-fluoren-9-yl)methoxy]carbonyl} amino)-4- |
| | iodobutanoate (CAS: 1354921-71-3) |
| | 2-((5-bromopyridin-2-yl)amino)acetamide (CAS: 1041607-80-0) |
| | ¹H NMR (500 MHz, DMSO-d₆) δ 11.48 (br, 1H), 7.95-7.85 (m, |
| | 2H), 7.77 (s, 1H), 7.75-7.70 (m, 2H), 7.54-7.07 (m, 7H), 6.95 (br |
| | s, 1H), 6.54-6.45 (m, 2H), 4.36-4.19 (m, 3H), 3.83-3.72 (m, |
| | 3H), 2.48-2.36 (m, 2H), 1.93-1.85 (m, 1H), 1.85-1.75 (m, 1H). |
| | LC-MS (ESI, m/z): [M + H]⁺ 475. |
| 21-24 | 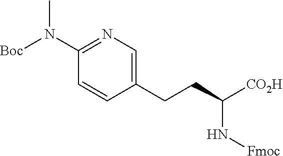 |
| | (S)-2-((((9H-fluoren-9-yl)methoxy)carbonyl)amino)-4-(6-((tert- |
| | butoxycarbonyl)(methyl)amino)pyridin-3-yl)butanoic acid |
| | methyl (2S)-2-({[(9H-fluoren-9-yl)methoxy]carbonyl}amino)-4- |
| | iodobutanoate (CAS: 1354921-71-3) |
| | tert-butyl (5-bromopyridin-2-yl)(methyl)carbamate |
| | (CAS: 227939-01-7) |
| | ¹H NMR (500 MHz, DMSO-d₆) δ 12.65 (br, 1H), 8.24-8.16 (m, |
| | 1H), 7.94-7.84 (m, 2H), 7.80-7.70 (m, 3H), 7.65-7.48 (m, 2H), |
| | 7.48-7.23 (m, 4H), 4.39-4.12 (m, 3H), 3.95-3.86 (m, 1H), |
| | 3.29-3.21 (m, 3H), 2.70-2.54 (m, 2H), 2.05-1.81 (m, 2H), |
| | 1.47-1.42 (m, 9H) LC-MS (ESI, m/z): [M + H]⁺ 532. |
| 222221- 25 | 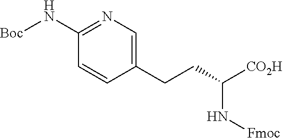 |
| | (R)-2-((((9H-fluoren-9-yl)methoxy)carbonyl)amino)-4-(6-((tert- |
| | butoxycarbonyl)amino)pyridin-3-yl)butanoic acid |
| | methyl (2R)-2-({[(9H-fluoren-9-yl)methoxy]carbonyl}amino)-4- |
| | iodobutanoate (CAS: 1323995-10-3) |
| | tert-Butyl (5-iodopyridin-2-yl)carbamate (CAS: 375853-79-5) |
| | ¹H NMR (500 MHz, DMSO-d₆) δ 12.64 (br, 1H), 9.64 (s, 1H), |
| | 8.05 (d, J = 2.0 Hz, 1H), 7.90 (d, J = 7.6 Hz, 2H), 7.78-7.68 (m, |
| | 4H), 7.58-7.54 (m, 1H), 7.46-7.25 (m, 4H), 4.38-4.22 (m, 3H), |
| | 3.93-3.82 (m, 1H), 2.67-2.52 (m, 2H), 2.00-1.80 (m, 2H), |
| | 1.46 (s, 9H). LC-MS (ESI, m/z): [M + H]⁺ 518. |
| 21-26 | 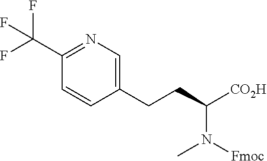 |
| | (S)-2-((((9H-fluoren-9-yl)methoxy)carbonyl)(methyl)amino)-4-(6- |
| | (trifluoromethyl)pyridin-3-yl)butanoic acid |
| | Reference example-1-04 |
| | 5-bromo-2-(trifluoromethyl)pyridine (CAS: 436799-32-5) |
| | ¹H NMR (500 MHZ, DMSO-d₆) δ 13.02 (br, 1H), 8.61 (s, 1H), |
| | 7.99-7.78 (m, 4H), 7.72-7.52 (m, 2H), 7.47-7.19 (m, 4H), |
| | 4.63-4.07 (m, 4H), 2.79-2.71 (m, 3H), 2.65-2.45 (m, 2H), |
| | 2.25-1.84 (m, 2H). LC-MS (ESI, m/z): [M + H]⁺ 485. |
| 21-27 | 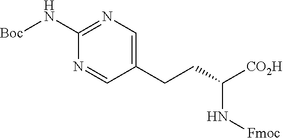 |
| | (R)-2-((((9H-fluoren-9-yl)methoxy)carbonyl)amino)-4-(2-((tert- |
| | butoxycarbonyl)amino)pyrimidin-5-yl)butanoic acid |
| | methyl (2R)-2-({[(9H-fluoren-9-yl)methoxy]carbonyl}amino)-4- |
| | iodobutanoate (CAS: 1323995-10-3) |
| | tert-butyl N-(5-iodopyrimidin-2-yl)carbamate (CAS: 1578264- |
| | 17-1) |
| | ¹H NMR (500 MHz, DMSO-d₆) δ 12.70 (br, 1H), 9.94 (s, 1H), |
| | 8.43 (s, 2H), 7.90 (d, J = 7.6 Hz, 2H), 7.76-7.72 (m, |
| | 3H), 7.47-7.26 (m,4H), 4.50-4.20 (m, 3H), |
| | 3.94-3.83 (m, 1H), 2.67-2.52 (m, 2H), 2.06-1.80 (m, |
| | 2H), 1.45 (s, 9H). LC-MS (ESI, m/z): [M + H]⁺ 519. |
| 21-28 | 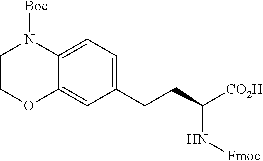 |
| | (S)-2-((((9H-fluoren-9-yl)methoxy)carbonyl)amino)-4-(4-(tert- |
| | butoxycarbonyl)-3,4-dihydro-2H-benzo[b][1,4]oxazin-7- |
| | yl)butanoic acid |
| | methyl (2S)-2-({[(9H-fluoren-9-yl)methoxy]carbonyl}amino)- |
| | 4-iodobutanoate (CAS: 1354921-71-3) |
| | tert-butyl 7-bromo-2,3-dihydro-4H-benzo[b][1,4]oxazine-4- |
| | carboxylate (CAS: 1220039-59-7) |
| | ¹H NMR (500 MHz, DMSO-d₆) δ 12.60 (br, 1H), 7.94-7.85 (m, |
| | 2H), 7.80-7.24 (m, 8H), 6.76-6.63 (m, 2H), 4.38-4.07 (m, |
| | 5H), 3.99-3.84 (m, 1H), 3.81-3.68 (m, 2H), |
| | 2.65-2.44 (m, 2H), 2.00-1.76 (m, 2H), |
| | 1.55-1.40 (m, 9H). LC-MS (ESI, m/z): [M + H]⁺ 559. |
| 21-29 | 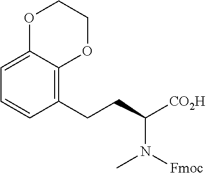 |
| | (S)-2-((((9H-fluoren-9-yl)methoxy)carbonyl)(methyl)amino)-4- |
| | (2,3-dihydrobenzo[b][1,4]dioxin-5-yl)butanoic acid |
| | Reference example-1-04 |
| | 5-bromo-2,3-dihydrobenzo[b][1,4]dioxine (CAS: 58328-39-5) |
| | ¹H NMR (500 MHz, DMSO-d₆) δ 12.84 (br, 1H), 7.94-7.86 (m, |
| | 2H), 7.70-7.65 (m, 1H), 7.65-7.54 (m, 1H), 7.44-7.37 (m, |
| | 2H), 7.36-7.26 (m, 2H), 6.75-6.66 (m, 3H), 4.54-4.16 (m, |
| | 8H), 2.82-2.75 (m, 3H), 2.48-2.31 (m, 2H), 2.11-2.03 (m, 1H), |
| | 1.99-1.86 (m, 1H). LC-MS (ESI, m/z): [M + H]⁺ 474. |
| 21-30 | 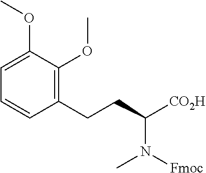 |
| | (S)-2-((((9H-fluoren-9-yl)methoxy)carbonyl)(methyl)amino)-4- |
| | (2,3-dimethoxyphenyl)butanoic acid |
| | Reference example-1-04 |
| | 1-bromo-2,3-dimethoxybenzene (CAS: 5424-43-1) |
| | ¹H NMR (500 MHz, DMSO-d₆) δ 12.85 (br, 1H), 7.93-7.86 (m, |
| | 2H), 7.71-7.65 (m, 1H), 7.64-7.54 (m, 1H), 7.44-7.26 (m, 4H), |
| | 7.00-6.94 (m, 1H), 6.91-6.86 (m, 1H), 6.74 (d, J = 7.6 Hz, |
| | 1H), 4.57-4.15 (m, 4H), 3.82-3.73 (m, 3H), 3.71-3.67 (m, 3H), |
| | 2.83-2.78 (m, 3H), 2.49-2.32 (m, 2H), 2.11-2.03 (m, 1H), |
| | 1.95-1.83 (m, 1H). LC-MS (ESI, m/z): [M + H]⁺ 476. |
| 21-31 | 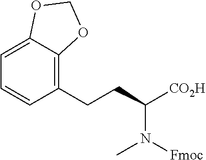 |
| | (S)-2-((((9H-fluoren-9-yl)methoxy)carbonyl)(methyl)amino)-4- |
| | (benzo[d][1,3]dioxol-4-yl)butanoic acid |
| | Reference example-1-04 |
| | 4-bromobenzo[d][1,3]dioxole (CAS: 6698-13-1) |
| | ¹H NMR (500 MHz, DMSO-d₆) δ 12.89 (br, 1H), |
| | 7.93-7.86 (m, 2H), 7.70-7.64 (m, 1H), |
| | 7.64-7.51 (m, 1H), 7.44-7.25 (m, 4H), 6.79-6.73 |
| | (m, 2H), 6.71-6.66 (m, 1H), 6.00-5.93 (m, 2H), 4.53-4.13 (m, |
| | 4H), 2.77 (s, 3H), 2.48-2.34 (m, 2H), 2.18-2.07 (m, 1H), |
| | 2.03-1.89 (m, 1H). LC-MS (ESI, m/z): [M + H]⁺ 460. |
| 21-32 | 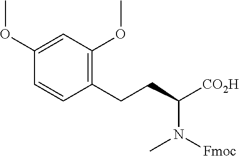 |
| | (S)-2-((((9H-fluoren-9-yl)methoxy)carbonyl)(methyl)amino)-4- |
| | (2,4-dimethoxyphenyl)butanoic acid |
| | Reference example-1-04 |
| | 1-bromo-2,4-dimethoxybenzene (CAS: 17715-69-4) |
| | ¹H NMR (500 MHz, DMSO-d₆) δ 12.81 (br, 1H), 7.93-7.87 (m, |
| | 2H), 7.71-7.53 (m, 2H), 7.44-7.37 (m, 2H), 7.36-7.22 (m, 2H), |
| | 7.00-6.96 (m, 1H), 6.51 (d, J = 2.4 Hz, 1H), 6.45-6.40 (m, |
| | 1H), 4.55-4.13 (m, 4H), 3.77-3.68 (m, 6H), 2.82-2.77 (m, |
| | 3H), 2.46-2.39 (m, 1H), 2.37-2.25 (m, 1H), 2.08-1.98 (m, |
| | 1H), 1.93-1.81 (m, 1H). LC-MS (ESI, m/z): [M + H]⁺ 476. |
| 21-33 | 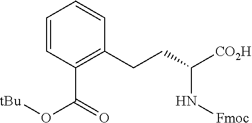 |
| | (R)-2-((((9H-fluoren-9-yl)methoxy)carbonyl)amino)-4-(2-(tert- |
| | butoxycarbonyl)phenyl)butanoic acid |
| | methyl (2R)-2-({[(9H-fluoren-9-yl)methoxy]carbonyl}amino)-4- |
| | iodobutanoate (CAS: 1323995-10-3) |
| | tert-butyl 2-bromobenzoate (CAS: 55666-42-7) |
| | ¹H NMR (500 MHz, DMSO-d₆) δ 12.59 (br, 1H), 7.90 (d, J = |
| | 7.6 Hz, 2H), 7.79-7.71 (m, 3H), 7.70-7.55 (m, |
| | 1H), 7.52-7.38 (m, 3H), 7.37-7.21 (m, 4H), |
| | 4.38-4.13 (m, 3H), 3.99-3.83 (m, 1H), 2.99-2.80 |
| | (m, 2H), 2.10-1.95 (m, 1H), 1.91-1.75 (m, 1H), 1.54 (s, 9H). |
| | LC-MS (ESI, m/z): [M − tBu + H]⁺ 446. |
| 21-34 | 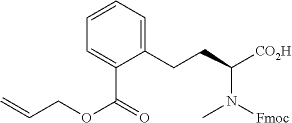 |
| | (S)-2-((((9H-fluoren-9-yl)methoxy)carbonyl)(methyl)amino)-4-(2- |
| | ((allyloxy)carbonyl)phenyl)butanoic acid |
| | Reference example-1-04 |
| | allyl 2-bromobenzoate (CAS: 101320-93-8) |
| | ¹H NMR (500 MHz, DMSO-d₆) δ 12.80 (br, 1H), 7.94-7.80 (m, |
| | 3H), 7.72-7.48 (m, 3H), 7.45-7.23 (m, 6H), 6.09-5.98 (m, 1H), |
| | 5.44-5.33 (m, 1H), 5.29-5.22 (m, 1H), 4.83-4.71 (m, 2H), |
| | 4.62-4.15 (m, 4H), 2.90-2.61 (m, 5H), 2.16-1.85 (m, |
| | 2H). LC-MS (ESI, m/z): [M + H]⁺ 500. |
| 21-35 | 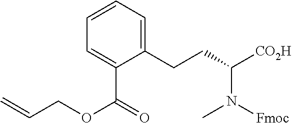 |
| | (R)-2-((((9H-fluoren-9-yl)methoxy)carbonyl)(methyl)amino)-4-(2- |
| | ((allyloxy)carbonyl)phenyl)butanoic acid |
| | Reference example-1-03 |
| | allyl 2-bromobenzoate (CAS: 101320-93-8) |
| | Substantialy identical to Example 21-34 |
| 21-36 | 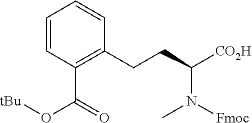 |
| | (S)-2-((((9H-fluoren-9-yl)methoxy)carbonyl)(methyl)amino)-4-(2- |
| | (tert-butoxycarbonyl)phenyl)butanoic acid |
| | Reference example-1-04 |
| | tert-butyl 2-bromobenzoate (CAS: 55666-42-7) |
| | ¹H NMR (500 MHz, DMSO-d₆) δ 12.90 (br, 1H), 7.95-7.83 (m, |
| | 2H), 7.72-7.54 (m, 3H), 7.51-7.22 (m, 7H), 4.63-4.13 (m, 4H), |
| | 2.86-2.67 (m, 5H), 2.20-2.05 (m, 1H), 2.01-1.85 (m, 1H), |
| | 1.53 (s, 9H). LC-MS (ESI, m/z): [M + H]⁺ 516. |
| 21-37 | 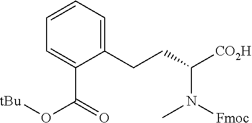 |
| | (R)-2-((((9H-fluoren-9-yl)methoxy)carbonyl)(methyl)amino)-4-(2- |
| | (tert-butoxycarbonyl)phenyl)butanoic acid |
| | Reference example-1-03 |
| | tert-butyl 2-bromobenzoate (CAS: 55666-42-7) |
| | Substantialy identical to Example 21-36 |
| 21-38 | 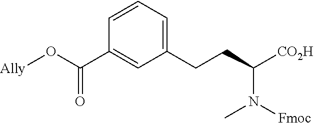 |
| | (S)-2-((((9H-fluoren-9-yl)methoxy)carbonyl)(methyl)amino)-4-(3- |
| | ((allyloxy)carbonyl)phenyl)butanoic acid |
| | Reference example-1-04 |
| | allyl 3-bromobenzoate (CAS: 1623435-72-2) |
| | ¹H NMR (500 MHz, DMSO-d₆) δ 12.90 (br, 1H), 7.95-7.75 (m, |
| | 4H), 7.69-7.22 (m, 8H), 6.09-5.96 (m, 1H), 5.43-5.32 (m, |
| | 1H), 5.30-5.19 (m, 1H), 4.82-4.74 (m, 2H), 4.52-4.09 (m, |
| | 4H), 2.81-2.69 (m, 3H), 2.61-2.39 (m, 2H), 2.21-1.89 (m, |
| | 2H). LC-MS (ESI, m/z): [M + H]⁺ 500. |
| 21-39 | 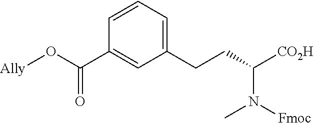 |
| | (R)-2-((((9H-fluoren-9-yl)methoxy)carbonyl)(methyl)amino)-4-(3- |
| | ((allyloxy)carbonyl)phenyl)butanoic acid |
| | Reference example-1-03 |
| | allyl 3-bromobenzoate (CAS: 1623435-72-2) |
| | Substantially identical to Example 21-38 |
| 21-40 | 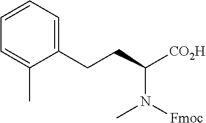 |
| | (S)-2-((((9H-fluoren-9-yl)methoxy)carbonyl)(methyl)amino)-4-(o- |
| | tolyl)butanoic acid |
| | Reference example-1-04 |
| | 1-bromo-2-methylbenzene (CAS: 95-46-5) |
| | ¹H NMR (500 MHz, DMSO-d₆) δ 12.85 (br, 1H), |
| | 7.95-7.83 (m, 2H), 7.71-7.55 (m, 2H), |
| | 7.46-7.23 (m, 4H), 7.18-7.03 (m, 4H), 4.58-4.16 |
| | (m, 4H), 2.82-2.76 (m, 3H), 2.49-2.31 (m, 2H), 2.23 (s, 3H), |
| | 2.12-1.80 (m, 2H). LC-MS (ESI, m/z): [M + H]⁺ 430. |
| 21-41 | 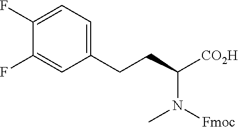 |
| | (S)-2-((((9H-fluoren-9-yl)methoxy)carbonyl)(methyl)amino)-4- |
| | (3,4-difluorophenyl)butanoic acid |
| | Reference example-1-04 |
| | 4-bromo-1,2-difluorobenzene (CAS: 348-61-8) |
| | ¹H NMR (500 MHz, DMSO-d₆) δ 12.88 (br, 1H), 8.00-7.81 (m, |
| | 2H), 7.73-7.52 (m, 2H), 7.49-7.21 (m, 6H), 7.09-6.95 (m, 1H), |
| | 4.61-4.12 (m, 4H), 2.83-2.67 (m, 3H), 2.49-2.30 (m, 2H), |
| | 2.20-1.81 (m, 2H). LC-MS (ESI, m/z): [M + H]⁺ 452. |
| 21-42 | 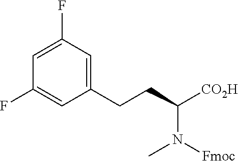 |
| | (S)-2-((((9H-fluoren-9-yl)methoxy)carbonyl)(methyl)amino)-4- |
| | (3,5-difluorophenyl)butanoic acid |
| | Reference example-1-04 |
| | 1-bromo-3,5-difluorobenzene (CAS: 461-96-1) |
| | ¹H NMR (500 MHz, DMSO-d₆) 12.89 (br, 1H), 7.93-7.86 (m, |
| | 2H), 7.70-7.54 (m, 2H), 7.44-7.26 (m, 4H), 7.06-6.91 (m, 3H), |
| | 4.55-4.14 (m, 4H), 2.78-2.73 (m, 3H), 2.50-2.36 (m, 2H), |
| | 2.16-1.88 (m, 2H). LC-MS (ESI, m/z): [M + H]⁺ 452. |
| 21-43 | 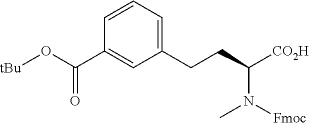 |
| | (S)-2-((((9H-fluoren-9-yl)methoxy)carbonyl)(methyl)amino)-4-(3- |
| | (tert-butoxycarbonyl)phenyl)butanoic acid |
| | Reference example-1-04 |
| | tert-butyl 3-bromobenzoate (CAS: 69038-74-0) |
| | ¹H NMR (500 MHz, DMSO-d₆) δ 12.87 (br, 1H), 7.94-7.83 (m, |
| | 2H), 7.75-7.49 (m, 4H), 7.49-7.22 (m, 6H), 4.53-4.11 (m, |
| | 4H), 2.81-2.71 (m, 3H), 2.60-2.39 (m, 2H), 2.18-1.90 (m, 2H), |
| | 1.59-1.42 (m, 9H). LC-MS (ESI, m/z): [M − tBu + H]⁺ 460. |
| 21-44 | 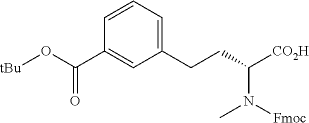 |
| | (R)-2-((((9H-fluoren-9-yl)methoxy)carbonyl)(methyl)amino)-4-(3- |
| | (tert-butoxycarbonyl)phenyl)butanoic acid |
| | Reference example-1-03 |
| | tert-butyl 3-bromobenzoate (CAS: 69038-74-0) |
| | ¹H NMR (500 MHz, DMSO-d₆) δ 12.87 (br, 1H), 7.92-7.84 (m, |
| | 2H), 7.75-7.64 (m, 3H), 7.63-7.49 (m, 1H), 7.46-7.24 (m, 6H), |
| | 4.54-4.11 (m, 4H), 2.79-2.74 (m, 3H), 2.58-2.36 (m, |
| | 2H; overlapped with DMSO), 2.17-1.91 (m, 2H), |
| | 1.54-1.48 (m, 9H). LC-MS (ESI, m/z): [M − tBu + H]⁺ 460. |
| 21-45 | 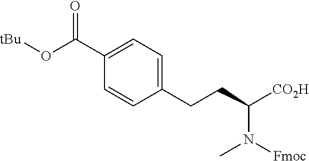 |
| | (S)-2-((((9H-fluoren-9-yl)methoxy)carbonyl)(methyl)amino)-4-(4- |
| | (tert-butoxycarbonyl)phenyl)butanoic acid |
| | Reference example-1-04 |
| | tert-butyl 4-bromobenzoate (CAS: 59247-47-1) |
| | ¹H NMR (500 MHz, DMSO-d₆) δ 12.88 (br, 1H), 7.94-7.78 (m, |
| | 4H), 7.70-7.48 (m, 2H), 7.46-7.22 (m, 6H), 4.51-4.07 (m, 4H), |
| | 2.79-2.69 (m, 3H), 2.61-2.39 (m, 2H), 2.19-1.90 (m, 2H), |
| | 1.57-1.43 (m, 9H). LC-MS (ESI, m/z): [M − tBu + H]⁺ 460. |
| 21-46 | 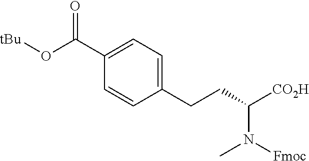 |
| | (R)-2-((((9H-fluoren-9-yl)methoxy)carbonyl)(methyl)amino)-4-(4- |
| | (tert-butoxycarbonyl)phenyl)butanoic acid |
| | Reference example-1-03 |
| | tert-butyl 4-bromobenzoate (CAS: 59247-47-1) |
| | ¹H NMR (500 MHz, DMSO-d₆) δ 12.99 (br, 1H), 7.97-7.79 (m, |
| | 4H), 7.70-7.47 (m, 2H), 7.44-7.24 (m, 6H), 4.49-4.08 (m, 4H), |
| | 2.77-2.72 (m, 3H), 2.56-2.52 (m, 1H), 2.47-2.40 (m, |
| | 1H), 2.17-2.08 (m, 1H), 2.06-1.91 (m, 1H), |
| | 1.54-1.42 (m, 9H). LC-MS (ESI, m/z): [M − tBu + H]⁺ 460. |
| 21-47 | 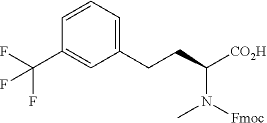 |
| | (S)-2-((((9H-fluoren-9-yl)methoxy)carbonyl)(methyl)amino)-4-(3- |
| | (trifluoromethyl)phenyl)butanoic acid |
| | Reference example-1-04 |
| | 1-bromo-3-(trifluoromethyl)benzene (CAS: 401-78-5) |
| | ¹H NMR (500 MHz, DMSO-d₆) δ 12.95 (br, 1H), 7.94-7.85 (m, |
| | 2H), 7.72-7.45 (m, 6H), 7.44-7.22 (m, 4H), 4.53-4.13 (m, 4H), |
| | 2.79-2.72 (m, 3H), 2.63-2.50 (m, 2H), 2.20-1.89 (m, 2H). |
| | LC-MS (ESI, m/z): [M + H]⁺ 484. |
| 21-48 | 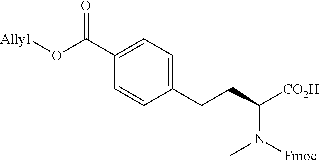 |
| | (S)-2-((((9H-fluoren-9-yl)methoxy)carbonyl)(methyl)amino)-4-(4- |
| | ((allyloxy)carbonyl)phenyl)butanoic acid |
| | Reference example-1-04 |
| | allyl 4-bromobenzoate (CAS: 360045-14-3) |
| | ¹H NMR (500 MHZ, DMSO-d₆) δ 12.89 (s, 1H), 7.94-7.83 (m, |
| | 4H), 7.71-7.63 (m, 1H), 7.61-7.50 (m, 1H), |
| | 7.46-7.22 (m, 6H), 6.11-5.93 (m, 1H), 5.43-5.32 (m, |
| | 1H), 5.30-5.20 (m, 1H), 4.81-4.72 (m, 2H), 4.53-4.10 (m, |
| | 4H), 2.78-2.72 (m, 3H), 2.61-2.40 (m, 2H; overlapped with |
| | DMSO), 2.21-1.90 (m, 2H). LC-MS (ESI, m/z): [M + H]⁺ 500. |
| 21-49 | 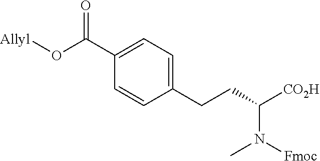 |
| | (R)-2-((((9H-fluoren-9-yl)methoxy)carbonyl)(methyl)amino)-4-(4- |
| | ((allyloxy)carbonyl)phenyl)butanoic acid |
| | Reference example-1-03 |
| | allyl 4-bromobenzoate (CAS: 360045-14-3) |
| | ¹H NMR (500 MHz, DMSO-d₆) δ 12.86 (br, 1H), 7.93-7.83 (m, |
| | 4H), 7.71-7.21 (m, 8H), 6.07-5.96 (m, 1H), 5.41-5.33 (m, |
| | 1H), 5.28-5.22 (m, 1H), 4.80-4.72 (m, 2H), 4.54-4.07 (m, 4H), |
| | 2.77-2.72 (m, 3H), 2.60-2.39 (m, 2H), 2.19-2.09 (m, 1H), |
| | 2.07-1.92 (m, 1H). LC-MS (ESI, m/z): [M + H]⁺ 500. |
| 21-50 | 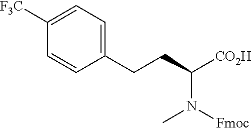 |
| | (S)-2-((((9H-fluoren-9-yl)methoxy)carbonyl)(methyl)amino)-4-(4- |
| | (trifluoromethyl)phenyl)butanoic acid |
| | Reference example-1-04 |
| | 1-bromo-4-(trifluoromethyl)benzene (CAS: 402-43-7) |
| | ¹H NMR (500 MHz, DMSO-d₆) δ 12.88 (br, 1H), 7.93-7.86 (m, |
| | 2H), 7.70-7.53 (m, 4H), 7.44-7.25 (m, 6H), 4.51-4.41 (m, |
| | 1H), 4.38-4.28 (m, 2H), 4.18-4.13 (m, 1H), 2.78-2.73 (m, 3H), |
| | 2.60-2.54 (m, 1H), 2.49-2.41 (m, 1H), 2.18-2.08 (m, 1H), |
| | 2.07-1.93 (m, 1H). LC-MS (ESI, m/z): [M + H]⁺ 484. |
| 21-51 | 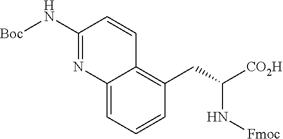 |
| | (R)-2-((((9H-fluoren-9-yl)methoxy)carbonyl)amino)-3-(2-((tert- |
| | butoxycarbonyl)amino)quinolin-5-yl)propanoic acid |
| | methyl (2S)-2-({[(9H-fluoren-9-yl)methoxy]carbonyl}amino)-3- |
| | iodopropanoate (CAS: 527696-77-1) |
| | tert-butyl (5-bromoquinolin-2-yl)carbamate (CAS: 1414959-06-0) |
| | ¹H NMR (500 MHz, DMSO-d₆) δ 12.88 (br, 1H), 10.11 (s, 1H), |
| | 8.50 (d, J = 9.5 Hz, 1H), 8.07 (d, J = 9.3 Hz, 1H), 7.91-7.77 (m, |
| | 3H), 7.71-7.52 (m, 4H), 7.44-7.22 (m, 5H), 4.32-4.00 (m, 4H), |
| | 3.63-3.53 (m, 1H), 3.35-3.25 (m, 1H), 1.49 (s, 9H). |
| | LC-MS (ESI, m/z): [M + H]⁺ 554. |
| 21-52 | 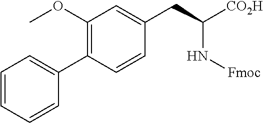 |
| | (S)-2-((((9H-fluoren-9-yl)methoxy)carbonyl)amino)-3-(2- |
| | methoxy-[1,1′-biphenyl]-4-yl)propanoic acid |
| | methyl (2R)-2-({[(9H-fluoren-9-yl)methoxy]carbonyl}amino)-3- |
| | iodopropanoate (CAS: 156017-42-4) |
| | 4-bromo-2-methoxy-1,1′-biphenyl (CAS: 227305-07-9) |
| | ¹H NMR (500 MHz, DMSO-d₆) δ 12.85 (br, 1H), 7.91-7.85 (m, |
| | 2H), 7.83-7.75 (m, 1H), 7.69-7.48 (m, 2H), 7.45-7.34 (m, 6H), |
| | 7.32-7.23 (m, 3H), 7.23-7.15 (m, 1H), 7.07 (s, 1H), 6.95-6.91 |
| | (m, 1H), 4.29-4.12 (m, 4H), 3.74 (s, 3H), 3.27-3.07 (m, 1H), |
| | 2.96-2.79 (m, 1H). LC-MS (ESI, m/z): [M + H]⁺ 494. |
| 21-53 | 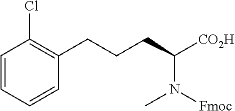 |
| | (S)-2-((((9H-fluoren-9-yl)methoxy)carbonyl)(methyl)amino)-5-(2- |
| | chlorophenyl)pentanoic acid |
| | Reference example-1-06 |
| | 1-bromo-2-chlorobenzene (CAS: 694-80-4) |
| | ¹H NMR (500 MHz, DMSO-d₆) δ 12.82 (br, 1H), 7.92-7.78 (m, |
| | 2H), 7.67-7.56 (m, 2H), 7.45-7.36 (m, 3H), 7.35-7.17 (m, 5H), |
| | 4.62-4.14 (m, 4H), 2.78-2.55 (m, 2H), 2.72-2.66 (m, 3H), |
| | 1.93-1.32 (m, 4H). LC-MS (ESI, m/z): [M + H]⁺ 464. |
| 21-54 | 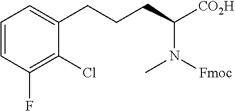 |
| | (S)-2-((((9H-fluoren-9-yl)methoxy)carbonyl)(methyl)amino)-5-(2- |
| | chloro-3-fluorophenyl)pentanoic acid |
| | Reference example-1-06 |
| | 1-bromo-2-chloro-3-fluorobenzene (CAS: 883499-24-9) |
| | ¹H NMR (500 MHz, DMSO-d₆) δ 12.82 (br, 1H), 7.97-7.09 (m, |
| | 11H), 4.62-4.17 (m, 4H), 2.85-2.54 (m, 5H), 1.92-1.11 (m, 4H). |
| | LC-MS (ESI, m/z): [M + H]⁺ 482. |
| 21-55 | 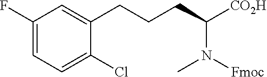 |
| | (S)-2-((((9H-fluoren-9-yl)methoxy)carbonyl)(methyl)amino)-5- |
| | (2-chloro-5-fluorophenyl)pentanoic acid |
| | Reference example-1-06 |
| | 2-bromo-1-chloro-4-fluorobenzene (CAS: 201849-15-2) |
| | ¹H NMR (500 MHz, DMSO-d₆) δ 12.82 (br, 1H), 7.94-7.07 (m, |
| | 11H), 4.60-4.21 (m, 4H), 2.78-2.53 (m, 5H), 1.92-1.29 (m, 4H). |
| | LC-MS (ESI, m/z): [M + H]⁺ 482. |
| 21-56 | 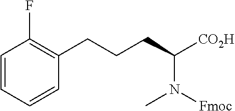 |
| | (S)-2-((((9H-fluoren-9-yl)methoxy)carbonyl)(methyl)amino)-5-(2- |
| | fluorophenyl)pentanoic acid |
| | Reference example-1-06 |
| | 1-bromo-2-fluorobenzene (CAS: 1072-85-1) |
| | ¹H NMR (500 MHz, DMSO-d₆) δ 12.80 (br, 1H), 7.92-7.80 (m, |
| | 2H), 7.66-7.56 (m, 2H), 7.42-7.10 (m, 8H), |
| | 4.59-4.17 (m, 4H), 2.70-2.66 (m, 3H), 2.65-2.52 (m, |
| | 2H), 1.90-1.55 (m, 2H), 1.50-1.40 (m, 1H), |
| | 1.40-1.29 (m, 1H). LC-MS (ESI, m/z): [M + H]⁺ 448. |
| 21-57 | 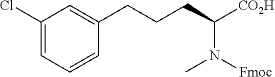 |
| | (S)-2-((((9H-fluoren-9-yl)methoxy)carbonyl)(methyl)amino)-5-(3- |
| | chlorophenyl)pentanoic acid |
| | Reference example-1-06 |
| | 1-bromo-3-chlorobenzene (CAS: 108-37-2) |
| | ¹H NMR (500 MHz, DMSO-d₆) δ 12.78 (br, 1H), 7.91-7.82 (m, |
| | 2H), 7.67-7.57 (m, 2H), 7.44-7.21 (m, 7H), 7.18-7.10 (m, 1H), |
| | 4.57-4.21 (m, 4H), 2.71-2.65 (m, 3H), 2.65-2.44 (m, 2H), |
| | 1.86-1.24 (m, 4H). LC-MS (ESI, m/z): [M + H]⁺ 463. |
| 21-58 | 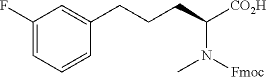 |
| | (S)-2-((((9H-fluoren-9-yl)methoxy)carbonyl)(methyl)amino)-5-(3- |
| | fluorophenyl)pentanoic acid |
| | Reference example-1-06 |
| | 1-bromo-3-fluorobenzene (CAS: 1073-06-9) |
| | ¹H NMR (500 MHz, DMSO-d₆) δ 12.80 (br, 1H), 7.94-7.81 (m, |
| | 2H), 7.69-7.56 (m, 2H), 7.48-7.21 (m, 5H), 7.08-6.95 (m, 3H), |
| | 4.57-4.20 (m, 4H), 2.77-2.50 (m, 5H), 1.88-1.21 (m, 4H). |
| | LC-MS (ESI, m/z): [M + H]⁺ 448. |
| 21-59 | 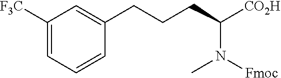 |
| | (S)-2-((((9H-fluoren-9-yl)methoxy)carbonyl)(methyl)amino)-5-(3- |
| | (trifluoromethyl)phenyl)pentanoic acid |
| | Reference example-1-06 |
| | 1-bromo-3-(trifluoromethyl)benzene (CAS: 401-78-5) |
| | ¹H NMR (500 MHz, DMSO-d₆) δ 12.81 (br, 1H), 7.95-7.77 (m, |
| | 2H), 7.71-7.43 (m, 6H), 7.43-7.30 (m, 2H), 7.30-7.17 (m, |
| | 2H), 4.66-4.16 (m, 4H), 2.80-2.53 (m, 5H), |
| | 1.90-1.28 (m, 4H) LC-MS (ESI, m/z): [M + H]⁺ 498. |
| 21-60 | 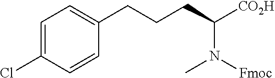 |
| | (S)-2-((((9H-fluoren-9-yl)methoxy)carbonyl)(methyl)amino)-5-(4- |
| | chlorophenyl)pentanoic acid |
| | Reference example-1-06 |
| | 1-bromo-4-chlorobenzene (CAS: 106-39-8) |
| | ¹H NMR (500 MHz, DMSO-d₆) δ 12.78 (br, 1H), 7.92-7.83 (m, |
| | 2H), 7.67-4.56-4.21 (m, 4 H),7.58 (m, 2H), 7.44-7.24 (m, 6H), |
| | 7.22-7.16 (m, 2H), 2.70-2.65 (m, 3H), 2.64-2.43 (m, 5H), |
| | 1.86-1.27 (m, 4H). LC-MS (ESI, m/z): [M + H]⁺ 464. |
| 21-61 | 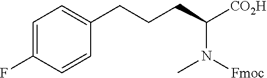 |
| | (S)-2-((((9H-fluoren-9-yl)methoxy)carbonyl)(methyl)amino)-5-(4- |
| | fluorophenyl)pentanoic acid |
| | Reference example-1-06 |
| | 1-bromo-4-fluorobenzene (CAS: 460-00-4) |
| | ¹H NMR (500 MHz, DMSO-d₆) δ 12.78 (br, 1 H), 7.92-7.83 (m, |
| | 2 H), 7.67-7.57 (m, 2 H), 7.43-7.35 (m, 2 H), 7.33-7.23 (m, |
| | 2 H), 7.23-7.15 (m, 2 H), 7.13-7.07 (m, 2 H), 5.77-5.74 (m, |
| | 1 H), 4.58-4.20 (m, 4 H), 2.71-2.65 (m, 3 H), 2.65-2.42 (m, |
| | 6 H), 1.86-1.28 (m, 4 H). LC-MS (ESI, m/z): [M + H]⁺ 448. |
| 21-62 | 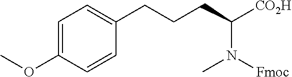 |
| | (S)-2-((((9H-fluoren-9-yl)methoxy)carbonyl)(methyl)amino)-5-(4- |
| | methoxyphenyl)pentanoic acid |
| | Reference example-1-06 |
| | 1-bromo-4-methoxybenzene (CAS: 104-92-7) |
| | ¹H NMR (500 MHz, DMSO-d₆) δ 12.75 (br, 1H), 7.94-7.82 (m, |
| | 2H), 7.69-7.56 (m, 2H), 7.46-7.23 (m, 4H), 7.13-7.03 (m, 2H), |
| | 6.91-6.80 (m, 2H), 4.58-4.20 (m, 4H), 3.71 (s, 3H), 2.74- |
| | 2.62 (m, 3H), 2.62-2.40 (m, 2H), 1.88-1.26 (m, 4H). |
| | LC-MS (ESI, m/z): [M + H]⁺ 460. |
| 21-63 | 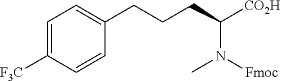 |
| | (S)-2-((((9H-fluoren-9-yl)methoxy)carbonyl)(methyl)amino)-5-(4- |
| | (trifluoromethyl)phenyl)pentanoic acid |
| | Reference example-1-06 |
| | 1-bromo-4-(trifluoromethyl)benzene (CAS: 402-43-7) |
| | ¹H NMR (500 MHz, DMSO-d₆) δ 12.80 (br, 1H), 7.95-7.81 (m, |
| | 2H), 7.70-7.54 (m, 4H), 7.46-7.32 (m, 4H), |
| | 7.32-7.19 (m, 2H), 4.59-4.19 (m, 4H), 2.78-2.53 (m, 5H), |
| | 1.90-1.29 (m, 4H). LC-MS (ESI, m/z): [M + H]⁺ 498. |
| 21-64 | 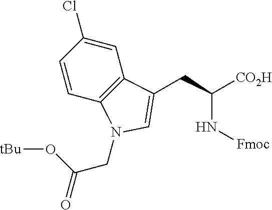 |
| | (S)-2-((((9H-fluoren-9-yl)methoxy)carbonyl)amino)-3-(1-(2- |
| | (tert-butoxy)-2-oxoethyl)-5-chloro-1H-indol-3-yl)propanoic acid |
| | methyl (2R)-2-({[(9H-fluoren-9-yl)methoxy]carbonyl} amino)-3- |
| | iodopropanoate (CAS: 156017-42-4) |
| | tert-butyl 2-(5-chloro-3-iodo-1H-indol-1-yl)acetate |
| | ¹H NMR (500 MHz, DMSO-d₆) δ 12.74 (br, 1H), 7.92-7.45 (m, |
| | 6H), 7.44-7.18 (m, 6H), 7.17-7.10 (m, 1H), 5.01-4.89 (m, 2H), |
| | 4.27-4.04 (m, 4H), 3.21-3.10 (m, 1H), 3.06-2.97 (m, 1H), |
| | 1.38 (s, 9H). LC-MS (ESI, m/z): [M + H]⁺ 575. |
| 21-65 | 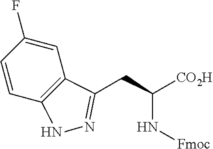 |
| | (S)-2-((((9H-fluoren-9-yl)methoxy)carbonyl)amino)-3-(5-fluoro- |
| | 1H-indazol-3-yl)propanoic acid |
| | methyl (2R)-2-({[(9H-fluoren-9-yl)methoxy]carbonyl}amino)-3- |
| | iodopropanoate (CAS: 156017-42-4) |
| | 5-fluoro-3-iodo-1H-indazole (CAS: 858629-06-8) |
| | ¹H NMR (500 MHz, DMSO-d₆) δ 12.80 (br, 2H), 7.90-7.82 (m, |
| | 2H), 7.72-7.10 (m, 9H), 6.85 (br, 1H), 4.36-3.98 (m, 4H), |
| | 3.47-3.22 (m, 2H). LC-MS (ESI, m/z): [M + H]⁺ 446. |
| 21-66 | 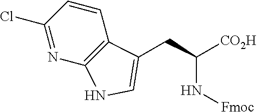 |
| | (S)-2-((((9H-fluoren-9-yl)methoxy)carbonyl)amino)-3-(6-chloro- |
| | 1H-pyrrolo[2,3-b]pyridin-3-yl)propanoic acid |
| | methyl (2R)-2-({[(9H-fluoren-9-yl)methoxy]carbonyl}amino)-3- |
| | iodopropanoate (CAS: 156017-42-4) |
| | 6-chloro-3-iodo-1H-pyrrolo[2,3-b]pyridine (CAS: 1190322-78-1) |
| | ¹H NMR (500 MHz, DMSO-d₆) δ 12.74 (br, 1H), 11.67 (s, 1H), |
| | 8.07-8.00 (m, 1H), 7.91-7.83 (m, 2H), 7.72 (d, J = 8.4 Hz, 1H), |
| | 7.62 (t, J = 7.4 Hz, 2H), 7.43-7.24 (m, 5H), 7.06 (d, J = |
| | 8.2 Hz, 1H), 4.27-4.05 (m, 4H), 3.22-3.15 (m, 1H), |
| | 3.08-2.96 (m, 1H). LCMS (m/z): [M + H]⁺ 462. |
| 21-67 | 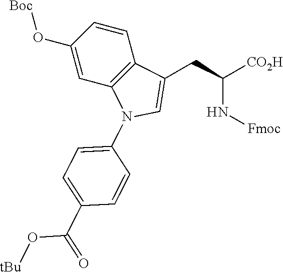 |
| | (S)-2-((((9H-fluoren-9-yl)methoxy)carbonyl)amino)-3-(6-((tert- |
| | butoxycarbonyl)oxy)-1-(4-(tert-butoxycarbonyl)phenyl)-1H-indol- |
| | 3-yl)propanoic acid |
| | methyl (2R)-2-({[(9H-fluoren-9-yl)methoxy]carbonyl} amino)-3- |
| | iodopropanoate (CAS: 156017-42-4) |
| | tert-butyl 4-(6-((tert-butoxycarbonyl)oxy)-3-iodo-1H-indol-1- |
| | yl)benzoate |
| | ¹H NMR (500 MHz, DMSO-d₆) δ 12.89 (br, 1H), 8.05-7.98 (m, |
| | 2H), 7.90-7.77 (m, 3H), 7.75-7.69 (m, 1H), 7.68-7.55 (m, |
| | 5H), 7.52-7.43 (m, 1H), 7.41-7.32 (m, 2H), |
| | 7.28-7.14 (m, 2H), 7.08-6.95 (m, 1H), 4.38-4.27 (m, 1H), |
| | 4.24-4.09 (m, 3H), 3.31-3.23 (m, 1H), 3.18-3.08 (m, 1H), 1.58 |
| | (s, 9H), 1.47 (s, 9H). LC-MS (ESI, m/z): [M − tBu + H]⁺ 663. |
| 21-68 | 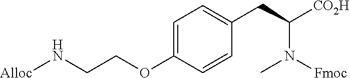 |
| | (S)-2-((((9H-fluoren-9-yl)methoxy)carbonyl)(methyl)amino)-3-(4- |
| | (2-(((allyloxy)carbonyl)amino)ethoxy)phenyl)propanoic acid |
| | methyl (2R)-2-({[(9H-fluoren-9-yl)methoxy]carbonyl}(methyl) |
| | amino)-3-iodopropanoate (CAS: 2973756-19-1) |
| | tert-butyl (2-(4-bromophenoxy)ethyl)carbamate (CAS: 1593201- |
| | 17-2) |
| | ¹H NMR (500 MHz, DMSO-d₆) δ 12.93 (br, 1H), |
| | 7.95-7.84 (m, 2H), 7.62-7.46 (m, 2H), 7.46-7.37 (m, |
| | 3H), 7.37-7.25 (m, 2H), 7.14-6.91 (m, 2H), 6.87-6.72 (m, |
| | 2H), 5.99-5.84 (m, 1H), 5.35-5.23 (m, 1H), 5.22-5.12 (m, |
| | 1H), 4.83-4.56 (m, 1H), 4.54-4.43 (m, 2H), 4.37-4.13 |
| | (m, 3H), 4.00-3.83 (m, 2H), 3.32-3.26 (m, 2H), 3.20-2.72 (m, |
| | 2H), 2.72-2.62 (m, 3H). LC-MS (ESI, m/z): [M + H]⁺ 545. |
| 21-69 | 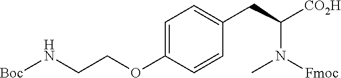 |
| | (S)-2-((((9H-fluoren-9-yl)methoxy)carbonyl)(methyl)amino)-3-(4- |
| | (2-((tert-butoxycarbonyl)amino)ethoxy)phenyl)propanoic acid |
| | methyl (2R)-2-({[(9H-fluoren-9-yl)methoxy]carbonyl}(methyl) |
| | amino)-3-iodopropanoate (CAS: 2973756-19-1) |
| | tert-butyl (2-(4-bromophenoxy)ethyl)carbamate (CAS: 1593201- |
| | 17-2) |
| | ¹H NMR (500 MHz, DMSO-d₆) δ 12.82 (br, 1H), 7.94 - 7.83 |
| | (m, 2H), 7.63-7.46 (m, 2H), 7.46-7.37 (m, 2H), 7.37-7.24 (m, |
| | 2H), 7.16-6.90 (m, 3H), 6.86-6.54 (m, 2H), 4.85-4.53 (m, 1H), |
| | 4.38-4.11 (m, 3H), 3.96-3.79 (m, 2H), 3.29-3.21 (m, 2H), |
| | 3.19-2.72 (m, 2H), 2.72-2.61 (m, 3H), 1.43-1.31 (m, 9H). |
| | LC-MS (ESI, m/z): [M + H]⁺ 561. |
| 21-70 | 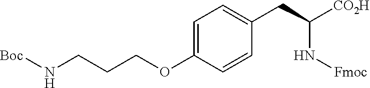 |
| | (S)-2-((((9H-fluoren-9-yl)methoxy)carbonyl)amino)-3-(4-(3-((tert- |
| | butoxycarbonyl)amino)propoxy)phenyl)propanoic acid |
| | methyl (2R)-2-({[(9H-fluoren-9-yl)methoxy]carbonyl}amino)-3- |
| | iodopropanoate (CAS: 156017-42-4) |
| | 1,1-Dimethylethyl N-[3-(4-bromophenoxy)propyl]carbamate |
| | (CAS: 1052138-95-0) |
| | ¹H NMR (500 MHz, DMSO-d₆) δ 12.71 (br, 1H), 7.90-7.87 (m, |
| | 2H), 7.74-7.45 (m, 3H), 7.43-7.37 (m, 2H), 7.34-7.25 (m, 2H), |
| | 7.24-7.12 (m, 2H), 6.93-6.75 (m, 3H), 4.27-4.00 (m, 4H), 3.96- |
| | 3.86 (m, 2H), 3.10-2.89 (m, 3H), 2.83-2.62 (m, 1H), 1.85- |
| | 1.75 (m, 2H), 1.37 (s, 9H). LC-MS (ESI, m/z): [M + H]⁺ 561. |

### Example-22

The following compounds can be synthesized as outlined for the preparation of Reference example-4-01, Reference example-4-02, or Reference example-4-03 employing appropriate starting materials in the table.

| | Structure and IUPAC name |
|---|---|
| | Starting material-A |
| | Starting material-B |
| Example | NMR and MS |
| 22-01 | 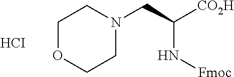 |
| | (S)-2-((((9H-fluoren-9-yl)methoxy)carbonyl)amino)-3- |
| | morpholinopropanoic acid hydrochloride |
| | 3-((9H-fluoren-9-yl)methyl) 4-(tert-butyl) (S)-1,2,3-oxathiazolidine-3,4- |
| | dicarboxylate 2,2-dioxide (CAS: 2417280-85-2) |
| | morpholine (CAS: 110-91-8) |
| | ¹H NMR (500 MHz, DMSO-d₆) δ 13.89-12.88 (m, 1H), 10.98-10.48 (m, |
| | 1H), 8.08-7.87 (m, 3H), 7.79-7.63 (m, 2H), 7.49-7.26 (m, 4H), 4.62- |
| | 4.22 (m, 4H), 4.12-3.70 (m, 4H), 3.70-2.85 (m, 6H). LC-MS (ESI, m/z): |
| | [M + H]⁺ 397. |
| 22-02 | 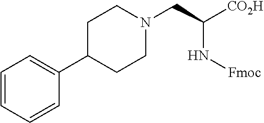 |
| | (S)-2-((((9H-fluoren-9-yl)methoxy)carbonyl)amino)-3-(4-phenylpiperidin- |
| | 1-yl)propanoic acid hydrochloride |
| | 3-((9H-fluoren-9-yl)methyl) 4-(tert-butyl) (S)-1,2,3-oxathiazolidine-3,4- |
| | dicarboxylate 2,2-dioxide (CAS: 2417280-85-2) |
| | 4-phenylpiperidine (CAS: 771-99-3) |
| | ¹H NMR (500 MHz, DMSO-d₆) δ 13.39 (br, 1H), 10.20 (br, 1H), 8.01 (d, J = |
| | 8.4 Hz, 1H), 7.91 (d, J = 7.6 Hz, 2H), 7.74 (dd, J = 4.1, 7.3 Hz, 2H), 7.45- |
| | 7.40 (m, 2H), 7.37-7.31 (m, 4H), 7.28-7.21 (m, 3H), 4.57 (br, 1H), 4.43- |
| | 4.33 (m, 2H), 4.31-4.23 (m, 1H), 3.63-3.38 (m, 4H), 3.21-3.07 (m, 2H), |
| | 2.80 (br, 1H), 2.16-1.86 (m, 4H). LC-MS (ESI, m/z): [M + H]⁺ 471. |
| 22-03 | 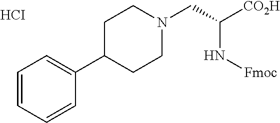 |
| | (R)-2-((((9H-fluoren-9-yl)methoxy)carbonyl)amino)-3-(4-phenylpiperidin- |
| | 1-yl)propanoic acid hydrochloride |
| | Reference example-3-04 |
| | 4-phenylpiperidine (CAS: 771-99-3) |
| | ¹H NMR (500 MHz, DMSO-d₆) δ 7.95 (br, 1H), 7.90 (d, J = 7.6 Hz, 2H), |
| | 7.74 (dd, J = 2.7, 7.4 Hz, 2H), 7.45-7.40 (m, 2H), 7.34 (dd, J = 7.2, 7.2 Hz, |
| | 4H), 7.27-7.13 (m, 3H), 4.58-4.46 (m, 1H), 4.42-4.32 (m, 2H), 4.31- |
| | 4.23 (m, 1H), 3.51-3.45 (m, 2H), 3.38-3.29 (m, 2H), 3.05 (br, 2H), 2.82- |
| | 2.72 (m, 1H), 2.12-1.88 (m, 4H). LC-MS (ESI, m/z): [M + H]⁺ 471. |
| 22-04 | 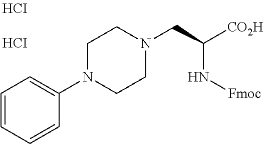 |
| | (S)-2-((((9H-fluoren-9-yl)methoxy)carbonyl)amino)-3-(4-phenylpiperazin- |
| | 1-yl)propanoic acid dihydrochloride |
| | 3-((9H-fluoren-9-yl)methyl) 4-(tert-butyl) (S)-1,2,3-oxathiazolidine-3,4- |
| | dicarboxylate 2,2-dioxide (CAS: 2417280-85-2) |
| | 1-phenylpiperazine (CAS: 92-54-6) |
| | ¹H NMR (500 MHz, DMSO-d₆) δ 13.38 (br, 1H) 10.85 (br, 1H), 8.1 - |
| | 7.48 (m, 5H), 7.47-7.40 (m, 2H), 7.38-7.32 (m, 2H), 7.31-7.24 (m, 2H), |
| | 7.07-7.97 (m, 2H), 6.91-6.83 (m, 2H), 4.63-4.54 (m, 1H), 4.45-4.33 |
| | (m, 2H), 4.31-4.23 (m, 1H), 3.90-2.99 (m, 10H). LC-MS (ESI, m/z): |
| | [M + H]⁺ 472. |
| 22-05 | 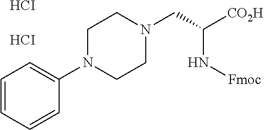 |
| | (R)-2-((((9H-fluoren-9-yl)methoxy)carbonyl)amino)-3-(4-phenylpiperazin- |
| | 1-yl)propanoic acid dihydrochloride |
| | Reference example-3-04 |
| | 1-phenylpiperazine (CAS: 92-54-6) |
| | Substantially identical to Example 22-04 |
| 22-06 | 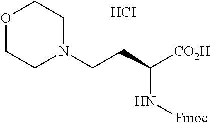 |
| | (S)-2-((((9H-fluoren-9-yl)methoxy)carbonyl)amino)-4-morpholinobutanoic |
| | acid hydrochloride |
| | 4-tert-butyl 3-(9H-fluoren-9-yl)methyl (4S)-2,2-dioxo-1,2lambda6,3- |
| | oxathiazinane-3,4-dicarboxylate (CAS: 2973756-52-2) |
| | morpholine (CAS: 110-91-8) |
| | ¹H NMR (500 MHz, DMSO-d₆) δ 12.92 (br, 1H), 11.01 (br, 1H), 7.91 (d, J = |
| | 7.5 Hz, 2H), 7.82-7.76 (m, 1H), 7.75-7.69 (m, 2H), 7.46-7.40 (m, 2H), |
| | 7.38-7.30 (m, 2H), 4.39-4.20 (m, 3H), 4.09-4.02 (m, 1H), 3.99-3.71 (m, |
| | 4H), 3.4-2.95 (m, 6H, overlapped with water), 2.30-1.99 (m, 2H). LC- |
| | MS (ESI, m/z): [M + H]⁺ 411. |
| 22-07 | 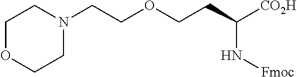 |
| | (2S)-2-({[(9H-fluoren-9-yl)methoxy]carbonyl}amino)-4-[2-(morpholin-4- |
| | yl)ethoxy]butanoic acid |
| | 4-tert-butyl 3-(9H-fluoren-9-yl)methyl (4S)-2,2-dioxo-1,2lambda6,3- |
| | oxathiazinane-3,4-dicarboxylate (CAS: 2973756-52-2) |
| | 4-Morpholineethanol (CAS: 622-40-2) |
| | ¹H NMR (500 MHz, DMSO-d₆) δ 7.94-7.28 (m, 9H), 4.34-4.01 (m, 4H), |
| | 3.57-3.36 (m, 8H), 2.47-2.29 (m, 6H), 2.02-1.71 (m, 2H). LCMS (m/z) |
| | [M + H]⁺ 455. |
| 22-08 | 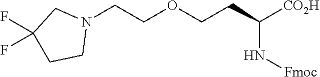 |
| | (2S)-4-[2-(3,3-difluoropyrrolidin-1-yl)ethoxy]-2-({[(9H-fluoren-9- |
| | yl)methoxy]carbonyl} amino)butanoic acid |
| | 4-tert-butyl 3-(9H-fluoren-9-yl)methyl (4S)-2,2-dioxo-1,2lambda6,3- |
| | oxathiazinane-3,4-dicarboxylate (CAS: 2973756-52-2) |
| | 3,3-Difluoro-1-pyrrolidineethanol (CAS: 950662-61-0) |
| | ¹H NMR (500 MHz, DMSO-d₆) δ 12.62 (br s, 1H), 7.90 (d, J = 7.5 Hz, 2H), |
| | 7.75-7.69 (m, 2H), 7.62 (d, J = 8.2 Hz, 1H), 7.42 (t, J = 7.4 Hz, 2H), 7.33 |
| | (t, J = 7.4 Hz, 2H), 4.31-4.01 (m, 4H), 3.51-3.40 (m, 5H), 3.01-2.59 (m, |
| | 5H), 2.25-2.14 (m, 2H), 2.03-1.91 (m, 1H), 1.86-1.71 (m, 1H). LC-MS |
| | (ESI, m/z): [M + H]⁺ 475. |
| 22-09 | 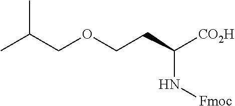 |
| | (2S)-2-({[(9H-fluoren-9-yl)methoxy]carbonyl}amino)-4-(2- |
| | methylpropoxy)butanoic acid |
| | 4-tert-butyl 3-(9H-fluoren-9-yl)methyl (4S)-2,2-dioxo-1,2lambda6,3- |
| | oxathiazinane-3,4-dicarboxylate (CAS: 2973756-52-2) |
| | Isobutanol (CAS: 78-83-1) |
| | ¹H NMR (500 MHz, DMSO-d₆) δ 12.59 (br, 1H), 7.96-7.83 (m, 2H), 7.77- |
| | 7.61 (m, 2H), 7.61-7.53 (m, 1H), 7.49-7.37 (m, 2H), 7.37-7.26 (m, 2H), |
| | 4.38-4.02 (m, 4H), 3.54-3.36 (m, 2H), 3.21-3.00 (m, 2H), 2.09-1.90 (m, |
| | 1H), 1.88-1.65 (m, 2H), 0.92-0.74 (m, 6H). LC-MS (ESI, m/z): [M + H]⁺ |
| | 398. |
| 22-10 | 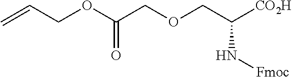 |
| | (2R)-2-({[(9H-fluoren-9-y1)methoxy]carbonyl}amino)-3-[2-oxo-2-(prop-2- |
| | en-1-yloxy)ethoxy]propanoic acid |
| | Reference example-3-04 |
| | Allyl glycolate (CAS: 4704-23-8) |
| | ¹H NMR (500 MHz, DMSO-d₆) δ 12.85 (br, 1H), 7.90 (d, J = 7.6Hz, 2H), |
| | 7.74 (d, J=7.5Hz, 2H), 7.63 (d, J = 8.2Hz, 1H), 7.46-7.39 (m, 2H), 7.37-7.29 |
| | (m, 2H), 6.00-5.84 (m, 1H), 5.37-5.27 (m, 1H), 5.26-5.16 (m, 1H), 4.61 |
| | (d, J = 5.5Hz, 2H), 4.32-4.26 (m, 2H), 4.26-4.17 (m, 4H), 3.84-3.67 (m, |
| | 2H). LC-MS (ESI, m/z): [M + H]⁺ 426. |
| 22-11 | 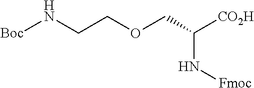 |
| | (2R)-3-(2-{[(tert-butoxy)carbonyl]amino}ethoxy)-2-({[(9H-fluoren-9- |
| | yl)methoxy]carbonyl}amino)propanoic acid |
| | 3-(9H-fluoren-9-yl)methyl 4-methyl (4R)-2,2-dioxo-1,2lambda6,3- |
| | oxathiazolidine-3,4-dicarboxylate (CAS: 3054045-94-9) |
| | benzyl (2-hydroxyethyl)carbamate (CAS: 77987-49-6) |
| | ¹H NMR (500 MHz, DMSO-d₆) δ 12.72 (br, 1H), 7.90 (d, J = 7.6 Hz, 2H), |
| | 7.77-7.62 (m, 3H), 7.45-7.28 (m, 4H), 6.89-6.79 (m, 1H), 4.33-4.17 |
| | (m, 4H), 3.71-3.60 (m, 2H), 3.44-3.36 (m, 2H), 3.14-3.00 (m, 2H), 1.37 |
| | (s, 9H). LC-MS (ESI, m/z): [M − Boc + H]⁺ 371. |
| 22-12 | 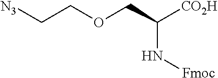 |
| | (2S)-3-(2-azidoethoxy)-2-({[(9H-fluoren-9- |
| | yl)methoxy]carbonyl}amino)propanoic acid |
| | 3-((9H-fluoren-9-yl)methyl) 4-(tert-butyl) (S)-1,2,3-oxathiazolidine-3,4- |
| | dicarboxylate 2,2-dioxide (CAS: 2417280-85-2) |
| | Azidoethanol (CAS: 1517-05-1) |
| | ¹H NMR (500 MHz, DMSO-d₆) δ 12.87 (br, 1H), 7.89 (d, J = 7.6 Hz, 2H), |
| | 7.73 (d, J = 7.5 Hz, 2H), 7.58 (d, J = 8.2 Hz, 1H), 7.45-7.39 (m, 2H), 7.35- |
| | 7.31 (m, 2H), 4.34-4.15 (m, 4H), 3.72 (d, J = 5.6 Hz, 2H), 3.65-3.57 (m, |
| | 2H), 3.42-3.38 (m, 2H). LCMS (m/z) [M + H]⁺ 397. |
| 22-13 | 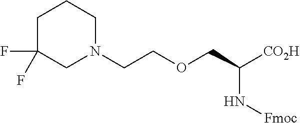 |
| | (2S)-3-[2-(3,3-difluoropiperidin-1-yl)ethoxy]-2-({[(9H-fluoren-9- |
| | yl)methoxy]carbonyl}amino)propanoic acid |
| | 3-((9H-fluoren-9-yl)methyl) 4-(tert-butyl) (S)-1,2,3-oxathiazolidine-3,4- |
| | dicarboxylate 2,2-dioxide (CAS: 2417280-85-2) |
| | 2-(3,3-difluoropiperidin-1-yl)ethan-1-ol (CAS: 1894442-02-4) |
| | ¹H NMR (500 MHz, DMSO-d₆) δ 12.80 (br s, 1H), 7.92-7.87 (m, 2H), 7.78- |
| | 7.70 (m, 2H), 7.66-7.58 (m, 1H), 7.46-7.38 (m, 2H), 7.36-7.30 (m, 2H), |
| | 4.32-4.05 (m, 4H), 3.72-3.60 (m, 2H), 3.58-3.50 (m, 2H), 2.81-2.28 (m, |
| | 11H), 1.90-1.75 (m, 2H), 1.69-1.51 (m, 2H). LC-MS (ESI, m/z): [M + H]⁺ |
| | 475. |
| 22-14 | 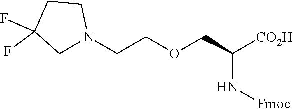 |
| | (2S)-3-[2-(3,3-difluoropyrrolidin-1-yl)ethoxy]-2-({ [(9H-fluoren-9- |
| | yl)methoxy]carbonyl } amino)propanoic acid |
| | 3-((9H-fluoren-9-yl)methyl)4-(tert-butyl) (S)-1,2,3-oxathiazolidine-3,4- |
| | dicarboxylate 2,2-dioxide (CAS: 2417280-85-2) |
| | 3,3-Difluoro-1-pyrrolidineethanol (CAS: 950662-61-0) |
| | ¹H NMR (500 MHz, DMSO-d₆) δ 12.70 (br, 1H), 7.92-7.88 (m, 2H), 7.76- |
| | 7.71 (m, 2H), 7.69-7.60 (m, 1H), 7.45-7.39 (m, 2H), 7.36-7.30 (m, 2H), |
| | 4.31-4.18 (m, 4H), 3.71-3.60 (m, 2H), 3.56-3.45 (m, 2H), 2.94-2.84 (m, |
| | 2H), 2.72-2.66 (m, 2H), 2.61-2.56 (m, 2H), 2.22-2.10 (m, 2H). LC-MS |
| | (ESI, m/z): [M + H]⁺ 461. |
| 22-15 | 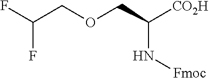 |
| | (2S)-3-(2,2-difluoroethoxy)-2-({[(9H-fluoren-9- |
| | yl)methoxy]carbonyl }amino)propanoic acid |
| | 3-((9H-fluoren-9-yl)methyl) 4-(tert-butyl) (S)-1,2,3-oxathiazolidine-3,4- |
| | dicarboxylate 2,2-dioxide (CAS: 2417280-85-2) |
| | 2,2-Difluoroethanol (CAS: 359-13-7) |
| | ¹H NMR (500 MHz, DMSO-d₆) δ 12.88 (br, 1H), 7.92-7.88 (m, 2H), 7.76- |
| | 7.70 (m, 3H), 7.45-7.39 (m, 2H), 7.35-7.31 (m, 2H), 6.26-5.98 (m, 1H), |
| | 4.32-4.27 (m, 2H), 4.27-4.20 (m, 2H), 3.81-3.65 (m, 4H). LC-MS (ESI, |
| | m/z): [M + H]⁺ 392. |
| 22-16 | 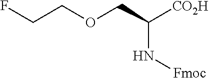 |
| | (2S)-2-({[(9H-fluoren-9-y1)methoxy]carbonyl}amino)-3-(2- |
| | fluoroethoxy)propanoic acid |
| | 3-((9H-fluoren-9-yl)methyl) 4-(tert-butyl) (S)-1,2,3-oxathiazolidine-3,4- |
| | dicarboxylate 2,2-dioxide (CAS: 2417280-85-2) |
| | 2-Fluoroethanol (CAS: 371-62-0) |
| | ¹H NMR (500 MHz, DMSO-d₆) δ 12.84 (br, 1H), 7.92-7.88 (m, 2H), 7.77- |
| | 7.72 (m, 2H), 7.71-7.63 (m, 1H), 7.45-7.39 (m, 2H), 7.36-7.31 (m, 2H), |
| | 4.60-4.44 (m, 2H), 4.32-4.19 (m, 4H), 3.75-3.59 (m, 4H). LC-MS (ESI, |
| | m/z): [M + H]⁺ 374. |
| 22-17 | 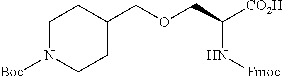 |
| | (2S)-3-({1-[(tert-butoxy)carbonyl]piperidin-4-yl}methoxy)-2-({[(9H- |
| | fluoren-9-y1)methoxy]carbonyl}amino)propanoic acid |
| | 3-((9H-fluoren-9-yl)methyl) 4-(tert-butyl) (S)-1,2,3-oxathiazolidine-3,4- |
| | dicarboxylate 2,2-dioxide (CAS: 2417280-85-2) |
| | tert-butyl 4-(hydroxymethyl)piperidine-1-carboxylate (CAS: 123855-51-6) |
| | ¹H NMR (500 MHz, METHANOL-d₄) δ 7.58-7.29 (m, 8H), 4.50-4.35 (m, |
| | 1H), 4.34-4.20 (m, 2H), 4.11-4.01 (m, 2H), 3.79-3.16 (m,7H), 1.85-1.69 |
| | (m, 3H), 1.48-1.43 (m, 9H), 1.21-1.04 (m, 2H). LC-MS (ESI, m/z): [M + H − |
| | Boc]⁺ 425. |
| 22-18 | 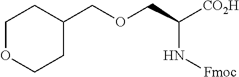 |
| | (2S)-2-({[(9H-fluoren-9-y1)methoxy]carbonyl}amino)-3-[(oxan-4- |
| | yl)methoxy]propanoic acid |
| | 3-((9H-fluoren-9-yl)methyl) 4-(tert-butyl)(S)-1,2,3-oxathiazolidine-3,4- |
| | dicarboxylate 2,2-dioxide (CAS: 2417280-85-2) |
| | (tetrahydro-2H-pyran-4-yl)methanol (CAS: 14774-37-9) |
| | ¹H NMR (500 MHz, DMSO-d₆) δ 12.80 (br, 1H), 7.90 (d, J = 7.5 Hz, 2H), |
| | 7.74 (d, J = 6.4 Hz, 2H), 7.62 (d, J = 8.2 Hz, 1H), 7.42 (t, J = 7.5 Hz, 2H), |
| | 7.32 (t, J = 7.5 Hz, 2H), 4.35-4.10 (m, 4H), 3.88-3.79 (m, 2H), 3.69-3.50 |
| | (m, 2H), 3.30-3.20 (m, 4H), 1.83-1.69 (m, 1H), 1.57-1.50 (m, 2H), 1.21- |
| | 1.08 (m, 2H). LC-MS (ESI, m/z): [M + H]⁺ 426. |
| 22-19 | 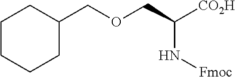 |
| | (2S)-3-(cyclohexylmethoxy)-2-({[(9H-fluoren-9- |
| | yl)methoxy]carbonyl}amino)propanoic acid |
| | 3-((9H-fluoren-9-yl)methyl) 4-(tert-butyl)(S)-1,2,3-oxathiazolidine-3,4- |
| | dicarboxylate 2,2-dioxide (CAS: 2417280-85-2) |
| | cyclohexylmethanol (CAS: 100-49-2) |
| | ¹H NMR (500 MHz, DMSO-d₆) δ 12.77 (br, 1H), 7.90 (d, J = 7.5 Hz, 2H), |
| | 7.74 (d, J = 7.5 Hz, 2H), 7.65-7.57 (m, 1H), 7.42 (t, J = 7.5 Hz, 2H), 7.32 |
| | (t, J = 7.4 Hz, 2H), 4.32-4.14 (m, 4H), 3.66-3.48 (m, 2H), 3.26-3.13 (m, |
| | 2H), 1.72-1.56 (m, 5H), 1.54-1.45 (m, 1H), 1.25-1.03 (m, 3H), 0.92- |
| | 0.80 (m, 2H). LC-MS (ESI, m/z): [M + H]⁺ 424. |
| 22-20 | 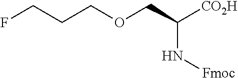 |
| | (2S)-2-({[(9H-fluoren-9-yl)methoxy]carbonyl}amino)-3-(3- |
| | fluoropropoxy)propanoic acid |
| | 3-((9H-fluoren-9-yl)methyl)4-(tert-butyl)(S)-1,2,3-oxathiazolidine-3,4- |
| | dicarboxylate 2,2-dioxide (CAS: 2417280-85-2) |
| | 3-fluoropropan-1-ol (CAS: 462-43-1) |
| | ¹H NMR (500 MHz, DMSO-d₆) δ 12.81 (br, 1H), 7.90 (d, J = 7.5 Hz, 2H), |
| | 7.74 (d, J = 7.3 Hz, 2H), 7.67 (d, J = 8.2 Hz, 1H), 7.45-7.40 (m, 2H), 7.35- |
| | 7.30 (m, 2H), 4.58-4.39 (m, 2H), 4.32-4.18 (m, 4H), 3.70-3.60 (m, 2H), |
| | 3.56-3.41 (m, 2H), 1.92-1.81 (m, 2H). LC-MS (ESI, m/z): [M + H]⁺ 388. |

### Example-23

The following compounds can be synthesized as outlined for the preparation of Reference example-6-01, Reference example-6-02, Reference example-6-03, Reference example-6-04, Reference example-6-05, Example-4-01 or the methods described in Tetrahedron: Asymmetry 2001, 12, 481. employing appropriate starting materials in the table.

| | Structure and IUPAC name |
|---|---|
| | Starting material-A |
| | Starting material-B |
| Example | NMR and MS |
| 23-01 | 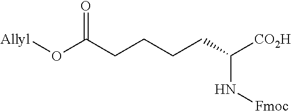 |
| | (R)-2-((((9H-fluoren-9-yl)methoxy)carbonyl)amino)-7-(allyloxy)-7- |
| | oxoheptanoic acid |
| | (2S)-3,6-dimethoxy-2-(propan-2-yl)-2,5-dihydropyrazine (CAS: 78342-42- |
| | 4) |
| | 2-Propen-1-yl 5-bromopentanoate (CAS: 105464-35-5) |
| | ¹H NMR (500 MHz, DMSO-d₆) δ 12.60 (br, 1H), 7.90 (d, J = 7.6 Hz, 2H), |
| | 7.78-7.10 (m, 7H), 5.98-5.83 (m, 1H), 5.33-5.24 (m, 1H), 5.24-5.14 (m, |
| | 1H), 4.58-4.48 (m, 2H), 4.34-4.16 (m, 3H), 3.95-3.82 (m, 1H), 2.38- |
| | 2.28 (m, 2H), 1.76-1.44 (m, 4H), 1.41-1.22 (m, 2H). LC-MS (ESI, m/z): |
| | [M + H]⁺ 438. |

### Example-24

The following compounds can be synthesized as outlined for the preparation of Reference example-7-01, Reference example-7-02, Reference example-7-03, Reference example-7-04, or Reference example-7-05 employing appropriate starting materials in the table.

| | Structure and IUPAC name |
|---|---|
| | Starting material-A |
| | Starting material-B |
| Example | NMR and MS |
| 24-01 | 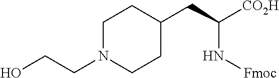 |
| | (S)-2-((((9H-fluoren-9-yl)methoxy)carbonyl)amino)-3-(1-(2- |
| | hydroxyethyl)piperidin-4-yl)propanoic acid |
| | (S)-2-((((9H-fluoren-9-yl)methoxy)carbonyl)amino)-3-(piperidin-4- |
| | yl)propanoic acid hydrochloride (CAS: 2803482-17-7) |
| | 2-Bromoethanol (CAS: 540-51-2) |
| | ¹H NMR (500 MHz, METHANOL-d₄) δ 7.69 (d, J = 7.5 Hz, 2H), 7.60-7.51 |
| | (m, 2H), 7.- 7.24 (m, 2H), 7.24-7.18 (m, 2H), 4.33-4.18 (m, 2H), 4.14- |
| | 4.06 (m, 1H), 4.02-3.93 (m, 1H), 3.79-3.68 (m, 2H), 3.39-3.32 (m, 1H), |
| | 3.25-3.18 (m, 1H), 3.07-2.99 (m, 2H), 2.88-2.68 (m, 2H), 2.00-1.89 (m, |
| | 1H), 1.87-1.77 (m, 1H), 1.70-1.36 (m, 5H). LC-MS (ESI, m/z): [M + H]⁺ |
| | 439. |
| 24-02 | 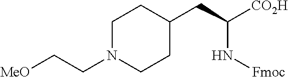 |
| | (S)-2-((((9H-fluoren-9-yl)methoxy)carbonyl)amino)-3-(1-(2- |
| | methoxyethyl)piperidin-4-yl)propanoic acid |
| | (S)-2-((((9H-fluoren-9-yl)methoxy)carbonyl)amino)-3-(piperidin-4- |
| | yl)propanoic acid hydrochloride (CAS: 2803482-17-7) |
| | Bromoethyl methyl ether (CAS: 6482-24-2) |
| | ¹H NMR (500 MHz, DMSO-d₆) δ 12.43 (br, 1H), 7.90 (d, J = 7.5 Hz, 2H), |
| | 7.71 (d, J = 7.3 Hz, 2H), 7.58-7.48 (m, 1H), 7.47-7.38 (m, 2H), 7.36-7.29 |
| | (m, 2H), 4.32-4.19 (m, 3H), 3.99-3.87 (m, 1H), 3.44-3.38 (m, 2H), 3.22 |
| | (s, 3H), 2.86-2.79 (m, 2H), 2.45-2.40 (m, 2H), 1.95-1.82 (m, 2H), 1.69- |
| | 1.44 (m, 4H), 1.36-0.96 (m, 3H). LC-MS (ESI, m/z): [M + H]⁺ 453. |
| 24-03 | 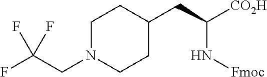 |
| | (S)-2-((((9H-fluoren-9-yl)methoxy)carbonyl)amino)-3-(1-(2,2,2- |
| | trifluoroethyl)piperidin-4-yl)propanoic acid |
| | (S)-2-((((9H-fluoren-9-yl)methoxy)carbonyl)amino)-3-(piperidin-4- |
| | yl)propanoic acid hydrochloride (CAS: 2803482-17-7) |
| | 2-Bromo-1,1,1-trifluoroethane (CAS: 421-06-7) |
| | ¹H NMR (500 MHz, DMSO-d₆) δ 12.56 (br, 1H), 7.90 (d, J = 7.5 Hz, 2H), |
| | 7.72 (d, J = 7.5 Hz, 2H), 7.64 (d, J = 8.1 Hz, 1H), 7.45-7.39 (m, 2H), 7.37- |
| | 7.28 (m, 2H), 4.38-4.16 (m, 3H), 4.02-3.92 (m, 1H), 3.18-3.04 (m, 2H), |
| | 2.80-2.90 (m, 2H), 2.32-2.17 (m, 2H), 1.65-1.45 (m, 4H), 1.39-1.27 (m, |
| | 1H), 1.26-1.00 (m, 2H). LC-MS (ESI, m/z): [M + H]⁺ 477. |
| 24-04 | 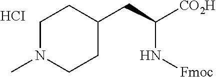 |
| | (S)-2-((((9H-fluoren-9-yl)methoxy)carbonyl)amino)-3-(1-methylpiperidin- |
| | 4-y1)propanoic acid hydrochloride |
| | (S)-2-((((9H-fluoren-9-yl)methoxy)carbonyl)amino)-3-(piperidin-4- |
| | yl)propanoic acid hydrochloride (CAS: 2803482-17-7) |
| | Iodomethane (CAS: 74-88-4) |
| | ¹H NMR (500 MHz, DMSO-d₆) δ 12.70 (br, 1H), 10.39-10.11 (m, 1H), |
| | 7.93-7.86 (m, 2H), 7.75-7.57 (m, 3H), 7.47-7.39 (m, 2H), 7.38-7.29 (m, |
| | 2H), 4.38-4.18 (m, 3H), 4.04-3.96 (m, 1H), 3.40-3.29 (m, 2H), 2.95- |
| | 2.79 (m, 2H), 2.75-2.62 (m, 3H), 1.90-1.29 (m, 7H). LC-MS (ESI, m/z): |
| | [M + H]⁺ 409. |
| 24-05 | 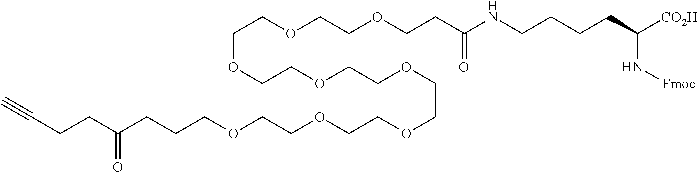 |
| | (S)-39-((((9H-fluoren-9-yl)methoxy)carbonyl)amino)-5,33-dioxo- |
| | 9,12,15,18,21,24,27,30-octaoxa-6,34-diazatetracont-1-yn-40-oic acid |
| | N2-(((9H-fluoren-9-y1)methoxy)carbonyl)-N6-(tert-butoxycarbonyl)-L- |
| | lysine (CAS: 71989-26-9) |
| | 29-oxo-4,7,10,13,16,19,22,25-octaoxa-28-azatritriacont-32-ynoic acid |
| | ¹H NMR (500 MHz, DMSO-d₆) δ 12.50 (br, 1H), 8.00-7.92 (m, 1H), 7.91- |
| | 7.88 (m, 2H), 7.85-7.77 (m, 1H), 7.75-7.70 (m, 2H), 7.65-7.56 (m, 1H), |
| | 7.44-7.40 (m, 2H), 7.36-7.31 (m, 2H), 4.31-4.26 (m, 2H), 4.25-4.19 (m, |
| | 1H), 3.94-3.84 (m, 1H), 3.60-3.55 (m, 2H), 3.54-3.43 (m, 28H), 3.43- |
| | 3.37 (m, 2H), 3.24-3.15 (m, 2H), 3.07-2.98 (m, 2H), 2.78-2.71 (m, 1H), |
| | 2.36-2.25 (m, 6H), 1.77-1.55 (m, 2H), 1.43-1.27 (m, 4H). LC-MS (ESI, |
| | m/z): [M + H]⁺ 873. |
| 24-06 | 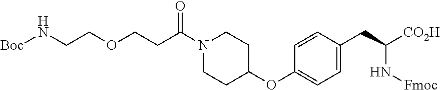 |
| | (S)-2-((((9H-fluoren-9-yl)methoxy)carbonyl)amino)-3-(4-((1-(3-(2-((tert- |
| | butoxycarbonyl)amino)ethoxy)propanoyl)piperidin-4- |
| | yl)oxy)phenyl)propanoic acid |
| | (S)-2-((((9H-fluoren-9-yl)methoxy)carbonyl)amino)-3-(4-((1-(tert- |
| | butoxycarbonyl)piperidin-4-yl)oxy)phenyl)propanoic acid |
| | 3-[2-[(1,1-Dimethylethoxy)carbonyl]amino]ethoxy]propanoic acid (CAS: |
| | 1260092-44-1) |
| | ¹H NMR (500 MHz, DMSO-d6) 8 12.72 (br, 1H), 7.89 (d, J = 7.5 Hz, 2H), |
| | 7.71 (d, J = 8.5 Hz, 1H), 7.68-7.62 (m, 2H), 7.44-7.38 (m, 2H), 7.34-7.26 |
| | (m, 2H), 7.18 (d, J = 8.5 Hz, 2H), 6.93-6.82 (m, 2H), 6.77-6.69 (m, 1H), |
| | 4.58-4.45 (m, 1H), 4.22-4.09 (m, 4H), 3.85-3.76 (m, 1H), 3.70-3.56 (m, |
| | 3H), 3.38-3.33 (m, 2H), 3.31-3.12 (m, 2H), 3.08-2.98 (m, 3H), 2.84- |
| | 2.74 (m, 1H), 2.58-2.51 (m, 2H), 1.94-1.74 (m, 2H), 1.58-1.40 (m, 2H), |
| | 1.37 (s, 9H). LC-MS (ESI, m/z): [M +H]⁺ 702. |

### Example-25

The following compounds can be synthesized as outlined for the preparation of Reference example-8-01, Reference example-8-02, or Reference example-8-03 employing appropriate starting materials in the table.

| | Structure and IUPAC name |
|---|---|
| | Starting material-A |
| | Starting material-B |
| Example | NMR and MS |
| 25-01 | 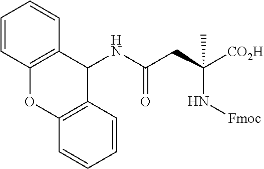 |
| | (S)-2-((((9H-fluoren-9-yl)methoxy)carbonyl)amino)-4-((9H-xanthen-9- |
| | yl)amino)-2-methyl-4-oxobutanoic acid |
| | (S)-2-((((9H-fluoren-9-yl)methoxy)carbonyl)amino)-4-(tert-butoxy)-2- |
| | methyl-4-oxobutanoic acid (CAS: 1072845-47-6) |
| | 9H-xanthen-9-amine (CAS: 35598-63-1) |
| | ¹H NMR (500 MHz, DMSO-d₆) δ 12.76 (br, 1H), 9.27-8.84 (m, 1H), 7.99- |
| | 7.83 (m, 2H), 7.78-7.65 (m, 2H), 7.47-7.39 (m, 2H), 7.38-7.22 (m, 7H), |
| | 7.21-7.07 (m, 2H), 7.07-6.91 (m, 2H), 6.33-6.22 (m, 1H), 4.28-4.15 (m, |
| | 3H), 2.89-2.71 (m, 2H), 1.46 (br, 3H). LC-MS (ESI, m/z): [M + H]⁺ 549. |
| 25-02 | 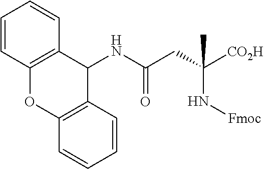 |
| | (R)-2-((((9H-fluoren-9-yl)methoxy)carbonyl)amino)-4-((9H-xanthen-9- |
| | yl)amino)-2-methyl-4-oxobutanoic acid |
| | (R)-2-((((9H-fluoren-9-yl)methoxy)carbonyl)amino)-4-(tert-butoxy)-2- |
| | methyl-4-oxobutanoic acid (CAS: 1231709-26-4) |
| | 9H-xanthen-9-amine (CAS: 35598-63-1) |
| | Substantially identical to Example 25-01 |
| 25-03 | 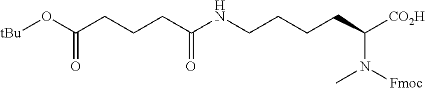 |
| | (2S)-6-[5-(tert-butoxy)-5-oxopentanamido]-2-({[(9H-fluoren-9- |
| | yl)methoxy]carbonyl}(methyl)amino)hexanoic acid |
| | allyl N2-(((9H-fluoren-9-yl)methoxy)carbonyl)-N2-methyl-L-lysinate |
| | hydrochloride (CAS: 2973752-98-4) |
| | 5-(tert-butoxy)-5-oxopentanoic acid (CAS: 63128-51-8) |
| | ¹H NMR (500 MHz, DMSO-d₆) δ 12.76 (br, 1H), 7.97-7.87 (m, 2H), 7.80- |
| | 7.72 (m, 1H), 7.70-7.58 (m, 2H), 7.48-7.38 (m, 2H), 7.37-7.23 (m, 2H), |
| | 4.56-4.18 (m, 4H), 3.05-2.94 (m, 2H), 2.73-2.70 (m, 3H), 2.18-2.12 (m, |
| | 2H), 2.08-2.03 (m, 2H), 1.85-1.52 (m, 4H), 1.49-1.26 (m, 11H), 1.21- |
| | 1.02 (m, 2H). LC-MS (ESI, m/z): [M + H]⁺ 553. |
| 25-04 | 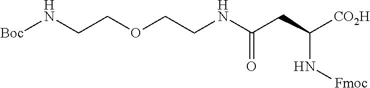 |
| | (S)-14-((((9H-fluoren-9-yl)methoxy)carbonyl)amino)-2,2-dimethyl-4,12- |
| | dioxo-3,8-dioxa-5,11-diazapentadecan-15-oic acid |
| | (S)-3-((((9H-fluoren-9-yl)methoxy)carbonyl)amino)-4-(allyloxy)-4- |
| | oxobutanoic acid (CAS: 144120-53-6) |
| | tert-butyl (2-(2-aminoethoxy)ethyl)carbamate (CAS: 127828-22-2) |
| | ¹H NMR (500 MHz, DMSO-d₆) δ 12.69 (br, 1H), 7.98 (br, 1H), 7.89 (d, J = |
| | 7.5 Hz, 2H), 7.70 (d, J = 7.5 Hz, 2H), 7.51-7.45 (m, 1H), 7.44-7.39 (m, |
| | 2H), 7.35-7.30 (m, 2H), 6.83-6.74 (m, 1H), 4.35-4.16 (m, 4H), 3.39- |
| | 3.34 (m, 4H), 3.24-3.14 (m, 2H), 3.11-3.02 (m, 2H), 2.61-2.56 (m, 1H), |
| | 2.52-2.45 (m, 1H), 1.37 (s, 9H). LC-MS (ESI, m/z): [M + H]⁺ 542. |
| 25-05 | 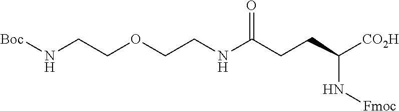 |
| | (S)-15-((((9H-fluoren-9-yl)methoxy)carbonyl)amino)-2,2-dimethyl-4,12- |
| | dioxo-3,8-dioxa-5,11-diazahexadecan-16-oic acid |
| | (S)-4-((((9H-fluoren-9-yl)methoxy)carbonyl)amino)-5-(allyloxy)-5- |
| | oxopentanoic acid (CAS: 144120-54-7) |
| | tert-butyl (2-(2-aminoethoxy)ethyl)carbamate (CAS: 127828-22-2) |
| | ¹H NMR (500 MHz, DMSO-d₆) δ 12.57 (br, 1H), 7.93-7.85 (m, 3H), 7.73 |
| | (d, J = 7.5 Hz, 2H), 7.69-7.60 (m, 1H), 7.44-7.40 (m, 2H), 7.36-7.31 (m, |
| | 2H), 6.81-6.73 (m, 1H), 4.31-4.18 (m, 3H), 3.99-3.89 (m, 1H), 3.39- |
| | 3.34 (m, 4H), 3.29-3.16 (m, 2H), 3.10-3.02 (m, 2H), 2.22-2.15 (m, 2H), |
| | 2.03-1.91 (m, 1H), 1.83-1.73 (m, 1H), 1.37 (s, 9H). LC-MS (ESI, m/z): |
| | [M + H]⁺ 556. |

### Example-26

The following compounds can be synthesized as outlined for the preparation of Reference example-9-01, Reference example-9-02, Reference example-9-03, or Example-7-01 employing appropriate starting materials in the table.

| | Structure and IUPAC name |
|---|---|
| | Starting material-A |
| Ex- | Starting material-B |
| ample | NMR and MS |
| 26-01 | 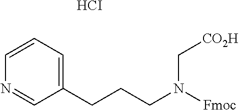 |
| | 2-({[(9H-fluoren-9-yl)methoxy]carbonyl}[3-(pyridin-3- |
| | yl)propyl]amino)acetic acid hydrochloride |
| | tert-butyl 2-bromoacetate (CAS: 5292-43-3) |
| | 3-Pyridinepropanamine (CAS: 41038-69-1) |
| | ¹H NMR (500 MHz, DMSO-d₆) δ 13.21-12.30 (m, 1H), 8.82-8.46 |
| | (m, 2H), 8.32-8.04 (m, 1H), 7.94-7.71 (m, 3H), 7.68-7.54 (m, |
| | 2H), 7.47-7.20 (m, 4H), 4.49 (d, J = 5.3 Hz, 1H), 4.30-4.17 (m, |
| | 2H), 3.97 (s, 1H), 3.83 (s, 1H), 3.35-3.27 (m, 1H), 3.03-2.97 (m, |
| | 1H), 2.76-2.69 (m, 1H), 2.41-2.36 (m, 1H), 1.87-1.79 (m, 1H), |
| | 1.55-1.46 (m, 1H). LC-MS (ESI, m/z): [M + H]⁺ 417. |
| 26-02 | 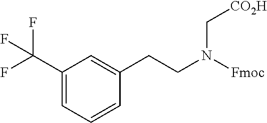 |
| | 2-({[(9H-fluoren-9-yl)methoxy]carbonyl}({2-[3- |
| | (trifluoromethyl)phenyl]ethyl})amino)acetic acid |
| | 2-oxoacetic acid hydrate (CAS: 1152495-30-1) |
| | 2-(3-Trifluoromethylphenyl)ethylamine (CAS: 52516-30-0) |
| | ¹H NMR (500 MHz, DMSO-d₆) δ 12.71 (br, 1H), 7.93-7.84 (m, |
| | 2H), 7.68-7.46 (m, 5H), 7.45-7.14 (m, 5H), 4.53 (d, J = 5.2 Hz, |
| | 1H), 4.28-4.16 (m, 2H), 4.02-3.76 (m, 2H), 3.53-3.45 (m, |
| | 1H), 3.16-3.07 (m, 1H), 2.93-2.86 (m, 1H), 2.49-2.43 (m, |
| | 1H). LC-MS (ESI, m/z): [M + H]⁺ 470. |
| 26-03 | 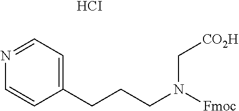 |
| | 2-({[(9H-fluoren-9-yl)methoxy]carbonyl}[3-(pyridin-4- |
| | yl)propyl]amino)acetic acid hydrochloride |
| | tert-butyl 2-bromoacetate (CAS: 5292-43-3) |
| | 4-Pyridinepropanamine (CAS: 30532-36-6) |
| | ¹H NMR (500 MHz, DMSO-d₆) δ 13.19-12.26 (m, 1H), 8.89-8.70 |
| | (m, 2H), 7.94-7.84 (m, 3H), 7.73 (d, J = 6.4 Hz, 1H), 7.66-7.58 (m, |
| | 2H), 7.44-7.27 (m, 4H), 4.51 (d, J = 5.2 Hz, 1H), 4.31-4.17 (m, |
| | 2H), 3.97 (s, 1H), 3.81 (s, 1H), 3.35-3.30 (m, 1H), 3.02-2.95 (m, |
| | 1H), 2.87-2.81 (m, 1H), 2.49-2.43 (m, 1H), 1.91-1.81 (m, |
| | 1H), 1.57-1.47 (m, 1H). LC-MS (ESI, m/z): [M + H]⁺ 417. |
| 26-04 | 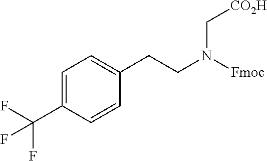 |
| | 2-({[(9H-fluoren-9-yl)methoxy]carbonyl}({2-[4- |
| | (trifluoromethyl)phenyl]ethyl})amino)acetic acid |
| | 2-oxoacetic acid hydrate (CAS: 1152495-30-1) |
| | 4-Trifluoromethylphenethylamine (CAS: 775-00-8) |
| | ¹H NMR (500 MHz, DMSO-d₆) δ 12.89-12.52 (m, 1H), 7.97-7.83 |
| | (m, 2H), 7.70-7.08 (m, 10H), 4.55-4.51 (m, 1H), 4.33-3.73 (m, 4H), |
| | 3.54-3.05 (m, 2H), 2.95-2.40 (m, 2H). LC-MS (ESI, m/z): |
| | [M + H]⁺ 470. |
| 26-05 | 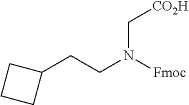 |
| | 2-[(2-cyclobutylethyl)({[(9H-fluoren-9- |
| | yl)methoxy\|carbonyl})amino]acetic acid |
| | 2-oxoacetic acid hydrate (CAS: 1152495-30-1) |
| | Cyclobutaneethanamine (CAS: 60637-97-0) |
| | ¹H NMR (500 MHz, DMSO-d₆) δ 12.65 (br, 1H), 7.89 (d, J = 7.5 |
| | Hz, 2H), 7.69-7.58 (m, 2H), 7.44-7.38 (m, 2H), 7.36-7.29 (m, 2H), |
| | 4.52 (d, J = 5.2 Hz, 1H), 4.31-4.16 (m, 2H), 3.95-3.74 (m, 2H), |
| | 3.18-2.71 (m, 2H), 2.25-1.14 (m, 9H). LC-MS (ESI, m/z): |
| | [M + H]⁺ 380. |
| 26-06 | 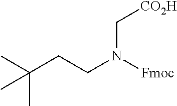 |
| | 2-[(3,3-dimethylbutyl)({[(9H-fluoren-9- |
| | yl)methoxy]carbonyl})amino]acetic acid |
| | 2-oxoacetic acid hydrate (CAS: 1152495-30-1) |
| | 3,3-Dimethylbutylamine (CAS: 15673-00-4) |
| | ¹H NMR (500 MHz, DMSO-d₆) δ 12.66 (br, 1H), 7.90-7.86 (m, |
| | 2H), 7.70-7.58 (m, 2H), 7.44-7.38 (m, 2H), 7.35-7.29 (m, 2H), |
| | 4.60-4.46 (m, 1H), 4.29-4.18 (m, 2H), 4.00-3.68 (m, 2H), |
| | 3.29-2.83 (m, 2H), 1.43-0.96 (m, 2H), 0.92-0.61 (m, 9H). |
| | LC-MS (ESI, m/z): [M + H]⁺ 382. |

### Example-27

The following compounds can be synthesized as outlined for the preparation of Reference example-10-01, Reference example-10-02, Reference example-10-03, Reference example-10-04, or Reference example-6-05 employing appropriate starting materials in the table.

| | Structure and IUPAC name |
|---|---|
| Ex- | Starting material-A |
| am- | Starting material-B |
| ple | NMR and MS |
| 27- 01 | 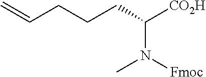 |
| | (R)-2-((((9H-fluoren-9-yl)methoxy)carbonyl)(methyl)amino)hept-6- |
| | enoic acid |
| | (R)-2-((((9H-fluoren-9-yl)methoxy)carbonyl)amino)hept-6-enoic |
| | acid (CAS: 1093645-21-6) |
| | Paraformaldehyde (CAS: 30525-89-4) |
| | ¹H NMR (500 MHz, DMSO-d₆) δ 12.80 (br, 1H), 7.95-7.85 (m, |
| | 2H), 7.68-7.59 (m, 2H), 7.48-7.21 (m, 4H), 5.85-5.72 (m, 1H), |
| | 5.11-4.88 (m, 2H), 4.54-4.21 (m, 4H), 2.77-2.65 (m, 3H), 2.13- |
| | 1.90 (m, 2H), 1.86-1.46 (m, 2H), 1.32-1.09 (m, 2H). LC-MS (ESI, |
| | m/z): [M + H]⁺ 380. |
| 27- 02 | 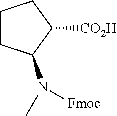 |
| | (1S,2S)-2-((((9H-fluoren-9- |
| | yl)methoxy)carbonyl)(methyl)amino)cyclopentane-1-carboxylic acid |
| | (1S,2S)-2-((((9H-fluoren-9-yl)methoxy)carbonyl)amino)cyclo- |
| | pentane-1-carboxylic acid (CAS: 359586-64-4) |
| | Paraformaldehyde (CAS: 30525-89-4) |
| | ¹H NMR (500 MHz, DMSO-d₆) δ 12.15 (br, 1H), 7.94-7.86 (m, |
| | 2H), 7.68-7.60 (m, 2H), 7.46-7.30 (m, 4H), 4.60-4.21 (m, 4H), |
| | 2.84-2.54 (m, 4H), 1.96-1.34 (m, 6H). LCMS (ESI, m/z): |
| | [M + H]⁺ 366 |
| 27- 03 | 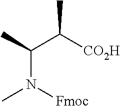 |
| | (2R,3S)-3-((((9H-fluoren-9-yl)methoxy)carbonyl)(methyl)amino)-2- |
| | methylbutanoic acid |
| | (2R,3S)-3-((((9H-fluoren-9-yl)methoxy)carbonyl)amino)-2- |
| | methylbutanoic acid (CAS: 266318-76-7) |
| | Paraformaldehyde (CAS: 30525-89-4) |
| | ¹H NMR (500 MHz, DMSO-d₆) δ 12.39 (br, 1H), 7.93-7.85 (m, |
| | 2H), 7.73-7.61 (m, 2H), 7.45-7.38 (m, 2H), 7.37-7.28 (m, |
| | 2H), 4.49-4.24 (m, 3H), 4.18-3.99 (m, 1H), 2.66-2.53 (m, |
| | 3H), 2.47-2.27 (m, 1H), 1.05-0.93 (m, 3H), |
| | 0.90-0.76 (m, 3H). LC-MS (ESI, m/z): [M + H]⁺ 354. |
| 27- 04 | 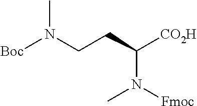 |
| | (S)-2-((((9H-fluoren-9-yl)methoxy)carbonyl)(methyl)amino)-4-((tert- |
| | butoxycarbonyl)(methyl)amino)butanoic acid |
| | (S)-2-((((9H-fluoren-9-yl)methoxy)(hydroxy)methylene)amino)-4- |
| | ((tert-butoxycarbonyl)(methyl)amino)butanoic acid |
| | (CAS: 446847-81-0) |
| | Paraformaldehyde (CAS: 30525-89-4) |
| | ¹H NMR (500 MHz, DMSO-d₆) δ 12.92 (br, 1H), 7.93-7.85 (m, |
| | 2H), 7.70-7.59 (m, 2H), 7.45-7.38 (m, 2H), 7.37-7.28 (m, |
| | 2H), 4.49-4.19 (m, 4H), 3.20-2.83 (m, 2H), 2.82-2.68 (m, 6H), |
| | 2.13-1.97 (m, 1H), 1.88-1.71 (m, 1H), 1.36 (s, 9H). |
| | LC-MS (ESI, m/z): [M + H]⁺ 469. |
| 27- 05 | 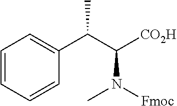 |
| | (2S,3S)-2-((((9H-fluoren-9-yl)methoxy)carbonyl)(methyl)amino)-3- |
| | phenylbutanoic acid |
| | (2S,3S)-2-((((9H-fluoren-9-yl)methoxy)carbonyl)amino)-3- |
| | phenylbutanoic acid (CAS: 247175-69-5) |
| | Paraformaldehyde (CAS: 30525-89-4) |
| | ¹H NMR (500 MHz, DMSO-d₆) δ 12.91 (br, 1H), 7.96-7.89 (m, |
| | 1H), 7.76-7.60 (m, 1H), 7.49-7.19 (m, 8H), 7.18-7.11(m, 2H), |
| | 7.00-6.91 (m, 1H), 4.85-4.39 (m, 1H), 4.37-4.26 (m, 1H), 4.25- |
| | 4.14 (m, 1H), 4.11-3.96 (m, 1H), 3.42-3.13 (m, 1H), 2.64-2.51 |
| | (m, 3H), 1.32-1.06 (m, 3H). LC-MS (ESI, m/z): [M + H]⁺ 416. |
| 27- 06 | 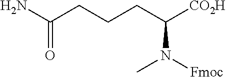 |
| | (S)-2-((((9H-fluoren-9-yl)methoxy)carbonyl)(methyl)amino)-6- |
| | amino-6-oxohexanoic acid |
| | (S)-2-((((9H-fluoren-9-yl)methoxy)carbonyl)amino)-6-amino-6- |
| | oxohexanoic acid (CAS: 190270-82-7) |
| | Paraformaldehyde (CAS: 30525-89-4) |
| | ¹H NMR (500 MHz, DMSO-d₆) δ 12.80 (br, 1H), 7.94- |
| | 7.86 (m, 2H), 7.69-7.57 (m, 2H), 7.47-7.38 (m, 2H), 7.38-7.28 |
| | (m, 2H), 7.25 (s, 1H), 6.73 (s, 1H), 4.60-4.22 (m, 4H), 2.84-2.68 |
| | (m, 3H), 2.10-2.00 (m, 2H), 1.86-1.54 (m, 2H), 1.48-1.26 (m, |
| | 2H). LC-MS (ESI, m/z): [M + H]⁺ 397. |
| 27- 07 | 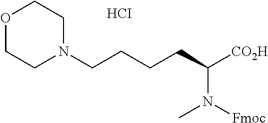 |
| | (S)-2-((((9H-fluoren-9-yl)methoxy)carbonyl)(methyl)amino)-6- |
| | morpholinohexanoic acid hydrochloride |
| | (S)-2-((((9H-fluoren-9-yl)methoxy)carbonyl)amino)-6- |
| | morpholinohexanoic acid (CAS: 2349553-17-7) |
| | Paraformaldehyde (CAS: 30525-89-4) |
| | ¹H NMR (500 MHz, DMSO-d₆) δ 11.18-10.75 (m, 1H), 7.93-7.89 |
| | (m, 2H), 7.69-7.60 (m, 2H), 7.46-7.39 (m, 2H), 7.37-7.30 (m, |
| | 2H), 4.80 (br, 1H), 4.54-4.21 (m, 4H), 3.98-3.89 (m, |
| | 2H), 3.85-3.75 (m, 2H), 3.45-3.33 (m, 2H), 3.13-2.91 (m, 4H), |
| | 2.75-2.71 (m, 3H), 1.89-1.54 (m, 4H), 1.30-0.98 (m, 2H). |
| | LC-MS (ESI, m/z): [M + H]⁺ 453. |
| 27- 08 | 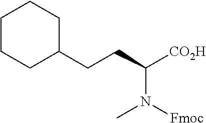 |
| | (S)-2-((((9H-fluoren-9-yl)methoxy)carbonyl)(methyl)amino)-4- |
| | cyclohexylbutanoic acid |
| | (S)-2-((((9H-fluoren-9-yl)methoxy)carbonyl)amino)-4- |
| | cyclohexylbutanoic acid (CAS: 269078-73-1) |
| | Paraformaldehyde (CAS: 30525-89-4) |
| | ¹H NMR (500 MHz, DMSO-d₆) δ 12.77 (br, 1H), 7.94-7.85 (m, |
| | 2H), 7.68-7.59 (m, 2H), 7.49-7.38 (m, 2H), 7.37-7.27 (m, |
| | 2H), 4.50-4.20 (m, 4H), 2.75-2.67 (m, 3H), 1.89-1.73 (m, |
| | 1H), 1.72-1.51 (m, 6H), 1.26-0.93 (m, 6H), 0.93-0.77 (m, 2H). |
| | LC-MS (ESI, m/z): [M + H]⁺ 422. |
| 27- 09 | 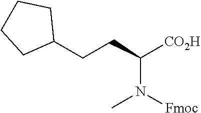 |
| | (S)-2-((((9H-fluoren-9-yl)methoxy)carbonyl)(methyl)amino)-4- |
| | cyclopentylbutanoic acid |
| | (S)-2-((((9H-fluoren-9-yl)methoxy)carbonyl)amino)-4- |
| | cyclopentylbutanoic acid (CAS: 2349655-85-0) |
| | Paraformaldehyde (CAS: 30525-89-4) |
| | ¹H NMR (500 MHz, DMSO-d₆) δ 12.77 (br, 1H), 7.95-7.84 (m, |
| | 2H), 7.68-7.59 (m, 2H), 7.50-7.37 (m, 2H), 7.37-7.25 (m, 2H), |
| | 4.51-4.21 (m, 4H), 2.75-2.67 (m, 3H), 1.89-1.41 (m, 9H), |
| | 1.28-0.93 (m, 4H). LC-MS (ESI, m/z): [M + H]⁺ 408. |
| 27- 10 | 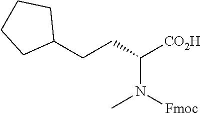 |
| | (R)-2-((((9H-fluoren-9-yl)methoxy)carbonyl)(methyl)amino)-4- |
| | cyclopentylbutanoic acid |
| | (R)-2-((((9H-fluoren-9-yl)methoxy)carbonyl)amino)-4- |
| | cyclopentylbutanoic acid (CAS: 2349970-55-2) |
| | Paraformaldehyde (CAS: 30525-89-4) |
| | ¹H NMR (500 MHz, DMSO-d₆) δ 12.75 (br, 1H), 7.95-7.83 (m, |
| | 2H), 7.69-7.55 (m, 2H), 7.50-7.37 (m, 2H), 7.37-7.24 (m, 2H), |
| | 4.53-4.21 (m, 4H), 2.77-2.66 (m, 3H), 1.89-1.41 (m, 9H), |
| | 1.27-0.94 (m, 4H). LC-MS (ESI, m/z): [M + H]⁺ 408. |
| 27- 11 | 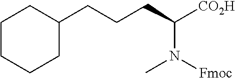 |
| | (S)-2-((((9H-fluoren-9-yl)methoxy)carbonyl)(methyl)amino)-5- |
| | cyclohexylpentanoic acid |
| | (S)-2-((((9H-fluoren-9-yl)methoxy)carbonyl)amino)-5- |
| | cyclohexylpentanoic acid (CAS: 2350058-24-9) |
| | Paraformaldehyde (CAS: 30525-89-4) |
| | ¹H NMR (500 MHz, DMSO-d₆) δ 12.77 (br, 1H), 7.95-7.71 (m, |
| | 2H), 7.71-7.53 (m, 2H), 7.49-7.37 (m, 2H), 7.37-7.24 (m, 2H), |
| | 4.57-4.18 (m, 4H), 2.78-2.60 (m, 3H), 1.85-1.48 (m, 7H), |
| | 1.27-1.02 (m, 8H), 0.94-0.73 (m, 2H). LC-MS (ESI, m/z): |
| | [M + H]⁺ 436. |
| 27- 12 | 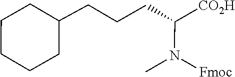 |
| | (R)-2-((((9H-fluoren-9-yl)methoxy)carbonyl)(methyl)amino)-5- |
| | cyclohexylpentanoic acid |
| | (R)-2-((((9H-fluoren-9-yl)methoxy)carbonyl)amino)-5- |
| | cyclohexylpentanoic acid (CAS: 2349497-68-1) |
| | Paraformaldehyde (CAS: 30525-89-4) |
| | Substantially identical to Example 27-11 |
| 27- 13 | 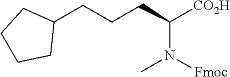 |
| | (S)-2-((((9H-fluoren-9-yl)methoxy)carbonyl)(methyl)amino)-5- |
| | cyclopentylpentanoic acid |
| | (S)-2-((((9H-fluoren-9-yl)methoxy)carbonyl)amino)-5- |
| | cyclopentylpentanoic acid (CAS: 2349606-67-1) |
| | Paraformaldehyde (CAS: 30525-89-4) |
| | ¹H NMR (500 MHz, DMSO-d₆) δ 12.78 (br, 1H), 7.95-7.85 (m, |
| | 2H), 7.69-7.58 (m, 2H), 7.47-7.38 (m, 2H), 7.38-7.26 (m, |
| | 2H), 4.55-4.20 (m, 4H), 2.77-2.66 (m, 3H), 1.87-1.36 (m, 9H), |
| | 1.36-0.90 (m, 6H). LC-MS (ESI, m/z): [M + H]⁺ 422. |
| 27- 14 | 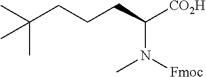 |
| | (S)-2-((((9H-fluoren-9-yl)methoxy)carbonyl)(methyl)amino)-6,6- |
| | dimethylheptanoic acid |
| | (S)-2-((((9H-fluoren-9-yl)methoxy)carbonyl)amino)-6,6- |
| | dimethylheptanoic acid (CAS: 2349756-34-7) |
| | Paraformaldehyde (CAS: 30525-89-4) |
| | ¹H NMR (500 MHz, DMSO-d₆) δ 12.77 (br, 1H), 7.93-7.86 (m, |
| | 2H), 7.68-7.59 (m, 2H), 7.47-7.38 (m, 2H), 7.38-7.26 (m, 2H), |
| | 4.58-4.21 (m, 4H), 2.79-2.70 (m, 3H), 1.83-1.52 (m, 2H), 1.29- |
| | 1.05 (m, 4H) ,0.88-0.75 (m, 9H). LC-MS (ESI, m/z): |
| | [M + H]⁺ 410. |
| 27- 15 | 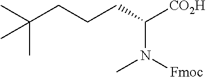 |
| | (R)-2-((((9H-fluoren-9-yl)methoxy)carbonyl)(methyl)amino)-6,6- |
| | dimethylheptanoic acid |
| | (R)-2-((((9H-fluoren-9-yl)methoxy)carbonyl)amino)-6,6- |
| | dimethylheptanoic acid (CAS: 2350170-64-6) |
| | Paraformaldehyde (CAS: 30525-89-4) |
| | Substantially identical to Example 27-14 |
| 27- 16 | 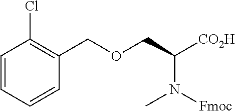 |
| | (2S)-3-[(2-chlorophenyl)methoxy]-2-({[(9H-fluoren-9- |
| | yl)methoxy]carbonyl }(methyl)amino)propanoic acid |
| | O-[(2-Chlorophenyl)methyl]-N-[(1,1-dimethylethoxy)carbonyl]-L- |
| | serine (CAS: 147325-13-1) |
| | Paraformaldehyde (CAS: 30525-89-4) |
| | ¹H NMR (500 MHz, DMSO-d₆) δ 13.05 (br, 1H), 7.95- |
| | 7.82 (m, 2H),7.73-7.58 (m, 2H), 7.52-7.19 (m, 8H), 4.86-4.74 (m, |
| | 1H), 4.65-4.49 (m, 2H), 4.38-4.18 (m, 3H), 3.97-3.76 (m, 2H), |
| | 2.94-2.78 (m, 3H). LC-MS (ESI, m/z): [M + H]⁺ 466. |
| 27- 17 | 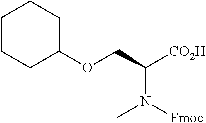 |
| | (2S)-3-(cyclohexyloxy)-2-({[(9H-fluoren-9- |
| | yl)methoxy]carbonyl}(methyl)amino)propanoic acid |
| | N-(((9H-fluoren-9-yl)methoxy)carbonyl)-O-cyclohexyl-L-serine |
| | (CAS: 221057-20-1) |
| | Paraformaldehyde (CAS: 30525-89-4) |
| | ¹H NMR (500 MHz, DMSO-d₆) δ 12.93 (br, 1H), |
| | 7.94-7.86 (m, 2H), 7.69-7.60 (m, 2H), 7.46-7.39 (m, 2H), |
| | 7.37-7.28 (m, 2H), 4.75-4.62 (m, 1H), 4.39-4.21 (m, 3H), |
| | 3.85-3.67 (m, 2H), 3.33-3.20 (m, 1H), 2.94-2.79 (m, 3H), |
| | 1.84-1.69 (m, 2H), 1.69-1.53 (m, 2H), 1.53-1.38 |
| | (m, 1H), 1.31-1.11 (m, 5H). LC-MS (ESI, m/z): [M + H]⁺ 424. |
| 27- 18 | 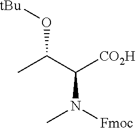 |
| | (2S,3S)-3-(tert-butoxy)-2-({[(9H-fluoren-9- |
| | yl)methoxy]carbonyl}(methyl)amino)butanoic acid |
| | N-(((9H-fluoren-9-yl)methoxy)carbonyl)-O-(tert-butyl)-L- |
| | allothreonine (CAS: 201481-37-0) |
| | Paraformaldehyde (CAS: 30525-89-4) |
| | ¹H NMR (500 MHz, DMSO-d₆) δ 12.69 (br, 1H), 7.92- |
| | 7.87 (m, 2H), 7.70-7.61 (m, 2H), 7.44-7.38 (m, 2H), |
| | 7.36-7.28 (m, 2H), 4.51-4.24 (m, 4H), 4.16-3.98 (m, 1H), |
| | 2.84-2.78 (m, 3H), 1.13-1.09 (m, 9H), |
| | 1.01-0.95 (m, 3H). LC-MS (ESI, m/z): [M − tBu + H]⁺ 356. |
| 27- 19 | 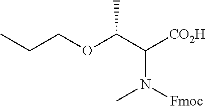 |
| | (2S,3R)-2-({[(9H-fluoren-9-yl)methoxy]carbonyl}(methyl)amino)-3- |
| | propoxybutanoic acid |
| | N-(((9H-fluoren-9-yl)methoxy)carbonyl)-O-propyl-L-threonine |
| | (CAS: 2865160-87-6) |
| | Paraformaldehyde (CAS: 30525-89-4) |
| | ¹H NMR (500 MHz, DMSO-d₆) δ 12.89 (br, 1H), 7.96-7.85 (m, |
| | 2H), 7.69-7.59 (m, 2H), 7.46-7.37 (m, 2H), 7.37-7.27 (m, |
| | 2H), 4.67-3.86 (m, 5H), 3.50-3.11 (m, 2H), 2.94 - 2.82 (m, |
| | 3H), 1.49-1.36 (m, 2H), 1.08-0.77 (m, 6H). LC-MS (ESI, m/z): |
| | [M + H]⁺ 398. |

### Example-28-01: (R)-3-((((9H-fluoren-9-yl)methoxy)carbonyl)(methyl)amino)-5-((3-methylpentan-3-yl)oxy)-5-oxopentanoic acid

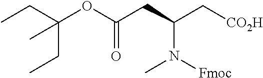

To a solution of (S)-2-((((9H-fluoren-9-yl)methoxy)carbonyl)(methyl)amino)-4-((3-methylpentan-3-yl)oxy)-4-oxobutanoic acid (CAS: 2973754-43-5, 7.2 g, 15.9 mmol) in THF (95 ml) at −15° C. were added triethylamine (2.43 ml, 17.5 mmol) and isobutyl chloroformate (2.29 ml, 17.5 mmol). The mixture was stirred at 0° C. for 1 h. The precipitation was filtered off and washed with THF. The filtrate was concentrated and the residue was dissolved in CH₃CN (63.5 ml). To the solution at 0° C. was added 0.6M (diazomethyl)trimethylsilane in hexane (52.9 ml, 31.8 mmol) dropwise over 10 min. The reaction mixture was gradually warmed up to rt and stirred for 17 h. The mixture was slowly added to a cooled solution of citric acid (15.2 g) in water (150 mL). Brine was added and extracted twice with EtOAc. The combined organic extracts were washed with satd. aq. NaHCO₃, and brine, dried over Na₂SO₄, filtered, and concentrated. The residue was purified by flash column chromatography (gradient, heptane/EtOAc=90/10 to 60/40). Appropriate fractions were concentrated. To a solution of the obtained material (4.30 g, 9.00 mmol) in 1,4-dioxane (64 mL) and H₂O (26 mL) at rt was added silver(I) benzoate (CAS: 532-31-0, 0.08 g, 0.36 mmol). The mixture was stirred at 70° C. for 2 h, then the reaction mixture was concentrated. The residue was diluted with water and acidified with 1M aq. HCl. The mixture was extracted twice with EtOAc. The combined organic extracts were washed with brine, dried over Na₂SO₄, filtered, and concentrated. The residue was purified by flash column chromatography (gradient, heptane/EtOAc=90/10 to 60/40, then isocratic, CH₂Cl₂/MeOH=95/5) to afford the title compound (3.01 g, 6.44 mmol). ¹H NMR (500 MHz, DMSO-d₆) δ 12.29 (br, 1H), 7.92-7.85 (m, 2H), 7.74-7.59 (m, 2H), 7.45-7.28 (m, 4H), 4.61-4.18 (m, 4H), 2.82-2.68 (m, 3H), 2.66-2.18 (m, 4H), 1.83-1.57 (m, 4H), 1.34-1.21 (m, 3H), 0.82-0.69 (m, 6H). LC-MS (ESI, m/z): [M-Mpe+H]⁺ 384.

### Example-28

The following compounds can be synthesized as outlined for the preparation of Reference example-17-01 or Example-28-01 employing appropriate starting materials in the table.

| Ex- | Structure and IUPAC name |
|---|---|
| am- | Starting material-A |
| ple | NMR and MS |
| 28- 02 | 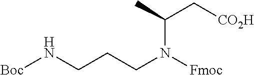 |
| | (S)-3-((((9H-fluoren-9-yl)methoxy)carbonyl)(3-((tert- |
| | butoxycarbonyl)amino)propyl)amino)butanoic acid |
| | N-(((9H-fluoren-9-yl)methoxy)carbonyl)-N-(3-((tert- |
| | butoxycarbonyl)amino)propyl)-L-alanine (CAS: 174799-80-5) |
| | ¹H NMR (500 MHz, DMSO-d₆) δ 12.14 (br, 1H), 7.88 (d, J = 7.5 |
| | Hz, 2H), 7.71-7.59 (m, 2H), 7.45-7.26 (m, 4H), 6.80-6.64 (m, 1H), |
| | 4.51-4.36 (m, 2H), 4.31-4.23 (m, 1H), 4.16-3.96 (m, 1H), 3.16- |
| | 2.55 (m, 4H), 2.50-2.32 (m, 2H), 2.26-2.06 (m, 1H), 1.58-1.43 (m, |
| | 1H), 1.42-0.78 (m, 12H). LC-MS (ESI, m/z): [M + H]⁺ 483. |
| 28- 03 | 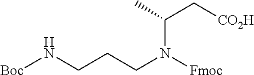 |
| | (R)-3-((((9H-fluoren-9-yl)methoxy)carbonyl)(3-((tert- |
| | butoxycarbonyl)amino)propyl)amino)butanoic acid |
| | N-(((9H-fluoren-9-yl)methoxy)carbonyl)-N-(3-((tert- |
| | butoxycarbonyl)amino)propyl)-D-alanine (CAS: 174799-82-7) |
| | Substantially identical to Example 28-02 |
| 28- 04 | 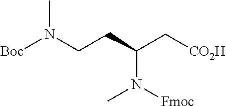 |
| | (S)-3-((((9H-fluoren-9-yl)methoxy)carbonyl)(methyl)amino)-5-((tert- |
| | butoxycarbonyl)(methyl)amino)pentanoic acid |
| | Example 27-04 |
| | ¹H NMR (500 MHz, DMSO-d₆) δ 12.22 (br, 1H), |
| | 7.91-7.86 (m, 2H), 7.69-7.60 (m, 2H), 7.44-7.38 (m, 2H), |
| | 7.37-7.29 (m, 2H), 4.47- 4.06 (m, 4H), 3.06-2.77 (m, 2H), |
| | 2.75-2.60 (m, 6H), 2.47- 2.27 (m, 2H), 1.70-1.47 (m, 2H), |
| | 1.36 (s, 9H). LC-MS (ESI, m/z): [M + H]⁺ 483. |
| 28- 05 | 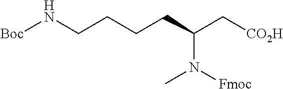 |
| | (S)-3-((((9H-fluoren-9-yl)methoxy)carbonyl)(methyl)amino)-7-((tert- |
| | butoxycarbonyl)amino)heptanoic acid |
| | N2-(((9H-fluoren-9-yl)methoxy)carbonyl)-N6-(tert-butoxycarbonyl)- |
| | N2-methyl-L-lysine (CAS: 197632-76-1) |
| | ¹H NMR (500 MHz, DMSO-d₆) δ 12.17 (br, 1H), 7.9-7.86 (m, |
| | 2H), 7.70-7.66 (m, 1H), 7.65-7.59 (m, 1H), 7.46-7.38 (m, |
| | 2H), 7.37-7.28 (m, 2H), 6.81-6.34 (m, 1H), |
| | 4.45-4.09 (m, 4H), 2.91-2.70 (m, 2H), 2.67-2.60 (m, 3H), |
| | 2.41-2.21 (m, 2H), 1.35 (s, 9H), 1.53-1.24 (m, 4H), |
| | 1.12-0.98 (m, 2H). LC-MS (ESI, m/z): [M + H]⁺ 497. |
| 28- 06 | 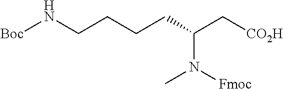 |
| | (R)-3-((((9H-fluoren-9-yl)methoxy)carbonyl)(methyl)amino)-7-((tert- |
| | butoxycarbonyl)amino)heptanoic acid |
| | N2-(((9H-fluoren-9-yl)methoxy)carbonyl)-N6-(tert-butoxycarbonyl)- |
| | N2-methyl-D-lysine (CAS: 1793105-27-7) |
| | Substantially identical to Example 28-05 |
| 28- 07 | 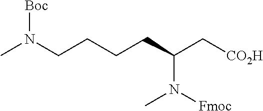 |
| | (S)-3-((((9H-fluoren-9-yl)methoxy)carbonyl)(methyl)amino)-7-((tert- |
| | butoxycarbonyl)(methyl)amino)heptanoic acid |
| | N2-(((9H-fluoren-9-yl)methoxy)carbonyl)-N6-(tert-butoxycarbonyl)- |
| | N2,N6-dimethyl-L-lysine (CAS: 2973756-55-5) |
| | ¹H NMR (500 MHz, DMSO-d₆) δ 12.18 (br, 1H), 7.92-7.86 (m, |
| | 2H), 7.71-7.59 (m, 2H), 7.46-7.28 (m, 4H), 4.48-4.10 (m, 4H), |
| | 3.16-3.04 (m, 2H), 2.73 (br, 3H), 2.66-2.61 (m, 3H), 2.41-2.20 (m, |
| | 2H), 1.70-1.25 (m, 4H), 1.35 (s, 9H), 1.12 - 0.93 (m, 2H). |
| | LC-MS (ESI, m/z): [M + H]⁺ 511. |

### Example-29

The following compounds can be synthesized as outlined for the preparation of Reference example-15-01 employing appropriate starting materials in the table.

| | Structure and IUPAC name |
|---|---|
| | Starting material-A |
| | Starting material-B |
| Example | NMR and MS |
| 29-01 | 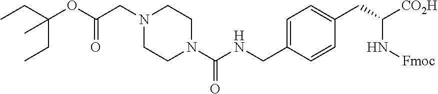 |
| | (R)-2-((((9H-fluoren-9-yl)methoxy)carbonyl)amino)-3-(4-((4-(2-((3- |
| | methylpentan-3-yl)oxy)-2-oxoethyl)piperazine-1- |
| | carboxamido)methyl)phenyl)propanoic acid |
| | (R)-2-((((9H-Fluoren-9-yl)methoxy)carbonyl)amino)-3-(4-(((tert- |
| | butoxycarbonyl)amino)methyl)phenyl)propanoic acid (CAS: 268731-06-2) |
| | 1-ethyl-1-methylpropyl 1-Piperazineacetate (CAS: 2973756-40-8) |
| | 1HNMR (500 MHz, DMSO-d₆) δ 12.77 (br, 1H), 7.88 (d, J = 7.5 Hz, 2H), |
| | 7.75-7.50 (m, 3H), 7.45-7.37 (m, 2H), 7.35-7.23 (m, 2H), 7.22-7.09 (m, |
| | 4H), 7.05-6.92 (m, 1H), 4.33-4.05 (m, 6H), 3.32-3.29 (m, 4H), 3.14 (s, |
| | 2H), 3.08-2.97 (m, 1H), 2.90-2.78 (m, 1H), 2.48-2.38 (m, 4H), 1.87- |
| | 1.75 (m, 2H), 1.75-1.61 (m, 2H), 1.32 (s, 3H), 0.87-0.72 (m, 6H). LC-MS |
| | (ESI, m/z): [M + H]⁺ 671. |
| 29-02 | 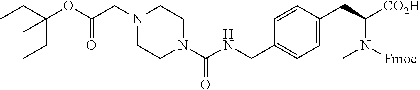 |
| | (S)-2-((((9H-fluoren-9-yl)methoxy)carbonyl)(methyl)amino)-3-(4-((4-(2- |
| | ((3-methylpentan-3-yl)oxy)-2-oxoethyl)piperazine-1- |
| | carboxamido)methyl)phenyl)propanoic acid |
| | (2S)-2-({[(9H-fluoren-9-yl)methoxy]carbonyl}(methyl)amino)-3-(4-{[(4- |
| | {2-[(3-methylpentan-3-yl)oxy]-2-oxoethyl}piperazine-1- |
| | carbonyl)amino]methyl}phenyl)propanoic acid (CAS: 2973754-54-8) |
| | 1-ethyl-1-methylpropyl 1-Piperazineacetate (CAS: 2973756-40-8) |
| | ¹H NMR (500 MHz, DMSO-d₆) δ 12.90 (br, 1H), 7.88 (d, J = 7.6 Hz, 2H), |
| | 7.60-7.50 (m, 2H), 7.44-7.38 (m, 2H), 7.3-7.28 (m, 2H), 7.15-7.09 (m, |
| | 3H), 7.03-6.94 (m, 2H), 4.77-4.61 (m, 1H), 4.37-4.13 (m, 5H), 3.31- |
| | 3.26 (m, 4H), 3.19-2.77 (m, 4H), 2.71-2.62 (m, 3H), 2.47-2.42 (m, 4H), |
| | 1.85-1.78 (m, 2H), 1.73-1.64 (m, 2H), 1.31 (s, 3H), 0.80 (t, J = 7.5 Hz, |
| | 6H). LCMS (m/z): [M + H]⁺ 685. |
| 29-03 | 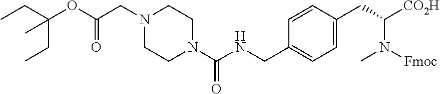 |
| | (R)-2-((((9H-fluoren-9-yl)methoxy)carbonyl)(methyl)amino)-3-(4-((4-(2- |
| | ((3-methylpentan-3-yl)oxy)-2-oxoethyl)piperazine-1- |
| | carboxamido)methyl)phenyl)propanoic acid |
| | (2R)-3-[4-({[(tert-butoxy)carbonyl]amino}methyl)phenyl]-2-({[(9H- |
| | fluoren-9-yl)methoxy]carbonyl}(methyl)amino)propanoic acid |
| | 1-ethyl-1-methylpropyl 1-Piperazineacetate (CAS: 2973756-40-8) |
| | Substantially identical to Example 29-02 |
| 29-04 | 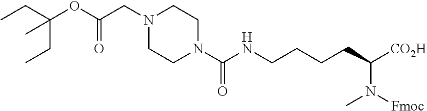 |
| | (2S)-2-({[(9H-fluoren-9-yl)methoxy]carbonyl}(methyl)amino)-6-[(4-{2- |
| | [(3-methylpentan-3-yl)oxy]-2-oxoethyl}piperazine-1- |
| | carbonyl)amino]hexanoic acid |
| | N2-(((9H-fluoren-9-yl)methoxy)carbonyl)-N2-methyl-L-lysine |
| | hydrochloride (CAS: 2991253-50-8) |
| | 1-ethyl-1-methylpropyl 1-Piperazineacetate (CAS: 2973756-40-8) |
| | ¹H NMR (500 MHz, DMSO-d₆) δ 12.74 (br, 1H), 7.92-7.87 (m, 2H), 7.67- |
| | 7.60 (m, 2H), 7.45-7.38 (m, 2H), 7.36-7.28 (m, 2H), 6.43 (br, 1H), 4.52- |
| | 4.45 (m, 1H), 4.40-4.23 (m, 3H), 3.28-3.21 (m, 4H), 3.12 (s, 2H), 3.04- |
| | 2.95 (m, 2H), 2.76-2.70 (m, 3H), 2.44-2.39 (m, 4H), 1.84-1.64 (m, 6H), |
| | 1.47-1.34 (m, 2H), 1.30 (s, 3H), 1.20-1.07 (m, 2H), 0.83-0.76 (m, 6H). |
| | LC-MS (ESI, m/z): [M + H]⁺ 637. |
| 29-05 | 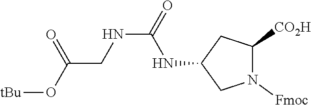 |
| | (2S,4R)-1-(((9H-fluoren-9-yl)methoxy)carbonyl)-4-(3-(2-(tert-butoxy)-2- |
| | oxoethyl)ureido)pyrrolidine-2-carboxylic acid |
| | (2S,4R)-1-(((9H-fluoren-9-yl)methoxy)carbonyl)-4-((tert- |
| | butoxycarbonyl)amino)pyrrolidine-2-carboxylic acid (CAS: 273222-06-3) |
| | tert-butyl 2-aminoacetate (CAS: 6456-74-2) |
| | ¹H NMR (500 MHz, DMSO-d₆) δ 13.13-12.56 (m, 1H), 7.92-7.88 (m, |
| | 2H), 7.69-7.62 (m, 2H), 7.45-7.39 (m, 2H), 7.37-7.31 (m, 2H), 6.63- |
| | 6.56 (m, 1H), 6.12-6.05 (m, 1H), 4.53-4.08 (m, 5H), 3.71-3.57 (m, 3H), |
| | 3.30-3.21 (m, 1H), 2.24-2.03 (m, 2H), 1.40 (s, 9H). LC-MS (ESI, m/z): |
| | [M + H]⁺ 510. |
| 29-06 | 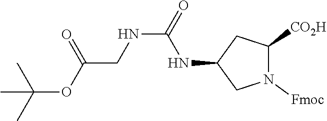 |
| | (2S,4S)-1-(((9H-fluoren-9-yl)methoxy)carbonyl)-4-(3-(2-(tert-butoxy)-2- |
| | oxoethyl)ureido)pyrrolidine-2-carboxylic acid |
| | (2S,4S)-1-(((9H-fluoren-9-yl)methoxy)carbonyl)-4-((tert- |
| | butoxycarbonyl)amino)pyrrolidine-2-carboxylic acid (CAS: 221352-74-5) |
| | tert-butyl 2-aminoacetate (CAS: 6456-74-2) |
| | ¹H NMR (500 MHz, DMSO-d₆) δ 12.85 (br, 1H), 7.95-7.86 (m, 2H), 7.70- |
| | 7.61 (m, 2H), 7.47-7.39 (m, 2H), 7.38-7.29 (m, 2H), 6.44-6.36 (m, 1H), |
| | 6.29-6.19 (m, 1H), 4.40-4.23 (m, 2H), 4.23-4.07 (m, 3H), 3.83-3.70 (m, |
| | 1H), 3.69-3.58 (m, 2H), 3.16-3.03 (m, 1H), 2.66-2.43 (m, 1H), 1.87- |
| | 1.66 (m, 1H), 1.40 (s, 9H). LC-MS (ESI, m/z): [M + H]⁺ 510. |
| 29-07 | 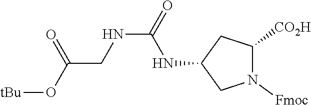 |
| | (2R,4R)-1-(((9H-fluoren-9-yl)methoxy)carbonyl)-4-(3-(2-(tert-butoxy)-2- |
| | oxoethyl)ureido)pyrrolidine-2-carboxylic acid |
| | (2R,4R)-1-(((9H-fluoren-9-yl)methoxy)carbonyl)-4-((tert- |
| | butoxycarbonyl)amino)pyrrolidine-2-carboxylic acid (CAS: 1820574-93-3) |
| | tert-butyl 2-aminoacetate (CAS: 6456-74-2) |
| | ¹H NMR (500 MHz, DMSO-d₆) δ 12.78 (br, 1H), 7.93-7.86 (m, 2H), 7.69- |
| | 7.63 (m, 2H), 7.45-7.39 (m, 2H), 7.38-7.29 (m, 2H), 6.39 (d, J = 7.1 Hz, |
| | 1H), 6.23 (t, J = 5.9 Hz, 1H), 4.37-4.07 (m, 5H), 3.80-3.58 (m, 3H), 3.14- |
| | 3.03 (m, 1H), 2.65-2.48 (m, 1H), 1.87-1.67 (m, 1H), 1.41 (s, 9H). LC- |
| | MS (ESI, m/z): [M + H]⁺ 510. |
| 29-08 | 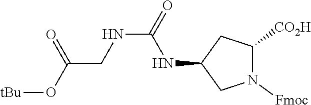 |
| | (2R,4S)-1-(((9H-fluoren-9-yl)methoxy)carbonyl)-4-(3-(2-(tert-butoxy)-2- |
| | oxoethyl)ureido)pyrrolidine-2-carboxylic acid |
| | (2R,4S)-1-(((9H-fluoren-9-yl)methoxy)carbonyl)-4-((tert- |
| | butoxycarbonyl)amino)pyrrolidine-2-carboxylic acid (CAS: 1253791-18-2) |
| | tert-butyl 2-aminoacetate (CAS: 6456-74-2) |
| | ¹H NMR (500 MHz, DMSO-d₆) δ 12.81 (br, 1H), 7.90 (t, J = 6.5 Hz, 2H), |
| | 7.63-7.71 (m, 2H), 7.29-7.46 (m, 4H), 6.59 (dd, J = 6.9, 4.4 Hz, 1H), 6.05- |
| | 6.11 (m, 1H) 4.10-4.47 (m, 5H), 3.57-3.71 (m, 3H), 3.22-3.31 (m, 1H), |
| | 2.02-2.26 (m, 2H), 1.34-1.46 (m, 9H). LC-MS (ESI, m/z): [M + H]⁺ 510. |
| 29-09 | 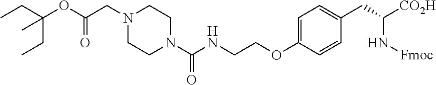 |
| | (R)-2-((((9H-fluoren-9-yl)methoxy)carbonyl)amino)-3-(4-(2-(4-(2-((3- |
| | methylpentan-3-yl)oxy)-2-oxoethyl)piperazine-1- |
| | carboxamido)ethoxy)phenyl)propanoic acid |
| | (R)-2-((((9H-fluoren-9-yl)methoxy)carbonyl)amino)-3-(4-(2-((tert- |
| | butoxycarbonyl)amino)ethoxy)phenyl)propanoic acid |
| | 1-ethyl-1-methylpropyl 1-Piperazineacetate (CAS: 2973756-40-8) |
| | ¹H NMR (500 MHz, DMSO-d₆) δ 12.71 (br, 1H), 7.88 (d, J = 7.5 Hz, 2H), |
| | 7.74-7.59 (m, 3H), 7.54-7.36 (m, 2H), 7.35-7.21 (m, 2H), 7.17 (d, J = |
| | 8.5 Hz, 2H), 6.84 (d, J = 8.7 Hz, 2H), 6.71 (br, 1H), 4.28-4.06 (m, 4H), |
| | 3.97-3.85 (m, 2H), 3.35 (br, 2H), 3.28 (br, 4H), 3.15 (br, 2H), 3.05-2.95 |
| | (m, 1H), 2.84-2.75 (m, 1H), 2.45 (br, 4H), 1.88-1.75 (m, 2H), 1.74- |
| | 1.63 (m, 2H), 1.31 (s, 3H), 0.85-0.73 (m, 6H). LC-MS (ESI, m/z): [M + H]⁺ |
| | 701. |
| 29-10 | 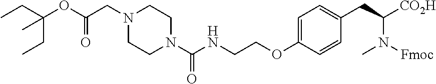 |
| | (S)-2-((((9H-fluoren-9-yl)methoxy)carbonyl)(methyl)amino)-3-(4-(2-(4-(2- |
| | ((3-methylpentan-3-yl)oxy)-2-oxoethyl)piperazine-1- |
| | carboxamido)ethoxy)phenyl)propanoic acid |
| | (S)-2-((((9H-fluoren-9-yl)methoxy)carbonyl)(methyl)amino)-3-(4-(2-((tert- |
| | butoxycarbonyl)amino)ethoxy)phenyl)propanoic acid |
| | 1-ethyl-1-methylpropyl 1-Piperazineacetate (CAS: 2973756-40-8) |
| | ¹H NMR (500 MHz, DMSO-d₆) δ 13.00 (br, 1H), 7.92-7.82 (m, 2H), 7.59- |
| | 7.51 (m, 1H), 7.51-7.45 (m, 1H), 7.44-7.37 (m, 2H), 7.35-7.26 (m, 2H), |
| | 7.10 (d, J = 8.5 Hz, 1H), 7.01-6.95 (m, 1H), 6.85-6.77 (m, 2H), 6.73 - |
| | 6.65 (m, 1H), 4.81-4.54 (m, 1H), 4.30-4.10 (m, 3H), 3.95-3.83 (m, 2H), |
| | 3.35-3.31 (m, 2H), 3.27 (br, 4H), 3.19-2.73 (m, 4H), 2.72-2.63 (m, 3H), |
| | 2.43 (br, 4H), 1.87-1.75 (m, 2H), 1.75-1.61 (m, 2H), 1.31 (s, 3H), 0.84- |
| | 0.74 (m, 6H). LC-MS (ESI, m/z): [M + H]⁺ 715. |
| 29-11 | 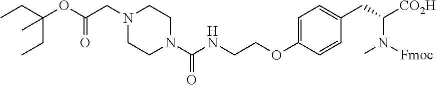 |
| | (R)-2-((((9H-fluoren-9-yl)methoxy)carbonyl)(methyl)amino)-3-(4-(2-(4-(2- |
| | ((3-methylpentan-3-yl)oxy)-2-oxoethyl)piperazine-1- |
| | carboxamido)ethoxy)phenyl)propanoic acid |
| | (R)-2-((((9H-fluoren-9-yl)methoxy)carbonyl)(methyl)amino)-3-(4-(2- |
| | ((tert-butoxycarbonyl)amino)ethoxy)phenyl)propanoic acid |
| | 1-ethyl-1-methylpropyl 1-Piperazineacetate (CAS: 2973756-40-8) |
| | Substantially identical to Example 29-10 |

### Example-30

The following compounds can be synthesized as outlined for the preparation of Reference example-19-01 employing appropriate starting materials in the table.

| | Structure and IUPAC name |
|---|---|
| | Starting material-A |
| Ex- | Starting material-B |
| ample | NMR and MS |
| 30-01 | 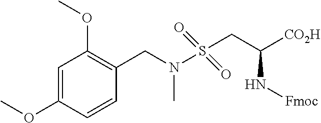 |
| | (2R)-3-{[(2,4-dimethoxyphenyl)methyl](methyl)sulfamoyl}-2- |
| | ({[(9H-fluoren-9-yl)methoxy]carbonyl}amino)propanoic acid |
| | (2R)-2-({[(9H-fluoren-9-yl)methoxy]carbonyl}amino)-3- |
| | [(triphenylmethyl)sulfanyl]propanoic acid (CAS: 103213-32-7) |
| | 2,4-Dimethoxy-N-methylbenzenemethanamine (CAS: 102503-23-1) |
| | ¹H NMR (500 MHz, DMSO-d₆) δ 13.21 (br, 1H), 7.95-7.85 (m, |
| | 3H), 7.76-7.69 (m, 2H), 7.44-7.37 (m, 2H), 7.34-7.25 (m, |
| | 2H), 7.13 (d, J = 8.2 Hz, 1H), 6.59-6.48 (m, 2H), 4.47-4.38 (m, |
| | 1H), 4.37-4.29 (m, 2H), 44.27-.10 (m, 3H), 3.78-3.69 (m, |
| | 6H), 3.54-3.46 (m, 2H), 2.63 (s, 3H). |
| | LC-MS (ESI, m/z): [M + H]⁺ Not detected. |

### Example-31-01: (2R,4R)-1-(((9H-fluoren-9-yl)methoxy)carbonyl)-4-(2-(tert-butoxy)-2-oxoethoxy)pyrrolidine-2-carboxylic acid

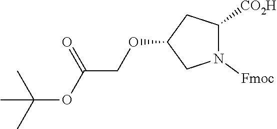

To a solution of (2R,4R)-1-[(9H-fluoren-9-ylmethoxy)carbonyl]-4-hydroxypyrrolidine-2-carboxylic acid (CAS: 214852-45-6, 50 g, 142 mmol) in DMF (500 ml) at rt was added KHCO₃ (28.3 g, 283 mmol). The mixture was stirred at rt for 5 min Allyl bromide (16.3 g, 134 mmol) was added at rt and the resulting mixture was stirred at rt overnight. The reaction was then quenched with 1 M aq. HCl and diluted with H₂O at 0° C. The mixture was extracted with EtOAc. The organic extracts were washed with satd. aq. NaHCO₃, and brine, dried over Na₂SO₄, filtered, and concentrated. The resulting residue was purified by silica gel flash column chromatography (isocratic, petroleum ether/EtOAc=75/25). Appropriate fractions were collected and concentrated. To a solution of the resulting residue in CH₂Cl₂ (450 mL) was added Rh₂(OAc)₄ (2.53 g, 5.72 mmol) at rt. To the mixture at rt was added tert-butyl 2-diazoacetate (CAS: 35059-50-8, 17.9 g, 126 mmol) in CH₂Cl₂ (30 ml) dropwise over 6 h. After stirred at rt overnight, this mixture was concentrated. The resulting residue was purified by silica gel flash column chromatography (isocratic, petroleum ether/EtOAc=75/25). Appropriate fractions were collected and concentrated. To a solution of the obtained compound (33.0 g, 65.0 mmol) and N-methylaniline (20.9 g, 195 mmol) in THF (330 mL) at 0° C. was added Pd(PPh₃)₄ (2.25 g, 1.95 mmol). After stirred at rt for 1 h, this mixture was concentrated. The resulting residue was purified by reversed phase silica gel flash column chromatography (C18 silica gel, gradient, H₂O/CH₃CN with 0.1% formic acid=90/10 to 0/100) to afford the title compound (18.7 g, 40.0 mmol). ¹H NMR (500 MHz, DMSO-d₆) δ 13.11-12.21 (m, 1H), 7.96-7.85 (m, 2H), 7.72-7.61 (m, 2H), 7.49-7.38 (m, 2H), 7.38-7.26 (m, 2H), 4.41-4.25 (m, 2H), 4.25-4.09 (m, 3H), 4.02-3.94 (m, 2H), 3.69-3.59 (m, 1H), 3.42-3.33 (m, 1H), 2.48-2.33 (m, 1H), 2.21-2.00 (m, 1H), 1.47-1.37 (m, 9H). LC-MS (ESI, m/z): [M-tBu+H]⁺ 412.

### Example-31

The following compounds can be synthesized as outlined for the preparation of Example-31-01 employing appropriate starting materials in the table.

| Ex- | Structure and IUPAC name |
|---|---|
| am- | Starting material-A |
| ple | NMR and MS |
| 31- 02 | 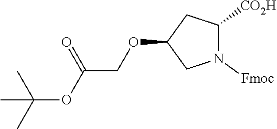 |
| | (2R,4S)-1-(((9H-fluoren-9-yl)methoxy)carbonyl)-4-(2-(tert-butoxy)- |
| | 2-oxoethoxy)pyrrolidine-2-carboxylic acid |
| | (2R,4S)-1-(((9H-fluoren-9-yl)methoxy)carbonyl)-4- |
| | hydroxypyrrolidine-2-carboxylic acid (CAS: 139262-20-7) |
| | ¹H NMR (500 MHz, DMSO-d₆) δ 13.36-12.19 (m, 1H), 7.94-7.86 |
| | (m, 2H), 7.70-7.62 (m, 2H), 7.47-.38 (m, 2H), 7.38-7.27 (m, |
| | 2H), 4.43-4.23 (m, 2H), 4.23-4.11 (m, 3H), 4.08-3.98 (m, |
| | 2H), 3.63-3.46 (m, 2H), 2.49-2.27 (m, 1H), 2.15-1.94 (m, 1H), |
| | 1.47-1.36 (m, 9H). LC-MS (ESI, m/z): [M + H]⁺ 468. |
| 31- 03 | 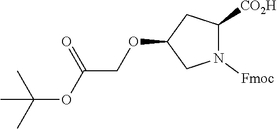 |
| | (2S,4S)-1-(((9H-fluoren-9-yl)methoxy)carbonyl)-4-(2-(tert-butoxy)- |
| | 2-oxoethoxy)pyrrolidine-2-carboxylic acid |
| | (2S,4S)-1-(((9H-fluoren-9-yl)methoxy)carbonyl)-4- |
| | hydroxypyrrolidine-2-carboxylic acid (CAS: 189249-10-3) |
| | ¹H NMR (500 MHz, DMSO-d₆) δ 13.17-12.11 (m, 1H), 7.94-7.83 |
| | (m, 2H), 7.72-7.61 (m, 2H), 7.48-7.39 (m, 2H), 7.38-7.28 (m, |
| | 2H), 4.41-4.25 (m, 2H), 4.25-4.10 (m, 3H), 4.02-3.94 (m, 2H), |
| | 3.69-3.59 (m, 1H), 3.41-3.33 (m, 1H), 2.48-2.33 (m, 1H), |
| | 1H), 2.21-2.01 (m, 1.47-1.35 (m, 9H). |
| | LC-MS (ESI, m/z): [M − tBu + H]⁺ 412. |

### Example-32-01: (S)-2-((((9H-fluoren-9-yl)methoxy)carbonyl)amino)-3-(7-(5-iodo pyrimidin-2-yl)-1H-indol-3-yl)propanoic acid

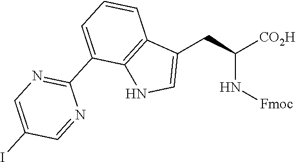

To a solution of (2S)-3-{1-[(tert-butoxy)carbonyl]-7-(pyrimidin-2-yl)-1H-indol-3-yl}-2-({[(9H-fluoren-9-yl)methoxy]carbonyl}amino)propanoic acid (5.00 g, 8.27 mmol) in MeCN (83 ml) at rt was added triethylamine (5.76 ml, 41.3 mmol). The mixture was stirred at 60° C. for 2 h and at rt for 17 h. The resulting mixture was concentrated and the residue was suspended in THF. The solid was collected by filtration and washed with THF. To the suspension of the obtained material in acetone (50 mL) at 0° C. were added Boc₂O (2.17 g, 9.92 mmol) and NaHCO₃ (2.78 g, 33.1 mmol) in H₂O (33 mL). The mixture was stirred at rt for 2.5 h, and then concentrated. The resulting aqueous suspension was diluted with EtOAc and extracted with water. The combined aqueous extracts were acidified with 1 N aq. HCl and extracted with EtOAc. The combined organic extracts were washed with brine, dried over Na₂SO₄, filtered, and then concentrated. To a solution of the obtained material (3.62 g, 7.50 mmol) in CH₂Cl₂ (30 ml) at 0° C. was added tert-butyl (Z)—N,N′-diisopropylcarbamimidate (CAS: 344286-67-5, 8.95 ml, 37.5 mmol). The mixture was stirred at rt for 4 h and at 40° C. for 5 h. The mixture was quenched with satd. aq. NaHCO₃ and diluted with EtOAc. Insoluble material was removed by filtration and washed with EtOAc. The filtrate was extracted with EtOAc. The organic extract was washed with brine, dried over Na₂SO₄, filtered, and then concentrated. The resulting residue was purified by silica gel flash column chromatography (gradient, Heptane/EtOAc=90/10 to 60/40). Appropriate fractions were concentrated. The mixture of (1,5-Cyclooctadiene)(methoxy)iridium(I) Dimer (CAS: 12148-71-9, 0.17 g, 0.26 mmol) and 4,4′-Di-tert-butyl-2,2′-bipyridyl (CAS: 72914-19-3, 0.14 g, 0.52 mmol) in THF (22.2 mL) was stirred at rt for 1 h under N₂ atmosphere. To a mixture of the obtained material (2.82 g, 5.24 mmol) and Bis(pinacolato)diboron (CAS: 73183-34-3, 1.26 g, 4.98 mmol) in THF (35 mL) at rt was added the catalyst solution prepared above via a syringe under N₂ atmosphere. The mixture was stirred at 80° C. for 8 h, and poured into cold satd. aq. NaHCO₃. The resulting mixture was extracted with EtOAc. The combined organic extracts were washed with brine, dried over Na₂SO₄, filtered, and then concentrated. The mixture of copper(I) oxide (CAS: 1317-39-1, 0.08 g, 0.58 mmol) and 28% aq. ammonia (1.96 ml, 29.0 mmol) in water (5.8 mL) was stirred at rt for 50 min. The suspension was added to a suspension of the obtained material in the last step (3.85 g, 5.80 mmol) in MeOH (52.2 mL). Potassium iodide (4.81 g, 29.0 mmol) was added and the mixture was stirred at 80° C. for 8 h. The reaction mixture was quenched with satd. aq. NH₄Cl, extracted with EtOAc, washed with brine, dried over Na₂SO₄, filtered, and then concentrated. The resulting residue was purified by silica gel flash column chromatography (gradient, Heptane/EtOAc=90/10 to 60/40), and then further purified by silica gel flash column chromatography (gradient, CH₂Cl₂/EtOAc=95/5 to 90/10).

Appropriate fractions were collected and concentrated. To a solution of the obtained material (1.94 g, 2.92 mmol) in CH₂Cl₂ (9 mL) at 0° C. was added TFA (9.0 mL, 117 mmol). The mixture was stirred at rt for 16 h, and then concentrated. To the suspension of the resulting residue in 1,4-dioxane (19.5 mL) at 0° C. were added Na₂CO₃ (1.24 g, 11.7 mmol) in H₂O (19.5 mL) and Fmoc-OSu (0.30 g, 0.88 mmol) in 1,4-dioxane (19.5 mL). The mixture was stirred at rt for 4 h. To the mixture at 0° C. were added Fmoc-OSu (0.30 g, 0.88 mmol) and Na₂CO₃ (0.31 g, 2.92 mmol). The mixture was stirred at rt for 23 h. The reaction was quenched with 1 N aq. HCl. The mixture was extracted with EtOAc. The organic extracts were washed with brine, dried over Na₂SO₄, filtered, and concentrated. The resulting residue was triturated with EtOAc and the resulting solid was collected by filtration to afford the title compound (1.68 g, 2.66 mmol). ¹H NMR (500 MHz, DMSO-d₆) δ 12.72 (br, 1H), 11.27-11.20 (m, 1H), 9.15 (s, 2H), 8.24 (d, J=7.5 Hz, 1H), 7.94-7.78 (m, 3H), 7.75 (d, J=8.2 Hz, 1H), 7.70-7.60 (m, 2H), 7.52-7.09 (m, 6H), 4.38-3.99 (m, 4H), 3.30-2.98 (m, 2H). LC-MS (ESI, m/z): [M+H]⁺ 631.

### Example-33-01: (2S)-4-{[2-(2,6-dioxopiperidin-3-yl)-1,3-dioxo-2,3-dihydro-1H-isoindol-4-yl]oxy}-2-({[(9H-fluoren-9-yl)methoxy]carbonyl}amino)butanoic acid

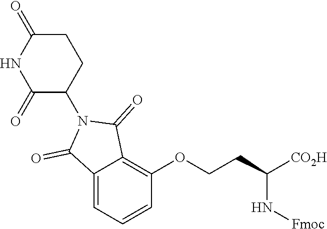

To a solution of (3S)-4-(tert-butoxy)-3-({[(9H-fluoren-9-yl)methoxy]carbonyl}amino)-4-oxobutanoic acid (CAS: 129460-09-9, 15.0 g, 36.5 mmol) in THF (148 ml) at 0° C. were added N-methylmorpholine (5.21 ml, 47.4 mmol) and ethyl chloroformate (3.85 ml, 40.1 mmol). The mixture was stirred at 0° C. for 1 h. To the mixture at 0° C. was added a solution of sodium borohydride (2.76 g, 72.9 mmol) in water (33.9 mL) dropwise. The mixture was stirred at 0° C. for 1 h. The mixture was poured into satd. aq. NH₄Cl. at 0° C. The mixture was extracted with EtOAc. The combined organic extracts were washed with brine, dried over Na₂SO₄, filtered, and concentrated. The resulting residue was purified by silica gel flash column chromatography (gradient, heptane/EtOAc=90/10 to 0/100). Appropriate fractions were collected and concentrated. To a solution of the obtained material (5.00 g, 12.6 mmol) in THF (126 mL) at rt was added triphenylphosphine (4.95 g, 18.9 mmol). To the mixture at 0° C. was added diisopropyl azodicarboxylate (CAS: 2446-83-5, 3.72 ml, 18.9 mmol). The resulting solution was stirred overnight at rt, and then the volatiles were removed under reduced pressure. The resulting residue was purified by reversed phase silica gel flash column chromatography (C18 silica gel, gradient, H₂O/CH₃CN=90/10 to 0/100). Appropriate fractions were collected and concentrated. To a solution of the obtained material (1.88 g, 2.88 mmol) in DCM (7.2 mL) at 0° C. was added TFA (7.2 mL). The mixture was stirred at rt for 4 h, and then concentrated, and azeotroped three times with toluene. The resulting residue was triturated with toluene and the resulting solid was collected by filtration to afford the title compound (1.56 g, 2.61 mmol). ¹H NMR (500 MHz, DMSO-d₆) δ 12.75 (br, 1H), 11.09 (s, 1H), 7.91-7.86 (m, 2H), 7.84-7.78 (m, 1H), 7.76-7.67 (in, 3H), 7.55-7.49 (m, 1H), 7.49-7.44 (m, 1H), 7.44-7.37 (m, 2H), 7.34-7.27 (m, 2H), 5.13-5.03 (m, 1H), 4.37-4.17 (m, 6H), 2.91-2.79 (m, 1H), 2.60-2.51 (m, 2H), 2.33-2.23 (m, 1H), 2.16-2.05 (m, 1H), 2.05-1.95 (in, 1H). LC-MS (m/z): [M+H]⁺ 598.

### Example-33

The following compounds can be synthesized as outlined for the preparation of Example-33-01 employing appropriate starting materials in the table.

| | Structure and IUPAC name |
|---|---|
| | Starting material-A |
| Example | NMR and MS |
| 33-02 | 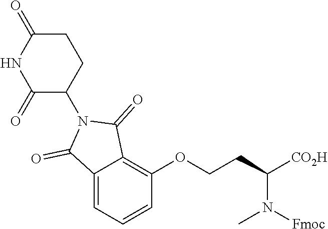 |
| | (2S)-4-{[2-(2,6-dioxopiperidin-3-yl)-1,3-dioxo-2,3-dihydro-1H-isoindol-4- |
| | yloxy}-2-({[(9H-fluoren-9-yl)methoxy]carbonyl}(methyl)amino)butanoic |
| | acid |
| | (3S)-4-(tert-butoxy)-3-({[(9H-fluoren-9-yl)methoxy]carbonyl}(methyl)ami- |
| | no)-4-oxobutanoic acid (CAS: 2271413-88-6) |
| | ¹H NMR (500 MHz, DMSO-d₆) δ 12.99 (s, 1H), 11.13-11.00 (m, |
| | 1H), 7.93-7.77 (m, 3H), 7.65-7.11 (m, 8H), 5.14-4.93 (m, 1H), |
| | 4.80-4.54 (m, 1H), 4.37-3.93 (m, 5H), 2.93-2.68 (m, 4H), 2.61- |
| | 2.53 (m, 1H), 2.47-2.18 (m, 3H), 2.11-1.62 (m, 1H). LC-MS |
| | (m/z): [M + H]⁺ 612. |
| 33-03 | 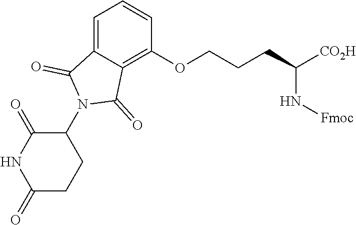 |
| | (2S)-5-{[2-(2,6-dioxopiperidin-3-yl)-1,3-dioxo-2,3-dihydro-1H-isoindol-4- |
| | yl]oxy}-2-({[(9H-fluoren-9-yl)methoxy]carbonyl}amino)pentanoic acid |
| | (4S)-5-(tert-butoxy)-4-({[(9H-fluoren-9-yl)methoxy]carbonyl}amino)-5-ox- |
| | opentanoic acid (CAS: 84793-07-7) |
| | ¹H NMR (500 MHz, DMSO-d₆) δ 12.63 (br, 1H), 11.10 (s, 1H), 7.93- |
| | 7.86 (m, 2H), 7.84-7.77 (m, 1H), 7.77-7.58 (m, 3H), 7.56- |
| | 7.49 (m, 1H), 7.48-7.37 (m, 3H), 7.36-7.28 (m, 2H), 5.13-5.03 |
| | (m, 1H), 4.36-4.15 (m, 5H), 4.07-3.97 (m, 1H), 2.94-2.80 (m, |
| | 1H), 2.67-2.51 (m, 2H), 2.08-1.91 (m, 2H), 1.90-1.74 (m, 3 H). |
| | LC-MS (m/z): [M + H]⁺ 612. |

What is claimed is:

1. An amino acid derivative selected from the group consisting of

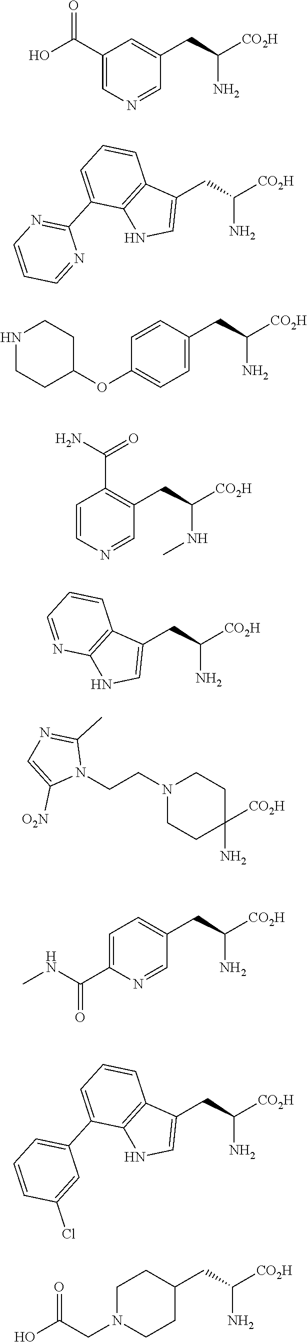

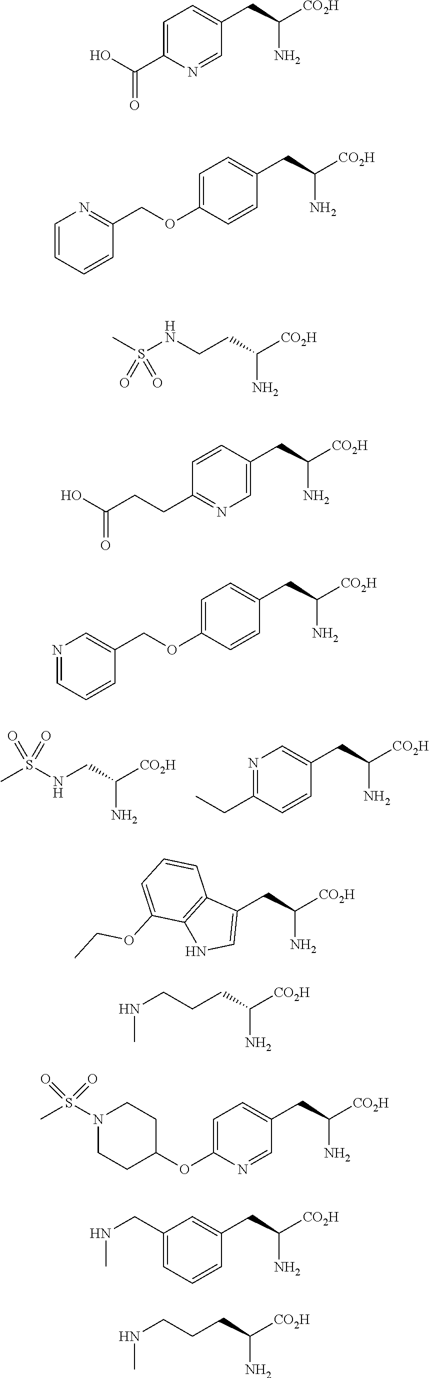

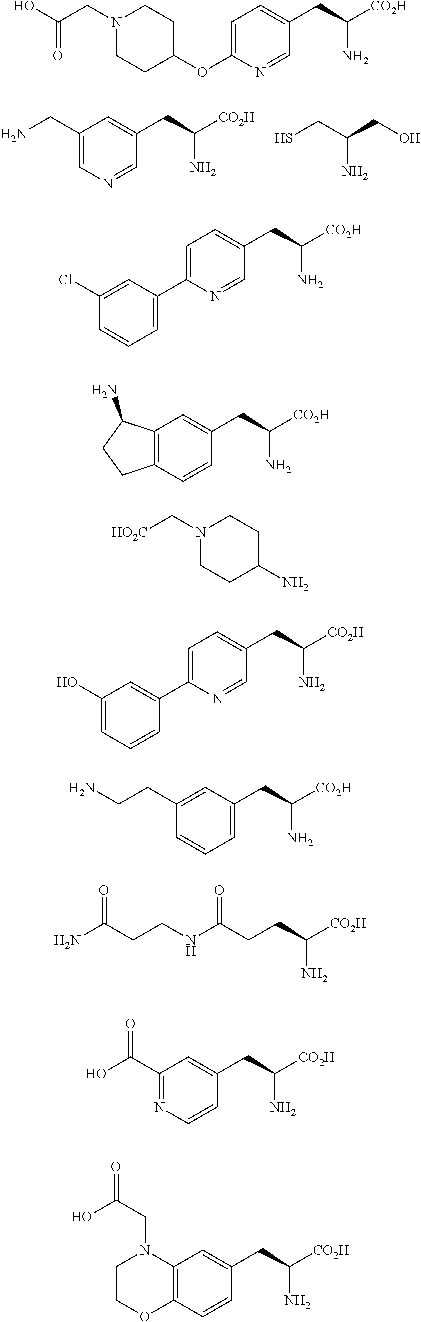

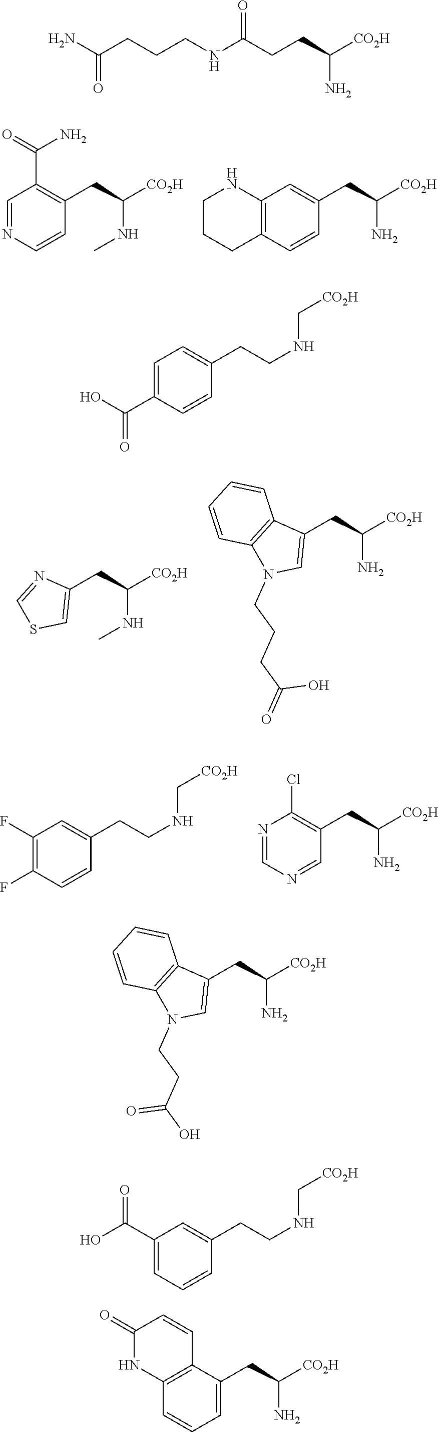

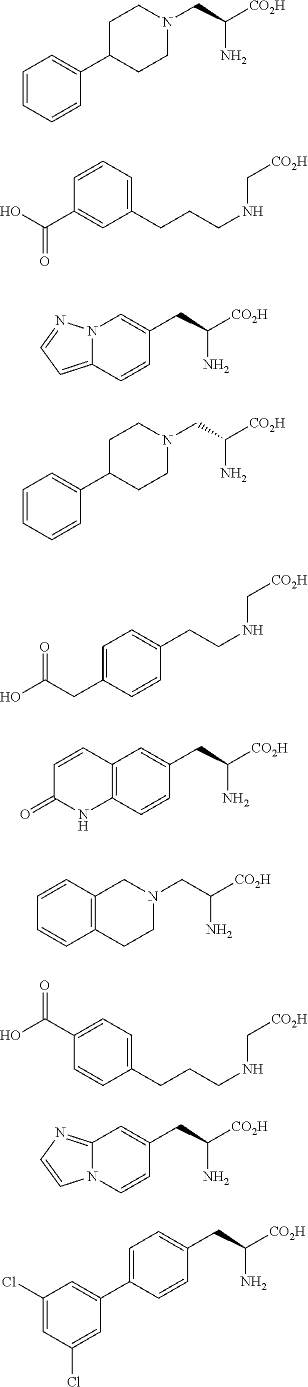

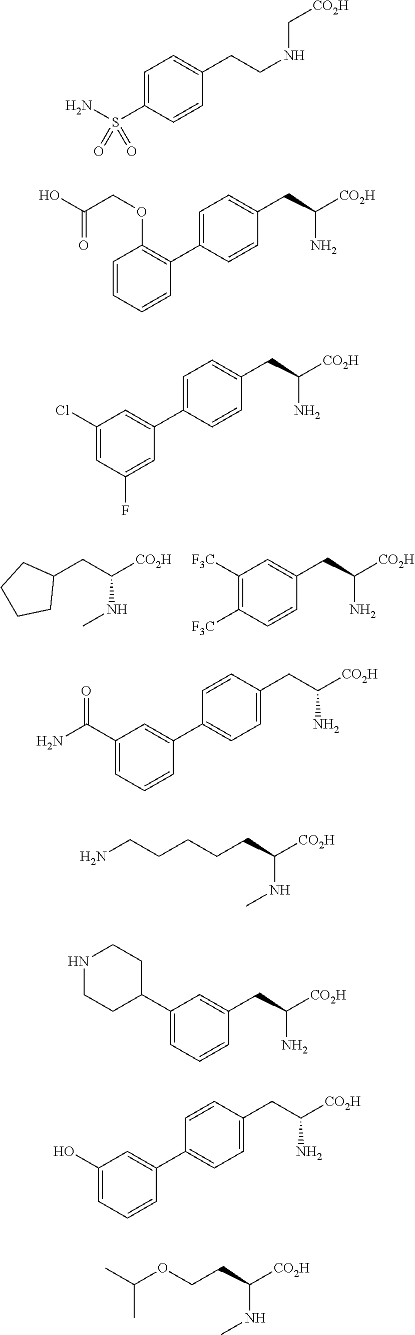

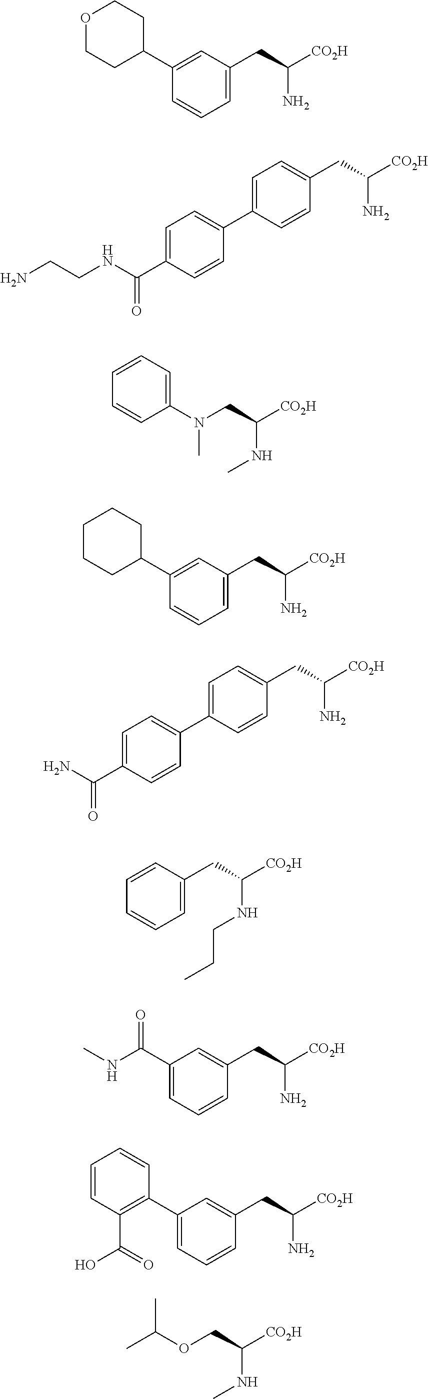

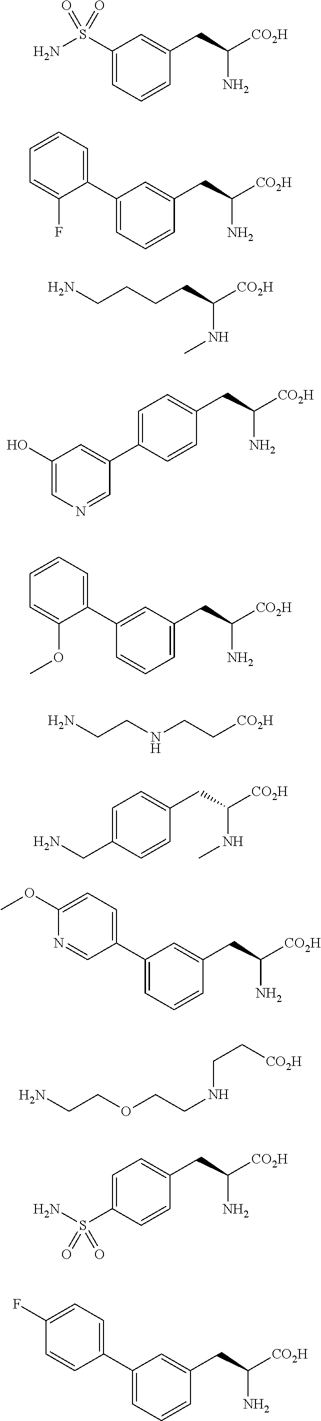

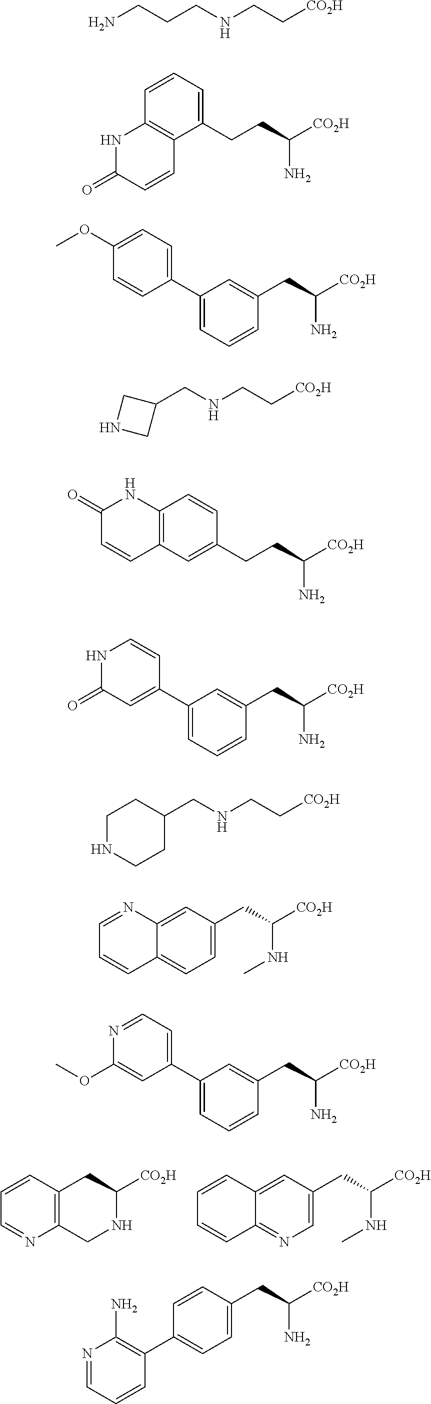

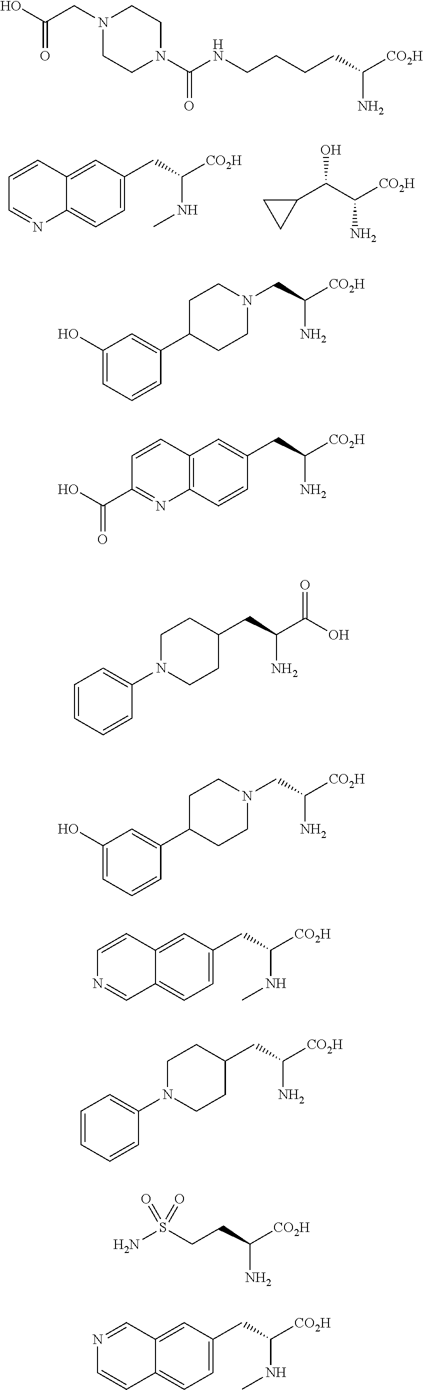

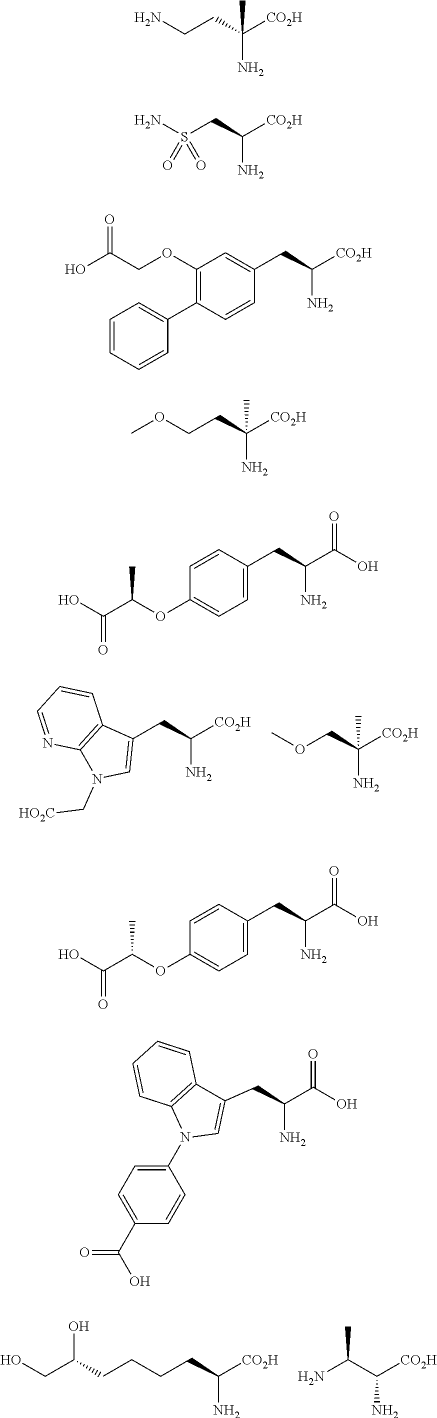

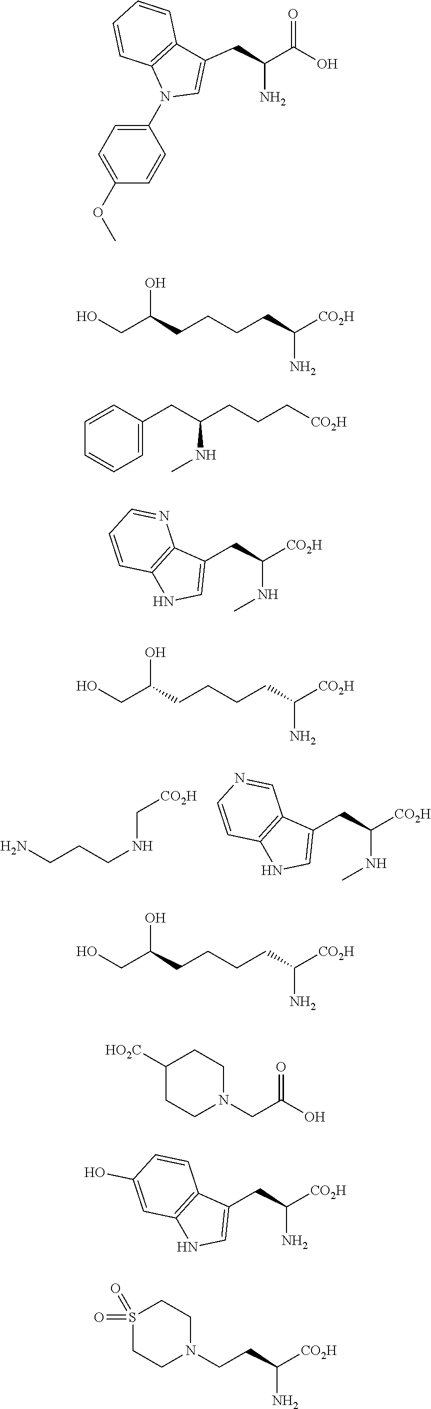

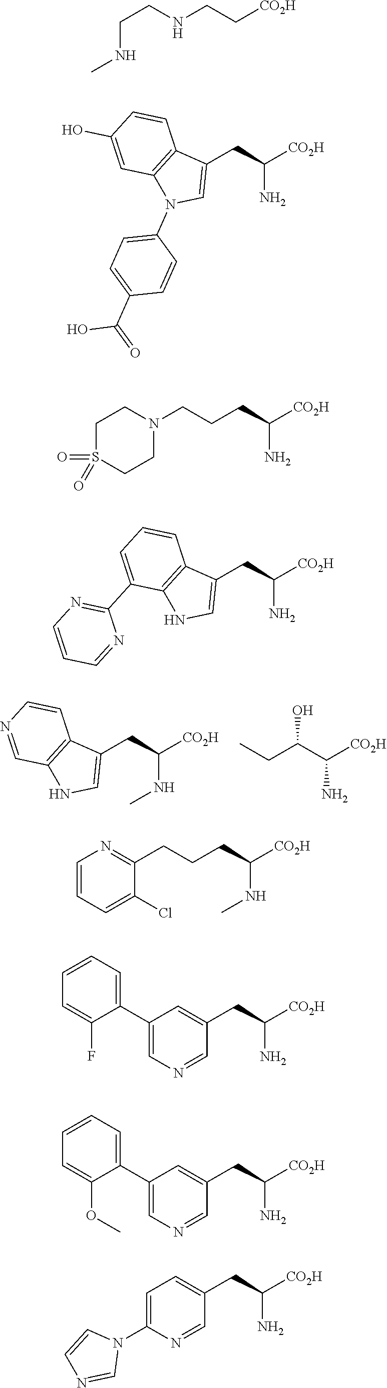

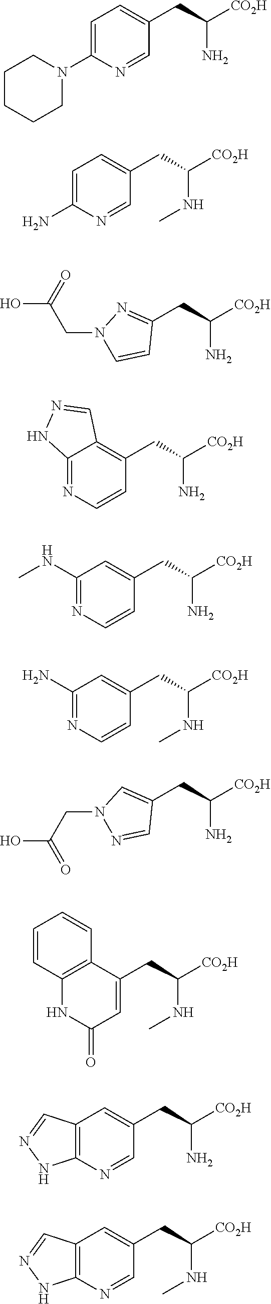

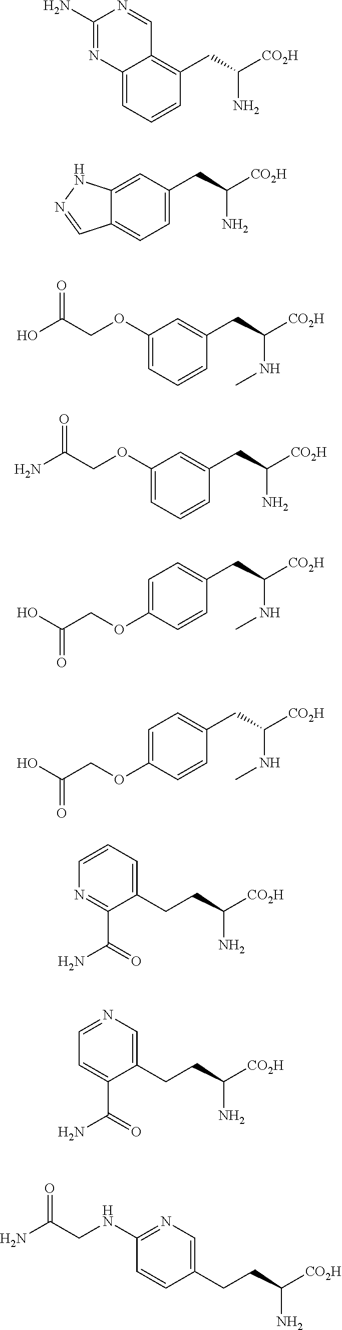

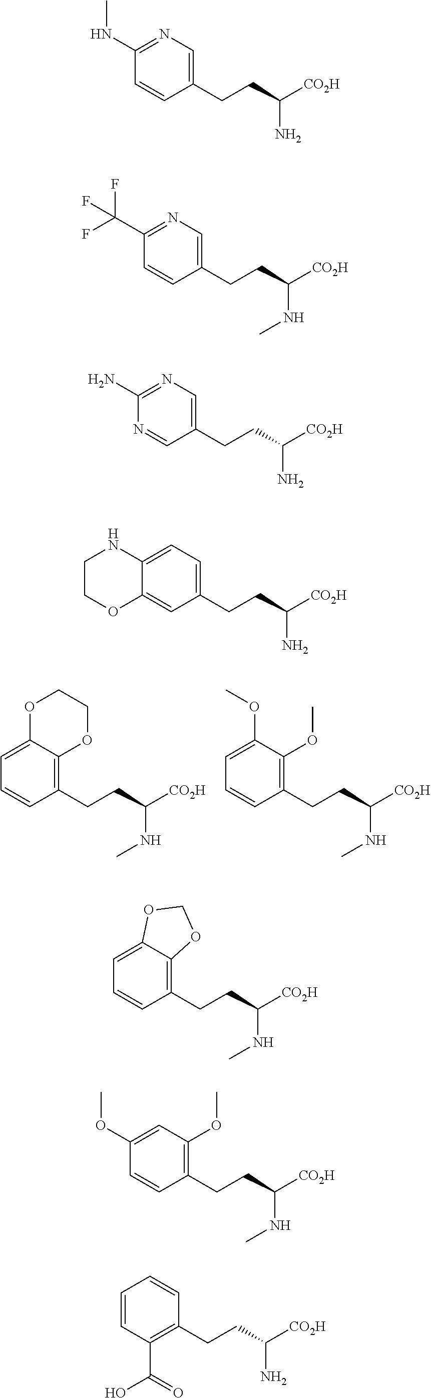

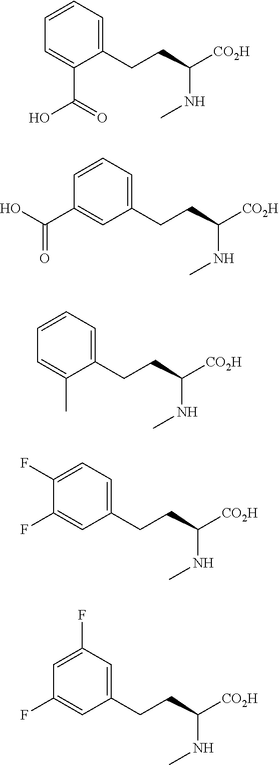

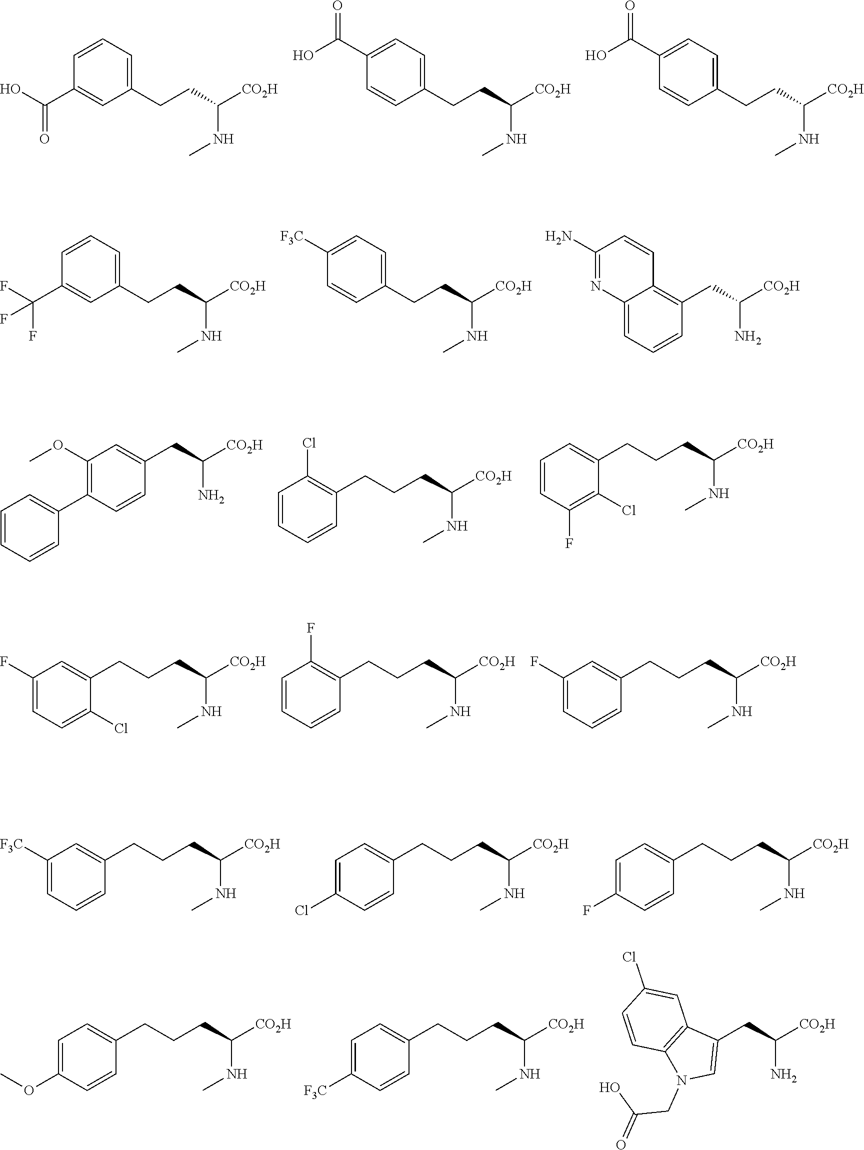

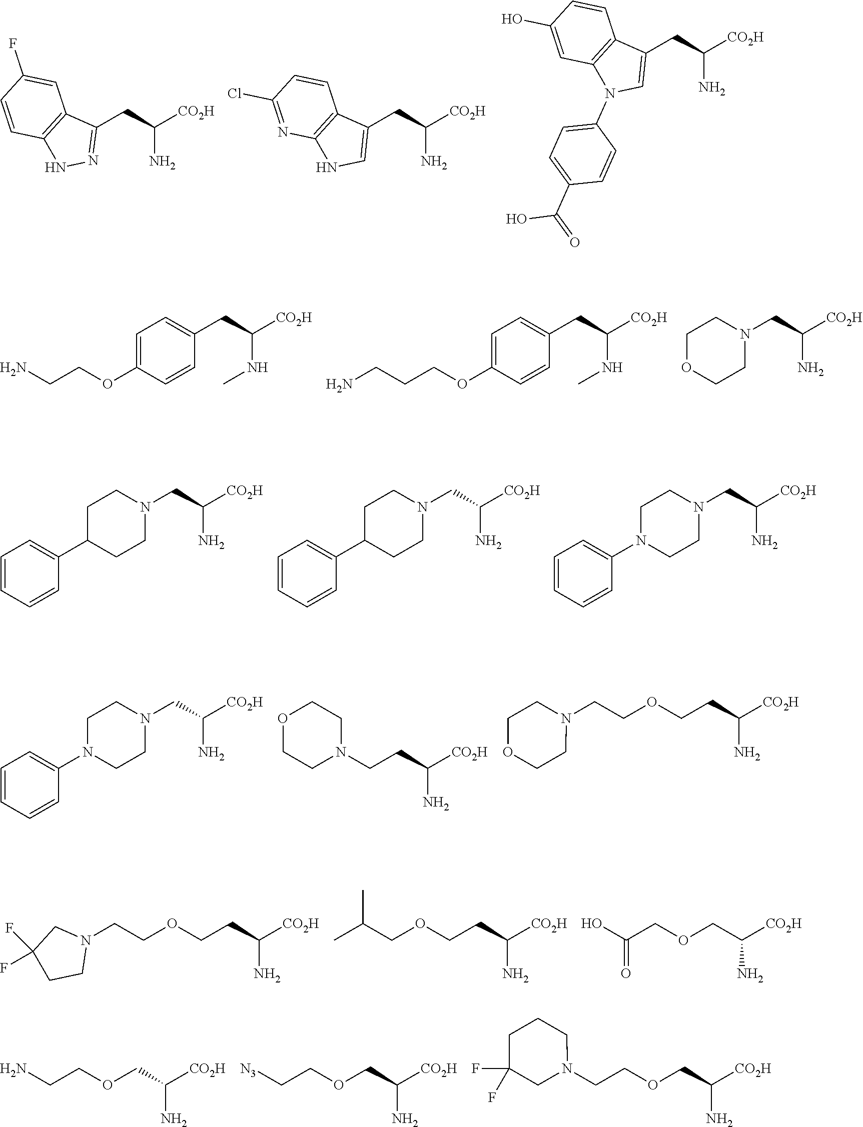

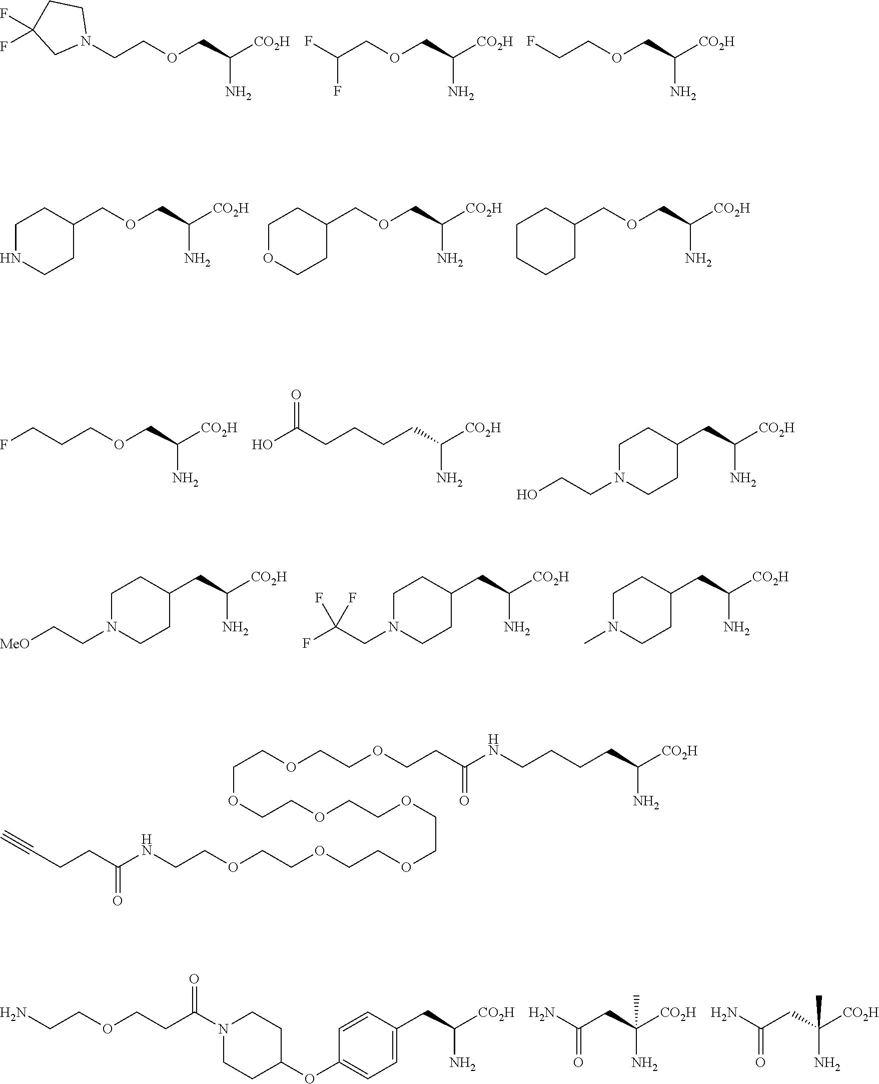

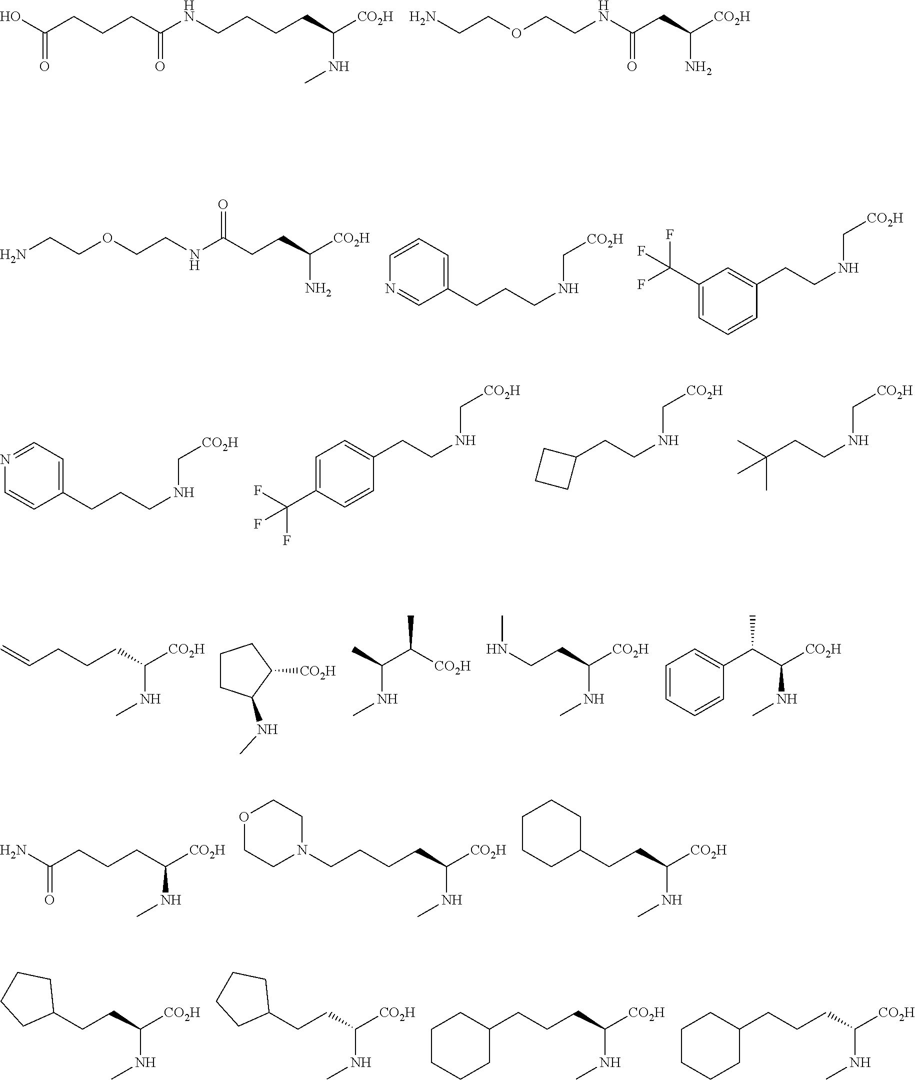

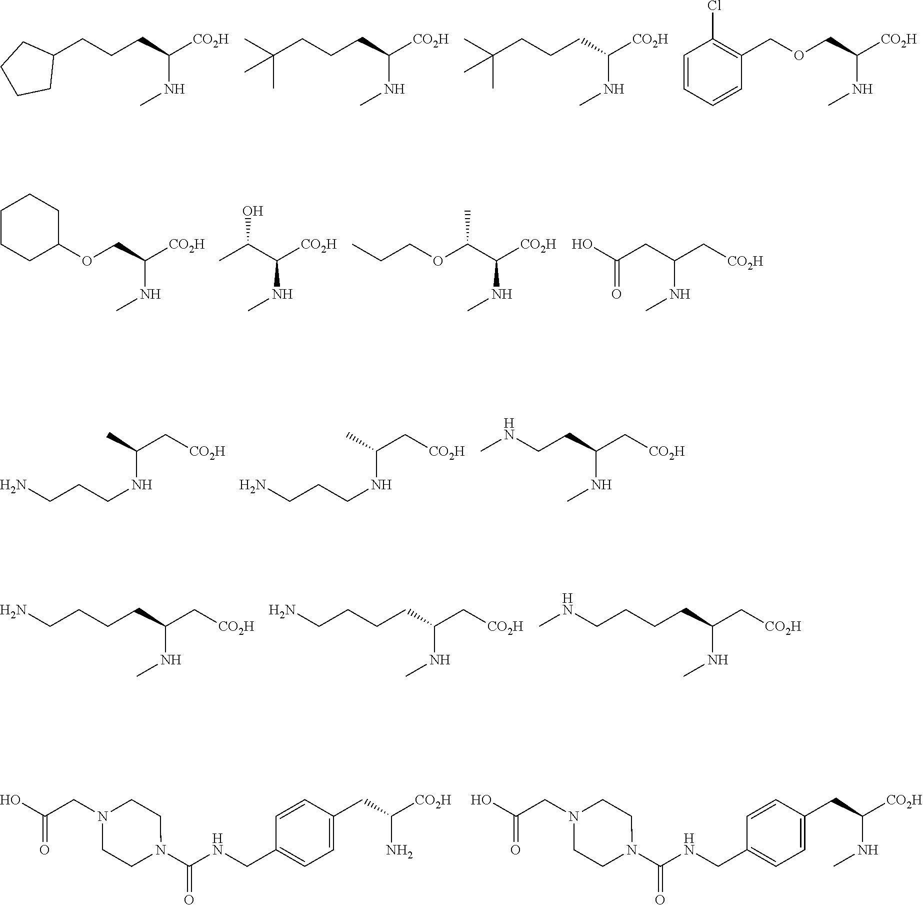

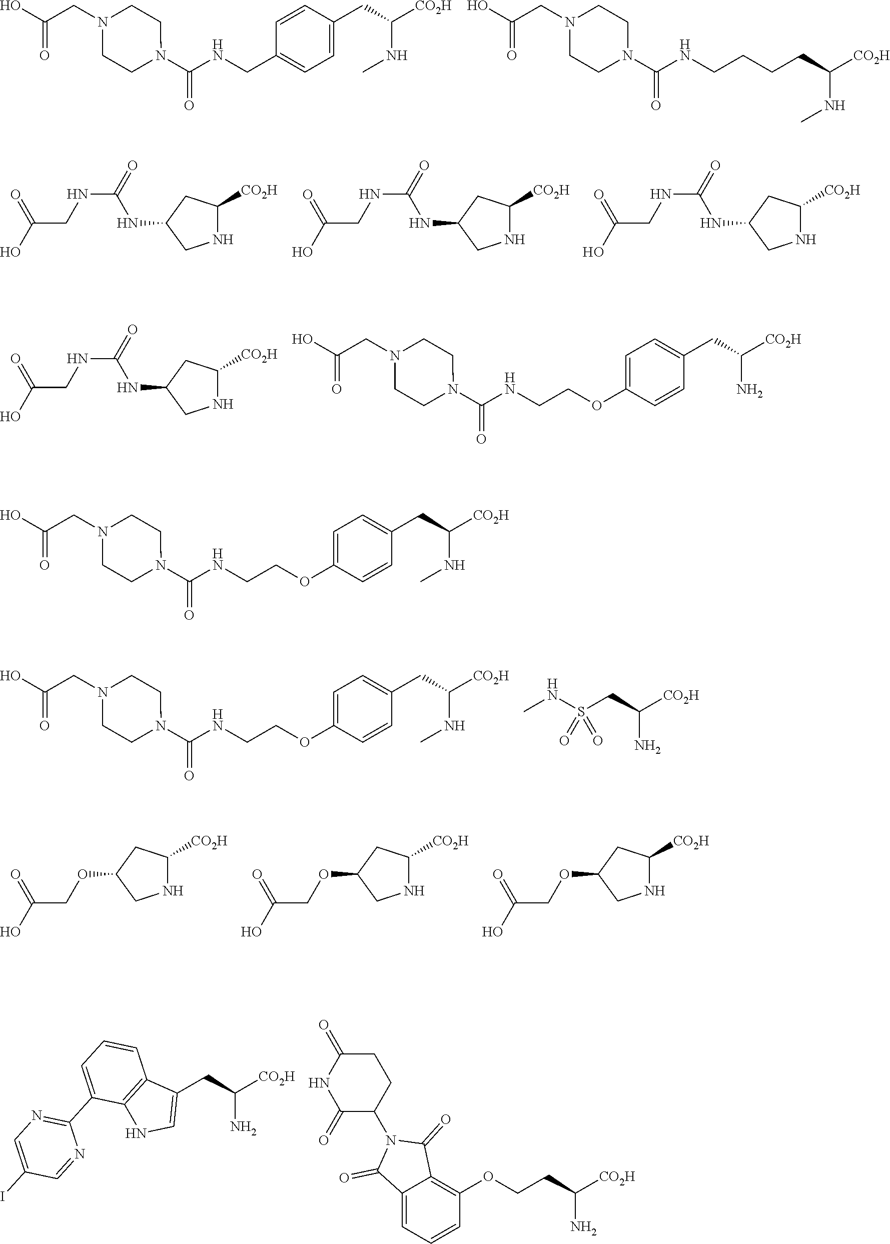

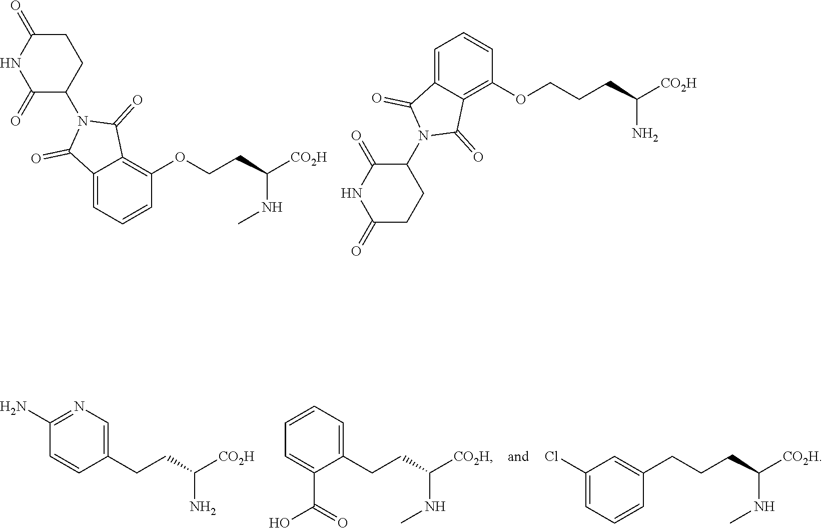 [SMILES: CN[C@@H](CCOc1cccc2c1C(=O)N(C1CCC(=O)NC1=O)C2=O)C(=O)O ; N[C@@H](CCCOc1cccc2c1C(=O)N(C1CCC(=O)NC1=O)C2=O)C(=O)O ; Nc1ccc(CC[C@@H](N)C(=O)O)cn1 ; CN[C@H](CCc1ccccc1C(=O)O)C(=O)O ; "and" ; CN[C@@H](CCCc1cccc(Cl)c1)C(=O)O]

2. An amino acid derivative selected from the group consisting of (S)-5-(2-amino-2-carboxyethyl)nicotinic acid, (S)-3-(4-carbamoylpyridin-3-yl)-2-(methylamino)propanoic acid, (S)-2-amino-3-(6-(methylcarbamoyl)pyridin-3-yl)propanoic acid, (S)-5-(2-amino-2-carboxyethyl)picolinic acid, (S)-2-amino-3-(6-(2-carboxyethyl)pyridin-3-yl)propanoic acid, (S)-2-amino-3-(6-ethylpyridin-3-yl)propanoic acid, (S)-2-amino-3-(6-((1-(methylsulfonyl)piperidin-4-yl)oxy)pyridin-3-yl)propanoic acid, (S)-2-amino-3-(6-((1-(carboxymethyl)piperidin-4-yl)oxy)pyridin-3-yl)propanoic acid, (S)-2-amino-3-(6-(3-chlorophenyl)pyridin-3-yl)propanoic acid, (S)-2-amino-3-(6-(3-hydroxyphenyl)pyridin-3-yl)propanoic acid, (S)-4-(2-amino-2-carboxyethyl)picolinic acid, (S)-3-(3-carbamoylpyridin-4-yl)-2-(methylamino)propanoic acid, (S)-2-(methylamino)-3-(thiazol-4-yl)propanoic acid, (S)-2-amino-3-(4-chloropyrimidin-5-yl)propanoic acid, (S)-2-amino-3-(2-oxo-1,2-dihydroquinolin-5-yl)propanoic acid, (S)-2-amino-3-(pyrazolo[1,5-a]pyridin-6-yl)propanoic acid, (S)-2-amino-3-(2-oxo-1,2-dihydroquinolin-6-yl)propanoic acid, (S)-2-amino-3-(imidazo[1,2-a]pyridin-7-yl)propanoic acid, (S)-2-amino-3-(2′-(carboxymethoxy)-[1,1′-biphenyl]-4-yl)propanoic acid, (S)-2-amino-3-(3,4-bis(trifluoromethyl)phenyl)propanoic acid, (S)-2-amino-3-(3-(piperidin-4-yl)phenyl)propanoic acid, (S)-2-amino-3-(3-(tetrahydro-2H-pyran-4-yl)phenyl)propanoic acid, (S)-2-amino-3-(3-cyclohexylphenyl)propanoic acid, (S)-2-amino-3-(3-(methylcarbamoyl)phenyl)propanoic acid, (S)-2-amino-3-(3-sulfamoylphenyl)propanoic acid, (S)-2-amino-3-(4-(5-hydroxypyridin-3-yl)phenyl)propanoic acid, (R)-3-(4-(aminomethyl)phenyl)-2-(methylamino)propanoic acid, (S)-2-amino-3-(4-sulfamoylphenyl)propanoic acid, (S)-2-amino-4-(2-oxo-1,2-dihydroquinolin-5-yl)butanoic acid, (S)-2-amino-4-(2-oxo-1,2-dihydroquinolin-6-yl)butanoic acid, (R)-2-(methylamino)-3-(quinolin-7-yl)propanoic acid, (R)-2-(methylamino)-3-(quinolin-3-yl)propanoic acid, (R)-2-(methylamino)-3-(quinolin-6-yl)propanoic acid, (S)-6-(2-amino-2-carboxyethyl)quinoline-2-carboxylic acid, (R)-3-(isoquinolin-6-yl)-2-(methylamino)propanoic acid, (R)-3-(isoquinolin-7-yl)-2-(methylamino)propanoic acid, (S)-2-amino-3-(2-(carboxymethoxy)-[1,1′-biphenyl]-4-yl)propanoic acid, (S)-2-amino-3-(1-(carboxymethyl)-1H-pyrrolo[2,3-b]pyridin-3-yl)propanoic acid, 4-{3-[(2S)-2-amino-2-carboxyethyl]-1H-indol-1-yl}benzoic acid, (2S)-2-amino-3-[1-(4-methoxyphenyl)-1H-indol-3-yl]propanoic acid, (S)-2-(methylamino)-3-(1H-pyrrolo[3,2-b]pyridin-3-yl)propanoic acid, (S)-2-(methylamino)-3-(1H-pyrrolo[3,2-c]pyridin-3-yl)propanoic acid, (S)-2-amino-3-(6-hydroxy-1H-indol-3-yl)propanoic acid, (S)-4-(3-(2-amino-2-carboxyethyl)-6-hydroxy-1H-indol-1-yl)benzoic acid, (S)-2-(methylamino)-3-(1H-pyrrolo[2,3-c]pyridin-3-yl)propanoic acid, (R)-2-amino-3-(7-(pyrimidin-2-yl)-1H-indol-3-yl)propanoic acid, (S)-2-amino-3-(1H-pyrrolo[2,3-b]pyridin-3-yl)propanoic acid, (S)-2-amino-3-(7-(3-chlorophenyl)-1H-indol-3-yl)propanoic acid, (S)-2-amino-3-(4-(pyridin-2-ylmethoxy)phenyl)propanoic acid, (S)-2-amino-3-(4-(pyridin-3-ylmethoxy)phenyl)propanoic acid, (S)-2-amino-3-(7-ethoxy-1H-indol-3-yl)propanoic acid, (S)-2-amino-3-(3-((methylamino)methyl)phenyl)propanoic acid, (S)-2-amino-3-(5-(aminomethyl)pyridin-3-yl)propanoic acid, (S)-2-amino-3-((R)-3-amino-2,3-dihydro-1H-inden-5-yl)propanoic acid, (S)-2-amino-3-(3-(2-aminoethyl)phenyl)propanoic acid, (S)-2-amino-3-(4-(carboxymethyl)-3,4-dihydro-2H-benzo[b][1,4]oxazin-6-yl)propanoic acid, (S)-2-amino-3-(1,2,3,4-tetrahydroquinolin-7-yl)propanoic acid, 4-{3-[(2S)-2-amino-2-carboxyethyl]-1H-indol-1-yl}butanoic acid, (2S)-2-amino-3-[1-(2-carboxyethyl)-1H-indol-3-yl]propanoic acid, (S)-2-amino-3-(4-phenylpiperidin-1-yl)propanoic acid, (R)-2-amino-3-(4-phenylpiperidin-1-yl)propanoic acid, 2-amino-3-(3,4-dihydroisoquinolin-2(1H)-yl)propanoic acid, (S)-2-amino-3-(3′,5′-dichloro-[1,1′-biphenyl]-4-yl)propanoic acid, (S)-2-amino-3-(3′-chloro-5′-fluoro-[1,1′-biphenyl]-4-yl)propanoic acid, (R)-2-amino-3-(3′-carbamoyl-[1,1′-biphenyl]-4-yl)propanoic acid, (R)-2-amino-3-(3′-hydroxy-[1,1′-biphenyl]-4-yl)propanoic acid, (R)-2-amino-3-(4′-((2-aminoethyl)carbamoyl)-[1,1′-biphenyl]-4-yl)propanoic acid, (R)-2-amino-3-(4′-carbamoyl-[1,1′-biphenyl]-4-yl)propanoic acid, (S)-3′-(2-amino-2-carboxyethyl)-[1,1′-biphenyl]-2-carboxylic acid, (S)-2-amino-3-(2′-fluoro-[1,1′-biphenyl]-3-yl)propanoic acid, (S)-2-amino-3-(2′-methoxy-[1,1′-biphenyl]-3-yl)propanoic acid, (S)-2-amino-3-(3-(6-methoxypyridin-3-yl)phenyl)propanoic acid, (S)-2-amino-3-(4′-fluoro-[1,1′-biphenyl]-3-yl)propanoic acid, (S)-2-amino-3-(4′-methoxy-[1,1′-biphenyl]-3-yl)propanoic acid, (S)-2-amino-3-(3-(2-oxo-1,2-dihydropyridin-4-yl)phenyl)propanoic acid, (S)-2-amino-3-(3-(2-methoxypyridin-4-yl)phenyl)propanoic acid, (S)-2-amino-3-(4-(2-aminopyridin-3-yl)phenyl)propanoic acid, (2R,3S)-2-amino-3-cyclopropyl-3-hydroxypropanoic acid, (S)-2-amino-3-(1-phenylpiperidin-4-yl)propanoic acid, (R)-2-amino-3-(1-phenylpiperidin-4-yl)propanoic acid, (R)-2,4-diamino-2-methylbutanoic acid, (S)-2-amino-4-methoxy-2-methylbutanoic acid, (S)-2-amino-3-methoxy-2-methylpropanoic acid, (2S,7R)-2-amino-7,8-dihydroxyoctanoic acid, (2S,7S)-2-amino-7,8-dihydroxyoctanoic acid, (2R,7R)-2-amino-7,8-dihydroxyoctanoic acid, (2R,7S)-2-amino-7,8-dihydroxyoctanoic acid, (S)-2-amino-4-(1,1-dioxidothiomorpholino)butanoic acid, (S)-2-amino-5-(1,1-dioxidothiomorpholino)pentanoic acid, (2R,3S)-2-amino-3-hydroxypentanoic acid, (S)-2-amino-3-(4-(piperidin-4-yloxy)phenyl)propanoic acid, 4-amino-1-(2-(2-methyl-5-nitro-1H-imidazol-1-yl)ethyl)piperidine-4-carboxylic acid, (R)-2-amino-3-(1-(carboxymethyl)piperidin-4-yl)propanoic acid, (R)-2-amino-4-(methylsulfonamido)butanoic acid, (R)-2-amino-3-(methylsulfonamido)propanoic acid, (R)-2-amino-5-(methylamino)pentanoic acid, (S)-2-amino-5-(methylamino)pentanoic acid, (R)-2-amino-3-mercaptopropan-1-ol, 2-(4-aminopiperidin-1-yl)acetic acid, (2S)-2-amino-4-[(2-carbamoylethyl)carbamoyl]butanoic acid, (2S)-2-amino-4-[(3-carbamoylpropyl)carbamoyl]butanoic acid, 4-(2-((carboxymethyl)amino)ethyl)benzoic acid, 2-{[2-(3,4-difluorophenyl)ethyl]amino}acetic acid, 3-(2-((carboxymethyl)amino)ethyl)benzoic acid, 3-(3-((carboxymethyl)amino)propyl)benzoic acid, 2-(4-(2-((carboxymethyl)amino)ethyl)phenyl)acetic acid, 4-(3-((carboxymethyl)amino)propyl)benzoic acid, 2-{[2-(4-sulfamoylphenyl)ethyl]amino}acetic acid, (R)-3-cyclopentyl-2-(methylamino)propanoic acid, (S)-7-amino-2-(methylamino)heptanoic acid, (2S)-2-(methylamino)-4-(propan-2-yloxy)butanoic acid, (S)-3-(methyl(phenyl)amino)-2-(methylamino)propanoic acid, (2R)-3-phenyl-2-(propylamino)propanoic acid, (2S)-2-(methylamino)-3-(propan-2-yloxy)propanoic acid, (2S)-6-amino-2-(methylamino)hexanoic acid, 3-((2-aminoethyl)amino)propanoic acid, 3-((2-(2-aminoethoxy)ethyl)amino)propanoic acid, 3-((3-aminopropyl)amino)propanoic acid, 3-((azetidin-3-ylmethyl)amino)propanoic acid, 3-((piperidin-4-ylmethyl)amino)propanoic acid, (S)-5,6,7,8-tetrahydro-1,7-naphthyridine-6-carboxylic acid, (2R)-2-amino-6-{[4-(carboxymethyl)piperazine-1-carbonyl]amino}hexanoic acid, (S)-2-amino-3-(4-(3-hydroxyphenyl)piperidin-1-yl)propanoic acid, (R)-2-amino-3-(4-(3-hydroxyphenyl)piperidin-1-yl)propanoic acid, (S)-2-amino-4-sulfamoylbutanoic acid, (2R)-2-amino-3-sulfamoylpropanoic acid, (S)-2-amino-3-(4-((R)-1-carboxyethoxy)phenyl)propanoic acid, (S)-2-amino-3-(4-((S)-1-carboxyethoxy)phenyl)propanoic acid, (2R,3S)-2,3-diaminobutanoic acid, (R)-5-(methylamino)-6-phenylhexanoic acid, 2-[(3-aminopropyl)amino]acetic acid, 1-(carboxymethyl)piperidine-4-carboxylic acid, and 3-((2-(methylamino)ethyl)amino)propanoic acid, (S)-2-amino-3-(7-(pyrimidin-2-yl)-1H-indol-3-yl)propanoic acid, (S)-5-(3-chloropyridin-2-yl)-2-(methylamino)pentanoic acid, (S)-2-amino-3-(5-(2-fluorophenyl)pyridin-3-yl)propanoic acid, (S)-2-amino-3-(5-(2-methoxyphenyl)pyridin-3-yl)propanoic acid, (S)-3-(6-(1H-imidazol-1-yl)pyridin-3-yl)-2-aminopropanoic acid, (S)-2-amino-3-(6-(piperidin-1-yl)pyridin-3-yl)propanoic acid, (R)-3-(6-aminopyridin-3-yl)-2-(methylamino)propanoic acid, (S)-2-amino-3-(1-(carboxymethyl)-1H-pyrazol-3-yl)propanoic acid, (R)-2-amino-3-(1H-pyrazolo[3,4-b]pyridin-4-yl)propanoic acid, (R)-2-amino-3-(2-(methylamino)pyridin-4-yl)propanoic acid, (R)-3-(2-aminopyridin-4-yl)-2-(methylamino)propanoic acid, (S)-2-amino-3-(1-(carboxymethyl)-1H-pyrazol-4-yl)propanoic acid, (S)-2-(methylamino)-3-(2-oxo-1,2-dihydroquinolin-4-yl)propanoic acid, (S)-2-amino-3-(1H-pyrazolo[3,4-b]pyridin-5-yl)propanoic acid, (S)-2-(methylamino)-3-(1H-pyrazolo[3,4-b]pyridin-5-yl)propanoic acid, (R)-2-amino-3-(2-aminoquinazolin-5-yl)propanoic acid, (S)-2-amino-3-(1H-indazol-6-yl)propanoic acid, (S)-3-(3-(carboxymethoxy)phenyl)-2-(methylamino)propanoic acid, (S)-2-amino-3-(3-(2-amino-2-oxoethoxy)phenyl)propanoic acid, (S)-3-(4-(carboxymethoxy)phenyl)-2-(methylamino)propanoic acid, (R)-3-(4-(carboxymethoxy)phenyl)-2-(methylamino)propanoic acid, (S)-2-amino-4-(2-carbamoylpyridin-3-yl)butanoic acid, (S)-2-amino-4-(4-carbamoylpyridin-3-yl)butanoic acid, (S)-2-amino-4-(6-((2-amino-2-oxoethyl)amino)pyridin-3-yl)butanoic acid, (S)-2-amino-4-(6-(methylamino)pyridin-3-yl)butanoic acid, (R)-2-amino-4-(6-aminopyridin-3-yl)butanoic acid, (S)-2-(methylamino)-4-(6-(trifluoromethyl)pyridin-3-yl)butanoic acid, (R)-2-amino-4-(2-aminopyrimidin-5-yl)butanoic acid, (S)-2-amino-4-(3,4-dihydro-2H-benzo[b][1,4]oxazin-7-yl)butanoic acid, (S)-4-(2,3-dihydrobenzo[b][1,4]dioxin-5-yl)-2-(methylamino)butanoic acid, (S)-4-(2,3-dimethoxyphenyl)-2-(methylamino)butanoic acid, (S)-4-(benzo[d][1,3]dioxol-4-yl)-2-(methylamino)butanoic acid, (S)-4-(2,4-dimethoxyphenyl)-2-(methylamino)butanoic acid, (R)-2-(3-amino-3-carboxypropyl)benzoic acid, (S)-2-(3-carboxy-3-(methylamino)propyl)benzoic acid, (R)-2-(3-carboxy-3-(methylamino)propyl)benzoic acid, (S)-3-(3-carboxy-3-(methylamino)propyl)benzoic acid, (R)-3-(3-carboxy-3-(methylamino)propyl)benzoic acid, (S)-2-(methylamino)-4-(o-tolyl)butanoic acid, (S)-4-(3,4-difluorophenyl)-2-(methylamino)butanoic acid, (S)-4-(3,5-difluorophenyl)-2-(methylamino)butanoic acid, (S)-4-(3-carboxy-3-(methylamino)propyl)benzoic acid, (R)-4-(3-carboxy-3-(methylamino)propyl)benzoic acid, (S)-2-(methylamino)-4-(3-(trifluoromethyl)phenyl)butanoic acid, (S)-2-(methylamino)-4-(4-(trifluoromethyl)phenyl)butanoic acid, (R)-2-amino-3-(2-aminoquinolin-5-yl)propanoic acid, (S)-2-amino-3-(2-methoxy-[1,1′-biphenyl]-4-yl)propanoic acid, (S)-5-(2-chlorophenyl)-2-(methylamino)pentanoic acid, (S)-5-(2-chloro-3-fluorophenyl)-2-(methylamino)pentanoic acid, (S)-5-(2-chloro-5-fluorophenyl)-2-(methylamino)pentanoic acid, (S)-5-(2-fluorophenyl)-2-(methylamino)pentanoic acid, (S)-5-(3-chlorophenyl)-2-(methylamino)pentanoic acid, (S)-5-(3-fluorophenyl)-2-(methylamino)pentanoic acid, (S)-2-(methylamino)-5-(3-(trifluoromethyl)phenyl)pentanoic acid, (S)-5-(4-chlorophenyl)-2-(methylamino)pentanoic acid, (S)-5-(4-fluorophenyl)-2-(methylamino)pentanoic acid, (S)-5-(4-methoxyphenyl)-2-(methylamino)pentanoic acid, (S)-2-(methylamino)-5-(4-(trifluoromethyl)phenyl)pentanoic acid, (S)-2-amino-3-(1-(carboxymethyl)-5-chloro-1H-indol-3-yl)propanoic acid, (S)-2-amino-3-(5-fluoro-1H-indazol-3-yl)propanoic acid, (S)-2-amino-3-(6-chloro-1H-pyrrolo[2,3-b]pyridin-3-yl)propanoic acid, (S)-4-(3-(2-amino-2-carboxyethyl)-6-hydroxy-1H-indol-1-yl)benzoic acid, (S)-3-(4-(2-aminoethoxy)phenyl)-2-(methylamino)propanoic acid, (S)-2-amino-3-(4-(3-aminopropoxy)phenyl)propanoic acid, (S)-2-amino-3-morpholinopropanoic acid, (S)-2-amino-3-(4-phenylpiperidin-1-yl)propanoic acid, (R)-2-amino-3-(4-phenylpiperidin-1-yl)propanoic acid, (S)-2-amino-3-(4-phenylpiperazin-1-yl)propanoic acid, (R)-2-amino-3-(4-phenylpiperazin-1-yl)propanoic acid, (S)-2-amino-4-morpholinobutanoic acid, (2S)-2-amino-4-[2-(morpholin-4-yl)ethoxy]butanoic acid, (2S)-2-amino-4-[2-(3,3-difluoropyrrolidin-1-yl)ethoxy]butanoic acid, (2S)-2-amino-4-(2-methylpropoxy)butanoic acid, (2R)-2-amino-3-(carboxymethoxy)propanoic acid, (2R)-2-amino-3-(2-aminoethoxy)propanoic acid, (2S)-2-amino-3-(2-azidoethoxy)propanoic acid, (2S)-2-amino-3-[2-(3,3-difluoropiperidin-1-yl)ethoxy]propanoic acid, (2S)-2-amino-3-[2-(3,3-difluoropyrrolidin-1-yl)ethoxy]propanoic acid, (2S)-2-amino-3-(2,2-difluoroethoxy)propanoic acid, (2S)-2-amino-3-(2-fluoroethoxy)propanoic acid, (2S)-2-amino-3-[(piperidin-4-yl)methoxy]propanoic acid, (2S)-2-amino-3-[(oxan-4-yl)methoxy]propanoic acid, (2S)-2-amino-3-(cyclohexylmethoxy)propanoic acid, (2S)-2-amino-3-(3-fluoropropoxy)propanoic acid, (R)-2-aminoheptanedioic acid, (S)-2-amino-3-(1-(2-hydroxyethyl)piperidin-4-yl)propanoic acid, (S)-2-amino-3-(1-(2-methoxyethyl)piperidin-4-yl)propanoic acid, (S)-2-amino-3-(1-(2,2,2-trifluoroethyl)piperidin-4-yl)propanoic acid, (S)-2-amino-3-(1-methylpiperidin-4-yl)propanoic acid, (S)-39-amino-5,33-dioxo-9,12,15,18,21,24,27,30-octaoxa-6,34-diazatetracont-1-yn-40-oic acid, (S)-2-amino-3-(4-((1-(3-(2-aminoethoxy)propanoyl)piperidin-4-yl)oxy)phenyl)propanoic acid (S)-2,4-diamino-2-methyl-4-oxobutanoic acid, (R)-2,4-diamino-2-methyl-4-oxobutanoic acid, (2S)-6-(4-carboxybutanamido)-2-(methylamino)hexanoic acid, (2S)-2-amino-3-{[2-(2-aminoethoxy)ethyl]carbamoyl}propanoic acid, (2S)-2-amino-4-{[2-(2-aminoethoxy)ethyl]carbamoyl}butanoic acid, 2-{[3-(pyridin-3-yl)propyl]amino}acetic acid, 2-({2-[3-(trifluoromethyl)phenyl]ethyl}amino)acetic acid, 2-{[3-(pyridin-4-yl)propyl]amino}acetic acid, 2-({2-[4-(trifluoromethyl)phenyl]ethyl}amino)acetic acid, 2-[(2-cyclobutylethyl)amino]acetic acid, 2-[(3,3-dimethylbutyl)amino]acetic acid, (R)-2-(methylamino)hept-6-enoic acid, (1S,2S)-2-(methylamino)cyclopentane-1-carboxylic acid, (2R,3S)-2-methyl-3-(methylamino)butanoic acid, (S)-2,4-bis(methylamino)butanoic acid, (2S,3S)-2-(methylamino)-3-phenylbutanoic acid, (S)-6-amino-2-(methylamino)-6-oxohexanoic acid, (S)-2-(methylamino)-6-morpholinohexanoic acid, (S)-4-cyclohexyl-2-(methylamino)butanoic acid, (S)-4-cyclopentyl-2-(methylamino)butanoic acid, (R)-4-cyclopentyl-2-(methylamino)butanoic acid, (S)-5-cyclohexyl-2-(methylamino)pentanoic acid, (R)-5-cyclohexyl-2-(methylamino)pentanoic acid, (S)-5-cyclopentyl-2-(methylamino)pentanoic acid, (S)-6,6-dimethyl-2-(methylamino)heptanoic acid, (R)-6,6-dimethyl-2-(methylamino)heptanoic acid, (2S)-3-[(2-chlorophenyl)methoxy]-2-(methylamino)propanoic acid, (2S)-3-(cyclohexyloxy)-2-(methylamino)propanoic acid, (2S,3S)-3-hydroxy-2-(methylamino)butanoic acid, (2S,3R)-2-(methylamino)-3-propoxybutanoic acid, 3-(methylamino)pentanedioic acid, (S)-3-((3-aminopropyl)amino)butanoic acid, (R)-3-((3-aminopropyl)amino)butanoic acid, (S)-3,5-bis(methylamino)pentanoic acid, (S)-7-amino-3-(methylamino)heptanoic acid, (R)-7-amino-3-(methylamino)heptanoic acid, (S)-3,7-bis(methylamino)heptanoic acid, (R)-2-amino-3-(4-((4-(carboxymethyl)piperazine-1-carboxamido)methyl)phenyl)propanoic acid, (S)-3-(4-((4-(carboxymethyl)piperazine-1-carboxamido)methyl)phenyl)-2-(methylamino)propanoic acid, (R)-3-(4-((4-(carboxymethyl)piperazine-1-carboxamido)methyl)phenyl)-2-(methylamino)propanoic acid, (2S)-6-{[4-(carboxymethyl)piperazine-1-carbonyl]amino}-2-(methylamino)hexanoic acid, (2S,4R)-4-(3-(carboxymethyl)ureido)pyrrolidine-2-carboxylic acid, (2S,4S)-4-(3-(carboxymethyl)ureido)pyrrolidine-2-carboxylic acid, (2R,4R)-4-(3-(carboxymethyl)ureido)pyrrolidine-2-carboxylic acid, (2R,4S)-4-(3-(carboxymethyl)ureido)pyrrolidine-2-carboxylic acid, (R)-2-amino-3-(4-(2-(4-(carboxymethyl)piperazine-1-carboxamido)ethoxy)phenyl)propanoic acid, (S)-3-(4-(2-(4-(carboxymethyl)piperazine-1-carboxamido)ethoxy)phenyl)-2-(methylamino)propanoic acid, (R)-3-(4-(2-(4-(carboxymethyl)piperazine-1-carboxamido)ethoxy)phenyl)-2-(methylamino)propanoic acid, (2R)-2-amino-3-(methylsulfamoyl)propanoic acid, (2R,4R)-4-(carboxymethoxy)pyrrolidine-2-carboxylic acid, (2R,4S)-4-(carboxymethoxy)pyrrolidine-2-carboxylic acid, (2S,4S)-4-(carboxymethoxy)pyrrolidine-2-carboxylic acid, (S)-2-amino-3-(7-(5-iodopyrimidin-2-yl)-1H-indol-3-yl)propanoic acid, (2S)-2-amino-4-{[2-(2,6-dioxopiperidin-3-yl)-1,3-dioxo-2,3-dihydro-1H-isoindol-4-yl]oxy}butanoic acid, (2S)-4-{[2-(2,6-dioxopiperidin-3-yl)-1,3-dioxo-2,3-dihydro-1H-isoindol-4-yl]oxy}-2-(methylamino)butanoic acid, and (2S)-2-amino-5-{[2-(2,6-dioxopiperidin-3-yl)-1,3-dioxo-2,3-dihydro-1H-isoindol-4-yl]oxy}pentanoic acid.

3. The amino acid derivative according to claim 1, wherein one or more functional groups within the amino acid are protected by protecting groups.

4. An amino acid derivative according to claim 3, wherein the amino acid is selected from the group consisting of (S)-2-((((9H-fluoren-9-yl)methoxy)carbonyl)amino)-3-(5-(((3-methylpentan-3-yl)oxy)carbonyl)pyridin-3-yl)propanoic acid, (S)-2-((((9H-fluoren-9-yl)methoxy)carbonyl)(methyl)amino)-3-(4-carbamoylpyridin-3-yl)propanoic acid, (S)-2-((((9H-fluoren-9-yl)methoxy)carbonyl)amino)-3-(6-(methylcarbamoyl)pyridin-3-yl)propanoic acid, (S)-2-((((9H-fluoren-9-yl)methoxy)carbonyl)amino)-3-(6-(((3-methylpentan-3-yl)oxy)carbonyl)pyridin-3-yl)propanoic acid, (S)-2-((((9H-fluoren-9-yl)methoxy)carbonyl)amino)-3-(6-(3-(tert-butoxy)-3-oxopropyl)pyridin-3-yl)propanoic acid, (S)-2-((((9H-fluoren-9-yl)methoxy)carbonyl)amino)-3-(6-ethylpyridin-3-yl)propanoic acid, (S)-2-((((9H-fluoren-9-yl)methoxy)carbonyl)amino)-3-(6-((1-(methylsulfonyl)piperidin-4-yl)oxy)pyridin-3-yl)propanoic acid, (S)-2-((((9H-fluoren-9-yl)methoxy)carbonyl)amino)-3-(6-((1-(2-(tert-butoxy)-2-oxoethyl)piperidin-4-yl)oxy)pyridin-3-yl)propanoic acid, (S)-2-((((9H-fluoren-9-yl)methoxy)carbonyl)amino)-3-(6-(3-chlorophenyl)pyridin-3-yl)propanoic acid, (S)-2-((((9H-fluoren-9-yl)methoxy)carbonyl)amino)-3-(6-(3-(tert-butoxy)phenyl)pyridin-3-yl)propanoic acid, (S)-2-((((9H-fluoren-9-yl)methoxy)carbonyl)amino)-3-(2-(((3-methylpentan-3-yl)oxy)carbonyl)pyridin-4-yl)propanoic acid, (S)-2-((((9H-fluoren-9-yl)methoxy)carbonyl)(methyl)amino)-3-(3-carbamoylpyridin-4-yl)propanoic acid, (S)-2-((((9H-fluoren-9-yl)methoxy)carbonyl)(methyl)amino)-3-(thiazol-4-yl)propanoic acid hydrochloride, (S)-2-((((9H-fluoren-9-yl)methoxy)carbonyl)amino)-3-(4-chloropyrimidin-5-yl)propanoic acid, (S)-2-((((9H-fluoren-9-yl)methoxy)carbonyl)amino)-3-(2-oxo-1,2-dihydroquinolin-5-yl)propanoic acid, (S)-2-((((9H-fluoren-9-yl)methoxy)carbonyl)amino)-3-(pyrazolo[1,5-a]pyridin-6-yl)propanoic acid, (S)-2-((((9H-fluoren-9-yl)methoxy)carbonyl)amino)-3-(2-oxo-1,2-dihydroquinolin-6-yl)propanoic acid, (S)-2-((((9H-fluoren-9-yl)methoxy)carbonyl)amino)-3-(imidazo[1,2-a]pyridin-7-yl)propanoic acid, (S)-2-((((9H-fluoren-9-yl)methoxy)carbonyl)amino)-3-(2′-(2-(tert-butoxy)-2-oxoethoxy)-[1,1′-biphenyl]-4-yl)propanoic acid, (S)-2-((((9H-fluoren-9-yl)methoxy)carbonyl)amino)-3-(3,4-bis(trifluoromethyl)phenyl)propanoic acid, (S)-2-((((9H-fluoren-9-yl)methoxy)carbonyl)amino)-3-(3-(1-(tert-butoxycarbonyl)piperidin-4-yl)phenyl)propanoic acid, (S)-2-((((9H-fluoren-9-yl)methoxy)carbonyl)amino)-3-(3-(tetrahydro-2H-pyran-4-yl)phenyl)propanoic acid, (S)-2-((((9H-fluoren-9-yl)methoxy)carbonyl)amino)-3-(3-cyclohexylphenyl)propanoic acid, (S)-2-((((9H-fluoren-9-yl)methoxy)carbonyl)amino)-3-(3-(methylcarbamoyl)phenyl)propanoic acid, (S)-2-((((9H-fluoren-9-yl)methoxy)carbonyl)amino)-3-(3-(N,N-bis(2,4-dimethoxybenzyl)sulfamoyl)phenyl)propanoic acid, (S)-2-((((9H-fluoren-9-yl)methoxy)carbonyl)amino)-3-(4-(5-(tert-butoxy)pyridin-3-yl)phenyl)propanoic acid, (R)-2-((((9H-fluoren-9-yl)methoxy)carbonyl)(methyl)amino)-3-(4-((((allyloxy)carbonyl)amino)methyl)phenyl)propanoic acid, (S)-2-((((9H-fluoren-9-yl)methoxy)carbonyl)amino)-3-(4-(N,N-bis(2,4-dimethoxybenzyl)sulfamoyl)phenyl)propanoic acid, (S)-2-((((9H-fluoren-9-yl)methoxy)carbonyl)amino)-4-(2-oxo-1,2-dihydroquinolin-5-yl)butanoic acid, (S)-2-((((9H-fluoren-9-yl)methoxy)carbonyl)amino)-4-(2-oxo-1,2-dihydroquinolin-6-yl)butanoic acid, (R)-2-((((9H-fluoren-9-yl)methoxy)carbonyl)(methyl)amino)-3-(quinolin-7-yl)propanoic acid, (R)-2-((((9H-fluoren-9-yl)methoxy)carbonyl)(methyl)amino)-3-(quinolin-3-yl)propanoic acid, (R)-2-((((9H-fluoren-9-yl)methoxy)carbonyl)(methyl)amino)-3-(quinolin-6-yl)propanoic acid, (S)-2-((((9H-fluoren-9-yl)methoxy)carbonyl)amino)-3-(2-(tert-butoxycarbonyl)quinolin-6-yl)propanoic acid, (R)-2-((((9H-fluoren-9-yl)methoxy)carbonyl)(methyl)amino)-3-(isoquinolin-6-yl)propanoic acid, (R)-2-((((9H-fluoren-9-yl)methoxy)carbonyl)(methyl)amino)-3-(isoquinolin-7-yl)propanoic acid, (S)-2-((((9H-fluoren-9-yl)methoxy)carbonyl)amino)-3-(2-(2-(tert-butoxy)-2-oxoethoxy)-[1,1′-biphenyl]-4-yl)propanoic acid, (S)-2-((((9H-fluoren-9-yl)methoxy)carbonyl)amino)-3-(1-(2-(tert-butoxy)-2-oxoethyl)-1H-pyrrolo[2,3-b]pyridin-3-yl)propanoic acid, (2S)-3-(1-{4-[(tert-butoxy)carbonyl]phenyl}-1H-indol-3-yl)-2-({[(9H-fluoren-9-yl)methoxy]carbonyl}amino)propanoic acid, (2S)-2-({[(9H-fluoren-9-yl)methoxy]carbonyl}amino)-3-[1-(4-methoxyphenyl)-1H-indol-3-yl]propanoic acid, (S)-2-((((9H-fluoren-9-yl)methoxy)carbonyl)(methyl)amino)-3-(1-(tert-butoxycarbonyl)-1H-pyrrolo[3,2-b]pyridin-3-yl)propanoic acid, (S)-2-((((9H-fluoren-9-yl)methoxy)carbonyl)(methyl)amino)-3-(1-(tert-butoxycarbonyl)-1H-pyrrolo[3,2-c]pyridin-3-yl)propanoic acid, (S)-2-((((9H-fluoren-9-yl)methoxy)carbonyl)amino)-3-(1-(tert-butoxycarbonyl)-6-((4-methoxybenzyl)oxy)-1H-indol-3-yl)propanoic acid, (S)-2-((((9H-fluoren-9-yl)methoxy)carbonyl)amino)-3-(1-(tert-butoxycarbonyl)-6-((tert-butoxycarbonyl)oxy)-1H-indol-3-yl)propanoic acid, (S)-2-((((9H-fluoren-9-yl)methoxy)carbonyl)amino)-3-(1-(4-(tert-butoxycarbonyl)phenyl)-6-((4-methoxybenzyl)oxy)-1H-indol-3-yl)propanoic acid, (S)-2-((((9H-fluoren-9-yl)methoxy)carbonyl)(methyl)amino)-3-(1-(tert-butoxycarbonyl)-1H-pyrrolo[2,3-c]pyridin-3-yl)propanoic acid, (R)-2-((((9H-fluoren-9-yl)methoxy)carbonyl)amino)-3-(1-(tert-butoxycarbonyl)-7-(pyrimidin-2-yl)-1H-indol-3-yl)propanoic acid, (S)-2-((((9H-fluoren-9-yl)methoxy)carbonyl)amino)-3-(1-(2,4-dimethoxybenzyl)-1H-pyrrolo[2,3-b]pyridin-3-yl)propanoic acid, (S)-2-((((9H-fluoren-9-yl)methoxy)carbonyl)amino)-3-(1-((2,4,6-triisopropylphenyl)sulfonyl)-1H-pyrrolo[2,3-b]pyridin-3-yl)propanoic acid, (S)-2-((((9H-fluoren-9-yl)methoxy)carbonyl)amino)-3-(1-(mesitylsulfonyl)-1H-pyrrolo[2,3-b]pyridin-3-yl)propanoic acid, (S)-2-((((9H-fluoren-9-yl)methoxy)carbonyl)amino)-3-(1-(4-methoxybenzyl)-1H-pyrrolo[2,3-b]pyridin-3-yl)propanoic acid, (S)-2-((((9H-fluoren-9-yl)methoxy)carbonyl)amino)-3-(7-(3-chlorophenyl)-1H-indol-3-yl)propanoic acid, (S)-2-((((9H-fluoren-9-yl)methoxy)carbonyl)amino)-3-(4-(pyridin-2-ylmethoxy)phenyl)propanoic acid, (S)-2-((((9H-fluoren-9-yl)methoxy)carbonyl)amino)-3-(4-(pyridin-3-ylmethoxy)phenyl)propanoic acid, (S)-2-((((9H-fluoren-9-yl)methoxy)carbonyl)amino)-3-(1-(tert-butoxycarbonyl)-7-ethoxy-1H-indol-3-yl)propanoic acid, (S)-2-((((9H-fluoren-9-yl)methoxy)carbonyl)amino)-3-(3-(((tert-butoxycarbonyl)(methyl)amino)methyl)phenyl)propanoic acid, (S)-2-((((9H-fluoren-9-yl)methoxy)carbonyl)amino)-3-(5-(((tert-butoxycarbonyl)amino)methyl)pyridin-3-yl)propanoic acid, (S)-2-((((9H-fluoren-9-yl)methoxy)carbonyl)amino)-3-((R)-3-((tert-butoxycarbonyl)amino)-2,3-dihydro-1H-inden-5-yl)propanoic acid, (S)-2-((((9H-fluoren-9-yl)methoxy)carbonyl)amino)-3-(3-(2-((tert-butoxycarbonyl)amino)ethyl)phenyl)propanoic acid, (S)-2-((((9H-fluoren-9-yl)methoxy)carbonyl)amino)-3-(4-(2-(tert-butoxy)-2-oxoethyl)-3,4-dihydro-2H-benzo[b][1,4]oxazin-6-yl)propanoic acid, (S)-2-((((9H-fluoren-9-yl)methoxy)carbonyl)amino)-3-(1-(tert-butoxycarbonyl)-1,2,3,4-tetrahydroquinolin-7-yl)propanoic acid, (2S)-3-{1-[4-(tert-butoxy)-4-oxobutyl]-1H-indol-3-yl}-2-({[(9H-fluoren-9-yl)methoxy]carbonyl}amino)propanoic acid, (2S)-3-{1-[3-(tert-butoxy)-3-oxopropyl]-1H-indol-3-yl}-2-({[(9H-fluoren-9-yl)methoxy]carbonyl}amino)propanoic acid, (S)-2-((((9H-fluoren-9-yl)methoxy)carbonyl)amino)-3-(4-phenylpiperidin-1-yl)propanoic acid, (R)-2-((((9H-fluoren-9-yl)methoxy)carbonyl)amino)-3-(4-phenylpiperidin-1-yl)propanoic acid, 2-((((9H-fluoren-9-yl)methoxy)carbonyl)amino)-3-(3,4-dihydroisoquinolin-2(1H)-yl)propanoic acid, (S)-2-((((9H-fluoren-9-yl)methoxy)carbonyl)amino)-3-(3′,5′-dichloro-[1,1′-biphenyl]-4-yl)propanoic acid, (S)-2-((((9H-fluoren-9-yl)methoxy)carbonyl)amino)-3-(3′-chloro-5′-fluoro-[1,1′-biphenyl]-4-yl)propanoic acid, (R)-2-((((9H-fluoren-9-yl)methoxy)carbonyl)amino)-3-(3′-carbamoyl-[1,1′-biphenyl]-4-yl)propanoic acid, (R)-2-((((9H-fluoren-9-yl)methoxy)carbonyl)amino)-3-(3′-(tert-butoxy)-[1,1′-biphenyl]-4-yl)propanoic acid, (R)-2-((((9H-fluoren-9-yl)methoxy)carbonyl)amino)-3-(4′-((2-((tert-butoxycarbonyl)amino)ethyl)carbamoyl)-[1,1′-biphenyl]-4-yl)propanoic acid, (R)-2-((((9H-fluoren-9-yl)methoxy)carbonyl)amino)-3-(4′-carbamoyl-[1,1′-biphenyl]-4-yl)propanoic acid, (S)-2-((((9H-fluoren-9-yl)methoxy)carbonyl)amino)-3-(2′-(tert-butoxycarbonyl)-[1,1′-biphenyl]-3-yl)propanoic acid, (S)-2-((((9H-fluoren-9-yl)methoxy)carbonyl)amino)-3-(2′-fluoro-[1,1′-biphenyl]-3-yl)propanoic acid, (S)-2-((((9H-fluoren-9-yl)methoxy)carbonyl)amino)-3-(2′-methoxy-[1,1′-biphenyl]-3-yl)propanoic acid, (S)-2-((((9H-fluoren-9-yl)methoxy)carbonyl)amino)-3-(3-(6-methoxypyridin-3-yl)phenyl)propanoic acid, (S)-2-((((9H-fluoren-9-yl)methoxy)carbonyl)amino)-3-(4′-fluoro-[1,1′-biphenyl]-3-yl)propanoic acid, (S)-2-((((9H-fluoren-9-yl)methoxy)carbonyl)amino)-3-(4′-methoxy-[1,1′-biphenyl]-3-yl)propanoic acid, (S)-2-((((9H-fluoren-9-yl)methoxy)carbonyl)amino)-3-(3-(2-oxo-1,2-dihydropyridin-4-yl)phenyl)propanoic acid, (S)-2-((((9H-fluoren-9-yl)methoxy)carbonyl)amino)-3-(3-(2-methoxypyridin-4-yl)phenyl)propanoic acid, (S)-2-((((9H-fluoren-9-yl)methoxy)carbonyl)amino)-3-(4-(2-((tert-butoxycarbonyl)amino)pyridin-3-yl)phenyl)propanoic acid, (2R,3S)-2-((((9H-fluoren-9-yl)methoxy)carbonyl)amino)-3-cyclopropyl-3-hydroxypropanoic acid, (S)-2-((((9H-fluoren-9-yl)methoxy)carbonyl)amino)-3-(1-phenylpiperidin-4-yl)propanoic acid, (R)-2-((((9H-fluoren-9-yl)methoxy)carbonyl)amino)-3-(1-phenylpiperidin-4-yl)propanoic acid, (R)-2-((((9H-fluoren-9-yl)methoxy)carbonyl)amino)-4-((tert-butoxycarbonyl)amino)-2-methylbutanoic acid, (S)-2-((((9H-fluoren-9-yl)methoxy)carbonyl)amino)-4-methoxy-2-methylbutanoic acid, (S)-2-((((9H-fluoren-9-yl)methoxy)carbonyl)amino)-3-methoxy-2-methylpropanoic acid, (S)-2-((((9H-fluoren-9-yl)methoxy)carbonyl)amino)-6-((R)-2,2-dimethyl-1,3-dioxolan-4-yl)hexanoic acid, (S)-2-((((9H-fluoren-9-yl)methoxy)carbonyl)amino)-6-((S)-2,2-dimethyl-1,3-dioxolan-4-yl)hexanoic acid, (R)-2-((((9H-fluoren-9-yl)methoxy)carbonyl)amino)-6-((R)-2,2-dimethyl-1,3-dioxolan-4-yl)hexanoic acid, (R)-2-((((9H-fluoren-9-yl)methoxy)carbonyl)amino)-6-((S)-2,2-dimethyl-1,3-dioxolan-4-yl)hexanoic acid, (S)-2-((((9H-fluoren-9-yl)methoxy)carbonyl)amino)-4-(1,1-dioxidothiomorpholino)butanoic acid hydrochloride, (S)-2-((((9H-fluoren-9-yl)methoxy)carbonyl)amino)-5-(1,1-dioxidothiomorpholino)pentanoic acid hydrochloride, (2R,3S)-2-((((9H-fluoren-9-yl)methoxy)carbonyl)amino)-3-hydroxypentanoic acid, (S)-2-((((9H-fluoren-9-yl)methoxy)carbonyl)amino)-3-(4-((1-((allyloxy)carbonyl)piperidin-4-yl)oxy)phenyl)propanoic acid, 4-((((9H-fluoren-9-yl)methoxy)carbonyl)amino)-1-(2-(2-methyl-5-nitro-1H-imidazol-1-yl)ethyl)piperidine-4-carboxylic acid, (R)-2-((((9H-fluoren-9-yl)methoxy)carbonyl)amino)-3-(1-(2-(allyloxy)-2-oxoethyl)piperidin-4-yl)propanoic acid, (R)-2-((((9H-fluoren-9-yl)methoxy)carbonyl)amino)-4-(methylsulfonamido)butanoic acid, (R)-2-((((9H-fluoren-9-yl)methoxy)carbonyl)amino)-3-(methylsulfonamido)propanoic acid, (R)-2-((((9H-fluoren-9-yl)methoxy)carbonyl)amino)-5-(((allyloxy)carbonyl)(methyl)amino)pentanoic acid, (S)-2-((((9H-fluoren-9-yl)methoxy)carbonyl)amino)-5-(((allyloxy)carbonyl)(methyl)amino)pentanoic acid, (9H-fluoren-9-yl)methyl (R)-(1-(tert-butoxy)-3-mercaptopropan-2-yl)carbamate, 2-(4-((((9H-fluoren-9-yl)methoxy)carbonyl)amino)piperidin-1-yl)acetic acid hydrochloride, (2S)-2-({[(9H-fluoren-9-yl)methoxy]carbonyl}amino)-4-({2-[(9H-xanthen-9-yl)carbamoyl]ethyl}carbamoyl)butanoic acid, (2S)-2-({[(9H-fluoren-9-yl)methoxy]carbonyl}amino)-4-({3-[(9H-xanthen-9-yl)carbamoyl]propyl}carbamoyl)butanoic acid, 2-[(2-{4-[(tert-butoxy)carbonyl]phenyl}ethyl)({[(9H-fluoren-9-yl)methoxy]carbonyl})amino]acetic acid, 2-{[2-(3,4-difluorophenyl)ethyl]({[(9H-fluoren-9-yl)methoxy]carbonyl})amino}acetic acid, 2-[(2-{3-[(tert-butoxy)carbonyl]phenyl}ethyl)({[(9H-fluoren-9-yl)methoxy]carbonyl})amino]acetic acid, 2-[(3-{3-[(tert-butoxy)carbonyl]phenyl}propyl)({[(9H-fluoren-9-yl)methoxy]carbonyl})amino]acetic acid, 2-[(2-{4-[2-(tert-butoxy)-2-oxoethyl]phenyl}ethyl)({[(9H-fluoren-9-yl)methoxy]carbonyl})amino]acetic acid, 2-[(3-{4-[(tert-butoxy)carbonyl]phenyl}propyl)({[(9H-fluoren-9-yl)methoxy]carbonyl})amino]acetic acid, 2-{[2-(4-{bis[(2,4-dimethoxyphenyl)methyl]sulfamoyl}phenyl)ethyl]({[(9H-fluoren-9-yl)methoxy]carbonyl})amino}acetic acid, (R)-2-((((9H-fluoren-9-yl)methoxy)carbonyl)(methyl)amino)-3-cyclopentylpropanoic acid, (S)-2-((((9H-fluoren-9-yl)methoxy)carbonyl)(methyl)amino)-7-((tert-butoxycarbonyl)amino)heptanoic acid, (2S)-2-({[(9H-fluoren-9-yl)methoxy]carbonyl}(methyl)amino)-4-(propan-2-yloxy)butanoic acid, (S)-2-((((9H-fluoren-9-yl)methoxy)carbonyl)(methyl)amino)-3-(methyl(phenyl)amino)propanoic acid, (2R)-2-({[(9H-fluoren-9-yl)methoxy]carbonyl}(propyl)amino)-3-phenylpropanoic acid, (2S)-2-({[(9H-fluoren-9-yl)methoxy]carbonyl}(methyl)amino)-3-(propan-2-yloxy)propanoic acid, (2S)-6-({[(9H-fluoren-9-yl)methoxy]carbonyl}amino)-2-{methyl[(prop-2-en-1-yloxy)carbonyl]amino}hexanoic acid, 3-((((9H-fluoren-9-yl)methoxy)carbonyl)(2-((tert-butoxycarbonyl)amino)ethyl)amino)propanoic acid, 11-(((9H-fluoren-9-yl)methoxy)carbonyl)-2,2-dimethyl-4-oxo-3,8-dioxa-5,11-diazatetradecan-14-oic acid, 3-((((9H-fluoren-9-yl)methoxy)carbonyl)(3-(((allyloxy)carbonyl)amino)propyl)amino)propanoic acid, 3-((((9H-fluoren-9-yl)methoxy)carbonyl)((1-(tert-butoxycarbonyl)azetidin-3-yl)methyl)amino)propanoic acid, 3-((((9H-fluoren-9-yl)methoxy)carbonyl)((1-(tert-butoxycarbonyl)piperidin-4-yl)methyl)amino)propanoic acid, (S)-7-(((9H-fluoren-9-yl)methoxy)carbonyl)-5,6,7,8-tetrahydro-1,7-naphthyridine-6-carboxylic acid, (2R)-2-({[(9H-fluoren-9-yl)methoxy]carbonyl}amino)-6-[(4-{2-[(3-methylpentan-3-yl)oxy]-2-oxoethyl}piperazine-1-carbonyl)amino]hexanoic acid, (2S)-2-((((9H-fluoren-9-yl)methoxy)carbonyl)amino)-3-(4-(3-((tetrahydro-2H-pyran-2-yl)oxy)phenyl)piperidin-1-yl)propanoic acid, (2R)-2-((((9H-fluoren-9-yl)methoxy)carbonyl)amino)-3-(4-(3-((tetrahydro-2H-pyran-2-yl)oxy)phenyl)piperidin-1-yl)propanoic acid, (S)-2-((((9H-fluoren-9-yl)methoxy)carbonyl)amino)-4-(N-(2,4-dimethoxybenzyl)sulfamoyl)butanoic acid, (2R)-3-{bis[(2,4-dimethoxyphenyl)methyl]sulfamoyl}-2-({[(9H-fluoren-9-yl)methoxy]carbonyl}amino)propanoic acid, (S)-2-((((9H-fluoren-9-yl)methoxy)carbonyl)amino)-3-(4-(((R)-1-(tert-butoxy)-1-oxopropan-2-yl)oxy)phenyl)propanoic acid, (S)-2-((((9H-fluoren-9-yl)methoxy)carbonyl)amino)-3-(4-(((S)-1-(tert-butoxy)-1-oxopropan-2-yl)oxy)phenyl)propanoic acid, (2R,3S)-2-((((9H-fluoren-9-yl)methoxy)carbonyl)amino)-3-((tert-butoxycarbonyl)amino)butanoic acid, (R)-5-((((9H-fluoren-9-yl)methoxy)carbonyl)(methyl)amino)-6-phenylhexanoic acid, 2-{[3-({[(9H-fluoren-9-yl)methoxy]carbonyl}amino)propyl][(prop-2-en-1-yloxy)carbonyl]amino}acetic acid, 1-(2-(allyloxy)-2-oxoethyl)piperidine-4-carboxylic acid hydrochloride, 3-((2-((((9H-fluoren-9-yl)methoxy)carbonyl)(methyl)amino)ethyl)(tert-butoxycarbonyl)amino)propanoic acid, (S)-2-((((9H-fluoren-9-yl)methoxy)carbonyl)amino)-3-(1-(tert-butoxycarbonyl)-7-(pyrimidin-2-yl)-1H-indol-3-yl)propanoic acid, (S)-2-((((9H-fluoren-9-yl)methoxy)carbonyl)(methyl)amino)-5-(3-chloropyridin-2-yl)pentanoic acid, (S)-2-((((9H-fluoren-9-yl)methoxy)carbonyl)amino)-3-(5-(2-fluorophenyl)pyridin-3-yl)propanoic acid, (S)-2-((((9H-fluoren-9-yl)methoxy)carbonyl)amino)-3-(5-(2-methoxyphenyl)pyridin-3-yl)propanoic acid, (S)-3-(6-(1H-imidazol-1-yl)pyridin-3-yl)-2-((((9H-fluoren-9-yl)methoxy)carbonyl)amino)propanoic acid, (S)-2-((((9H-fluoren-9-yl)methoxy)carbonyl)amino)-3-(6-(piperidin-1-yl)pyridin-3-yl)propanoic acid, (R)-2-((((9H-fluoren-9-yl)methoxy)carbonyl)(methyl)amino)-3-(6-((tert-butoxycarbonyl)amino)pyridin-3-yl)propanoic acid, (S)-2-((((9H-fluoren-9-yl)methoxy)carbonyl)amino)-3-(1-(2-((3-methylpentan-3-yl)oxy)-2-oxoethyl)-1H-pyrazol-3-yl)propanoic acid, (R)-2-((((9H-fluoren-9-yl)methoxy)carbonyl)amino)-3-(1H-pyrazolo[3,4-b]pyridin-4-yl)propanoic acid, (R)-2-((((9H-fluoren-9-yl)methoxy)carbonyl)amino)-3-(2-((tert-butoxycarbonyl)(methyl)amino)pyridin-4-yl)propanoic acid, (R)-2-((((9H-fluoren-9-yl)methoxy)carbonyl)(methyl)amino)-3-(2-((tert-butoxycarbonyl)amino)pyridin-4-yl)propanoic acid, (S)-2-((((9H-fluoren-9-yl)methoxy)carbonyl)amino)-3-(1-(2-((3-methylpentan-3-yl)oxy)-2-oxoethyl)-1H-pyrazol-4-yl)propanoic acid, (S)-2-((((9H-fluoren-9-yl)methoxy)carbonyl)(methyl)amino)-3-(2-oxo-1,2-dihydroquinolin-4-yl)propanoic acid, (S)-2-((((9H-fluoren-9-yl)methoxy)carbonyl)amino)-3-(1H-pyrazolo[3,4-b]pyridin-5-yl)propanoic acid, (S)-2-((((9H-fluoren-9-yl)methoxy)carbonyl)(methyl)amino)-3-(1H-pyrazolo[3,4-b]pyridin-5-yl)propanoic acid, (R)-2-((((9H-fluoren-9-yl)methoxy)carbonyl)amino)-3-(2-aminoquinazolin-5-yl)propanoic acid, (S)-2-((((9H-fluoren-9-yl)methoxy)carbonyl)amino)-3-(1H-indazol-6-yl)propanoic acid, (S)-2-((((9H-fluoren-9-yl)methoxy)carbonyl)(methyl)amino)-3-(3-(2-(tert-butoxy)-2-oxoethoxy)phenyl)propanoic acid, (S)-2-((((9H-fluoren-9-yl)methoxy)carbonyl)amino)-3-(3-(2-amino-2-oxoethoxy)phenyl)propanoic acid, (S)-2-((((9H-fluoren-9-yl)methoxy)carbonyl)(methyl)amino)-3-(4-(2-(tert-butoxy)-2-oxoethoxy)phenyl)propanoic acid, (R)-2-((((9H-fluoren-9-yl)methoxy)carbonyl)(methyl)amino)-3-(4-(2-(tert-butoxy)-2-oxoethoxy)phenyl)propanoic acid, (S)-2-((((9H-fluoren-9-yl)methoxy)carbonyl)amino)-4-(2-carbamoylpyridin-3-yl)butanoic acid, (S)-2-((((9H-fluoren-9-yl)methoxy)carbonyl)amino)-4-(4-carbamoylpyridin-3-yl)butanoic acid, (S)-2-((((9H-fluoren-9-yl)methoxy)carbonyl)amino)-4-(6-((2-amino-2-oxoethyl)amino)pyridin-3-yl)butanoic acid, (S)-2-((((9H-fluoren-9-yl)methoxy)carbonyl)amino)-4-(6-((tert-butoxycarbonyl)(methyl)amino)pyridin-3-yl)butanoic acid, (R)-2-((((9H-fluoren-9-yl)methoxy)carbonyl)amino)-4-(6-((tert-butoxycarbonyl)amino)pyridin-3-yl)butanoic acid, (S)-2-((((9H-fluoren-9-yl)methoxy)carbonyl)(methyl)amino)-4-(6-(trifluoromethyl)pyridin-3-yl)butanoic acid, (R)-2-((((9H-fluoren-9-yl)methoxy)carbonyl)amino)-4-(2-((tert-butoxycarbonyl)amino)pyrimidin-5-yl)butanoic acid, (S)-2-((((9H-fluoren-9-yl)methoxy)carbonyl)amino)-4-(4-(tert-butoxycarbonyl)-3,4-dihydro-2H-benzo[b][1,4]oxazin-7-yl)butanoic acid, (S)-2-((((9H-fluoren-9-yl)methoxy)carbonyl)(methyl)amino)-4-(2,3-dihydrobenzo[b][1,4]dioxin-5-yl)butanoic acid, (S)-2-((((9H-fluoren-9-yl)methoxy)carbonyl)(methyl)amino)-4-(2,3-dimethoxyphenyl)butanoic acid, (S)-2-((((9H-fluoren-9-yl)methoxy)carbonyl)(methyl)amino)-4-(benzo[d][1,3]dioxol-4-yl)butanoic acid, (S)-2-((((9H-fluoren-9-yl)methoxy)carbonyl)(methyl)amino)-4-(2,4-dimethoxyphenyl)butanoic acid, (R)-2-((((9H-fluoren-9-yl)methoxy)carbonyl)amino)-4-(2-(tert-butoxycarbonyl)phenyl)butanoic acid, (S)-2-((((9H-fluoren-9-yl)methoxy)carbonyl)(methyl)amino)-4-(2-((allyloxy)carbonyl)phenyl)butanoic acid, (R)-2-((((9H-fluoren-9-yl)methoxy)carbonyl)(methyl)amino)-4-(2-((allyloxy)carbonyl)phenyl)butanoic acid, (S)-2-((((9H-fluoren-9-yl)methoxy)carbonyl)(methyl)amino)-4-(2-(tert-butoxycarbonyl)phenyl)butanoic acidm (R)-2-((((9H-fluoren-9-yl)methoxy)carbonyl)(methyl)amino)-4-(2-(tert-butoxycarbonyl)phenyl)butanoic acid, (S)-2-((((9H-fluoren-9-yl)methoxy)carbonyl)(methyl)amino)-4-(3-((allyloxy)carbonyl)phenyl)butanoic acid, (R)-2-((((9H-fluoren-9-yl)methoxy)carbonyl)(methyl)amino)-4-(3-((allyloxy)carbonyl)phenyl)butanoic acid, (S)-2-((((9H-fluoren-9-yl)methoxy)carbonyl)(methyl)amino)-4-(o-tolyl)butanoic acid, (S)-2-((((9H-fluoren-9-yl)methoxy)carbonyl)(methyl)amino)-4-(3,4-difluorophenyl)butanoic acid, (S)-2-((((9H-fluoren-9-yl)methoxy)carbonyl)(methyl)amino)-4-(3,5-difluorophenyl)butanoic acid, (S)-2-((((9H-fluoren-9-yl)methoxy)carbonyl)(methyl)amino)-4-(3-(tert-butoxycarbonyl)phenyl)butanoic acid, (R)-2-((((9H-fluoren-9-yl)methoxy)carbonyl)(methyl)amino)-4-(3-(tert-butoxycarbonyl)phenyl)butanoic acid, (S)-2-((((9H-fluoren-9-yl)methoxy)carbonyl)(methyl)amino)-4-(4-(tert-butoxycarbonyl)phenyl)butanoic acid, (R)-2-((((9H-fluoren-9-yl)methoxy)carbonyl)(methyl)amino)-4-(4-(tert-butoxycarbonyl)phenyl)butanoic acid, (S)-2-((((9H-fluoren-9-yl)methoxy)carbonyl)(methyl)amino)-4-(3-(trifluoromethyl)phenyl)butanoic acid, (S)-2-((((9H-fluoren-9-yl)methoxy)carbonyl)(methyl)amino)-4-(4-((allyloxy)carbonyl)phenyl)butanoic acid, (R)-2-((((9H-fluoren-9-yl)methoxy)carbonyl)(methyl)amino)-4-(4-((allyloxy)carbonyl)phenyl)butanoic acid, (S)-2-((((9H-fluoren-9-yl)methoxy)carbonyl)(methyl)amino)-4-(4-(trifluoromethyl)phenyl)butanoic acid, (R)-2-((((9H-fluoren-9-yl)methoxy)carbonyl)amino)-3-(2-((tert-butoxycarbonyl)amino)quinolin-5-yl)propanoic acid, (S)-2-((((9H-fluoren-9-yl)methoxy)carbonyl)amino)-3-(2-methoxy-[1,1′-biphenyl]-4-yl)propanoic acid, (S)-2-((((9H-fluoren-9-yl)methoxy)carbonyl)(methyl)amino)-5-(2-chlorophenyl)pentanoic acid, (S)-2-((((9H-fluoren-9-yl)methoxy)carbonyl)(methyl)amino)-5-(2-chloro-3-fluorophenyl)pentanoic acid, (S)-2-((((9H-fluoren-9-yl)methoxy)carbonyl)(methyl)amino)-5-(2-chloro-5-fluorophenyl)pentanoic acid, (S)-2-((((9H-fluoren-9-yl)methoxy)carbonyl)(methyl)amino)-5-(2-fluorophenyl)pentanoic acid, (S)-2-((((9H-fluoren-9-yl)methoxy)carbonyl)(methyl)amino)-5-(3-chlorophenyl)pentanoic acid, (S)-2-((((9H-fluoren-9-yl)methoxy)carbonyl)(methyl)amino)-5-(3-fluorophenyl)pentanoic acid, (S)-2-((((9H-fluoren-9-yl)methoxy)carbonyl)(methyl)amino)-5-(3-(trifluoromethyl)phenyl)pentanoic acid, (S)-2-((((9H-fluoren-9-yl)methoxy)carbonyl)(methyl)amino)-5-(4-chlorophenyl)pentanoic acid, (S)-2-((((9H-fluoren-9-yl)methoxy)carbonyl)(methyl)amino)-5-(4-fluorophenyl)pentanoic acid, (S)-2-((((9H-fluoren-9-yl)methoxy)carbonyl)(methyl)amino)-5-(4-methoxyphenyl)pentanoic acid, (S)-2-((((9H-fluoren-9-yl)methoxy)carbonyl)(methyl)amino)-5-(4-(trifluoromethyl)phenyl)pentanoic acid, (S)-2-((((9H-fluoren-9-yl)methoxy)carbonyl)amino)-3-(1-(2-(tert-butoxy)-2-oxoethyl)-5-chloro-1H-indol-3-yl)propanoic acid, (S)-2-((((9H-fluoren-9-yl)methoxy)carbonyl)amino)-3-(5-fluoro-1H-indazol-3-yl)propanoic acid, (S)-2-((((9H-fluoren-9-yl)methoxy)carbonyl)amino)-3-(6-chloro-1H-pyrrolo[2,3-b]pyridin-3-yl)propanoic acid, (S)-2-((((9H-fluoren-9-yl)methoxy)carbonyl)amino)-3-(6-((tert-butoxycarbonyl)oxy)-1-(4-(tert-butoxycarbonyl)phenyl)-1H-indol-3-yl)propanoic acid, (S)-2-((((9H-fluoren-9-yl)methoxy)carbonyl)(methyl)amino)-3-(4-(2-(((allyloxy)carbonyl)amino)ethoxy)phenyl)propanoic acid, (S)-2-((((9H-fluoren-9-yl)methoxy)carbonyl)(methyl)amino)-3-(4-(2-((tert-butoxycarbonyl)amino)ethoxy)phenyl)propanoic acid, (S)-2-((((9H-fluoren-9-yl)methoxy)carbonyl)amino)-3-(4-(3-((tert-butoxycarbonyl)amino)propoxy)phenyl)propanoic acid, (S)-2-((((9H-fluoren-9-yl)methoxy)carbonyl)amino)-3-morpholinopropanoic acid hydrochloride, (S)-2-((((9H-fluoren-9-yl)methoxy)carbonyl)amino)-3-(4-phenylpiperidin-1-yl)propanoic acid hydrochloride, (R)-2-((((9H-fluoren-9-yl)methoxy)carbonyl)amino)-3-(4-phenylpiperidin-1-yl)propanoic acid hydrochloride, (S)-2-((((9H-fluoren-9-yl)methoxy)carbonyl)amino)-3-(4-phenylpiperazin-1-yl)propanoic acid dihydrochloride, (R)-2-((((9H-fluoren-9-yl)methoxy)carbonyl)amino)-3-(4-phenylpiperazin-1-yl)propanoic acid dihydrochloride, (S)-2-((((9H-fluoren-9-yl)methoxy)carbonyl)amino)-4-morpholinobutanoic acid hydrochloride, (2S)-2-({[(9H-fluoren-9-yl)methoxy]carbonyl}amino)-4-[2-(morpholin-4-yl)ethoxy]butanoic acid, (2S)-4-[2-(3,3-difluoropyrrolidin-1-yl)ethoxy]-2-({[(9H-fluoren-9-yl)methoxy]carbonyl}amino)butanoic acid, (2S)-2-({[(9H-fluoren-9-yl)methoxy]carbonyl}amino)-4-(2-methylpropoxy)butanoic acid, (2R)-2-({[(9H-fluoren-9-yl)methoxy]carbonyl}amino)-3-[2-oxo-2-(prop-2-en-1-yloxy)ethoxy]propanoic acid, (2R)-3-(2-{[(tert-butoxy)carbonyl]amino}ethoxy)-2-({[(9H-fluoren-9-yl)methoxy]carbonyl}amino)propanoic acid, (2S)-3-(2-azidoethoxy)-2-({[(9H-fluoren-9-yl)methoxy]carbonyl}amino)propanoic acid, (2S)-3-[2-(3,3-difluoropiperidin-1-yl)ethoxy]-2-({[(9H-fluoren-9-yl)methoxy]carbonyl}amino)propanoic acid, (2S)-3-[2-(3,3-difluoropyrrolidin-1-yl)ethoxy]-2-({[(9H-fluoren-9-yl)methoxy]carbonyl}amino)propanoic acid, (2S)-3-(2,2-difluoroethoxy)-2-({[(9H-fluoren-9-yl)methoxy]carbonyl}amino)propanoic acid, (2S)-2-({[(9H-fluoren-9-yl)methoxy]carbonyl}amino)-3-(2-fluoroethoxy)propanoic acid, (2S)-3-({1-[(tert-butoxy)carbonyl]piperidin-4-yl}methoxy)-2-({[(9H-fluoren-9-yl)methoxy]carbonyl}amino)propanoic acid, (2S)-2-({[(9H-fluoren-9-yl)methoxy]carbonyl}amino)-3-[(oxan-4-yl)methoxy]propanoic acid, (2S)-3-(cyclohexylmethoxy)-2-({[(9H-fluoren-9-yl)methoxy]carbonyl}amino)propanoic acid, (2S)-2-({[(9H-fluoren-9-yl)methoxy]carbonyl}amino)-3-(3-fluoropropoxy)propanoic acid, (R)-2-((((9H-fluoren-9-yl)methoxy)carbonyl)amino)-7-(allyloxy)-7-oxoheptanoic acid, (S)-2-((((9H-fluoren-9-yl)methoxy)carbonyl)amino)-3-(1-(2-hydroxyethyl)piperidin-4-yl)propanoic acid, (S)-2-((((9H-fluoren-9-yl)methoxy)carbonyl)amino)-3-(1-(2-methoxyethyl)piperidin-4-yl)propanoic acid, (S)-2-((((9H-fluoren-9-yl)methoxy)carbonyl)amino)-3-(1-(2,2,2-trifluoroethyl)piperidin-4-yl)propanoic acid, (S)-2-((((9H-fluoren-9-yl)methoxy)carbonyl)amino)-3-(1-methylpiperidin-4-yl)propanoic acid hydrochloride, (S)-39-((((9H-fluoren-9-yl)methoxy)carbonyl)amino)-5,33-dioxo-9,12,15,18,21,24,27,30-octaoxa-6,34-diazatetracont-1-yn-40-oic acid, (S)-2-((((9H-fluoren-9-yl)methoxy)carbonyl)amino)-3-(4-((1-(3-(2-((tert-butoxycarbonyl)amino)ethoxy)propanoyl)piperidin-4-yl)oxy)phenyl)propanoic acid, (S)-2-((((9H-fluoren-9-yl)methoxy)carbonyl)amino)-4-((9H-xanthen-9-yl)amino)-2-methyl-4-oxobutanoic acid, (R)-2-((((9H-fluoren-9-yl)methoxy)carbonyl)amino)-4-((9H-xanthen-9-yl)amino)-2-methyl-4-oxobutanoic acid, (2S)-6-[5-(tert-butoxy)-5-oxopentanamido]-2-({[(9H-fluoren-9-yl)methoxy]carbonyl}(methyl)amino)hexanoic acid, (S)-14-((((9H-fluoren-9-yl)methoxy)carbonyl)amino)-2,2-dimethyl-4,12-dioxo-3,8-dioxa-5,11-diazapentadecan-15-oic acid, (S)-15-((((9H-fluoren-9-yl)methoxy)carbonyl)amino)-2,2-dimethyl-4,12-dioxo-3,8-dioxa-5,11-diazahexadecan-16-oic acid, 2-({[(9H-fluoren-9-yl)methoxy]carbonyl}[3-(pyridin-3-yl)propyl]amino)acetic acid hydrochloride, 2-({[(9H-fluoren-9-yl)methoxy]carbonyl}({2-[3-(trifluoromethyl)phenyl]ethyl})amino)acetic acid, 2-({[(9H-fluoren-9-yl)methoxy]carbonyl}[3-(pyridin-4-yl)propyl]amino)acetic acid hydrochloride, 2-({[(9H-fluoren-9-yl)methoxy]carbonyl}({2-[4-(trifluoromethyl)phenyl]ethyl})amino)acetic acid, 2-[(2-cyclobutylethyl)({[(9H-fluoren-9-yl)methoxy]carbonyl})amino]acetic acid, 2-[(3,3-dimethylbutyl)({[(9H-fluoren-9-yl)methoxy]carbonyl})amino]acetic acid, (R)-2-((((9H-fluoren-9-yl)methoxy)carbonyl)(methyl)amino)hept-6-enoic acid, (1S,2S)-2-((((9H-fluoren-9-yl)methoxy)carbonyl)(methyl)amino)cyclopentane-1-carboxylic acid, (2R,3S)-3-((((9H-fluoren-9-yl)methoxy)carbonyl)(methyl)amino)-2-methylbutanoic acid, (S)-2-((((9H-fluoren-9-yl)methoxy)carbonyl)(methyl)amino)-4-((tert-butoxycarbonyl)(methyl)amino)butanoic acid, (2S,3S)-2-((((9H-fluoren-9-yl)methoxy)carbonyl)(methyl)amino)-3-phenylbutanoic acid, (S)-2-((((9H-fluoren-9-yl)methoxy)carbonyl)(methyl)amino)-6-amino-6-oxohexanoic acid, (S)-2-((((9H-fluoren-9-yl)methoxy)carbonyl)(methyl)amino)-6-morpholinohexanoic acid hydrochloride, (S)-2-((((9H-fluoren-9-yl)methoxy)carbonyl)(methyl)amino)-4-cyclohexylbutanoic acid, (S)-2-((((9H-fluoren-9-yl)methoxy)carbonyl)(methyl)amino)-4-cyclopentylbutanoic acid, (R)-2-((((9H-fluoren-9-yl)methoxy)carbonyl)(methyl)amino)-4-cyclopentylbutanoic acid, (S)-2-((((9H-fluoren-9-yl)methoxy)carbonyl)(methyl)amino)-5-cyclohexylpentanoic acid, (R)-2-((((9H-fluoren-9-yl)methoxy)carbonyl)(methyl)amino)-5-cyclohexylpentanoic acid, (S)-2-((((9H-fluoren-9-yl)methoxy)carbonyl)(methyl)amino)-5-cyclopentylpentanoic acid, (S)-2-((((9H-fluoren-9-yl)methoxy)carbonyl)(methyl)amino)-6,6-dimethylheptanoic acid, (R)-2-((((9H-fluoren-9-yl)methoxy)carbonyl)(methyl)amino)-6,6-dimethylheptanoic acid, (2S)-3-[(2-chlorophenyl)methoxy]-2-({[(9H-fluoren-9-yl)methoxy]carbonyl}(methyl)amino)propanoic acid, (2S)-3-(cyclohexyloxy)-2-({[(9H-fluoren-9-yl)methoxy]carbonyl}(methyl)amino)propanoic acid, (2S,3S)-3-(tert-butoxy)-2-({[(9H-fluoren-9-yl)methoxy]carbonyl}(methyl)amino)butanoic acid, (2S,3R)-2-({[(9H-fluoren-9-yl)methoxy]carbonyl}(methyl)amino)-3-propoxybutanoic acid, (R)-3-((((9H-fluoren-9-yl)methoxy)carbonyl)(methyl)amino)-5-((3-methylpentan-3-yl)oxy)-5-oxopentanoic acid, (S)-3-((((9H-fluoren-9-yl)methoxy)carbonyl)(3-((tert-butoxycarbonyl)amino)propyl)amino)butanoic acid, (R)-3-((((9H-fluoren-9-yl)methoxy)carbonyl)(3-((tert-butoxycarbonyl)amino)propyl)amino)butanoic acid, (S)-3-((((9H-fluoren-9-yl)methoxy)carbonyl)(methyl)amino)-5-((tert-butoxycarbonyl)(methyl)amino)pentanoic acid, (S)-3-((((9H-fluoren-9-yl)methoxy)carbonyl)(methyl)amino)-7-((tert-butoxycarbonyl)amino)heptanoic acid, (R)-3-((((9H-fluoren-9-yl)methoxy)carbonyl)(methyl)amino)-7-((tert-butoxycarbonyl)amino)heptanoic acid, (S)-3-((((9H-fluoren-9-yl)methoxy)carbonyl)(methyl)amino)-7-((tert-butoxycarbonyl)(methyl)amino)heptanoic acid, (R)-2-((((9H-fluoren-9-yl)methoxy)carbonyl)amino)-3-(4-((4-(2-((3-methylpentan-3-yl)oxy)-2-oxoethyl)piperazine-1-carboxamido)methyl)phenyl)propanoic acid, (S)-2-((((9H-fluoren-9-yl)methoxy)carbonyl)(methyl)amino)-3-(4-((4-(2-((3-methylpentan-3-yl)oxy)-2-oxoethyl)piperazine-1-carboxamido)methyl)phenyl)propanoic acid, (R)-2-((((9H-fluoren-9-yl)methoxy)carbonyl)(methyl)amino)-3-(4-((4-(2-((3-methylpentan-3-yl)oxy)-2-oxoethyl)piperazine-1-carboxamido)methyl)phenyl)propanoic acid, (2S)-2-({[(9H-fluoren-9-yl)methoxy]carbonyl}(methyl)amino)-6-[(4-{2-[(3-methylpentan-3-yl)oxy]-2-oxoethyl}piperazine-1-carbonyl)amino]hexanoic acid, (2S,4R)-1-(((9H-fluoren-9-yl)methoxy)carbonyl)-4-(3-(2-(tert-butoxy)-2-oxoethyl)ureido)pyrrolidine-2-carboxylic acid, (2S,4S)-1-(((9H-fluoren-9-yl)methoxy)carbonyl)-4-(3-(2-(tert-butoxy)-2-oxoethyl)ureido)pyrrolidine-2-carboxylic acid, (2R,4R)-1-(((9H-fluoren-9-yl)methoxy)carbonyl)-4-(3-(2-(tert-butoxy)-2-oxoethyl)ureido)pyrrolidine-2-carboxylic acid, (2R,4S)-1-(((9H-fluoren-9-yl)methoxy)carbonyl)-4-(3-(2-(tert-butoxy)-2-oxoethyl)ureido)pyrrolidine-2-carboxylic acid, (R)-2-((((9H-fluoren-9-yl)methoxy)carbonyl)amino)-3-(4-(2-(4-(2-((3-methylpentan-3-yl)oxy)-2-oxoethyl)piperazine-1-carboxamido)ethoxy)phenyl)propanoic acid, (S)-2-((((9H-fluoren-9-yl)methoxy)carbonyl)(methyl)amino)-3-(4-(2-(4-(2-((3-methylpentan-3-yl)oxy)-2-oxoethyl)piperazine-1-carboxamido)ethoxy)phenyl)propanoic acid, (R)-2-((((9H-fluoren-9-yl)methoxy)carbonyl)(methyl)amino)-3-(4-(2-(4-(2-((3-methylpentan-3-yl)oxy)-2-oxoethyl)piperazine-1-carboxamido)ethoxy)phenyl)propanoic acid, (2R)-3-{[(2,4-dimethoxyphenyl)methyl](methyl)sulfamoyl}-2-({[(9H-fluoren-9-yl)methoxy]carbonyl}amino)propanoic acid, (2R,4R)-1-(((9H-fluoren-9-yl)methoxy)carbonyl)-4-(2-(tert-butoxy)-2-oxoethoxy)pyrrolidine-2-carboxylic acid, (2R,4S)-1-(((9H-fluoren-9-yl)methoxy)carbonyl)-4-(2-(tert-butoxy)-2-oxoethoxy)pyrrolidine-2-carboxylic acid, (2S,4S)-1-(((9H-fluoren-9-yl)methoxy)carbonyl)-4-(2-(tert-butoxy)-2-oxoethoxy)pyrrolidine-2-carboxylic acid, (S)-2-((((9H-fluoren-9-yl)methoxy)carbonyl)amino)-3-(7-(5-iodopyrimidin-2-yl)-1H-indol-3-yl)propanoic acid, (2S)-4-{[2-(2,6-dioxopiperidin-3-yl)-1,3-dioxo-2,3-dihydro-1H-isoindol-4-yl]oxy}-2-({[(9H-fluoren-9-yl)methoxy]carbonyl}amino)butanoic acid, (2S)-4-{[2-(2,6-dioxopiperidin-3-yl)-1,3-dioxo-2,3-dihydro-1H-isoindol-4-yl]oxy}-2-({[(9H-fluoren-9-yl)methoxy]carbonyl}(methyl)amino)butanoic acid, and (2S)-5-{[2-(2,6-dioxopiperidin-3-yl)-1,3-dioxo-2,3-dihydro-1H-isoindol-4-yl]oxy}-2-({[(9H-fluoren-9-yl)methoxy]carbonyl}amino)pentanoic acid.

5. An enantiomer, diastereomer or racemate of the amino acid derivative according to claim 1.
